SEQUENCE LISTING (70 sequences)

SEQ ID NO: 1 - protein, 478 aa, Homo sapiens
MAPLRPLLILALLAWVALADQESCKGRCTEGFNVDKKCQCDELCSYYQSCCTDYTAECKPQVTRGDVFTMPEDEYTVYDDGEEKNNATVHEQVGGPSLTSDLQAQSKGNPEQTPVLKPEEEAPAPEVGASKPEGIDSRPETLHPGRPQPPAEEELCSGKPFDAFTDLKNGSLFAFRGQYCYELDEKAVRPGYPKLIRDVWGIEGPIDAAFTRINCQGKTYLFKGSQYWRFEDGVLDPDYPRNISDGFDGIPDNVDAALALPAHSYSGRERVYFFKGKQYWEYQFQHQPSQEECEGSSLSAVFEHFAMMQRDSWEDIFELLFWGRTSAGTRQPQFISRDWHGVPGQVDAAMAGRIYISGMAPRPSLAKKQRFRHRNRKGYRSQRGHSRGRNQNSRRPSRATWLSLFSSEESNLGANNYDDYRMDWLVPATCEPIQSVFFFSGDKYYRVNLRTRRVDTVDPPYPRSIAQYWLGCPAPGHL

SEQ ID NO: 2 - protein, 302 aa, Homo sapiens
MCAERLGQFMTLALVLATFDPARGTDATNPPEGPQDRSSQQKGRLSLQNTAEIQHCLVNAGDVGCGVFECFENNSCEIRGLHGICMTFLHNAGKFDAQGKSFIKDALKCKAHALRHRFGCISRKCPAIREMVSQLQRECYLKHDLCAAAQENTRVIVEMIHFKDLLLHEPYVDLVNLLLTCGEEVKEAITHSVQVQCEQNWGSLCSILSFCTSAIQKPPTAPPERQPQVDRTKLSRAHHGEAGHHLPEPSSRETGRGAKGERGSKSHPNAHARGRVGGLGAQGPSGSSEWEDEQSEYSDIRR

SEQ ID NO: 3 - protein, 2068 aa, Homo sapiens
MAGRSHPGPLRPLLPLLVVAACVLPGAGGTCPERALERREEEANVVLTGTVEEILNVDPVQHTYSCKVRVWRYLKGKDLVARESLLDGGNKVVISGFGDPLICDNQVSTGDTRIFFVNPAPPYLWPAHKNELMLNSSLMRITLRNLEEVEFCVEDKPGTHFTPVPPTPPDACRGMLCGFGAVCEPNAEGPGRASCVCKKSPCPSVVAPVCGSDASTYSNECELQRAQCSQQRRIRLLSRGPCGSRDPCSNVTCSFGSTCARSADGLTASCLCPATCRGAPEGTVCGSDGADYPGECQLLRRACARQENVFKKFDGPCDPCQGALPDPSRSCRVNPRTRRPEMLLRPESCPARQAPVCGDDGVTYENDCVMGRSGAARGLLLQKVRSGQCQGRDQCPEPCRFNAVCLSRRGRPRCSCDRVTCDGAYRPVCAQDGRTYDSDCWRQQAECRQQRAIPSKHQGPCDQAPSPCLGVQCAFGATCAVKNGQAACECLQACSSLYDPVCGSDGVTYGSACELEATACTLGREIQVARKGPCDRCGQCRFGALCEAETGRCVCPSECVALAQPVCGSDGHTYPSECMLHVHACTHQISLHVASAGPCETCGDAVCAFGAVCSAGQCVCPRCEHPPPGPVCGSDGVTYGSACELREAACLQQTQIEEARAGPCEQAECGSGGSGSGEDGDCEQELCRQRGGIWDEDSEDGPCVCDFSCQSVPGSPVCGSDGVTYSTECELKKARCESQRGLYVAAQGACRGPTFAPLPPVAPLHCAQTPYGCCQDNITAARGVGLAGCPSACQCNPHGSYGGTCDPATGQCSCRPGVGGLRCDRCEPGFWNFRGIVTDGRSGCTPCSCDPQGAVRDDCEQMTGLCSCKPGVAGPKCGQCPDGRALGPAGCEADASAPATCAEMRCEFGARCVEESGSAHCVCPMLTCPEANATKVCGSDGVTYGNECQLKTIACRQGLQISIQSLGPCQEAVAPSTHPTSASVTVTTPGLLLSQALPAPPGALPLAPSSTAHSQTTPPPSSRPRTTASVPRTTVWPVLTVPPTAPSPAPSLVASAFGESGSTDGSSDEELSGDQEASGGGSGGLEPLEGSSVATPGPPVERASCYNSALGCCSDGKTPSLDAEGSNCPATKVFQGVLELEGVEGQELFYTPEMADPKSELFGETARSIESTLDDLFRNSDVKKDFRSVRLRDLGPGKSVRAIVDVHFDPTTAFRAPDVARALLRQIQVSRRRSLGVRRPLQEHVRFMDFDWFPAFITGATSGAIAAGATARATTASRLPSSAVTPRAPHPSHTSQPVAKTTAAPTTRRPPTTAPSRVPGRRPPAPQQPPKPCDSQPCFHGGTCQDWALGGGFTCSCPAGRGGAVCEKVLGAPVPAFEGRSFLAFPTLRAYHTLRLALEFRALEPQGLLLYNGNARGKDFLALALLDGRVQLRFDTGSGPAVLTSAVPVEPGQWHRLELSRHWRRGTLSVDGETPVLGESPSGTDGLNLDTDLFVGGVPEDQAAVALERTFVGAGLRGCIRLLDVNNQRLELGIGPGAATRGSGVGECGDHPCLPNPCHGGAPCQNLEAGRFHCQCPPGRVGPTCADEKSPCQPNPCHGAAPCRVLPEGGAQCECPLGREGTFCQTASGQDGSGPFLADFNGFSHLELRGLHTFARDLGEKMALEVVFLARGPSGLLLYNGQKTDGKGDFVSLALRDRRLEFRYDLGKGAAVIRSREPVTLGAWTRVSLERNGRKGALRVGDGPRVLGESPKSRKVPHTVLNLKEPLYVGGAPDFSKLARAAAVSSGFDGAIQLVSLGGRQLLTPEHVLRQVDVTSFAGHPCTRASGHPCLNGASCVPREAAYVCLCPGGFSGPHCEKGLVEKSAGDVDTLAFDGRTFVEYLNAVTESELANEIPVPETLDSGALHSEKALQSNHFELSLRTEATQGLVLWSGKATERADYVALAIVDGHLQLSYNLGSQPVVLRSTVPVNTNRWLRVVAHREQREGSLQVGNEAPVTGSSPLGATQLDTDGALWLGGLPELPVGPALPKAYGTGFVGCLRDVVVGRHPLHLLEDAVTKPELRPCPTP

SEQ ID NO: 4 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQLSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 5 - protein, 344 aa, Homo sapiens
MVRARHQPGGLCLLLLLLCQFMEDRSAQAGNCWLRQAKNGRCQVLYKTELSKEECCSTGRLSTSWTEEDVNDNTLFKWMIFNGGAPNCIPCKETCENVDCGPGKKCRMNKKNKPRCVCAPDCSNITWKGPVCGLDGKTYRNECALLKARCKEQPELEVQYQGRCKKTCRDVFCPGSSTCVVDQTNNAYCVTCNRICPEPASSEQYLCGNDGVTYSSACHLRKATCLLGRSIGLAYEGKCIKAKSCEDIQCTGGKKCLWDFKVGRGRCSLCDELCPDSKSDEPVCASDNATYASECAMKEAACSSGVLLEVKHSGSCNSISEDTEEEEEDEDQDYSFPISSILEW

SEQ ID NO: 6 - protein, 836 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYTTKIITKVVEPKIKVIEGSLQPIIKTEGPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 7 - protein, 216 aa, Homo sapiens
MGAARLLPNLTLCLQLLILCCQTQGENHPSPNFNQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 8 - protein, 961 aa, Homo sapiens
MLAPRGAAVLLLHLVLQRWLAAGAQATPQVFDLLPSSSQRLNPGALLPVLTDPALNDLYVISTFKLQTKSSATIFGLYSSTDNSKYFEFTVMGRLNKAILRYLKNDGKVHLVVFNNLQLADGRRHRILLRLSNLQRGAGSLELYLDCIQVDSVHNLPRAFAGPSQKPETIELRTFQRKPQDFLEELKLVVRGSLFQVASLQDCFLQQSEPLAATGTGDFNRQFLGQMTQLNQLLGEVKDLLRQQVKETSFLRNTIAECQACGPLKFQSPTPSTVVPPAPPAPPTRPPRRCDSNPCFRGVQCTDSRDGFQCGPCPEGYTGNGITCIDVDECKYHPCYPGVHCINLSPGFRCDACPVGFTGPMVQGVGISFAKSNKQVCTDIDECRNGACVPNSICVNTLGSYRCGPCKPGYTGDQIRGCKAERNCRNPELNPCSVNAQCIEERQGDVTCVCGVGWAGDGYICGKDVDIDSYPDEELPCSARNCKKDNCKYVPNSGQEDADRDGIGDACDEDADGDGILNEQDNCVLIHNVDQRNSDKDIFGDACDNCLSVLNNDQKDTDGDGRGDACDDDMDGDGIKNILDNCPKFPNRDQRDKDGDGVGDACDSCPDVSNPNQSDVDNDLVGDSCDTNQDSDGDGHQDSTDNCPTVINSAQLDTDKDGIGDECDDDDDNDGIPDLVPPGPDNCRLVPNPAQEDSNSDGVGDICESDFDQDQVIDRIDVCPENAEVTLTDFRAYQTVVLDPEGDAQIDPNWVVLNQGMEIVQTMNSDPGLAVGYTAFNGVDFEGTFHVNTQTDDDYAGFIFGYQDSSSFYVVMWKQTEQTYWQATPFRAVAEPGIQLKAVKSKTGPGEHLRNSLWHTGDTSDQVRLLWKDSRNVGWKDKVSYRWFLQHRPQVGYIRVRFYEGSELVADSGVTIDTTMRGGRLGVFCFSQENIIWSNLKYRCNDTIPEDFQEFQTQNFDRFDN

SEQ ID NO: 9 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 10 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 11 - protein, 180 aa, Homo sapiens
MGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 12 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 13 - protein, 216 aa, Homo sapiens
MRSGCVVVHVWILAGLWLAVAGRPLAFSDAGPHVHYGWGDPIRLRHLYTSGPHGLSSCFLRIRADGVVDCARGQSAHSLLEIKAVALRTVAIKGVHSVRYLCMGADGKMQGLLQYSEEDCAFEEEIRPDGYNVYRSEKHRLPVSLSSAKQRQLYKNRGFLPLSHFLPMLPMVPEEPEDLRGHLESDMFSSPLETDSMDPFGLVTGLEAVRSPSFEK

SEQ ID NO: 14 - protein, 147 aa, Homo sapiens
MVMGLGVLLLVFVLGLGLTPPTLAQDNSRYTHFLTQHYDAKPQGRDDRYCESIMRRRGLTSPCKDINTFIHGNKRSIKAICENKNGNPHRENLRISKSSFQVTTCKLHGGSPWPPCQYRATAGFRNVVVACENGLPVHLDQSIFRRP

SEQ ID NO: 15 - protein, 178 aa, Homo sapiens
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETNIA

SEQ ID NO: 16 - protein, 380 aa, Homo sapiens
MAFVCLAIGCLYTFLISTTFGCTSSSDTEIKVNPPQDFEIVDPGYLGYLYLQWQPPLSLDHFKECTVEYELKYRNIGSETWKTIITKNLHYKDGFDLNKGIEAKIHTLLPWQCTNGSEVQSSWAETTYWISPQGIPETKVQDMDCVYYNWQYLLCSWKPGIGVLLDTNYNLFYWYEGLDHALQCVDYIKADGQNIGCRFPYLEASDYKDFYICVNGSSENKPIRSSYFTFQLQNIVKPLPPVYLTFTRESSCEIKLKWSIPLGPIPARCFDYEIEIREDDTTLVTATVENETYTLKTTNETRQLCFVVRSKVNIYCSDDGIWSEWSDKQCWEGEDLSKKTLLRFWLPFGFILILVIFVTGLLLRKPNTYPKMIPEFFCDT

SEQ ID NO: 17 - protein, 551 aa, Homo sapiens
MLPLLLLPLLWGGSLQEKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAALGGAGVMALLCICLCLIFFLIVKARRKQAAGRPEKMDDEDPIMGTITSGSRKKPWPDSPGDQASPPGDAPPLEEQKELHYASLSFSEMKSREPKDQEAPSTTEYSEIKTSK

SEQ ID NO: 18 - protein, 380 aa, Homo sapiens
MEEGGDFDNYYGADNQSECEYTDWKSSGALIPAIYMLVFLLGTTGNGLVLWTVFRSSREKRRSADIFIASLAVADLTFVVTLPLWATYTYRDYDWPFGTFFCKLSSYLIFVNMYASVFCLTGLSFDRYLAIVRPVANARLRLRVSGAVATAVLWVLAALLAMPVMVLRTTGDLENTTKVQCYMDYSMVATVSSEWAWEVGLGVSSTTVGFVVPFTIMLTCYFFIAQTIAGHFRKERIEGLRKRRRLLSIIVVLVVTFALCWMPYHLVKTLYMLGSLLHWPCDFDLFLMNIFPYCTCISYVNSCLNPFLYAFFDPRFRQACTSMLCCGQSRCAGTSHSSSGEKSASYSSGHSQGPGPNMGKGGEQMHEKSIPYSQETLVVD

SEQ ID NO: 19 - protein, 325 aa, Homo sapiens
MLPRVGCPALPLPPPPLLPLLLLLLGASGGGGGARAEVLFRCPPCTPERLAACGPPPVAPPAAVAAVAGGARMPCAELVREPGCGCCSVCARLEGEACGVYTPRCGQGLRCYPHPGSELPLQALVMGEGTCEKRRDAEYGASPEQVADNGDDHSEGGLVENHVDSTMNMLGGGGSAGRKPLKSGMKELAVFREKVTEQHRQMGKGGKHHLGLEEPKKLRPPPARTPCQQELDQVLERISTMRLPDERGPLEHLYSLHIPNCDKHGLYNLKQCKMSLNGQRGECWCVNPNTGKLIQGAPTIRGDPECHLFYNEQQEARGVHTQRMQ

SEQ ID NO: 20 - protein, 450 aa, Homo sapiens
MGGMKYIFSLLFFLLLEGGKTEQVKHSETYCMFQDKKYRVGERWHPYLEPYGLVYCVNCICSENGNVLCSRVRCPNVHCLSPVHIPHLCCPRCPDSLPPVNNKVTSKSCEYNGTTYQHGELFVAEGLFQNRQPNQCTQCSCSEGNVYCGLKTCPKLTCAFPVSVPDSCCRVCRGDGELSWEHSDGDIFRQPANREARHSYHRSHYDPPPSRQAGGLSRFPGARSHRGALMDSQQASGTIVQIVINNKHKHGQVCVSNGKTYSHGESWHPNLRAFGIVECVLCTCNVTKQECKKIHCPNRYPCKYPQKIDGKCCKVCPGKKAKELPGQSFDNKGYFCGEETMPVYESVFMEDGETTRKIALETERPPQVEVHVWTIRKGILQHFHIEKISKRMFEELPHFKLVTRTTLSQWKIFTEGEAQISQMCSSRVCRTELEDLVKVLYLERSEKGHC

SEQ ID NO: 21 - protein, 576 aa, Homo sapiens
MWAPRCRRFWSRWEQVAALLLLLLLLGVPPRSLALPPIRYSHAGICPNDMNPNLWVDAQSTCRRECETDQECETYEKCCPNVCGTKSCVAARYMDVKGKKGPVGMPKEATCDHFMCLQQGSECDIWDGQPVCKCKDRCEKEPSFTCASDGLTYYNRCYMDAEACSKGITLAVVTCRYHFTWPNTSPPPPETTMHPTTASPETPELDMAAPALLNNPVHQSVTMGETVSFLCDVVGRPRPEITWEKQLEDRENVVMRPNHVRGNVVVTNIAQLVIYNAQLQDAGIYTCTARNVAGVLRADFPLSVVRGHQAAATSESSPNGTAFPAAECLKPPDSEDCGEEQTRWHFDAQANNCLTFTFGHCHRNLNHFETYEACMLACMSGPLAACSLPALQGPCKAYAPRWAYNSQTGQCQSFVYGGCEGNGNNFESREACEESCPFPRGNQRCRACKPRQKLVTSFCRSDFVILGRVSELTEEPDSGRALVTVDEVLKDEKMGLKFLGQEPLEVTLLHVDWACPCPNVTVSEMPLIIMGEVDGGMAMLRPDSFVGASSARRVRKLREVMHKKTCDVLKEFLGLH

SEQ ID NO: 22 - protein, 579 aa, Homo sapiens
MKDFSDVILCMEATESSKTEFCNPAFEPESGPPCPPPVFPEDASYSVPAPWHGRRPRGLRPDCRFSWLCVLLLSSLLLLLLGLLVAIILAQLQAAPPSGASHSPLPAGGLTTTTTTPTITTSQAAGTPKGQQESGVSPSPQSTCGGLLSGPRGFFSSPNYPDPYPPNTHCVWHIQVATDHAIQLKIEALSIESVASCLFDRLELSPEPEGPLLRVCGRVPPPTLNTNASHLLVVFVSDSSVEGFGFHAWYQAMAPGRGSCAHDEFRCDQLICLLPDSVCDGFANCADGSDETNCSAKFSGCGGNLTGLQGTFSTPSYLQQYPHQLLCTWHISVPAGHSIELQFHNFSLEAQDECKFDYVEVYETSSSGAFSLLGRFCGAEPPPHLVSSHHELAVLFRTDHGISSGGFSATYLAFNATENPCGPSELSCQAGGCKGVQWMCDMWRDCTDGSDDNCSGPLFPPPELACEPVQVEMCLGLSYNTTAFPNIWVGMITQEEVVEVLSGYKSLTSLPCYQHFRRLLCGLLVPRCTPLGSVLPPCRSVCQEAEHQCQSGLALLGTPWPFNCNRLPEAADLEACAQP

SEQ ID NO: 23 - protein, 578 aa, Homo sapiens
MLPCLVVLLAALLSLRLGSDAHGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTNVIIFFAFVLLLSGALAYCLALQLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 24 - protein, 373 aa, Homo sapiens
MRMEDEDYNTSISYGDEYPDYLDSIVVLEDLSPLEARVTRIFLVVVYSIVCFLGILGNGLVIIIATFKMKKTVNMVWFLNLAVADFLFNVFLPIHITYAAMDYHWVFGTAMCKISNFLLIHNMFTSVFLLTIISSDRCISVLLPVWSQNHRSVRLAYMACMVIWVLAFFLSSPSLVFRDTANLHGKISCFNNFSLSTPGSSSWPTHSQMDPVGYSRHMVVTVTRFLCGFLVPVLIITACYLTIVCKLQRNRLAKTKKPFKIIVTIIITFFLCWCPYHTLNLLELHHTAMPGSVFSLGLPLATALAIANSCMNPILYVFMGQDFKKFKVALFSRLVNALSEDTGHSSYPSHRSFTKMSSMNERTSMNERETGML

SEQ ID NO: 25 - protein, 208 aa, Homo sapiens
MKLLPSVVLKLFLAAVLSALVTGESLERLRRGLAAGTSNPDPPTVSTDQLLPLGGGRDRKVRDLQEADLDLLRVTLSSKPQALATPNKEEHGKRKKKGKGLGKKRDPCLRKYKDFCIHGECKYVKELRAPSCICHPGYHGERCHGLSLPVENRLYTYDHTTILAVVAVVLSSVCLLVIVGLLMFRYHRRGGYDVENEEKVKLGMTNSH

SEQ ID NO: 26 - protein, 208 aa, Homo sapiens
MALGQKLFITMSRGAGRLQGTLWALVFLGILVGMVVPSPAGTRANNTLLDSRGWGTLLSRSRAGLAGEIAGVNWESGYLVGIKRQRRLYCNVGIGFHLQVLPDGRISGTHEENPYSLLEISTVERGVVSLFGVRSALFVAMNSKGRLYATPSFQEECKFRETLLPNNYNAYESDLYQGTYIALSKYGRVKRGSKVSPIMTVTHFLPRI

SEQ ID NO: 27 - protein, 1390 aa, Homo sapiens
MKAPAVLAPGILVLLFTLVQRSNGECKEALAKSEMNVNMKYQLPNFTAETPIQNVILHEHHIFLGATNYIYVLNEEDLQKVAEYKTGPVLEHPDCFPCQDCSSKANLSGGVWKDNINMALVVDTYYDDQLISCGSVNRGTCQRHVFPHNHTADIQSEVHCIFSPQIEEPSQCPDCVVSALGAKVLSSVKDRFINFFVGNTINSSYFPDHPLHSISVRRLKETKDGFMFLTDQSYIDVLPEFRDSYPIKYVHAFESNNFIYFLTVQRETLDAQTFHTRIIRFCSINSGLHSYMEMPLECILTEKRKKRSTKKEVFNILQAAYVSKPGAQLARQIGASLNDDILFGVFAQSKPDSAEPMDRSAMCAFPIKYVNDFFNKIVNKNNVRCLQHFYGPNHEHCFNRTLLRNSSGCEARRDEYRTEFTTALQRVDLFMGQFSEVLLTSISTFIKGDLTIANLGTSEGRFMQVVVSRSGPSTPHVNFLLDSHPVSPEVIVEHTLNQNGYTLVITGKKITKIPLNGLGCRHFQSCSQCLSAPPFVQCGWCHDKCVRSEECLSGTWTQQICLPAIYKVFPNSAPLEGGTRLTICGWDFGFRRNNKFDLKKTRVLLGNESCTLTLSESTMNTLKCTVGPAMNKHFNMSIIISNGHGTTQYSTFSYVDPVITSISPKYGPMAGGTLLTLTGNYLNSGNSRHISIGGKTCTLKSVSNSILECYTPAQTISTEFAVKLKIDLANRETSIFSYREDPIVYEIHPTKSFISGGSTITGVGKNLNSVSVPRMVINVHEAGRNFTVACQHRSNSEIICCTTPSLQQLNLQLPLKTKAFFMLDGILSKYFDLIYVHNPVFKPFEKPVMISMGNENVLEIKGNDIDPEAVKGEVLKVGNKSCENIHLHSEAVLCTVPNDLLKLNSELNIEWKQAISSTVLGKVIVQPDQNFTGLIAGVVSISTALLLLLGFFLWLKKRKQIKDLGSELVRYDARVHTPHLDRLVSARSVSPTTEMVSNESVDYRATFPEDQFPNSSQNGSCRQVQYPLTDMSPILTSGDSDISSPLLQNTVHIDLSALNPELVQAVQHVVIGPSSLIVHFNEVIGRGHFGCVYHGTLLDNDGKKIHCAVKSLNRITDIGEVSQFLTEGIIMKDFSHPNVLSLLGICLRSEGSPLVVLPYMKHGDLRNFIRNETHNPTVKDLIGFGLQVAKGMKYLASKKFVHRDLAARNCMLDEKFTVKVADFGLARDMYDKEYYSVHNKTGAKLPVKWMALESLQTQKFTTKSDVWSFGVLLWELMTRGAPPYPDVNTFDITVYLLQGRRLLQPEYCPDPLYEVMLKCWHPKAEMRPSFSELVSRISAIFSTFIGEHYVHVNATYVNVKCVAPYPSLLSSEDNADDEVDTRPASFWETS

SEQ ID NO: 28 - protein, 1332 aa, Homo sapiens
MESHSRAGKSRKSAKFRSISRSLMLCNAKTSDDGSSPDEKYPDPFEISLAQGKEGIFHSSVQLADTSEAGPSSVPDLALASEAAQLQAAGNDRGKTCRRIFFMKESSTASSREKPGKLEAQSSNFLFPKACHQRARSNSTSVNPYCTREIDFPMTKKSAAPTDRQPYSLCSNRKSLSQQLDCPAGKAAGTSRPTRSLSTAQLVQPSGGLQASVISNIVLMKGQAKGLGFSIVGGKDSIYGPIGIYVKTIFAGGAAAADGRLQEGDEILELNGESMAGLTHQDALQKFKQAKKGLLTLTVRTRLTAPPSLCSHLSPPLCRSLSSSTCITKDSSSFALESPSAPISTAKPNYRIMVEVSLQKEAGVGLGIGLCSVPYFQCISGIFVHTLSPGSVAHLDGRLRCGDEIVEISDSPVHCLTLNEVYTILSHCDPGPVPIIVSRHPDPQVSEQQLKEAVAQAVENTKFGKERHQWSLEGVKRLESSWHGRPTLEKEREKNSAPPHRRAQKVMIRSSSDSSYMSGSPGGSPGSGSAEKPSSDVDISTHSPSLPLAREPVVLSIASSRLPQESPPLPESRDSHPPLRLKKSFEILVRKPMSSKPKPPPRKYFKSDSDPQKSLEERENSSCSSGHTPPTCGQEARELLPLLLPQEDTAGRSPSASAGCPGPGIGPQTKSSTEGEPGWRRASPVTQTSPIKHPLLKRQARMDYSFDTTAEDPWVRISDCIKNLFSPIMSENHGHMPLQPNASLNEEEGTQGHPDGTPPKLDTANGTPKVYKSADSSTVKKGPPVAPKPAWFRQSLKGLRNRASDPRGLPDPALSTQPAPASREHLGSHIRASSSSSSIRQRISSFETFGSSQLPDKGAQRLSLQPSSGEAAKPLGKHEEGRFSGLLGRGAAPTLVPQQPEQVLSSGSPAASEARDPGVSESPPPGRQPNQKTLPPGPDPLLRLLSTQAEESQGPVLKMPSQRARSFPLTRSQSCETKLLDEKTSKLYSISSQVSSAVMKSLLCLPSSISCAQTPCIPKEGASPTSSSNEDSAANGSAETSALDTGFSLNLSELREYTEGLTEAKEDDDGDHSSLQSGQSVISLLSSEELKKLIEEVKVLDEATLKQLDGIHVTILHKEEGAGLGFSLAGGADLENKVITVHRVFPNGLASQEGTIQKGNEVLSINGKSLKGTTHHDALAILRQAREPRQAVIVTRKLTPEAMPDLNSSTDSAASASAASDVSVESTAEATVCTVTLEKMSAGLGFSLEGGKGSLHGDKPLTINRIFKGAASEQSETVQPGDEILQLGGTAMQGLTRFEAWNIIKALPDGPVTIVIRRKSLQSKETTAAGDS

SEQ ID NO: 29 - protein, 459 aa, Homo sapiens [sequence omitted]

SEQ ID NO: 30 - protein, 259 aa, Homo sapiens
MARIPKTLKFVVVIVAVLLPVLAYSATTARQEEVPQQTVAPQQQRHSFKGEECPAGSHRSEHTGACNPCTEGVDYTNASNNEPSCFPCTVCKSDQKHKSSCTMTRDTVCQCKEGTFRNENSPEMCRKCSRCPSGEVQVSNCTSWDDIQCVEEFGANATVETPAAEETMNTSPGTPAPAAEETMNTSPGTPAPAAEETMTTSPGTPAPAAEETMTTSPGTPAPAAEETMITSPGTPASSHYLSCTIVGIIVLIVLLIVFV

SEQ ID NO: 31 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 32 - protein, 169 aa, Homo sapiens
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 33 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 34 - protein, 462 aa, Homo sapiens
MGTQALQGFLFLLFLPLLQPRGASAGSLHSPGLSECFQVNGADYRGHQNRTGPRGAGRPCLFWDQTQQHSYSSASDPHGRWGLGAHNFCRNPDGDVQPWCYVAETEEGIYWRYCDIPSCHMPGYLGCFVDSGAPPALSGPSGTSTKLTVQVCLRFCRMKGYQLAGVEAGYACFCGSESDLARGRLAPATDCDQICFGHPGQLCGGDGRLGVYEVSVGSCQGNWTAPQGVIYSPDFPDEYGPDRNCSWALGPPGAALELTFRLFELADPRDRLELRDAASGSLLRAFDGARPPPSGPLRLGTAALLLTFRSDARGHAQGFALTYRGLQDAAEDPEAPEGSAQTPAAPLDGANVSCSPRPGAPPAAIGARVFSTVTAVSVLLLLLLGLLRPLRRRSCLLAPGKGPPALGASRGPRRSWAVWYQQPRGVALPCSPGDPQAEGSAAGYRPLSASSQSSLRSLISAL

SEQ ID NO: 35 - protein, 386 aa, Homo sapiens
MGLWGQSVPTASSARAGRYPGARTASGTRPWLLDPKILKFVVFIVAVLLPVRVDSATIPRQDEVPQQTVAPQQQRRSLKEEECPAGSHRSEYTGACNPCTEGVDYTIASNNLPSCLLCTVCKSGQTNKSSCTTTRDTVCQCEKGSFQDKNSPEMCRTCRTGCPRGMVKVSNCTPRSDIKCKNESAASSTGKTPAAEETVTTILGMLASPYHYLIIIVVLVIILAVVVVGFSCRKKFISYLKGICSGGGGGPERVHRVLFRRRSCPSRVPGAEDNARNETLSNRYLQPTQVSEQEIQGQELAELTGVTVESPEEPQRLLEQAEAEGCQRRRLLVPVNDADSADISTLLDASATLEEGHAKETIQDQLVGSEKLFYEEDEAGSATSCL

SEQ ID NO: 36 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 37 - protein, 120 aa, Homo sapiens
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCVRMLKLDTRIKTRKN

SEQ ID NO: 38 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 39 - protein, 245 aa, Homo sapiens
MAAAIASSLIRQKRQAREREKSNACKCVSSPSKGKTSCDKNKLNVFSRVKLFGSKKRRRRRPEPQLKGIVTKLYSRQGYHLQLQADGTIDGTKDEDSTYTLFNLIPVGLRVVAIQGVQTKLYLAMNSEGYLYTSELFTPECKFKESVFENYYVTYSSMIYRQQQSGRGWYLGLNKEGEIMKGNHVKKNKPAAHFLPKPLKVAMYKEPSLHDLTEFSRSGSGTPTKSRSVSGVLNGGKSMSHNEST

SEQ ID NO: 40 - protein, 281 aa, Homo sapiens [sequence omitted]

SEQ ID NO: 41 - protein, 93 aa, Homo sapiens
MKISVAAIPFFLLITIALGTKTESSSRGPYHPSECCFTYTTYKIPRQRIMDYYETNSQCSKPGIVFITKRGHSVCTNPSDKWVQDYIKDMKEN

SEQ ID NO: 42 - protein, 282 aa, Homo sapiens
MERPSLRALLLGAAGLLLLLLPLSSSSSSDTCGPCEPASCPPLPPLGCLLGETRDACGCCPMCARGEGEPCGGGGAGRGYCAPGMECVKSRKRRKGKAGAAAGGPGVSGVCVCKSRYPVCGSDGTTYPSGCQLRAASQRAESRGEKAITQVSKGTCEQGPSIVTPPKDIWNVTGAQVYLSCEVIGIPTPVLIWNKVKRGHYGVQRTELLPGDRDNLAIQTRGGPEKHEVTGWVLVSPLSKEDAGEYECHASNSQGQASASAKITVVDALHEIPVKKGEGAEL

SEQ ID NO: 43 - protein, 206 aa, Homo sapiens
MSGPGTAAVALLPAVLLALLAPWAGRGGAAAPTAPNGTLEAELERRWESLVALSLARLPVAAQPKEAAVQSGAGDYLLGIKRLRRLYCNVGIGFHLQALPDGRIGGAHADTRDSLLELSPVERGVVSIFGVASRFFVAMSSKGKLYGSPFFTDECTFKEILLPNNYNAYESYKYPGMFIALSKNGKTKKGNRVSPTMKVTHFLPRL

SEQ ID NO: 44 - protein, 233 aa, Homo sapiens
MGSPRSALSCLLLHLLVLCLQAQEGPGRGPALGRELASLFRAGREPQGVSQQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 45 - DNA, 651 bp, Homo sapiens
  source 1..651: mol_type = other DNA
atgggagccgcccgcctgctgcccaacctcactctgtgcttacagctgctgattctctgctgtcaaactcagggggagaatcacccgtctcctaattttaaccagtacgtgagggaccagggcgccatgaccgaccagctgagcaggcggcagatccgcgagtaccaactctacagcaggaccagtggcaagcacgtgcaggtcaccgggcgtcgcatctccgccaccgccgaggacggcaacaagtttgccaagctcatagtggagacggacacgtttggcagccgggttcgcatcaaaggggctgagagtgagaagtacatctgtatgaacaagaggggcaagctcatcgggaagcccagcgggaagagcaaagactgcgtgttcacggagatcgtgctggagaacaactatacggccttccagaacgcccggcacgagggctggttcatggccttcacgcggcaggggcggccccgccaggcttcccgcagccgccagaaccagcgcgaggcccacttcatcaagcgcctctaccaaggccagctgcccttccccaaccacgccgagaagcagaagcagttcgagtttgtgggctccgcccccacccgccggaccaagcgcacacggcggccccagcccctcacgtag

SEQ ID NO: 46 - DNA, 2886 bp, Homo sapiens
  source 1..2886: mol_type = other DNA
atgctggccccgcgcggagccgccgtcctcctgctgcacctggtcctgcagcggtggctagcggcaggcgcccaggccaccccccaggtctttgaccttctcccatcttccagtcagaggctaaacccaggcgctctgctgccagtcctgacagaccccgccctgaatgatctctatgtgatttccaccttcaagctgcagactaaaagttcagccaccatcttcggtctttactcttcaactgacaacagtaaatattttgaatttactgtgatgggacgcttaaacaaagccatcctccgttacctgaagaacgatgggaaggtgcatttggtggttttcaacaacctgcagctggcagacggaaggcggcacaggatcctcctgaggctgagcaatttgcagcgaggggccggctccctagagctctacctggactgcatccaggtggattccgttcacaatctccccagggcctttgctggcccctcccagaaacctgagaccattgaattgaggactttccagaggaagccacaggacttcttggaagagctgaagctggtggtgagaggctcactgttccaggtggccagcctgcaagactgcttcctgcagcagagtgagccactggctgccacaggcacaggggactttaaccggcagttcttgggtcaaatgacacaattaaaccaactcctgggagaggtgaaggaccttctgagacagcaggttaaggaaacatcatttttgcgaaacaccatagctgaatgccaggcttgcggtcctctcaagtttcagtctccgaccccaagcacggtggtgcccccggctccccctgcaccgccaacacgcccacctcgtcggtgtgactccaacccatgtttccgaggtgtccaatgtaccgacagtagagatggcttccagtgtgggccctgccccgagggctacacaggaaacgggatcacctgtattgatgttgatgagtgcaaataccatccctgctacccgggcgtgcactgcataaatttgtctcctggcttcagatgtgacgcctgcccagtgggcttcacagggcccatggtgcagggtgttgggatcagttttgccaagtcaaacaagcaggtctgcactgacattgatgagtgtcgaaatggagcgtgcgttcccaactcgatctgcgttaatactttgggatcttaccgctgtgggccttgtaagccggggtatactggtgatcagataaggggatgcaaagcggaaagaaactgcagaaacccagagctgaacccttgcagtgtgaatgcccagtgcattgaagagaggcagggggatgtgacatgtgtgtgtggagtcggttgggctggagatggctatatctgtggaaaggatgtggacatcgacagttaccccgacgaagaactgccatgctctgccaggaactgtaaaaaggacaactgcaaatatgtgccaaattctggccaagaagatgcagacagagatggcattggcgacgcttgtgacgaggatgctgacggagatgggatcctgaatgagcaggataactgtgtcctgattcataatgtggaccaaaggaacagcgataaagatatctttggggatgcctgtgataactgcctgagtgtcttaaataacgaccagaaagacaccgatggggatggaagaggagatgcctgtgatgatgacatggatggagatggaataaaaaacattctggacaactgcccaaaatttcccaatcgtgaccaacgggacaaggatggtgatggtgtgggggatgcctgtgacagttgtcctgatgtcagcaaccctaaccagtctgatgtggataatgatctggttggggactcctgtgacaccaatcaggacagtgatggagatgggcaccaggacagcacagacaactgccccaccgtcattaacagtgcccagctggacaccgataaggatggaattggtgacgagtgtgatgatgatgatgacaatgatggtatcccagacctggtgccccctggaccagacaactgccggctggtccccaacccagcccaggaggatagcaacagcgacggagtgggagacatctgtgagtctgactttgaccaggaccaggtcatcgatcggatcgacgtctgcccagagaacgcagaggtcaccctgaccgacttcagggcttaccagaccgtggtcctggatcctgaaggggatgcccagatcgatcccaactgggtggtcctgaaccagggcatggagattgtacagaccatgaacagtgatcctggcctggcagtggggtacacagcttttaatggagttgacttcgaagggaccttccatgtgaatacccagacagatgatgactatgcaggctttatctttggctaccaagatagctccagcttctacgtggtcatgtggaagcagacggagcagacatattggcaagccaccccattccgagcagttgcagaacctggcattcagctcaaggctgtgaagtctaagacaggtccaggggagcatctccggaactccctgtggcacacgggggacaccagtgaccaggtcaggctgctgtggaaggactccaggaatgtgggctggaaggacaaggtgtcctaccgctggttcctacagcacaggccccaggtgggctacatcagggtacgattttatgaaggctctgagttggtggctgactctggcgtcaccatagacaccacaatgcgtggaggccgacttggcgttttctgcttctctcaagaaaacatcatctggtccaacctcaagtatcgctgcaatgacaccatccctgaggacttccaagagtttcaaacccagaatttcgaccgcttcgataattaa

SEQ ID NO: 47 - DNA, 3519 bp, Homo sapiens
  source 1..3519: mol_type = other DNA
atggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaacggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcatagacagcttcgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcggagaggaggcccgaggtgtgcgaacgctcgtgcgaggagctgggaaacatggtccaggagctctcggggctccacgtcctcgtgaaccagctcagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctccatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggctcctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggccacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgccggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagactggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaa

SEQ ID NO: 48 - DNA, 2886 bp, Homo sapiens; identical to SEQ ID NO: 46
  source 1..2886: mol_type = other DNA

SEQ ID NO: 49 - DNA, 543 bp, Homo sapiens
  source 1..543: mol_type = other DNA
atgggaatcccaatggggaagtcgatgctggtgcttctcaccttcttggccttcgcctcgtgctgcattgctgcttaccgccccagtgagaccctgtgcggcggggagctggtggacaccctccagttcgtctgtggggaccgcggcttctacttcagcaggcccgcaagccgtgtgagccgtcgcagccgtggcatcgttgaggagtgctgtttccgcagctgtgacctggccctcctggagacgtactgtgctacccccgccaagtccgagagggacgtgtcgacccctccgaccgtgcttccggacaacttccccagataccccgtgggcaagttcttccaatatgacacctggaagcagtccacccagcgcctgcgcaggggcctgcctgccctcctgcgtgcccgccggggtcacgtgctcgccaaggagctcgaggcgttcagggaggccaaacgtcaccgtcccctgattgctctacccacccaagaccccgcccacgggggcgcccccccagagatggccagcaatcggaagtga

SEQ ID NO: 50 - DNA, 489 bp, Homo sapiens
  source 1..489: mol_type = other DNA
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 51 - DNA, 849 bp, Homo sapiens
  source 1..849: mol_type = other DNA
atggagcggccgtcgctgcgcgccctgctcctcggcgccgctgggctgctgctcctgctcctgcccctctcctcttcctcctcttcggacacctgcggcccctgcgagccggcctcctgcccgcccctgcccccgctgggctgcctgctgggcgagacccgcgacgcgtgcggctgctgccctatgtgcgcccgcggcgagggcgagccgtgcgggggtggcggcgccggcagggggtactgcgcgccgggcatggagtgcgtgaagagccgcaagaggcggaagggtaaagccggggcagcagccggcggtccgggtgtaagcggcgtgtgcgtgtgcaagagccgctacccggtgtgcggcagcgacggcaccacctacccgagcggctgccagctgcgcgccgccagccagagggccgagagccgcggggagaaggccatcacccaggtcagcaagggcacctgcgagcaaggtccttccatagtgacgccccccaaggacatctggaatgtcactggtgcccaggtgtacttgagctgtgaggtcatcggaatcccgacacctgtcctcatctggaacaaggtaaaaaggggtcactatggagttcaaaggacagaactcctgcctggtgaccgggacaacctggccattcagacccggggtggcccagaaaagcatgaagtaactggctgggtgctggtatctcctctaagtaaggaagatgctggagaatatgagtgccatgcatccaattcccaaggacaggcttcagcatcagcaaaaattacagtggttgatgccttacatgaaataccagtgaaaaaaggtgaaggtgccgagctataa

SEQ ID NO: 52 - DNA, 1437 bp, Homo sapiens
  source 1..1437: mol_type = other DNA
atggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtag

SEQ ID NO: 53 - DNA, 2511 bp, Homo sapiens
  source 1..2511: mol_type = other DNA
atgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctatacaactaaaattataaccaaagttgtggaaccaaaaattaaagtgattgaaggcagtcttcagcctattatcaaaactgaaggacccacactaacaaaagtcaaaattgaaggtgaacctgaattcagactgattaaagaaggtgaaacaataactgaagtgatccatggagagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtga

SEQ ID NO: 54 - DNA, 1128 bp, Homo sapiens
  source 1..1128: mol_type = other DNA
atgaaggtctggctgctgcttggtcttctgctggtgcacgaagcgctggaggatgttactggccaacaccttcccaagaacaagcgtccaaaagaaccaggagagaatagaatcaaacctaccaacaagaaggtgaagcccaaaattcctaaaatgaaggacagggactcagccaattcagcaccaaagacgcagtctatcatgatgcaagtgctggataaaggtcgcttccagaaacccgccgctaccctgagtctgctggcggggcaaactgtagagcttcgatgtaaagggagtagaattgggtggagctaccctgcgtatctggacacctttaaggattctcgcctcagcgtcaagcagaatgagcgctacggccagttgactctggtcaactccacctcggcagacacaggtgaattcagctgctgggtgcagctctgcagcggctacatctgcaggaaggacgaggccaaaacgggctccacctacatcttttttacagagaaaggagaactctttgtaccttctcccagctacttcgatgttgtctacttgaacccggacagacaggctgtggttccttgtcgggtgaccgtgctgtcggccaaagtcacgctccacagggaattcccagccaaggagatcccagccaatggaacggacattgtttatgacatgaagcggggctttgtgtatctgcaacctcattccgagcaccagggtgtggtttactgcagggcggaggccgggggcagatctcagatctccgtcaagtaccagctgctctacgtggcggttcccagtggccctccctcaacaaccatcttggcttcttcaaacaaagtgaaaagtggggacgacatcagtgtgctctgcactgtcctgggggagcccgatgtggaggtggagttcacctggatcttcccagggcagaaggatgaaaggcctgtgacgatccaagacacttggaggttgatccacagaggactgggacacaccacgagaatctcccagagtgtcattacagtggaagactttgagacgattgatgcaggatattacatttgcactgctcagaatcttcaaggacagaccacagtagctaccactgttgagttttcctga

SEQ ID NO: 55 - protein, 375 aa, Homo sapiens
MKVWLLLGLLLVHEALEDVTGQHLPKNKRPKEPGENRIKPTNKKVKPKIPKMKDRDSANSAPKTQSIMMQVLDKGRFQKPAATLSLLAGQTVELRCKGSRIGWSYPAYLDTFKDSRLSVKQNERYGQLTLVNSTSADTGEFSCWVQLCSGYICRKDEAKTGSTYIFFTEKGELFVPSPSYFDVVYLNPDRQAVVPCRVTVLSAKVTLHREFPAKEIPANGTDIVYDMKRGFVYLQPHSEHQGVVYCRAEAGGRSQISVKYQLLYVAVPSGPPSTTILASSNKVKSGDDISVLCTVLGEPDVEVEFTWIFPGQKDERPVTIQDTWRLIHRGLGHTTRISQSVITVEDFETIDAGYYICTAQNLQGQTTVATTVEFS

SEQ ID NO: 56 - protein, 679 aa, Homo sapiens
MVPGVPGAVLTLCLWLAASSGCLAAGPGAAAARRLDESLSAGSVQRARCASRCLSLQITRISAFFQHFQNNGSLVWCQNHKQCSKLEPCKESGDLRKHQCQSFCEPLFPKKSYECLTSCEFLKYILLVKQGDCPAPEKASGFAAACVESCEVDNECSGVKKCCSNGCGHTCQVPKTLYKGVPLKPRKELRFTELQSGQLEVKWSSKFNISIEPVIYVVQRRWNYGIHPSEDDATHWQTVAQTTDERVQLTDIRPSRWYQFRVAAVNVHGTRGFTAPSKHFRSSKDPSAPPAPANLRLANSTVNSDGSVTVTIVWDLPEEPDIPVHHYKVFWSWMVSSKSLVPTKKKRRKTTDGFQNSVILEKLQPDCDYVVELQAITYWGQTRLKSAKVSLHFTSTHATNNKEQLVKTRKGGIQTQLPFQRRRPTRPLEVGAPFYQDGQLQVKVYWKKTEDPTVNRYHVRWFPEACAHNRTTGSEASSGMTHENYIILQDLSFSCKYKVTVQPIRPKSHSKAEAVFFTTPPCSALKGKSHKPVGCLGEAGHVLSKVLAKPENLSASFIVQDVNITGHFSWKMAKANLYQPMTGFQVTWAEVTTESRQNSLPNSIISQSQILPSDHYVLTVPNLRPSTLYRLEVQVLTPGGEGPATIKTFRTPELPPSSAHRSHLKHRHPHHYKPSPERY

SEQ ID NO: 57 - DNA, 2043 bp, Homo sapiens
  source 1..2043: mol_type = other DNA
atggtgcccggggtgcccggcgcggtcctgaccctctgcctctggctggcggcctccagcggctgcctggcggccggccccggcgcggctgctgcgcggcggctggacgagtcgctgtctgccgggagcgtccagcgcgctcgctgcgcctccaggtgcctgagcctgcagatcactcgcatctccgccttcttccagcacttccagaacaatggttccctggtttggtgccagaatcacaagcaatgttctaagtgcctggagccctgcaaggaatcaggggacctgaggaaacaccagtgccaaagcttttgtgagcctctcttccccaagaagagctacgaatgcttgaccagctgtgagttcctcaaatacatcctgttggtgaagcagggggactgtccggctcctgagaaagccagtggatttgcggccgcctgtgttgaaagctgcgaagttgacaatgagtgctctggggtgaagaaatgttgttcgaatgggtgtggacacacctgtcaagtacccaagactctgtacaaaggtgtccccctgaagcccagaaaagagttacgatttacagaactgcagtctggacagctggaggttaagtggtcctcgaaattcaatatttctattgagcctgtgatctatgtggtacaaagaagatggaattatggaatccatcctagcgaagatgacgccactcactggcagacagtggcccagaccacagacgagcgagttcaactgactgacataagacccagccgatggtaccagtttcgagtggctgctgtgaatgtgcatggaactcgaggcttcactgcccccagcaaacacttccgttcttccaaagatccatctgccccaccagcaccggctaacctccggctggccaactccaccgtcaacagtgatgggagtgtgaccgtcactatagtttgggatctccccgaggagccggacatccctgtgcatcattacaaggtcttttggagctggatggtcagcagtaagtctcttgtcccaacaaagaagaagcggagaaagactacggatgggtttcaaaattctgtgatcctggagaaactccagccagactgtgactatgttgtggaattgcaagccataacgtactggggacagacacggctgaagagtgcaaaggtgtcccttcacttcacatcgacacatgcaaccaacaacaaagaacagcttgtgaaaactagaaaaggtggaattcaaacacaactcccttttcaaagacgacgacccactcgcccgctggaagtcggagctcccttctatcaggatggccaactgcaagttaaagtctactggaagaagacagaagatcccactgtcaaccgatatcatgtgcggtggtttcctgaagcgtgtgcccacaacagaacaaccggatcagaggcatcatctggcatgacccacgaaaattacataattcttcaagatctgtcattttcctgcaagtataaggtgactgtccaaccaatacggccaaaaagtcactccaaggcagaagctgttttcttcactactccaccatgctctgctcttaaggggaagagccacaagcctgttggctgcctgggcgaagcaggtcatgttctttctaaggtgctagctaagcctgagaacctttctgcttcattcatcgtccaggatgtgaacatcaccggtcacttttcttggaagatggccaaggccaatctctatcagcccatgactgggtttcaagtgacttgggctgaggtcactacggaaagcagacagaacagcctacccaacagcattatttcacagtcccagatcctgccttccgatcattatgtcctaacagtgcccaatctgagaccatctactctttaccgactggaagtgcaagtgctgaccccaggaggggaggggccggccaccatcaagacgttccggacgccggagctcccaccctcttcagcacacagatctcatcttaagcatcgtcatccacatcattacaagccttctccagaaagatactaa

SEQ ID NO: 58 - protein, 1085 aa, Homo sapiens
MGLAWGLGVLFLMHVCGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 59 - protein, 205 aa, Homo sapiens
MGAARLLPNLTLCLQLLILCCQTQYVRDQGAMTDQLSRRQIREYQLYSRTSGKHVQVTGRRISATAEDGNKFAKLIVETDTFGSRVRIKGAESEKYICMNKRGKLIGKPSGKSKDCVFTEIVLENNYTAFQNARHEGWFMAFTRQGRPRQASRSRQNQREAHFIKRLYQGQLPFPNHAEKQKQFEFVGSAPTRRTKRTRRPQPLT

SEQ ID NO: 60 - protein, 779 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 61 - protein, 781 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEDTPVRKLQANKKVQGSRRRLREGRSQ

SEQ ID NO: 62 - protein, 751 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEDTPVRKLQANKKVQGSRRRLREGRSQ

SEQ ID NO: 63 - protein, 809 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYRPTLTKVKIEGEPEFRLIKEGETITEVIHGEPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 64 - protein, 749 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEEVTKVTKFIEGGDGHLFEDEEIKRLLQGDTPVRKLQANKKVQGSRRRLREGRSQ

SEQ ID NO: 65 - protein, 721 aa, Homo sapiens
MIPFLPMFSLLLLLIVNPINANNHYDKILAHSRIRGRDQGPNVCALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYSPEIKYTRISTGGGETEETLKKLLQEDTPVRKLQANKKVQGSRRRLREGRSQ

SEQ ID NO: 66 - protein, 183 aa, Homo sapiens
MGIPMGKSMLVLLTFLAFASCCIAAYRPSETLCGGELVDTLQFVCGDRGFYFRLPGRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 67 - protein, 236 aa, Homo sapiens
MVSPDPQIIVVAPETELASMQVQRTEDGVTIIQIFWVGRKGELLRRTPVSSAMQTPMGIPMGKSMLVLLTFLAFASCCIAAYRPSETLCGGELVDTLQFVCGDRGFYFSRPASRVSRRSRGIVEECCFRSCDLALLETYCATPAKSERDVSTPPTVLPDNFPRYPVGKFFQYDTWKQSTQRLRRGLPALLRARRGHVLAKELEAFREAKRHRPLIALPTQDPAHGGAPPEMASNRK

SEQ ID NO: 68 - protein, 135 aa, Homo sapiens
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 69 - protein, 6 aa, synthetic construct
  REGION 1..6: note = Description of Artificial Sequence: Synthetic 6xHis tag
HHHHHH

SEQ ID NO: 70 - protein, 10 aa, synthetic construct
  REGION 1..10: note = Description of Artificial Sequence: Synthetic 10xHis tag
HHHHHHHHHH

(19) United States
(12) Patent Application Publication
(10) Pub. No.: US 2025/0241991 A1
(43) Pub. Date: Jul. 31, 2025

(54) HEPARIN-ASSOCIATED POLYPEPTIDES AND USES THEREOF

(71) Applicant: Juvena Therapeutics, Inc., Redwood City, CA (US)

(72) Inventors: Hanadie YOUSEF, Palo Alto, CA (US); Jeremy O'CONNELL, Palo Alto, CA (US); Thach MAI, South San Francisco, CA (US)

(21) Appl. No.: 18/830,512

(22) Filed: Sep. 10, 2024

Related U.S. Application Data
(63) Continuation of application No. 17/072,636, filed on Oct. 16, 2020, now abandoned.
Application No. 17/072,636 is a continuation of application No. 16/455,445, filed on Jun. 27, 2019, now Pat. No. 10,821,155.
(60) Provisional application No. 62/809,479, filed on Feb. 22, 2019.
(60) Provisional application No. 62/690,796, filed on Jun. 27, 2018.

(51) Int. Cl. A61K 38/20; A61K 9/00; A61K 38/17; A61K 38/18; A61K 38/30; A61K 38/39; A61P 21/00

(52) U.S. Cl. CPC A61K 38/2086; A61K 9/0019; A61K 38/1709; A61K 38/1825; A61K 38/30; A61K 38/39; A61P 21/00

(57) ABSTRACT

Described herein are therapeutic compositions comprising heparin-associated polypeptides useful for the treatment of soft-tissue and muscle diseases, disorders, and injuries.

### PRIORITY

This application is a continuation of U.S. patent application Ser. No. 17/072,636, filed Oct. 16, 2020, which is a continuation application of U.S. patent application Ser. No. 16/455,445, filed Jun. 27, 2019, which claims priority to U.S. Provisional Application No. 62/690,796 filed Jun. 27, 2018, and U.S. Provisional Application No. 62/809,479 filed Feb. 22, 2019, both of which are incorporated by reference in their entirety herein.

### SEQUENCE LISTING

The instant application contains a Sequence Listing which has been submitted electronically in XML format and is hereby incorporated by reference in its entirety. Said XML copy, created on Apr. 17, 2025, is named 54275-702_303_SL.xml and is 109.069 bytes in size.

### BACKGROUND

As the average life span increases, increasing emphasis is placed upon “healthy aging.” Individuals would like to live more active lifestyles as they age, and as a result, many aging disorders can have a significant impact on the quality of life of aging individuals. Treatments directed to regenerative ends have utility for treating aging diseases. Additionally, many treatments for aging disorders can be applicable to younger individuals who have suffered illness, injury, or who possess genetic or developmental defects leading to premature tissue loss, wasting, or weakening.

### SUMMARY

As individuals age, tissue progenitor cells lose their regenerative potential. Described herein, in certain aspects, are heparin-associated polypeptides that can restore some or all of this regenerative potential, and are thus useful in the treatment of aging disorders that result in tissue loss or underperformance, and rehabilitation from injury. Described herein are therapeutic compositions comprising heparin-associated polypeptides and methods of treating disorders associated with aging, injury, or illness. In certain embodiments, the therapeutic compositions comprise one or more heparin-associated polypeptides that possess mitogenic (i.e., regenerative) and/or fusion promoting activity to a somatic cell, such as a tissue progenitor cell. In certain embodiments, the therapeutic compositions have activity towards muscle and soft tissue progenitor cells. In certain embodiments these compositions possess utility in treating sarcopenia, cachexia, muscular dystrophy, acute and chronic muscle wasting diseases, and muscle, ligament, or tendon injury, or any combination of these diseases or conditions.

In one aspect, described herein is a composition comprising. (a) a first therapeutic polypeptide comprising a first polypeptide of Table 2, and (b) a second therapeutic polypeptide comprising a second polypeptide of Table 2 In certain embodiments, the first polypeptide of Table 2 is a first polypeptide of Table 1 and/or the second polypeptide of Table 2 is a second polypeptide of Table 1. In certain embodiments, the first polypeptide of Table 2 comprises THBS1, THBS2, THBS4, FGF17, VTN, POSTN, IGF2, or IL-15, or any combination thereof. In certain embodiments, the second polypeptide of Table 2 comprises THBS1, THBS2, THBS4, FGF17, VTN, POSTN, IGF2, or IL-15, or any combination thereof.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises THBS2 and/or a sequence comprising at least about 90% homology to amino acids 19-1172 of SEQ ID NO: 4.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises THBS4 and/or a sequence comprising at least about 90% homology to amino acids 27-961 of SEQ ID NO: 8.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises FGF17 and/or a sequence comprising at least about 90% homology to amino acids 23-2165 of SEQ ID NO: 7.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises VTN and/or a sequence comprising at least about 90% homology to amino acids 20-478 of SEQ ID NO: 1.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises POSTN and/or a sequence comprising at least about 90% homology to amino acids 22-836 of SEQ ID NO: 6.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises IGF2 and/or a sequence comprising at least about 90% homology to amino acids 25-91 of SEQ ID NO: 11.

In certain embodiments, the first polypeptide of Table 2 comprises THBS1 and/or a sequence comprising at least about 90% homology to amino acids 19-1170 of SEQ ID NO: 9; and the second polypeptide of Table 2 comprises IL-15 and/or a sequence comprising at least about 90% homology to amino acids 49-162 of SEQ ID NO: 10.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises THBS4 and/or a sequence comprising at least about 90% homology to amino acids 27-961 of SEQ ID NO: 8. In certain embodiments, the composition further comprises IL-15 and/or a polypeptide comprising at least about 90% homology to amino acids 49-162 of SEQ ID NO: 10. In certain embodiments, the composition further comprises IGF2 and/or a polypeptide comprising at least about 90% homology to amino acids 25-91 of SEQ ID NO: 11.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises FGF17 and/or a sequence comprising at least about 90% homology to amino acids 23-2165 of SEQ ID NO: 7.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises VTN and/or a sequence comprising at least about 90% homology to amino acids 20-478 of SEQ ID NO: 1.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises POSTN and/or a sequence comprising at least about 90% homology to amino acids 22-836 of SEQ ID NO: 6.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises IGF2 and/or a sequence comprising at least about 90% homology to amino acids 25-91 of SEQ ID NO: 11.

In certain embodiments, the first polypeptide of Table 2 comprises THBS2 and/or a sequence comprising at least about 90% homology to amino acids 19-1172 of SEQ ID NO: 4; and the second polypeptide of Table 2 comprises IL-15 and/or a sequence comprising at least about 90% homology to amino acids 49-162 of SEQ ID NO: 10.

In certain embodiments, the first polypeptide of Table 2 comprises THBS4 and/or a sequence comprising at least about 90% homology to amino acids 27-961 of SEQ ID NO: 8; and the second polypeptide of Table 2 comprises FGF17 and/or a sequence comprising at least about 90% homology to amino acids 23-2165 of SEQ ID NO: 7.

In certain embodiments, the first polypeptide of Table 2 comprises THBS4 and/or a sequence comprising at least about 90% homology to amino acids 27-961 of SEQ ID NO: 8, and the second polypeptide of Table 2 comprises VTN and/or a sequence comprising at least about 90% homology to amino acids 20-478 of SEQ ID NO: 1.

In certain embodiments, the first polypeptide of Table 2 comprises THBS4 and/or a sequence comprising at least about 90% homology to amino acids 27-961 of SEQ ID NO: 8, and the second polypeptide of Table 2 comprises POSTN and/or a sequence comprising at least about 90% homology to amino acids 22-836 of SEQ ID NO: 6.

In certain embodiments, the first polypeptide of Table 2 comprises THBS4 and/or a sequence comprising at least about 90% homology to amino acids 27-961 of SEQ ID NO: 8; and the second polypeptide of Table 2 comprises IGF2 and/or a sequence comprising at least about 90% homology to amino acids 25-91 of SEQ ID NO: 11.

In certain embodiments, the first polypeptide of Table 2 comprises THBS4 and/or a sequence comprising at least about 90% homology to amino acids 27-961 of SEQ ID NO: 8; and the second polypeptide of Table 2 comprises IL-15 and/or a sequence comprising at least about 90% homology to amino acids 49-162 of SEQ ID NO: 10.

In certain embodiments, the first polypeptide of Table 2 comprises FGF17 and/or a sequence comprising at least about 90% homology to amino acids 23-2165 of SEQ ID NO: 7; and the second polypeptide of Table 2 comprises VTN and/or a sequence comprising at least about 90% homology to amino acids 20-478 of SEQ ID NO: 1.

In certain embodiments, the first polypeptide of Table 2 comprises FGF17 and/or a sequence comprising at least about 90% homology to amino acids 23-2165 of SEQ ID NO: 7; and the second polypeptide of Table 2 comprises POSTN and/or a sequence comprising at least about 90% homology to amino acids 22-836 of SEQ ID NO: 6.

In certain embodiments, the first polypeptide of Table 2 comprises FGF17 and/or a sequence comprising at least about 90% homology to amino acids 23-2165 of SEQ ID NO: 7; and the second polypeptide of Table 2 comprises IGF2 and/or a sequence comprising at least about 90% homology to amino acids 25-91 of SEQ ID NO: 11.

In certain embodiments, the first polypeptide of Table 2 comprises FGF17 and/or a sequence comprising at least about 90% homology to amino acids 23-2165 of SEQ ID NO: 7; and the second polypeptide of Table 2 comprises IL-15 and/or a sequence comprising at least about 90% homology to amino acids 49-162 of SEQ ID NO: 10.

In certain embodiments, the first polypeptide of Table 2 comprises VTN and/or a sequence comprising at least about 90% homology to amino acids 20-478 of SEQ ID NO: 1, and the second polypeptide of Table 2 comprises POSTN and/or a sequence comprising at least about 90% homology to amino acids 22-836 of SEQ ID NO: 6.

In certain embodiments, the first polypeptide of Table 2 comprises VTN and/or a sequence comprising at least about 90% homology to amino acids 20-478 of SEQ ID NO: 1, and the second polypeptide of Table 2 comprises IGF2 and/or a sequence comprising at least about 90% homology to amino acids 25-91 of SEQ ID NO: 11.

In certain embodiments, the first polypeptide of Table 2 comprises VTN and/or a sequence comprising at least about 90% homology to amino acids 20-478 of SEQ ID NO: 1; and the second polypeptide of Table 2 comprises IL-15 and/or a sequence comprising at least about 90% homology to amino acids 49-162 of SEQ ID NO: 10.

In certain embodiments, the first polypeptide of Table 2 comprises POSTN and/or a sequence comprising at least about 90% homology to amino acids 22-836 of SEQ ID NO: 6, and the second polypeptide of Table 2 comprises IGF2 and/or a sequence comprising at least about 90% homology to amino acids 25-91 of SEQ ID NO: 11.

In certain embodiments, the first polypeptide of Table 2 comprises POSTN and/or a sequence comprising at least about 90% homology to amino acids 22-836 of SEQ ID NO: 6; and the second polypeptide of Table 2 comprises IL-15 and/or a sequence comprising at least about 90% homology to amino acids 49-162 of SEQ ID NO: 10.

In certain embodiments, the first polypeptide of Table 2 comprises IGF2 and/or a sequence comprising at least about 90% homology to amino acids 25-91 of SEQ ID NO: 11; and the second polypeptide of Table 2 comprises IL-15 and/or a sequence comprising at least about 90% homology to amino acids 49-162 of SEQ ID NO: 10.

In certain embodiments, the first therapeutic polypeptide has been recombinantly produced, the second polypeptide has been recombinantly produced, or both the first and second therapeutic polypeptides have been recombinantly produced. In certain embodiments, the first therapeutic polypeptide has been produced in a mammalian, yeast, insect or bacteria cell, the second polypeptide has been produced in a mammalian, yeast, insect or bacteria cell, or both the first and second therapeutic polypeptides have been produced in a mammalian, yeast, insect or bacteria cell. In certain embodiments, the first therapeutic polypeptide and/or the second therapeutic polypeptide have been produced in a mammalian cell and the mammalian cell is a human cell. In some cases the human cell is a human embryonic kidney-derived epithelial cell (e.g., HEK293 cell). In certain embodiments, the first therapeutic polypeptide has been produced in a mammalian cell and the mammalian cell is a Chinese Hamster Ovary (CHO) cell or mouse myeloma cell. In certain embodiments, the second therapeutic polypeptide has been produced in a mammalian cell and the mammalian cell is a Chinese Hamster Ovary (CHO) cell or mouse myeloma cell. In certain embodiments, the first therapeutic polypeptide and/or the second therapeutic polypeptide has been purified from a human biological sample. In some cases, the human biological sample is human plasma. In certain embodiments, the composition further comprises a pharmaceutically acceptable excipient. In certain embodiments, the composition is formulated for administration by injection. In certain embodiments, the injection is intramuscular. In certain embodiments, the injection is subcutaneous. In certain embodiments, the injection is intravenous.

In another aspect, described herein is a composition comprising a polypeptide of Table 2 and a pharmaceutically acceptable excipient. In certain embodiments, the polypeptide comprises VTN and/or a sequence comprising at least about 90% homology to amino acids 20-478 of SEQ ID NO 1. In certain embodiments, the polypeptide comprises POSTN and/or a sequence comprising at least about 90% homology to amino acids 22-836 of SEQ ID NO: 6. In certain embodiments, the polypeptide comprises FGF17 and/or a sequence comprising at least about 90% homology to amino acids 23-2165 of SEQ ID NO: 7 In certain embodiments, the polypeptide comprises THBS2 and/or a sequence comprising at least about 90% homology to amino acids 19-1172 of SEQ ID NO: 4. In certain embodiments, the polypeptide comprises THBS4 and/or a sequence comprising at least about 90% homology to amino acids 27-961 of SEQ ID NO: 8. In certain embodiments, the polypeptide comprises THBS1 and/or a sequence comprising at least about 90% homology to amino acids 19-1170 of SEQ ID NO: 9. In certain embodiments, the polypeptide comprises IL-15 and/or a sequence comprising at least about 90% homology to amino acids 49-162 of SEQ ID NO: 10. In certain embodiments, the polypeptide comprises IGF2 and/or a sequence comprising at least about 90% homology to amino acids 25-91 of SEQ ID NO: 11. In certain embodiments, the polypeptide has been produced in a mammalian cell and the mammalian cell is a human cell. In some cases the human cell is a human embryonic kidney-derived epithelial cell (e.g., HEK293 cells). In certain embodiments, the polypeptide has been expressed from a Chinese Hamster Ovary (CHO) cell, or mouse myeloma (NS0) cell, insect, or a bacterial cell. In some cases the bacterial cell is an E. coli cell. In certain embodiments, the polypeptide has been purified from a human biological sample. In some cases, the human biological sample is human plasma. In certain embodiments, the polypeptide is synthetically produced. In certain embodiments, the polypeptide possesses mitogenic and/or fusion promoting activity. Further described is a nucleic acid encoding the polypeptide described herein. Further described is a cell line comprising the nucleic acid.

Further described herein is a method of treating a disease or condition in a subject in need thereof, the method comprising administering to the subject a composition described herein. In certain embodiments, the disease or condition comprises an aging disorder, muscle wasting disorder, muscle injury, or injury to connective tissue, or a combination thereof. In certain embodiments, the subject has the aging disorder and the aging disorder comprises sarcopenia. In certain embodiments, the subject has the muscle wasting disorder and the muscle wasting disorder comprises muscular dystrophy. In certain embodiments, the subject has the muscle wasting disorder and the muscle wasting is a result of obesity, disease progression, metabolic disorder, or therapeutic treatment, or a combination thereof. In certain embodiments, the metabolic disorder is diabetes. In some cases the diabetes is Type 2 Diabetes. In certain embodiments, the subject has the muscle wasting disorder and the muscle wasting is cachexia, e.g., muscular cachexia.

In another aspect, described herein is a composition comprising. (a) a first therapeutic polypeptide comprising THBS1, THBS2, THBS4, FGF17, VTN, POSTN, IGF2, or IL-15, and (b) a second therapeutic polypeptide comprising THBS1, THBS2, THBS4, FGF17, VTN, POSTN, IGF2, or IL-15. In certain embodiments, the first therapeutic polypeptide has been recombinantly produced, the second polypeptide has been recombinantly produced, or both the first and second therapeutic polypeptides have been recombinantly produced. In certain embodiments, the first therapeutic polypeptide has been produced in a mammalian, yeast, insect or bacteria cell, the second polypeptide has been produced in a mammalian, yeast, insect or bacteria cell, or both the first and second therapeutic polypeptides have been produced in a mammalian, yeast, insect or bacteria cell. In certain embodiments, the polypeptide has been produced in a mammalian cell and the mammalian cell is a human cell. In some cases the human cell is a human embryonic kidney-derived epithelial cell (e.g., HEK293 cells). In certain embodiments, the first therapeutic polypeptide has been produced in a mammalian cell and the mammalian cell is a Chinese Hamster Ovary (CHO) cell or mouse myeloma cell. In certain embodiments, the second therapeutic polypeptide has been produced in a mammalian cell and the mammalian cell is a Chinese Hamster Ovary (CHO) cell or mouse myeloma cell. In certain embodiments, the polypeptide has been purified from a human biological sample. In some cases, the human biological sample is human plasma.

In certain embodiments, the first therapeutic polypeptide comprises IL-15 and/or a sequence comprising at least about 90% homology to amino acids 49-162 of SEQ ID NO: 10; and the second therapeutic polypeptide comprises THBS2 and/or a sequence comprising at least about 90% homology to amino acids 19-1172 of SEQ ID NO: 4. In certain embodiments, the first therapeutic polypeptide comprises IL-15 and/or a sequence comprising at least about 90% homology to amino acids 49-162 of SEQ ID NO: 10; and the second therapeutic polypeptide comprises THBS4 and/or a sequence comprising at least about 90% homology to amino acids 27-961 of SEQ ID NO: 8. In certain embodiments, the first therapeutic polypeptide comprises IGF2 and/or a sequence comprising at least about 90% homology to amino acids 25-91 of SEQ ID NO: 11; and the second therapeutic polypeptide comprises THBS2 and/or a sequence comprising at least about 90% homology to amino acids 19-1172 of SEQ ID NO: 4. In certain embodiments, the first therapeutic polypeptide comprises IGF2 and/or a sequence comprising at least about 90% homology to amino acids 25-91 of SEQ ID NO: 11; and the second therapeutic polypeptide comprises THBS4 and/or a sequence comprising at least about 90% homology to amino acids 27-961 of SEQ ID NO: 8 In certain embodiments, the first therapeutic polypeptide comprises THBS4 and/or a sequence comprising at least about 90% homology to amino acids 27-961 of SEQ ID NO: 8; and the second therapeutic polypeptide comprises THBS2 and/or a sequence comprising at least about 90% homology to amino acids 19-1172 of SEQ ID NO: 4. In certain embodiments, the composition further comprises IL-15 and/or a sequence comprising at least about 90% homology to amino acids 49-162 of SEQ ID NO: 10 In certain embodiments, the composition further comprises IGF2 and/or a sequence comprising at least about 90% homology to amino acids 25-91 of SEQ ID NO: 11 In certain embodiments, the composition further comprises a pharmaceutically acceptable excipient. In certain embodiments, the composition is formulated for administration by injection. In certain embodiments, the injection is intramuscular. In certain embodiments, the injection is subcutaneous. In certain embodiments, the injection is intravenous.

In certain embodiments, described herein is a composition comprising THBS2 and THBS4. The composition may comprise VTN. The composition may comprise ANOS1. The composition may comprise IL-15 The composition may comprise IGF2. In certain embodiments, the composition comprises THBS2, THBS4, and VIN. In certain embodiments, the composition comprises THBS2, THBS4, and ANOS1 in certain embodiments, the composition comprises THBS2, THBS4, and IL-15. In certain embodiments, the composition comprises THBS2, THBS4, and IGF2. In certain embodiments, the composition further comprises a pharmaceutically acceptable excipient. In certain embodiments, the composition is formulated for administration by injection. In certain embodiments, the injection is intramuscular. In certain embodiments, the injection is subcutaneous. In certain embodiments, the injection is intravenous.

In certain embodiment, described herein is a composition comprising THBS1 and FGF17 in certain embodiment, described herein is a composition comprising THBS2 and VTN in certain embodiment, described herein is a composition comprising THBS1 and VTN. In certain embodiment, described herein is a composition comprising THBS1 and THBS2. In certain embodiment, described herein is a composition comprising THBS2 and FGF17. In certain embodiment, described herein is a composition comprising THBS1 and THBS4. In certain embodiment, described herein is a composition comprising VTN and FGF17. In certain embodiment, described herein is a composition comprising THBS4 and VTN. In certain embodiment, described herein is a composition comprising THBS4 and FGF17. In certain embodiments, the composition further comprises a pharmaceutically acceptable excipient. In certain embodiments, the composition is formulated for administration by injection. In certain embodiments, the injection is intramuscular. In certain embodiments, the injection is subcutaneous. In certain embodiments, the injection is intravenous.

Further described is a method of treating a disease or condition in a subject in need thereof, the method comprising administering to the subject a composition described herein. In certain embodiments, the disease or condition comprises an aging disorder, muscle wasting disorder, muscle injury, or injury to connective tissue, or a combination thereof. In certain embodiments, the subject has the aging disorder and the aging disorder comprises sarcopenia. In certain embodiments, the subject has the muscle wasting disorder and the muscle wasting disorder comprises muscular dystrophy. In certain embodiments, the subject has the muscle wasting disorder and the muscle wasting is a result of obesity, metabolic disorder, disease progression, or therapeutic treatment, or a combination thereof. In certain embodiments, the metabolic disorder is diabetes. In some cases the diabetes is Type 2 Diabetes. In certain embodiments, the subject has the muscle wasting disorder and the muscle wasting is cachexia, e.g., muscular cachexia.

In a certain aspect, described herein, is a composition comprising a mitogenic and/or fusion promoting polypeptide, wherein the polypeptide is a heparin-associated polypeptide secreted from a stem cell or a transformed cell line, wherein the heparin-associated polypeptide possesses mitogenic and/or fusion promoting activity. In certain embodiments, the composition comprises a plurality of mitogenic and/or fusion promoting polypeptides. In certain embodiments, the plurality comprises two, three, four, five, six, seven, eight, nine, ten or more polypeptides. In certain embodiments, the mitogenic and/or fusion promoting polypeptide or plurality of mitogenic and/or fusion promoting polypeptides are isolated and purified. In certain embodiments, the mitogenic and/or fusion promoting polypeptide or plurality of mitogenic and/or fusion promoting polypeptides are recombinantly produced. In certain embodiments, the mitogenic and/or fusion promoting polypeptide or plurality of mitogenic and/or fusion promoting polypeptides are secreted from a stem cell in certain embodiments, the stem cell is a pluripotent stem cell. In certain embodiments, the stem cell is an induced pluripotent stem cell. In certain embodiments, the mitogenic activity comprises the ability to increase proliferation in a somatic cell and/or increase the ability of another heparin-associated polypeptide to increase proliferation in a somatic cell. In certain embodiments, the fusion promoting activity comprises the ability to increase fusion of a plurality of somatic cells and/or increase the ability of another heparin—associated polypeptide to increase fusion in a plurality of somatic cells. In certain embodiments, the somatic cell is a muscle, muscle progenitor cell, tenocyte, or tenocyte precursor. In certain embodiments, the somatic cell is a mammalian cell. In certain embodiments, the somatic cell is a human cell. In certain embodiments, any one of the mitogenic and/or fusion promoting polypeptides or plurality of mitogenic and/or fusion promoting polypeptides are produced in a heterologous cellular production system. In certain embodiments, any one of the mitogenic and/or fusion promoting polypeptides or plurality of mitogenic and/or fusion promoting polypeptides are synthetically produced. In certain embodiments, the polypeptides are purified from human plasma. In certain embodiments, the polypeptides are purified from a mammalian cell, insect cell, or bacterial cell. The mammalian cell may be a human cell. In certain embodiments, the mitogenic and/or fusion promoting polypeptide or plurality of mitogenic and/or fusion promoting polypeptides comprises one or more modifications to improve activity, stability, or increase polypeptide yield from a heterologous cellular production system, or any combination thereof. In certain embodiments, the modification is an alteration of one or more amino acids in the polypeptide sequence of mitogenic and/or fusion promoting polypeptide compared to the wildtype polypeptide sequence of the mitogenic and/or fusion promoting polypeptide. In certain embodiments, the mitogenic and/or fusion promoting polypeptide(s) are encoded by a nucleic acid. In certain embodiments, the cell line comprises the nucleic acid encoding the mitogenic and/or fusion promoting polypeptide(s). In certain embodiments, the cell line is a eukaryotic cell line. In certain embodiments, the composition comprises the mitogenic and/or fusion promoting polypeptide or plurality of mitogenic and/or fusion promoting polypeptides and a pharmaceutically acceptable excipient, carrier, or diluent in certain embodiments, the composition is formulated in injectable form. In certain embodiments, the composition is for use in a method of treating an aging disorder, a muscle wasting disorder, a muscle injury, or an injury to a connective tissue, or any combination thereof. In certain embodiments, the aging disorder is sarcopenia. In certain embodiments, the muscle wasting disorder is muscular dystrophy in certain embodiments, the muscle wasting disorder is cachexia, e.g., muscular cachexia. In certain embodiments, described herein, is a method of treating an individual with aging disorder, a muscle wasting disorder, a muscle injury, or an injury to a connective tissue, or any combination thereof, the method comprising administering to the individual the composition of mitogenic and/or fusion promoting polypeptides. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of producing a composition suitable for the treatment of an aging disorder comprising admixing a pharmaceutically acceptable excipient, carrier, or diluent with the mitogenic and/or fusion promoting polypeptide or plurality of mitogenic and/or fusion promoting polypeptides described herein. In certain embodiments, described herein, is a method of producing a mitogenic and/or fusion promoting polypeptide comprising culturing a cell line comprising a nucleic acid expressing a mitogenic and/or fusion promoting polypeptide under conditions sufficient to produce the mitogenic and/or fusion promoting polypeptide.

In another aspect, described herein, is a composition comprising a mitogenic and/or fusion promoting polypeptide, wherein the mitogenic and/or fusion promoting polypeptide is identified by: (a) identifying at least one polypeptide in a mixture of a plurality of polypeptides secreted from a stem cell or a transformed cell line that can be purified with heparin coated matrix; and (b) determining the mitogenic and/or fusion promoting activity of the at least one polypeptide for a somatic cell, wherein the at least one polypeptide is identified as a mitogenic and/or fusion promoting polypeptide if the at least one polypeptide exhibits mitogenic and/or fusion promoting activity. In certain embodiments, the composition comprises a plurality of mitogenic and/or fusion promoting polypeptides in certain embodiments, the plurality comprises three, four, five, six, seven, eight, nine, ten or more polypeptides. In certain embodiments, the mitogenic and/or fusion promoting polypeptide or plurality of mitogenic and/or fusion promoting polypeptides are isolated and purified. In certain embodiments, the mitogenic and/or fusion promoting polypeptide or plurality of mitogenic and/or fusion promoting polypeptides are recombinantly produced. In certain embodiments, the mitogenic and/or fusion promoting polypeptide or plurality of mitogenic and/or fusion promoting polypeptides are secreted from a stem cell. In certain embodiments, the stem cell is a pluripotent stem cell. In certain embodiments, the stem cell is an induced pluripotent stem cell. In certain embodiments, the mitogenic and/or fusion promoting activity comprises the ability to increase proliferation in a somatic cell or increase the ability of another heparin-associated polypeptide to increase proliferation in a somatic cell. In certain embodiments, the fusion promoting activity comprises the ability to increase fusion of a plurality of somatic cells or increase the ability of another heparin-associated polypeptide to increase fusion in a plurality of somatic cells. In certain embodiments, the somatic cell is a muscle, muscle progenitor cell, tenocyte, or tenocyte precursor. In certain embodiments, the somatic cell is a mammalian cell. In certain embodiments, the somatic cell is a human cell. In certain embodiments, any one of the mitogenic and/or fusion promoting polypeptides or plurality of mitogenic and/or fusion promoting polypeptides are produced in a heterologous cellular production system. In certain embodiments, any one of the mitogenic and/or fusion promoting polypeptides or plurality of mitogenic and/or fusion promoting polypeptides are synthetically produced. In certain embodiments, the polypeptides are purified from human plasma. In certain embodiments, the polypeptides are purified from a mammalian cell, insect cell, or bacterial cell. The mammalian cell may be a human cell. In certain embodiments, the mitogenic and/or fusion promoting polypeptide or plurality of mitogenic and/or fusion promoting polypeptides comprises one or more modifications to improve activity, stability, or increase polypeptide yield from a heterologous cellular production system. In certain embodiments, the modification is an alteration of one or more amino acids in the polypeptide sequence of the mitogenic and/or fusion promoting polypeptide compared to the wildtype polypeptide sequence of the mitogenic and/or fusion promoting polypeptide. In certain embodiments, the mitogenic and/or fusion promoting polypeptide(s) are encoded by a nucleic acid. In certain embodiments, the cell line comprises the nucleic acid encoding the mitogenic and/or fusion promoting polypeptide(s). In certain embodiments, the cell line is a eukaryotic cell line. In certain embodiments, the composition comprises the mitogenic and/or fusion promoting polypeptide or plurality of mitogenic and/or fusion promoting polypeptides and a pharmaceutically acceptable excipient, carrier, or diluent. In certain embodiments, the composition is formulated in injectable form. In certain embodiments, the composition is for use in a method of treating an aging disorder, a muscle wasting disorder, a muscle injury, or an injury to a connective tissue, or any combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of treating an individual with aging disorder, a muscle wasting disorder, a muscle injury, or an injury to a connective tissue, or any combination thereof, the method comprising administering to the individual the composition of mitogenic and/or fusion promoting polypeptides. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of producing a composition suitable for the treatment of an aging disorder comprising admixing a pharmaceutically acceptable excipient, carrier, or diluent with the mitogenic and/or fusion promoting polypeptide or plurality of mitogenic and/or fusion promoting polypeptides described herein. In certain embodiments, described herein, is a method of producing a mitogenic and/or fusion promoting polypeptide comprising culturing a cell line comprising a nucleic acid expressing a mitogenic and/or fusion promoting polypeptide under conditions sufficient to produce the mitogenic and/or fusion promoting polypeptide.

In another aspect, described herein, is a composition comprising a mitogenic and/or fusion promoting polypeptide, wherein the mitogenic and/or fusion promoting polypeptide comprises ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COIA1A1, LAMB2, CCDC80, COL1A2, LTF, CFH, COL2A1, MATN2, CLEC3B, COL3A1, MDK, COL25A1, COLSA1, MST1, COL5A3, COLSA2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, a protein listed in Table 2, or a protein listed in Table 1, or any combination thereof. In certain embodiments, the mitogenic and/or fusion promoting polypeptide comprises VTN, STC2, AGRN, POSTN, FGF17, THBS2, FST, THBS4, IGF2, IL-15, or THBS1, or a combination thereof. In certain embodiments, the mitogenic and/or fusion promoting polypeptide comprises one or more of VTN, POSTN, FGF17, THBS2, IGF2, IL-15, THBS1, and THBS4. In certain embodiments, the mitogenic and/or fusion promoting polypeptide comprises VTN. In certain embodiments, the mitogenic and/or fusion promoting polypeptide comprises STC2. In certain embodiments, the mitogenic and/or fusion promoting polypeptide comprises AGRN in certain embodiments, the mitogenic and/or fusion promoting polypeptide comprises THBS2. In certain embodiments, the mitogenic and/or fusion promoting polypeptide comprises FST. In certain embodiments, the mitogenic and/or fusion promoting polypeptide comprises POSTN. In certain embodiments, the mitogenic and/or fusion promoting polypeptide comprises FGF17. In certain embodiments, the mitogenic and/or fusion promoting polypeptide comprises THBS4. In certain embodiments, the mitogenic and/or fusion promoting polypeptide comprises IGF2. In certain embodiments, the mitogenic and/or fusion promoting polypeptide comprises IL-15 in certain embodiments, the mitogenic and/or fusion promoting polypeptide comprises THBS1. In certain embodiments, the composition comprises a plurality of mitogenic and/or fusion promoting polypeptides. In certain embodiments, the plurality comprises three, four, five, six, seven, eight, nine, ten or more polypeptides. The plurality of polypeptides may comprise one, two, three, four, five, six, seven, eight, nine, ten or more polypeptides of Table 2. The plurality of polypeptides may comprise one, two, three, four, five, six, seven, eight, nine, ten or more polypeptides of Table 1. In certain embodiments, the plurality of mitogenic and/or fusion promoting polypeptides comprises IGF2, THBS2, and THBS4. In certain embodiments, the plurality of mitogenic and/or fusion promoting polypeptides comprises IL-15 THBS2, and THBS4. In certain embodiments, the plurality of mitogenic and/or fusion promoting polypeptides comprises THBS2 and THBS4. In certain embodiments, the plurality of mitogenic and/or fusion promoting polypeptides comprises THBS2, THBS4, and VTN. In certain embodiments, the plurality of mitogenic and/or fusion promoting polypeptides comprises THBS2, THBS4, and ANOS1. In certain embodiments, the plurality of mitogenic and/or fusion promoting polypeptides comprises THBS2, THBS4, and IL-15. In certain embodiments, the plurality of mitogenic and/or fusion promoting polypeptides comprises THBS2, THBS4, and IGF2. In certain embodiments, the plurality of mitogenic and/or fusion promoting polypeptides comprises THBS1 and FGF17. In certain embodiments, the plurality of mitogenic and/or fusion promoting polypeptides comprises THBS2 and VTN. In certain embodiments, the plurality of mitogenic and/or fusion promoting polypeptides comprises THBS1 and VTN. In certain embodiments, the plurality of mitogenic and/or fusion promoting polypeptides comprises THBS1 and THBS2 In certain embodiments, the plurality of mitogenic and/or fusion promoting polypeptides comprises THBS2 and FGF17. In certain embodiments, the plurality of mitogenic and/or fusion promoting polypeptides comprises THBS1 and THBS4 In certain embodiments, the plurality of mitogenic and/or fusion promoting polypeptides comprises VTN and FGF17. In certain embodiments, the plurality of mitogenic and/or fusion promoting polypeptides comprises THBS4 and VTN. In certain embodiments, the plurality of mitogenic and/or fusion promoting polypeptides comprises THBS4 and FGF17. In certain embodiments, the mitogenic and/or fusion promoting polypeptide or plurality of mitogenic and/or fusion promoting polypeptides are isolated and purified. In certain embodiments, the mitogenic and/or fusion promoting polypeptide or plurality of mitogenic and/or fusion promoting polypeptides are recombinantly produced. In certain embodiments, the mitogenic and/or fusion promoting polypeptide or plurality of mitogenic and/or fusion promoting polypeptides are secreted from a stem cell. In certain embodiments, the stem cell is a pluripotent stem cell. In certain embodiments, the stem cell is an induced pluripotent stem cell. In certain embodiments, the mitogenic activity comprises the ability to increase proliferation in a somatic cell or increase the ability of another heparin-associated polypeptide to increase proliferation in a somatic cell. In certain embodiments, the fusion promoting activity comprises the ability to increase fusion of a plurality of somatic cells and/or increase the ability of another heparin-associated polypeptide to increase fusion in a plurality of somatic cells. In certain embodiments, the somatic cell is a muscle, muscle progenitor cell, tenocyte, or tenocyte precursor. In certain embodiments, the somatic cell is a mammalian cell. In certain embodiments, the somatic cell is a human cell. In certain embodiments, any one of the mitogenic and/or fusion promoting polypeptides or plurality of mitogenic and/or fusion promoting polypeptides are produced in a heterologous cellular production system. In certain embodiments, any one of the mitogenic and/or fusion promoting polypeptides or plurality of mitogenic and/or fusion promoting polypeptides are synthetically produced. In certain embodiments, the polypeptides are purified from human plasma. In certain embodiments, the polypeptides are purified from a mammalian cell, insect cell, or bacterial cell. The mammalian cell may be a human cell. In certain embodiments, the mitogenic and/or fusion promoting polypeptide or plurality of mitogenic and/or fusion promoting polypeptides comprises one or more modifications to improve activity, stability, or increase polypeptide yield from a heterologous cellular production system. In certain embodiments, the modification is an alteration of one or more amino acids in the polypeptide sequence of the mitogenic and/or fusion promoting polypeptide compared to the wildtype polypeptide sequence of the mitogenic and/or fusion promoting polypeptide. In certain embodiments, the mitogenic and/or fusion promoting polypeptide(s) are encoded by a nucleic acid. In certain embodiments, the cell line comprises the nucleic acid encoding the mitogenic and/or fusion promoting polypeptide(s). In certain embodiments, the cell line is a eukaryotic cell line. In certain embodiments, the composition comprises the mitogenic and/or fusion promoting polypeptide or plurality of mitogenic and/or fusion promoting polypeptides and a pharmaceutically acceptable excipient, carrier, or diluent. In certain embodiments, the composition is formulated in injectable form. In certain embodiments, the composition is for use in a method of treating an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of treating an individual with aging disorder, a muscle wasting disorder, a muscle injury, or an injury to a connective tissue, or a combination thereof, the method comprising administering to the individual the composition of mitogenic and/or fusion promoting polypeptides. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of producing a composition suitable for the treatment of an aging disorder comprising admixing a pharmaceutically acceptable excipient, carrier, or diluent with the mitogenic and/or fusion promoting polypeptide or plurality of mitogenic and/or fusion promoting polypeptides described herein. In certain embodiments, described herein, is a method of producing a mitogenic and/or fusion promoting polypeptide comprising culturing a cell line comprising a nucleic acid expressing a mitogenic and/or fusion promoting polypeptide under conditions sufficient to produce the mitogenic and/or fusion promoting polypeptide.

In another aspect, described herein, is a composition comprising a mitogenic and/or fusion promoting polypeptide, wherein the mitogenic and/or fusion promoting polypeptide comprises VTN, STC2, AGRN, POSTN, FGF17, THBS2, FST, THBS4, IGF2, IL-15, or THBS1, or a combination thereof. In certain embodiments, the mitogenic and/or fusion promoting polypeptide comprises one or more of VTN, POSTN, FGF17, THBS2, THBS4, IGF2, IL-15, and THBS1. In certain embodiments, the mitogenic and/or fusion promoting polypeptide comprises VTN. In certain embodiments, the mitogenic and/or fusion promoting polypeptide comprises STC2. In certain embodiments, the mitogenic and/or fusion promoting polypeptide comprises AGRN. In certain embodiments, the mitogenic and/or fusion promoting polypeptide comprises THBS2. In certain embodiments, the mitogenic and/or fusion promoting polypeptide comprises FST. In certain embodiments, the mitogenic and/or fusion promoting polypeptide comprises POSTN. In certain embodiments, the mitogenic and/or fusion promoting polypeptide comprises FGF17. In certain embodiments, the mitogenic and/or fusion promoting polypeptide comprises THBS4. In certain embodiments, the mitogenic and/or fusion promoting polypeptide comprises IGF2. In certain embodiments, the mitogenic and/or fusion promoting polypeptide comprises IL-15. In certain embodiments, the mitogenic and/or fusion promoting polypeptide comprises THBS1. In certain embodiments, the composition comprises a mixture of a plurality of different mitogenic and/or fusion promoting polypeptides. In certain embodiments, the plurality of different mitogenic and/or fusion promoting polypeptides comprise three, four, or five different mitogenic and/or fusion promoting polypeptides. The plurality of polypeptides may comprise one, two, three, four, five, six, seven, eight, nine, ten or more polypeptides of Table 2. The plurality of polypeptides may comprise one, two, three, four, five, six, seven, eight, nine, ten or more polypeptides of Table 1. In certain embodiments, the plurality of mitogenic and/or fusion promoting polypeptides comprises IGF2, THBS2, and THBS4. In certain embodiments, the plurality of mitogenic and/or fusion promoting polypeptides comprises IL-15, THBS2, and THBS4. In certain embodiments, the plurality of mitogenic and/or fusion promoting polypeptides comprises THBS2 and THBS4. In certain embodiments, the plurality of mitogenic and/or fusion promoting polypeptides comprises THBS2, THBS4, and VTN in certain embodiments, the plurality of mitogenic and/or fusion promoting polypeptides comprises THBS2, THBS4, and ANOS1. In certain embodiments, the plurality of mitogenic and/or fusion promoting polypeptides comprises THBS2, THBS4, and IL-15. In certain embodiments, the plurality of mitogenic and/or fusion promoting polypeptides comprises THBS2, THBS4, and IGF2. In certain embodiments, the plurality of mitogenic and/or fusion promoting polypeptides comprises THBS1 and FGF17. In certain embodiments, the plurality of mitogenic and/or fusion promoting polypeptides comprises THBS2 and VTN. In certain embodiments, the plurality of mitogenic and/or fusion promoting polypeptides comprises THBS1 and VTN. In certain embodiments, the plurality of mitogenic and/or fusion promoting polypeptides comprises THBS1 and THBS2 in certain embodiments, the plurality of mitogenic and/or fusion promoting polypeptides comprises THBS2 and FGF17. In certain embodiments, the plurality of mitogenic and/or fusion promoting polypeptides comprises THBS1 and THBS4 in certain embodiments, the plurality of mitogenic and/or fusion promoting polypeptides comprises VTN and FGF17. In certain embodiments, the plurality of mitogenic and/or fusion promoting polypeptides comprises THBS4 and VTN. In certain embodiments, the plurality of mitogenic and/or fusion promoting polypeptides comprises THBS4 and FGF17. In certain embodiments, the mitogenic and/or fusion promoting polypeptide or plurality of mitogenic and/or fusion promoting polypeptides are isolated and purified. In certain embodiments, the mitogenic and/or fusion promoting polypeptide or plurality of mitogenic and/or fusion promoting polypeptides are recombinantly or synthetically produced. In certain embodiments, the polypeptides are purified from human plasma. In certain embodiments, the polypeptides are purified from a mammalian cell, insect cell, or bacterial cell. The mammalian cell may be a human cell. In certain embodiments, the mitogenic and/or fusion promoting polypeptide or plurality of mitogenic and/or fusion promoting polypeptides are secreted from a stem cell. In certain embodiments, the stem cell is a pluripotent stem cell. In certain embodiments, the stem cell is an induced pluripotent stem cell. In certain embodiments, the mitogenic activity comprises increasing proliferation in a muscle cell precursor in certain embodiments, the fusion promoting activity comprises the ability to increase fusion of a plurality of somatic cells and/or increase the ability of another heparin-associated polypeptide to increase fusion in a plurality of somatic cells. In certain embodiments, the muscle cell precursor is a myoblast. In certain embodiments, the somatic cell is a mammalian cell. In certain embodiments, the somatic cell is a human cell. In certain embodiments, the mitogenic and/or fusion promoting polypeptide or plurality of mitogenic and/or fusion promoting polypeptides comprise one or more modifications to improve activity, stability, or increase polypeptide yield from a heterologous cellular production system. In certain embodiments, the modification is an alteration of one or more amino acids in the polypeptide sequence of the mitogenic and/or fusion promoting polypeptide compared to the wildtype polypeptide sequence of the mitogenic and/or fusion promoting polypeptide. In certain embodiments, the modification is a fusion of a mitogenic and/or fusion promoting polypeptide to a non-mitogenic or fusion promoting polypeptide in certain embodiments, the non-mitogenic or fusion promoting polypeptide comprises an immunoglobulin Fc region or serum albumin. In certain embodiments, the non-mitogenic or fusion promoting polypeptide is a human polypeptide in certain embodiments, the mitogenic and/or fusion promoting polypeptide or plurality of mitogenic and/or fusion promoting polypeptides are concatemerized. In certain embodiments, the concatemerized polypeptides are separated by a polypeptide linker. In certain embodiments, the concatemerized polypeptides are the same polypeptide. In certain embodiments, the concatemerized polypeptides are different polypeptides. In certain embodiments, the concatemerized polypeptides are covalently concatemerized through a non-peptide linkage. In certain embodiments, the concatemerized polypeptides are non-covalently concatemerized. In certain embodiments, a nucleic acid encodes the mitogenic and/or fusion promoting polypeptide. In certain embodiments, a cell line comprises the nucleic acid. In certain embodiments, the cell line is a eukaryotic cell line. In certain embodiments, the mitogenic and/or fusion promoting polypeptide or plurality of mitogenic and/or fusion promoting polypeptides comprises a pharmaceutically acceptable excipient, carrier, or diluent. In certain embodiments, the pharmaceutically acceptable excipient, carrier, or diluent increases the function of the mitogenic and/or fusion promoting polypeptide or plurality of mitogenic and/or fusion promoting polypeptides. In certain embodiments, the function comprises increasing proliferation in a muscle cell precursor, increasing stability of the mitogenic and/or fusion promoting polypeptide or plurality of mitogenic and/or fusion promoting polypeptides, or increasing bioavailability of the mitogenic and/or fusion promoting polypeptide or plurality of mitogenic and/or fusion promoting polypeptides. In certain embodiments, the composition is for subcutaneous, intravenous, intramuscular, or topical administration. In certain embodiments, the composition is for use in a method of treating an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia. In certain embodiments, described herein, is a method of treating an individual with an aging disorder, a muscle wasting disorder, a muscle injury, or an injury to a connective tissue, or a combination thereof, the method comprising administering to the individual the composition. In certain embodiments, described herein, is a method of increasing proliferation of a muscle cell or connective tissue cell precursor in an individual comprising administering to the individual the composition. In certain embodiments, the individual is afflicted with or suspected of being afflicted with an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia.

Also described is a method of producing a composition suitable for the treatment of an aging disorder, a muscle wasting disorder, a muscle injury, or an injury to a connective tissue, or a combination thereof, the method comprising admixing a pharmaceutically acceptable excipient, carrier, or diluent with the mitogenic and/or fusion promoting polypeptide or plurality of mitogenic and/or fusion promoting polypeptides. Also described is a method of producing a mitogenic and/or fusion promoting polypeptide comprising culturing a cell line comprising a nucleic acid encoding mitogenic and/or fusion promoting polypeptide or plurality of mitogenic and/or fusion promoting polypeptides under conditions sufficient to produce the mitogenic and/or fusion promoting polypeptide.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1 shows histology and immunofluorescence staining of injured and regenerated muscle tissue from aged mice treated with heparin-associated polypeptides or vehicle control.

FIG. 2A shows time-points for dosing and analysis of the effects of the entire pool of heparin-associated polypeptides in an acute injury model in aged mice. Squares denote injury inducing intramuscular injection (IM) with Barium Chloride, circles denote administration of treatment or vehicle.

FIG. 2B shows the results of the experiment outlined in FIG. 2A. Administration of hPSC derived factors resulted in improved new fiber formation (regenerative index, left) and reduced scaring (fibrotic index, right) in aged mice to levels similar to those in young mice, both of which were markedly better than vehicle-treated old mice. Stars indicate degree of significance from one-way ANOVA tests.

FIG. 2C shows time-points for dosing and analysis of the effects of the entire pool of heparin-associated polypeptides in an acute injury model in transgenic mice prone to obesity. Squares denote injury inducing intramuscular injection (IM) with Barium Chloride, circles denote administration of treatment or vehicle.

FIG. 2D shows the results of the experiment outlined in FIG. 2C. Administration of hPSC derived factors resulted in improved new fiber formation (regenerative index, left, p<3.5E-6) and reduced scaring (fibrotic index, right, p<8.44E-3) in transgenic obese mice to levels similar to those in positive control (8 ng FGF19) treated mice (p<2.3E-5 and p<2.32E-2, respectively), both of which were markedly better than vehicle-treated transgenic obese mice. Stars indicate degree of significance from one-way ANOVA tests.

FIGS. 3A to 3D. Heparin-column enrichment of secreted factors from hESC and differentiated cells can be deeply profiled in an unbiased way using isobaric labeling, online reverse phase fractionation, and an SPS-MS3 instrument method. FIG. 3A depicts the methodology for targeted heparin purification of secreted proteins from media supernatant, isobaric labeling, reverse phase fractionation, and SPS-MS3 mass spectrometry instrument method. FIG. 3B depicts a silver stain gel of heparin purified proteins (left two lanes) compared to buffer only (right most lane), demonstrating enrichment of many protein species by their molecular weight. FIG. 3C depicts a 21) heat map of the intensity (color) of peptides separated by m/z (y-axis) compared to elution from reverse phase nHPLC separation (x-axis) FIG. 3D depicts a K-means clustered heat map of all proteins found in a representative experiment demonstrating the differential abundance of many proteins specifically in the secretome of undifferentiated hPSCs. FIG. 3E depicts Principle Component Analysis of proteins from the same run demonstrating again the differential expressionless (PC1) as well as a high relative degree of reproducibility from undifferentiated hPSCs compared to differentiated cells.

FIGS. 4A and 4B illustrate representative results from an in vitro assay useful to validate the regenerative capacity of factors identified by mass spectroscopy. FIG. 4A shows representative micrographs taken from cells treated with fusion media (neg. control), defined growth media (pos. control), Optimem, supernatant from differentiated hESC, supernatant from undifferentiated hESC, heparin binding proteins eluted from supernatant of undifferentiated hESC under two different conditions, and supernatant of undifferentiated hESC that has been depleted of heparin binding proteins. FIG. 4B shows data expressed as % of nuclei stained with BrdU or % cells stained for embryonic myosin heavy chain (eMyHC).

FIGS. 5A-5B show quantitation and representative images demonstrating the proliferation effect of IGFBP7 (330 ng/mL), POSTN (330 ng/mL), SPON1 (330 ng/mL), MST1 (330 ng/mL), and RARRES2 (330 ng/mL) (FIG. 5A); and VTN (10 ug/mL), FGF17 (500 ng/mL), IGF2 (2 ug/mL), FGF4 (500 ng/mL), FiF1 (500 ng/mL), and FGF6 (1 ug/mL) (FIG. 5B) in injury activated primary mouse myoblasts grown in vitro. FIG. 5C shows quantitation and representative images demonstrating the increased cellular fusion effect of THBS1 (330 ng/mL), THBS2 (330 ng/mL), and STC2 (875 ng/mL) in injury activated primary mouse myoblasts grown in vitro.

FIGS. 6A-6E show quantitation and representative immunofluorescent stained cell images demonstrating the proliferation effect specific heparin-associated polypeptides FIG. 6A shows the effect of IGFBP5 at 1 ug/mL, FIG. 6B show the effect of THBS4 at 1 ug/mL, FIG. 6C shows the effect of VTN at 10 ug/mL, FIG. 6D shows the effect of FGF17 at 250 ng/mL, and FIG. 6E shows the effect of IGFBP7 at 500 ng/mL—all demonstrated notable effects in injury activated primary human myoblasts, young (18 years old) and aged (both 68 years old), grown in vitro. FIGS. 6F-6H, show quantitation and representative images demonstrating the increased cellular fusion effect of SPON1 (1 ug/mL) (FIG. 6F), POSTN (1 ug/mL) (FIG. 6G), PDGFRL (5 ug/mL) (FIG. 6H) in injury activated primary human myoblasts, young (18 years) and old (both 69 years old), grown in vitro.

FIG. 7A provides an exemplary proliferative dose response of mouse myoblasts cultured with hPSC-derived factors Thrombospondin 1 (THBS1) applied at 125 ng/mL, 250 ng/mL, and 500 ng/mL, 1000 ng/ml and 200 ng/ml, and Fibroblast growth factor 17 (FGF17) applied at 12.5 ng/ml, 25 ng/ml, 50 ng/ml, 100 ng/ml, and 200 ng/ml. FIG. 7B shows that while THBS1 showed non-significant effects on proliferation of myoblasts (top), FGF17 produced a linear dose-dependent increase in proliferation of myoblasts (bottom). FIG. 7C shows that the combination of the two produced potentiation type synergy (CI<0.68, p<7.92E-7).

FIGS. 7D-7M provide examples of synergistic combinations of heparin-associated polypeptides relative to the vehicle only control (_FM) or to treatment with either of the individual heparin-associated polypeptides. Combination Index (CI) values and probability values (p-values) from statistical tests for the synergy models are reported in Table 10. FIG. 7D shows the synergistic effects of THBS1 with VTN FIG. 7E shows the synergistic effects of THBS1 with THBS2. FIG. 7F shows the synergistic effects of THBS1 with THBS4. FIG. 7G shows the synergistic effects of lower concentrations of THBS2 with varying concentrations of THBS4. FIG. 7H shows the synergistic effects higher concentrations of THBS2 with varying concentrations of THBS4. FIG. 7I shows the synergistic effects of THBS2 with VTN. FIG. 7J shows the synergistic effects of THBS2 with FGF17. FIG. 7K shows the synergistic effects of THBS4 with VTN. FIG. 7L shows the synergistic effects of THBS4 with FGF17. FIG. 7M shows the synergistic effects of VTN with FGF17.

FIG. 8 shows an example of the dose dependent increasing cellular fusion of mouse myoblasts cultured with a heparin-associated polypeptide. In this case Platelet derived growth factor-like (PDGFRL) proteins were applied at 625 ng/mL, 1250 ng/mL, 2500 ng/mL, 5000 ng/mL, and 10000 ng/mL.

FIG. 9A shows the experimental schematic of time-points for dosing and analysis using an acute injury model in aged mice of the effects of individual heparin-associated polypeptides with proliferative effects in vitro. Squares denote injury inducing intramuscular injection (IM) with Barium Chloride; circles denote administration of treatment or vehicle.

FIG. 9B shows the results of the experiment outlined in FIG. 9A Administration of 20 ul of heparin-associated polypeptides FGF7 (500 ng/mL, p<2.23E-4), THBS1 (2 ug/mL, p<5.83E-5), THBS2 (2 ug/mL, p<2.67E-4), and VTN (10 ug/mL, p<1.13E-2) resulted in improved new fiber formation (regenerative index) in aged mice compared to vehicle-treated aged mice to levels similar or better than young mice. Stars indicate degree of significance from one-way ANOVA tests.

FIG. 9C Representative images of immunofluorescence staining of sectioned mouse muscle (tibialis anterior) demonstrated increased muscle regeneration for injured, aged mouse muscle treated with THBS1 (2 ug/mL) compared to young and vehicle-treated, aged mouse muscle.

FIG. 9D shows the experimental schematic of time-points for dosing and analysis using an acute injury model in aged mice of the effects of individual heparin-associated polypeptides with fusion enhancing effects in vitro. Squares denote injury inducing intramuscular injection (IM) with Barium Chloride; circles denote administration of treatment or vehicle.

FIG. 9E shows the results of the experiment outlined in FIG. 9D. Administration of 20 ul of heparin-associated polypeptides PPDGFRL (5 ug/mL, p<3.85E-2) and IGFBP7 (1 ug/mL, p<6.63E-3) resulted in improved new fiber formation (regenerative index compared to vehicle-treated aged mice. Stars indicate degree of significance from one-way ANOVA tests.

FIG. 9F provides representative images of immunofluorescence staining of sectioned mouse muscle (tibialis anterior) demonstrating increased muscle regeneration for injured, aged mouse muscle treated with POSTN (1 ug/mL) or IGFBP7 (1 ug/mL) compared to vehicle-treated, aged mouse muscle.

FIG. 10A shows the experimental schematic of time-points for in vivo injury and individual heparin-associated polypeptide administration followed by muscle excision, dissociation, ex vivo culturing of activated myoblasts and quantitation by chemical and immunofluorescent labelling.

FIG. 10B provides resulting quantitation that demonstrates the regenerative effect of heparin-associated polypeptide administration (FGF17) of 20 ul at 500 ng/ml improved the regeneration of new myoblasts in aged mice above the vehicle-treated aged mice (p<7.57E-8) to a level similar to those seen in young mice.

FIG. 10C provides representative immunofluorescence images for ex vivo cultured, injury activated myoblasts used for quantitative evaluation of factor efficacy.

FIG. 11A shows the experimental schematic of time-points for in vivo injury followed by muscle excision, dissociation, ex vivo culturing of activated myoblasts, then individual heparin-associated polypeptide administration and quantitation by chemical and immunofluorescent labelling.

FIG. 11B provides resulting quantitation that demonstrates the regenerative effect of administration of heparin-associated polypeptides FGF17 (500 ng/ml) and THBS4 (2 ug/mL)—each improved the regeneration of new myoblasts in aged mice above the vehicle-treated aged mice (p<1.57E-2, 4.64E-2 respectively, one-sided test) compared to vehicle treated control.

FIG. 11C provides representative immunofluorescence images for ex vivo cultured, injury activated myoblasts used for quantitative evaluation of factor efficacy.

FIG. 12A provides representative quantitation of immunofluorescence images demonstrating the proliferation enhancing effects of heparin-associated polypeptides administration at various does on primary human myoblasts derived from a patient with type 1 myotonic dystrophy.

FIG. 12B provides representative quantitation of immunofluorescence images demonstrating the hypertrophy enhancing effects of heparin-associated polypeptides administration at various does on primary human myoblasts derived from a patient with type 1 myotonic dystrophy.

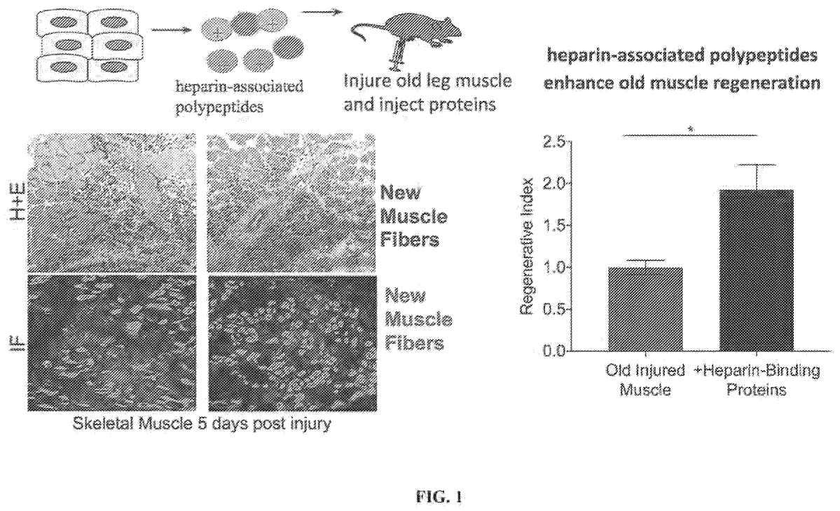

### DETAILED DESCRIPTION

In one aspect, described herein is a composition comprising a mitogenic and/or fusion promoting polypeptide, wherein the mitogenic and/or fusion promoting polypeptide is identified by: (a) identifying at least one polypeptide in a mixture of a plurality of polypeptides secreted from a stem cell or a transformed cell line that can be enriched through association of the mixture with heparin-conjugated beads; and (b) determining the mitogenic and/or fusion promoting activity of the at least one polypeptide for a somatic cell, wherein at least one polypeptide is identified as a mitogenic and/or fusion promoting polypeptide if at least one polypeptide exhibits mitogenic and/or fusion promoting activity.

In another aspect, described herein is a composition comprising a mitogenic and/or fusion promoting polypeptide, wherein the polypeptide is a heparin-associated polypeptide secreted from a stem cell or a transformed cell line, wherein the heparin-associated polypeptide possesses mitogenic and/or fusion promoting activity. In certain embodiments, the composition is for use in a method of treating an aging disorder, a muscle wasting disorder, a muscle injury, or an injury to a connective tissue, or a combination thereof. In certain embodiments, the composition is for use in a method of increasing proliferation of a muscle cell or connective tissue cell precursor in an individual. In certain embodiments, the individual is afflicted with or suspected of being afflicted with an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia. In another aspect, described herein, is a composition comprising a mitogenic and/or fusion promoting polypeptide, wherein the mitogenic and/or fusion promoting polypeptide comprises ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, a protein listed in Table 2, or a protein listed in Table 1, or any combination thereof. In certain embodiments, the mitogenic and/or fusion promoting polypeptide comprises one or more of VTN, POSTN, FGF17, THBS2, THBS4, IGF2, IL-15, and THBS1. In certain embodiments, the mitogenic and/or fusion promoting polypeptide comprises VTN. In certain embodiments, the mitogenic and/or fusion promoting polypeptide comprises THBS2. In certain embodiments, the mitogenic and/or fusion promoting polypeptide comprises POSTN. In certain embodiments, the mitogenic and/or fusion promoting polypeptide comprises FGF17. In certain embodiments, the mitogenic and/or fusion promoting polypeptide comprises THBS4. In certain embodiments, the mitogenic and/or fusion promoting polypeptide comprises IGF2. In certain embodiments, the mitogenic and/or fusion promoting polypeptide comprises IL-15. In certain embodiments, the mitogenic and/or fusion promoting polypeptide comprises THBS1. In certain embodiments, the composition comprises a mixture of a plurality of different mitogenic and/or fusion promoting polypeptides. In certain embodiments, the plurality of different mitogenic and/or fusion promoting polypeptides comprise three, four, or five different mitogenic and/or fusion promoting polypeptides. The plurality of polypeptides may comprise one, two, three, four, five, six, seven, eight, nine, ten or more polypeptides of Table 2. The plurality of polypeptides may comprise one, two, three, four, five, six, seven, eight, nine, ten or more polypeptides of Table 1. In certain embodiments, the plurality of mitogenic and/or fusion promoting polypeptides comprises IGF2, THBS2, and THBS4. In certain embodiments, the plurality of mitogenic and/or fusion promoting polypeptides comprises IL-15, THBS2, and THBS4. In certain embodiments, the plurality of mitogenic and/or fusion promoting polypeptides comprises THBS2 and THBS4. In certain embodiments, the plurality of mitogenic and/or fusion promoting polypeptides comprises THBS2, THBS4, and VTN. In certain embodiments, the plurality of mitogenic and/or fusion promoting polypeptides comprises THBS2, THBS4, and ANOS1. In certain embodiments, the plurality of mitogenic and/or fusion promoting polypeptides comprises THBS2, THBS4, and IL-15. In certain embodiments, the plurality of mitogenic and/or fusion promoting polypeptides comprises THBS2, THBS4, and IGF2. In certain embodiments, the plurality of mitogenic and/or fusion promoting polypeptides comprises THBS1 and FGF17. In certain embodiments, the plurality of mitogenic and/or fusion promoting polypeptides comprises THBS2 and VTN. In certain embodiments, the plurality of mitogenic and/or fusion promoting polypeptides comprises THBS1 and VTN. In certain embodiments, the plurality of mitogenic and/or fusion promoting polypeptides comprises THBS1 and THBS2 In certain embodiments, the plurality of mitogenic and/or fusion promoting polypeptides comprises THBS2 and FGF17. In certain embodiments, the plurality of mitogenic and/or fusion promoting polypeptides comprises THBS1 and THBS4 In certain embodiments, the plurality of mitogenic and/or fusion promoting polypeptides comprises VTN and FGF17. In certain embodiments, the plurality of mitogenic and/or fusion promoting polypeptides comprises THBS4 and VTN. In certain embodiments, the plurality of mitogenic and/or fusion promoting polypeptides comprises THBS4 and FGF17. In certain embodiments, the composition is for use in a method of treating an aging disorder, a muscle wasting disorder, a muscle injury, or an injury to a connective tissue, or any combination thereof. In certain embodiments, the composition is for use in a method of increasing proliferation of a muscle cell and/or connective tissue cell precursor in an individual. In certain embodiments, the individual is afflicted with or suspected of being afflicted with an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia.

In another aspect, described herein, is a composition comprising a mitogenic and/or fusion promoting polypeptide, wherein the mitogenic and/or fusion promoting polypeptide comprises VTN, STC2, AGRN, POSTN, FGF17, THBS2, FST, or THBS4, or a combination thereof. In certain embodiments, the mitogenic of fusion promoting polypeptide comprises at least one of VTN, POSTN, FGF17, THBS2, and THBS4. In certain embodiments, the mitogenic and/or fusion promoting polypeptide comprises one or more of VTN, POSTN, FGF17, THBS2, THBS4, IGF2, IL-15, and THBS1. In certain embodiments, the mitogenic and/or fusion promoting polypeptide comprises VTN. In certain embodiments, the mitogenic and/or fusion promoting polypeptide comprises THBS2. In certain embodiments, the mitogenic and/or fusion promoting polypeptide comprises POSTN. In certain embodiments, the mitogenic and/or fusion promoting polypeptide comprises FGF17. In certain embodiments, the mitogenic and/or fusion promoting polypeptide comprises THBS4. In certain embodiments, the mitogenic and/or fusion promoting polypeptide comprises IGF2. In certain embodiments, the mitogenic and/or fusion promoting polypeptide comprises IL-5. In certain embodiments, the mitogenic and/or fusion promoting polypeptide comprises THBS1 In certain embodiments, the composition comprises a mixture of a plurality of different mitogenic and/or fusion promoting polypeptides. In certain embodiments, the plurality of different mitogenic and/or fusion promoting polypeptides comprise three, four, or five different mitogenic and/or fusion promoting polypeptides. The plurality of polypeptides may comprise one, two, three, four, five, six, seven, eight, nine, ten or more polypeptides of Table 2. The plurality of polypeptides may comprise one, two, three, four, five, six, seven, eight, nine, ten or more polypeptides of Table 1. In certain embodiments, the plurality of mitogenic and/or fusion promoting polypeptides comprises IGF2, THBS2, and THBS4. In certain embodiments, the plurality of mitogenic and/or fusion promoting polypeptides comprises IL-15, THBS2, and THBS4 In certain embodiments, the plurality of mitogenic and/or fusion promoting polypeptides comprises THBS2 and THBS4. In certain embodiments, the plurality of mitogenic and/or fusion promoting polypeptides comprises THBS2, THBS4, and VTN. In certain embodiments, the plurality of mitogenic and/or fusion promoting polypeptides comprises THBS2, THBS4, and ANOS1. In certain embodiments, the plurality of mitogenic and/or fusion promoting polypeptides comprises THBS2. THBS4, and IL-15 In certain embodiments, the plurality of mitogenic and/or fusion promoting polypeptides comprises THBS2, THBS4, and IGF2. In certain embodiments, the plurality of mitogenic and/or fusion promoting polypeptides comprises THBS1 and FGF17. In certain embodiments, the plurality of mitogenic and/or fusion promoting polypeptides comprises THBS2 and VTN. In certain embodiments, the plurality of mitogenic and/or fusion promoting polypeptides comprises THBS1 and VTN in certain embodiments, the plurality of mitogenic and/or fusion promoting polypeptides comprises THBS1 and THBS2. In certain embodiments, the plurality of mitogenic and/or fusion promoting polypeptides comprises THBS2 and FGF17. In certain embodiments, the plurality of mitogenic and/or fusion promoting polypeptides comprises THBS1 and THBS4. In certain embodiments, the plurality of mitogenic and/or fusion promoting polypeptides comprises VTN and FGF17 In certain embodiments, the plurality of mitogenic and/or fusion promoting polypeptides comprises THBS4 and VTN. In certain embodiments, the plurality of mitogenic and/or fusion promoting polypeptides comprises THBS4 and FGF17. In certain embodiments, the composition is for use in a method of treating an aging disorder, a muscle wasting disorder, a muscle injury, or an injury to a connective tissue, or a combination thereof. In certain embodiments, the composition is for use in a method of increasing proliferation of a muscle cell and/or connective tissue cell precursor in an individual. In certain embodiments, the individual is afflicted with or suspected of being afflicted with an aging disorder, a muscle wasting disorder, a muscle injury, or an injury to a connective tissue, or a combination thereof. In certain embodiments, the aging disorder is sarcopenia. In certain embodiments, the muscle wasting disorder is muscular dystrophy. In certain embodiments, the muscle wasting disorder is cachexia, e.g., muscular cachexia.

In the following description, certain specific details are set forth in order to provide a thorough understanding of various embodiments. However, one skilled in the art will understand that the embodiments provided may be practiced without these details. Unless the context requires otherwise, throughout the specification and claims that follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein a composition that is “consisting essentially” of the recited components is a composition that only has the recited elements as active ingredients, but can comprise other non-active components that do not appreciably modify the function or activity of the recited components. Any list disclosed herein that is recited as “comprising” can be recited as “consisting essentially,” to exclude non-recited polypeptide or protein components.

As used herein “heparin-associated polypeptide” means any polypeptide that directly binds to heparin with a K_{D} of less than 1 micromolar, or any polypeptide that associates with one or more polypeptides that bind directly to heparin with a K_{D} of less than 1 micromolar. This K_{D} can be measured using a method such as surface plasmon resonance. See e.g., Nguyen et al., “Surface plasmon resonance: a versatile technique for biosensor applications.” Sensors (Basel) 2015 May 5; 15(5):10481-510. Alternatively, a heparin-associated polypeptide is one that is enriched by a factor of at least 5-fold, 10-fold, 100-fold, or 1,000 from a complex mixture of polypeptides (e.g., a cell supernatant) by the use of heparin bound to a bead or other matrix support, or co-purifies with such a polypeptide.

As used herein “heparin-binding polypeptide” means any polypeptide that directly binds to heparin with a K_{D} of less than 1 micromolar. Heparin-binding polypeptides can interact with heparin at steady-state under normal growth conditions, but in other instances heparin-binding polypeptides may interact with heparin transiently under normal growth conditions or only under certain conditions as a result of a signaling or environmental stimulus. Heparin binding-polypeptides may interact with heparin as a result of post-translational modifications such as phosphorylation, dephosphorylation, acetylation, deacetylation, lipidation, delipidation, glycosylation, or deglycosylation, or combinations thereof.

As used herein “pluripotent stem cell” or “pluripotent cell” (PSC) means a cell that has the ability to differentiate into several different cell types that are derivatives of all of the three germinal layers (endoderm, mesoderm, and ectoderm). Pluripotent stem cells are capable of forming teratomas. Examples of pluripotent stem cells are embryonic stem cells (ESCs), embryonic germ stem cells (EGCs), embryonic Carcinoma Cells (ECCs), and induced pluripotent stem cells (iPSCs). PSC may be from any organism of interest, including, primate, human, canine; feline, murine, equine; porcine; avian; camel, bovine; ovine, and so on.

As used herein “somatic cell” means any cell of an organism that, in the absence of experimental manipulation, does not ordinarily give rise to all types of cells in an organism. In other words, somatic cells are cells that have differentiated sufficiently that they will not naturally generate cells of all three germ layers of the body, i.e., ectoderm, mesoderm and endoderm. For example, somatic cells would include muscle cells and muscle progenitor cells, the latter of which may be able to self-renew and naturally give rise to all or some cell types of the skeletal, cardiac, or smooth muscle but cannot give rise to cells of the ectoderm or endoderm lineages.

As used herein the term “about” refers to an amount that is near the stated amount by 10% or less.

As used herein the terms “individual” “subject,” and “patient” are interchangeable. The individual can be mammal such as a horse, cow, pig, chicken, goat, rabbit, mouse, rat, dog, or cat. In certain embodiments, the individual is a human person.

The terms “polypeptide” and “protein” are used interchangeably to refer to a polymer of amino acid residues. Polypeptides, including the provided polypeptide chains and other peptides, e.g., linkers and binding peptides, may include amino acid residues including natural and/or non-natural amino acid residues. The terms also include post-translational modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts that produce the proteins, errors due to PCR amplification, or errors in protein translation.

In some embodiments, a recombinant protein is a protein expressed in a system other than a human, e.g., the protein is expressed from bacteria, yeast, or mammalian cells in culture. In some cases, the protein is expressed from Chinese Hamster Ovary cells (CHO cells). In some cases, the protein is expressed from mouse myeloma cells, e.g., (NS0) cells. In some cases, the protein is expressed from E. coli.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U S Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Exogenous” with respect to a nucleic acid or polynucleotide indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example using genetic recombination. Genetic recombination is the breaking and rejoining of DNA strands to form new molecules of DNA encoding a novel set of genetic information Often exogenous nucleic acids will include a translatable sequence lacking introns that has been cloned from a cDNA.

As described herein a “mitogenic polypeptide” is one that induces one or more stages of mitosis, including interphase, prophase, metaphase, anaphase, and telophase. In certain embodiments, a mitogenic polypeptide is one that induces mitosis in any one or more of a soft-tissue cell, a soft-tissue precursor cell, a muscle cell, a muscle precursor cell, or a tenocyte.

As described herein a “fusion promoting” polypeptide is one that promotes fusion of muscle cells or muscle cell precursors. Fusion of muscle precursors like C2C12 cells is an experimental marker of differentiation and can be monitored by increases in eMyHC or increased number of nuclei per eMyHC positive cell a by a statistically measurable change of at least 25% magnitude (p<0.05) relative to vehicle treated cells grown in otherwise identical conditions.

In some embodiments, reference to a fusion, fusion polypeptide, or fusion protein refers to a synthetically and/or recombinantly produced molecule in which two or more amino acid sequences are connected, e.g., by a peptide bond and/or linker. In some cases, the two or more amino acid sequences are linked via a linker comprising one or more amino acids. In other cases, the two or more amino acid sequences are not linked via a linker, e.g., the two sequences are directly connected by a peptide bond. In some cases, at least one of the two or more amino acid sequences comprises a polypeptide described herein. For example, the polypeptide described herein is a polypeptide comprising VTN, POSTN, FGF17, THBS2, THBS4, THBS1, IL-15, or IGF2, or a combination thereof.

In some embodiments, reference to a conjugate, polypeptide conjugate, or protein conjugate refers to a synthetically and/or recombinantly produced molecule comprising a chemical entity covalently bound to one or more amino acids of an amino acid sequence. In some cases, the conjugation is selective such that the chemical entity is connected to a specific amino acid of the amino acid sequence. In some embodiments, the amino acid sequence comprises a polypeptide described herein. For example, the polypeptide described herein is a polypeptide comprising VTN, POSTN, FGF7, THBS2, THBS4, THBS1, IL-15, or IGF2, or a combination thereof.

In some embodiments, a polypeptide described herein is a proteoform of a protein listed in Table 2. In some embodiments, as used herein a proteoform describes a molecular form of a protein product arising from a gene encoding a protein, such as a protein listed in Table 2. In some cases, a proteoform includes proteins that arise from the same gene as a result of genetic variation, alternatively spliced RNA transcripts, post-translational modifications, or polypeptide cleavage event.

### Heparin-Associated Polypeptides

In one aspect, polypeptides described herein that are useful for treating an aging disease or injury comprise one or more polypeptides secreted from an induced pluripotent stem cell, an embryonic stem cell, a tissue progenitor cell, or a transformed cell line that bind to heparin. In certain embodiments, a plurality of 1, 2, 3, 4, 5, 6, 7, 8, 9, 10 or more heparin-associated polypeptides are included in a composition comprising a pharmaceutically acceptable excipient, carrier, or diluent. In some cases the composition comprises one, two, three, four, five, six, seven, eight, nine, ten or more polypeptides of Table 2. In some cases the composition comprises one, two, three, four, five, six, seven, eight, nine, ten or more polypeptides of Table 1.

In certain aspects, there are three biochemical features that are common across all potential therapeutic heparin-associated polypeptides: 1) they are secreted by human pluripotent stem cells; 2) they can be purified by heparin agarose beads from a complex mixture, and 3) their molecular weight equals or exceeds 3.5 kDa.

In certain aspects, there are certain structure-function relationships that potentially link disparate therapeutic polypeptides into a genus of heparin-associated therapeutic polypeptides. Included among these are the ability to be secreted, which may require: 1) an N-terminal signal sequence (approx 15-30 amino acids in length); and/or 2) the presence of one or more post translational modifications added in the Endoplasmic Reticulum or the Golgi apparatus to promote stability, such as glycosylation or the presence of disulfide bonds. It is estimated that 2,000 to 3,000 polypeptides encoded by the human genome can be secreted by one or more cell types. In addition to being secretory polypeptides the therapeutic polypeptides may comprise a heparin-binding domain, or, alternatively associate with heparin-binding domain comprising polypeptides Heparin is a linear polymer of saccharides in 1-4 alpha linkages that form a spiraling chain, commonly associated with its role in binding plasma proteins to reduce clotting (See Capila and Lindhart, “Heparin-protein interactions” Angew Chem Int Ed Engl. 2002 Feb. 1; 41(3):391-412). Currently, predicting heparin-binding from protein sequence alone is a challenge for the field due to the structural heterogeneity of heparin polymers and the large and variable number of shallow binding pockets thought to be important for stabilizing the interaction Several hundred heparin-associated polypeptides have been empirically tested for heparin binding, using a few heparin chain configurations. Based on these studies many binding motifs have been proposed, but none have been proven necessary and sufficient One common motif appears to be a sequence of repeating basic residues that orient onto a common surface of the secondary structure for interacting with the matching pattern of sulfate groups on heparin chains. Therefore many heparin-binding therapeutic polypeptides may contain patterns of basic residues (arginine or lysine) clustered in some part of the protein, though agnostic to the exact sequence.

In certain embodiments, the heparin-associated therapeutic polypeptide is a secreted polypeptide. In certain embodiments, the heparin-associated therapeutic polypeptide is a secreted polypeptide that comprises 1, 2, 3, 4, 5, 6, 7, 8, 9, 10 or more disulfide bonds. In certain embodiments, the heparin-associated therapeutic polypeptide is a secreted polypeptide that comprises 1, 2, 3, 4, 5, 6, 7, 8, 9, 10 or N-liked or O-linked glycans. In certain embodiments, the heparin-associated therapeutic polypeptide is greater than about 3.5 kilodaltons. In other embodiments, the heparin-associated is greater than about 5, 7.5, 10, 15, or 20 kilodaltons. In certain embodiments, the heparin-associated therapeutic polypeptide is one that comprises a region exhibiting enrichment for basic amino acids arginine or lysine. The region can be about 5, 10, 15, 20, 25, 30, 35, 40, 45, 50, 55, 60, 65, 70, 75, 80, 85, 90, 95, or 100 amino acids in length, and comprise an amount of basic residues that is greater than would be 10%, 20%, 30%, 40%, 50%, 60%, 70%, 80%, 100%, 150%, or 200% greater than expected given random chance. In certain embodiments, the heparin-associated therapeutic polypeptide does not comprise a basic DNA binding motif, such as those found in bZIP transcription factors. In a certain embodiment, the heparin-associated polypeptide is heparin binding polypeptide.

The heparin-associated polypeptides, described herein, can comprise one or more amino acid modifications that promote stability and/or facilitate production. In certain embodiments, the polypeptide can comprise one or more covalent modifications that promote stability (e.g., PEGylation). Other modifications of the heparin-associated polypeptide(s) are contemplated herein. For example, the heparin-associated polypeptide(s) may be linked to one of a variety of non-proteinaceous polymers, e.g., polyethylene glycol, polypropylene glycol, polyoxyalkylenes, or copolymers of polyethylene glycol, polysialic acid, glycolic acid, or polypropylene glycol. In certain embodiments, the heparin-associated polypeptide may be fused or conjugated to another protein to increase stability and or bioavailability. In certain embodiments, the heparin-associated polypeptide may be a fusion with an Fc region of an immunoglobulin or with serum albumin.

The heparin-associated polypeptides described herein can be encapsulated in nanospheres or nanoparticles to increase stability. In certain embodiments, the nanospheres or nanoparticles are biodegradable or bioabsorbable. Certain types of nanospheres can be deployed such as polylactic acid (PLA), polyglycolic acid (PGA), or Poly(D,L-lactic-coglycolic-acid) (PLGA) microspheres or nanospheres. In certain embodiments, the heparin-associated polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA) in certain embodiments, the biodegradable or bioabsorable carrier comprises Poly(D,L-lactic-coglycolic-acid) (PLGA).

In certain embodiments, the heparin-associated-polypeptide may be concatemerized to increase stability and or bioavailability. In certain embodiments, the heparin-associated polypeptide(s) comprises concatemers of the same or of different heparin-associated binding polypeptides. Concatemers can be separated by polypeptide linkers, for example a Gly-Ser linker of any suitable length. In certain embodiments, the Gly-Ser liker comprises a G₄S₁ linker. In certain embodiments, the concatemers comprise 1, 2, 3, 4, 5 or more of the same heparin-associated polypeptide as a single polypeptide separated by a Gly-Ser linker. In certain embodiments, the concatemers comprise 1, 2, 3, 4, 5 or more different heparin-associated polypeptides as a single polypeptide separated by a Gly-Ser linker. In certain embodiments, the concatemers comprise 1, 2, 3, 4, 5 or more of the same heparin-associated polypeptide covalently linked through a non-peptide linkage, such as for example a disulfide bridge. In certain embodiments, the concatemers comprise 1, 2, 3, 4, 5 or more different heparin-associated polypeptides covalently linked through a non-peptide linkage, such as for example a disulfide bridge. In certain embodiments, the concatemers comprise 1, 2, 3, 4, 5 or more of the same heparin-associated polypeptide non-covalently linked, such as for example, by a streptavidin-biotin interaction or protein-protein interaction. In certain embodiments, the concatemers comprise 1, 2, 3, 4, 5 or more different heparin-associated polypeptides non-covalently linked such as for example, by a streptavidin-biotin interaction or protein-protein interaction.

Additional modifications to heparin-associated polypeptide comprise deletions of 1, 2, 3, 4, 5, 6, 7, 8, 9, 10, 15, 20, 25, 30, 35, 40, 45, 50, 55, 60, 65, 70, 75, 80, 85, 90, 95, 100 amino acids from the N-terminal or C-terminal ends of the heparin-associated polypeptide. In certain embodiments, the heparin-associated polypeptide comprises the deletion of known inhibitory domains or deletion of domains not associated with the heparin-associated-polypeptides functions in inducing proliferation of muscle, connective, or soft-tissue cell precursors.

The heparin-associated polypeptides herein can comprise cleavage products of a pro-protein. Cleavage of a pro-protein can result in activation or higher activity of said pro-protein. In certain embodiments, heparin-associated polypeptides are produced that correspond to a cleaved or active form of the pro-protein. In certain embodiments, the heparin-associated polypeptides comprise only the active domain of a heparin associated pro-protein (e.g., the minimal portion sufficient to create a biological effect).

In certain embodiments, the heparin-associated polypeptide comprises one or more of the polypeptides listed in Table 1 and/or Table 2. In certain embodiments, the polypeptide is at least about 85%, 90%, 95%, 97%, 98%, 99%, or 100% identical to a polypeptide listed in Table 1 and/or Table 2, or an isoform thereof. In certain embodiments, the polypeptide is at least about 85%, 90%, 95%, 97%, 98%, 99%, or 100% identical to a sequence selected from SEQ ID NOS: 1-44, 55, 56, and 58-70.

TABLE 1
Exemplary Therapeutic Polypeptides

| | SEQ | |
|---|---|---|
| Polypeptide | ID | Modified Amino Acid Sequences Tested |
| Vitronectin (VTN) | 1 | Asp20 to Leu478, purified from Human plasma-derived |
| Periostin (POSTN) | 6 | Asn22 to Gln836, with a C-terminal 6-His tag (SEQ ID NO: 69), |
| | | purified from Mouse myeloma cell line, NS0 |
| Thrombospondin 2 | 4 | Gly19 to Ile1172, with a C-terminal 10-His tag (SEQ ID NO: 70), |
| (THBS2) | | purified from Mouse myeloma cell line, NS0 |
| Thrombospondin 4 | 8 | Ala22 to Asn961, with a C-terminal 10-His tag (SEQ ID NO: 70), |
| (THBS4) | | purified from Chinese Hamster Ovary cell line |
| Fibroblast growth factor 2 | 12 | Pro143 to Ser288, purified E. coli |
| (FGF 2) | | |
| Fibroblast growth factor | 13 | Leu25 to Lys216, purified from E. coli |
| 19 (FGF 19) | | |
| Angiogenin (ANG) | 14 | Gln25 to Pro147, purified from E. coli |
| Probetacellulin (BTC) | 15 | Asp32 to Tyr111, purified from E. coli |
| Interleukin -13 receptor | 16 | Arg21 to Ala228, purified from mouse myeloma cell line, NS0 |
| alpha 2 | | |
| Siglec-5/CD170 | 17 | Glu17 to Thr434, purified from mouse myeloma cell line, NS0 |
| Interleukin-15 | 10 | Asn49 to Ser162, purified from E. coli |
| Apelin receptor (APJ) | 18 | |
| Insulin-like growth factor- | 19 | Glu40 to Gln328, purified from mouse myeloma cell line, NS0 |
| binding protein 2 (IGFBP- | | |
| 2) | | |
| Chordin-Like 1 | 20 | Glu22-Cys450, purified from mouse myeloma cell line, NS0 |
| (CHRDL1) | | |
| WAP, Kazal, | 21 | Leu35 to His 576, purified from mouse myeloma cell line, NS0 |
| immunoglobulin, Kunitz | | |
| and NTR domain- | | |
| containing protein 2 | | |
| Membrane frizzled-related | 22 | Ser101 to Pro579, purified from mouse myeloma cell line, NS0 |
| protein (MFRP) | | |
| Interleukin -10 receptor | 23 | His22 to Asn235, purified from human cell line HEK293 |
| alpha | | |
| Chemokine like receptor 1, | 24 | |
| Chemerin Receptor 23 | | |
| (Chem R23) | | |
| HB-EGF | 25 | Asp63 to Leu148, purified from insect cells |
| fibroblast growth factor 6 | 26 | Gly67 to Ile208, purified from E. coli |
| Hepatocyte Growth Factor | 27 | Gln32 to Ser728, purified from insect cells |
| Interleukin-16 | 28 | Pro2 to Ser130, purified from E. coli |
| Interleukin-7 receptor | 29 | Glu21 to Lys261, purified from mouse myeloma cell line, NS0 |
| alpha | | |
| Tumor necrosis factor | 30 | Ala26 to Ala221, purified from mouse myeloma cell line, NS0 |
| receptor superfamily | | |
| member 10C | | |
| Bone morphogenetic | 31 | Gln382 to His51, 3 purified from E. coli |
| protein 6 | | |
| Interleukin-36 gamma | 32 | Ser18 to Asp169, purified from E. coli |
| interleukin-1 receptor | 33 | Val2 to Asp155, purified from E. coli |
| antagonist | | |
| (IL-1RA) | | |
| Kremen protein 2 | 34 | Gln19 to Ala364, purified from mouse myeloma cell line, NS0 |
| Tumor necrosis factor | 35 | |
| receptor superfamily | | |
| member 10D | | |
| C-X-C chemokine receptor | 36 | |
| type 1 | | |
| C-C motif chemokine 23 | 37 | |
| Catenin, Beta | 38 | |
| Fibroblast growth factor | 39 | |
| 13, 1B | | |
| Tumor necrosis factor | 40 | Glu108 to Leu261, purified from E. coli |
| ligand superfamily | | |
| member 10 | | |
| C-C motif chemokine 14 | 41 | Ser35 to Glu111, purified from E. coli |
| Thrombospondin 1 | 9 | Asn19-Pro1170, with Thr523Ala substitution plus 10His tag, |
| (THBS1) | | purified from mouse myeloma cell line, NS0 |
| Insulin-like growth factor | 42 | Asp30 to Leu282 with a K95R mutation with an N-terminal 10- |
| binding protein 7 | | His tag (SEQ ID NO: 70), purified from Mouse myeloma cell |
| | | line, NS0 |
| Fibroblast growth factor 4 | 43 | Ser54 to Leu206, purified from E. coli |
| Fibroblast growth factor 17 | 7 | Thr23 to Thr216, purified from E. coli |
| (FGF17) | | |
| Fibroblast growth factor 8 | 44 | Gln23 to Arg204, purified from E. coli |
| Insulin-like growth factor | 11 | Ala25 to Glu91, purified from E. coli |
| 2 (IGF2) | | |
| THBS1 isoform 2 | 58 | |
| FGF17 isoform 2 | 59 | |
| POSTN isoform 2 | 60 | |
| POSTN isoform 3 | 61 | |
| POSTN isoform 4 | 62 | |
| POSTN isoform 5 | 63 | |
| POSTN isoform 6 | 64 | |
| POSTN isoform 7 | 65 | |
| IGF2 isoform 2 | 66 | |
| IGF2 isoform 3 | 67 | |
| IL15 isoform 2 | 68 | |
| STC2 | 2 | |
| AGRN | 3 | |
| FST | 5 | |
| PDGFRL | 55 | Gln22 to Ser375, purified from HEK293 cells |
| ANOS1 | 56 | Ala25 to Tyr680, purified from CHO cells |

TABLE 2
Factors enriched in the supernatants of undifferentiated
human pluripotent stem cells.

| Gene | | Entrez | | Peptide |
|---|---|---|---|---|
| Name | Uniprot ID | Gene ID | Ensembl ID | No. |
| A1BG | P04217 | 1 | ENSG00000121410 | 1 |
| A2M | P01023 | 2 | ENSG00000175899 | 2 |
| ABCF1 | Q8NE71 | 23 | ENSG00000204574 | 3 |
| ACADVL | P49748 | 37 | ENSG00000072778 | 4 |
| ACLY | P53396 | 47 | ENSG00000131473 | 5 |
| ACP1 | P24666 | 52 | ENSG00000143727 | 6 |
| ACP5 | P13686 | 54 | ENSG00000102575 | 7 |
| ACTG1 | P63261 | 71 | ENSG00000184009 | 8 |
| ACTN1 | P12814 | 87 | ENSG00000072110 | 9 |
| ACTR3 | P61158 | 10096 | ENSG00000115091 | 10 |
| ADAMTS1 | Q9UHI8 | 9510 | ENSG00000154734 | 11 |
| ADAMTS12 | P58397 | 81792 | ENSG00000151388 | 12 |
| ADAMTS19 | Q8TE59 | 171019 | ENSG00000145808 | 13 |
| ADAMTS7 | Q9UKP4 | 11173 | ENSG00000136378 | 14 |
| ADAMTS8 | Q9UP79 | 11095 | ENSG00000134917 | 15 |
| ADRM1 | Q16186 | 11047 | ENSG00000130706 | 16 |
| AEBP1 | Q81UX7 | 165 | ENSG00000106624 | 17 |
| AFM | P43652 | 173 | ENSG00000079557 | 18 |
| AFP | P02771 | 174 | ENSG00000081051 | 19 |
| AGPS | O00116 | 8540 | ENSG00000018510 | 20 |
| AGRN | O00468 | 375790 | ENSG00000188157 | 21 |
| AGT | P01019 | 183 | ENSG00000135744 | 22 |
| AHCYL2 | Q96HN2 | 23382 | ENSG00000158467 | 23 |
| AHSG | P02765 | 197 | ENSG00000145192 | 24 |
| AIMP1 | Q12904 | 9255 | ENSG00000164022 | 25 |
| ALB | P02768 | 213 | ENSG00000163631 | 26 |
| ALCAM | Q13740 | 214 | ENSG00000170017 | 27 |
| ALDH9A1 | P49189 | 223 | ENSG00000143149 | 28 |
| ALDOA | P04075 | 226 | ENSG00000149925 | 29 |
| ALPL | P05186 | 249 | ENSG00000162551 | 30 |
| AMBP | P02760 | 259 | ENSG00000106927 | 31 |
| ANG | P03950 | 283 | ENSG00000214274 | 32 |
| ANGPTL4 | Q9BY76 | 51129 | ENSG00000167772 | 33 |
| ANOS1 | P23352 | 3730 | ENSG00000011201 | 34 |
| ANXA1 | P04083 | 301 | ENSG00000135046 | 35 |
| ANXA2 | P07355 | 302 | ENSG00000182718 | 36 |
| ANXA2P2 | A6NMY6 | | | 37 |
| AOC1 | P19801 | 26 | ENSG00000002726 | 38 |
| AP2A1 | O95782 | 160 | ENSG00000196961 | 39 |
| AP2A2 | O94973 | 161 | ENSG00000183020 | 40 |
| AP3D1 | O14617 | 8943 | ENSG00000065000 | 41 |
| APLP2 | Q06481 | 334 | ENSG00000084234 | 42 |
| APOA1 | P02647 | 335 | ENSG00000118137 | 43 |
| APOA2 | P02652 | 336 | ENSG00000158874 | 44 |
| APOB | P04114 | 338 | ENSG00000084674 | 45 |
| APOC3 | P02656 | 345 | ENSG00000110245 | 46 |
| APOD | P05090 | 347 | ENSG00000189058 | 47 |
| APOE | P02649 | 348 | ENSG00000130203 | 48 |
| APOH | P02749 | 350 | ENSG00000091583 | 49 |
| APOM | O95445 | 55937 | ENSG00000204444 | 50 |
| ARCN1 | P48444 | 372 | ENSG00000095139 | 51 |
| ARHGEF1 | Q92888 | 9138 | ENSG00000076928 | 52 |
| ARHGEF28 | Q8N1W1 | 64283 | ENSG00000214944 | 53 |
| ARPC1B | O15143 | 10095 | ENSG00000130429 | 54 |
| ARRB1 | P49407 | 408 | ENSG00000137486 | 55 |
| ARSK | Q6UWY0 | 153642 | ENSG00000164291 | 56 |
| ART4 | Q93070 | 420 | ENSG00000111339 | 57 |
| ASNA1 | O43681 | 439 | ENSG00000198356 | 58 |
| ASNS | P08243 | 440 | ENSG00000070669 | 59 |
| ATP6AP2 | O75787 | 10159 | ENSG00000182220 | 60 |
| ATRN | O75882 | 8455 | ENSG00000088812 | 61 |
| AZGP1 | P25311 | 563 | ENSG00000160862 | 62 |
| B3GALT6 | Q96L58 | 126792 | ENSG00000176022 | 63 |
| B3GNT7 | Q8NFL0 | 93010 | ENSG00000156966 | 64 |
| B4GALT1 | P15291 | 2683 | ENSG00000086062 | 65 |
| B4GALT4 | O60513 | 8702 | ENSG00000121578 | 66 |
| B4GAT1 | O43505 | 11041 | ENSG00000174684 | 67 |
| BCAM | P50895 | 4059 | ENSG00000187244 | 68 |
| BGN | P21810 | 633 | ENSG00000182492 | 69 |
| BLVRB | P30043 | 645 | ENSG00000090013 | 70 |
| BMP1 | P13497 | 649 | ENSG00000168487 | 71 |
| BMP7 | P18075 | 655 | ENSG00000101144 | 72 |
| BOC | Q9BWV1 | 91653 | ENSG00000144857 | 73 |
| BRD3 | Q15059 | 8019 | ENSG00000169925 | 74 |
| BSG | P35613 | 682 | ENSG00000172270 | 75 |
| BTBD17 | A6NE02 | 388419 | ENSG00000204347 | 76 |
| BTD | P43251 | 686 | ENSG00000169814 | 77 |
| BZW2 | Q9Y6E2 | 28969 | ENSG00000136261 | 78 |
| C11orf24 | Q96F05 | 53838 | ENSG00000171067 | 79 |
| C1QA | P02745 | 712 | ENSG00000173372 | 80 |
| C1QBP | Q07021 | 708 | ENSG00000108561 | 81 |
| C1QC | P02747 | 714 | ENSG00000159189 | 82 |
| C1QTNF3 | Q9BXJ4 | 114899 | ENSG00000082196 | 83 |
| C1QTNF3- | E9PGA6 | | ENSG00000273294 | 84 |
| AMACR | | | | |
| C1QTNF4 | Q9BXJ3 | 114900 | ENSG00000172247 | 85 |
| C1RL | Q9NZP8 | 51279 | ENSG00000139178 | 86 |
| C1S | P09871 | 716 | ENSG00000182326 | 87 |
| C20orf27 | Q9GZN8 | 54976 | ENSG00000101220 | 88 |
| C3 | P01024 | 718 | ENSG00000125730 | 89 |
| C4A | P0C0L4 | 720 | ENSG00000206340 | 90 |
| C4B | A0A140TA29 | | ENSG00000236625 | 91 |
| C4BPA | P04003 | 722 | ENSG00000123838 | 92 |
| C5 | P01031 | 727 | ENSG00000106804 | 93 |
| C7 | P10643 | 730 | ENSG00000112936 | 94 |
| C8B | P07358 | 732 | ENSG00000021852 | 95 |
| C9 | P02748 | 735 | ENSG00000113600 | 96 |
| CA11 | O75493 | 770 | ENSG00000063180 | 97 |
| CALM2 | P0DP24 | 801 | ENSG00000143933 | 98 |
| CALR | P27797 | 811 | ENSG00000179218 | 99 |
| CALU | O43852 | 813 | ENSG00000128595 | 100 |
| CAND1 | Q86VP6 | 55832 | ENSG00000111530 | 101 |
| CANT1 | Q8WVQ1 | 124583 | ENSG00000171302 | 102 |
| CANX | P27824 | 821 | ENSG00000127022 | 103 |
| CAPG | P40121 | 822 | ENSG00000042493 | 104 |
| CAPN1 | P07384 | 823 | ENSG00000014216 | 105 |
| CAPZA2 | P47755 | 830 | ENSG00000198898 | 106 |
| CARM1 | Q86X55 | 10498 | ENSG00000142453 | 107 |
| CARS | P49589 | 833 | ENSG00000110619 | 108 |
| CBL | P22681 | 867 | ENSG00000110395 | 109 |
| CBX3 | Q13185 | 11335 | ENSG00000122565 | 110 |
| CCAR2 | Q8N163 | 57805 | ENSG00000158941 | 111 |
| CCBE1 | Q6UXH8 | 147372 | ENSG00000183287 | 112 |
| CCDC80 | Q76M96 | 151887 | ENSG00000091986 | 113 |
| CCK | P06307 | 885 | ENSG00000187094 | 114 |
| CCT2 | P78371 | 10576 | ENSG00000166226 | 115 |
| CCT4 | P50991 | 10575 | ENSG00000115484 | 116 |
| CCT7 | Q99832 | 10574 | ENSG00000135624 | 117 |
| CD5 | P06127 | 921 | ENSG00000110448 | 118 |
| CDC40 | O60508 | 51362 | ENSG00000168438 | 119 |
| CDH1 | P12830 | 999 | ENSG00000039068 | 120 |
| CDH9 | Q9ULB4 | 1007 | ENSG00000113100 | 121 |
| CDON | Q4KMG0 | 50937 | ENSG00000064309 | 122 |
| CDSN | Q15517 | 1041 | ENSG00000137197 | 123 |
| CENPV | Q7Z7K6 | 201161 | ENSG00000166582 | 124 |
| CFB | P00751 | 629 | ENSG00000241253 | 125 |
| CFC1 | P0CG37 | 55997 | ENSG00000136698 | 126 |
| CFD | P00746 | 1675 | ENSG00000197766 | 127 |
| CFH | P08603 | 3075 | ENSG00000000971 | 128 |
| CF1 | P05156 | 3426 | ENSG00000205403 | 129 |
| CHAD | O15335 | 1101 | ENSG00000136457 | 130 |
| CHD4 | Q14839 | 1108 | ENSG00000111642 | 131 |
| CHD8 | Q9HCK8 | 57680 | ENSG00000100888 | 132 |
| CHGA | P10645 | 1113 | ENSG00000100604 | 133 |
| CHID1 | Q9BWS9 | 66005 | ENSG00000177830 | 134 |
| CHRDL1 | Q9BU40 | 91851 | ENSG00000101938 | 135 |
| CHST11 | Q9NPF2 | 50515 | ENSG00000171310 | 136 |
| CHST6 | Q9GZX3 | 4166 | ENSG00000183196 | 137 |
| CHSY1 | Q86X52 | 22856 | ENSG00000131873 | 138 |
| CHSY3 | Q70JA7 | 337876 | ENSG00000198108 | 139 |
| CILP2 | Q8IUL8 | 148113 | ENSG00000160161 | 140 |
| CKAP5 | Q14008 | 9793 | ENSG00000175216 | 141 |
| CKMT1A | C9J8F6 | | ENSG00000223572 | 142 |
| CKMT2 | P17540 | 1160 | ENSG00000131730 | 143 |
| CLDN6 | P56747 | 9074 | ENSG00000184697 | 144 |
| CLEC3B | P05452 | 7123 | ENSG00000163815 | 145 |
| CLPX | O76031 | 10845 | ENSG00000166855 | 146 |
| CLSTN3 | Q9BQT9 | 9746 | ENSG00000139182 | 147 |
| CLTC | Q00610 | 1213 | ENSG00000141367 | 148 |
| CLU | P10909 | 1191 | ENSG00000120885 | 149 |
| CNOT1 | A5YKK6 | 23019 | ENSG00000125107 | 150 |
| COCH | O43405 | 1690 | ENSG00000100473 | 151 |
| COL11A1 | P12107 | 130 | ENSG00000060718 | 152 |
| COL11A2 | P13942 | 1302 | ENSG00000227801 | 153 |
| COL12A1 | Q99715 | 1303 | ENSG00000111799 | 154 |
| COL14A1 | Q05707 | 7373 | ENSG00000187955 | 155 |
| COL16A1 | Q07092 | 1307 | ENSG00000084636 | 156 |
| COL18A1 | P39060 | 80781 | ENSG00000182871 | 157 |
| COL1A1 | P02452 | 1277 | ENSG00000108821 | 158 |
| COL1A2 | P08123 | 1278 | ENSG00000164692 | 159 |
| COL22A1 | Q8NFW1 | 169044 | ENSG00000169436 | 160 |
| COL25A1 | Q9BXS0 | 84570 | ENSG00000188517 | 161 |
| COL26A1 | Q96A83 | 136227 | ENSG00000160963 | 162 |
| COL2A1 | P02458 | 1280 | ENSG00000139219 | 163 |
| COL3A1 | P02461 | 1281 | ENSG00000168542 | 164 |
| COL4A1 | P02462 | 1282 | ENSG00000187498 | 165 |
| COL4A2 | P08572 | 1284 | ENSG00000134871 | 166 |
| COL4A3 | Q01955 | 1285 | ENSG00000169031 | 167 |
| COL4A6 | Q14031 | 1288 | ENSG00000197565 | 168 |
| COL5A1 | P20908 | 1289 | ENSG00000130635 | 169 |
| COL5A2 | P05997 | 1290 | ENSG00000204262 | 170 |
| COL5A3 | P25940 | 50509 | ENSG00000080573 | 171 |
| COL6A1 | P12109 | 1291 | ENSG00000142156 | 172 |
| COL6A2 | P12110 | 1292 | ENSG00000142173 | 173 |
| COL6A3 | P12111 | 1293 | ENSG00000163359 | 174 |
| COL9A2 | Q14055 | 1298 | ENSG00000049089 | 175 |
| COLEC10 | Q9Y6Z7 | 10584 | ENSG00000184374 | 176 |
| COMP | P49747 | 1311 | ENSG00000105664 | 177 |
| COPA | P53621 | 1314 | ENSG00000122218 | 178 |
| COTL1 | Q14019 | 23406 | ENSG00000103187 | 179 |
| CP | P00450 | 1356 | ENSG00000047457 | 180 |
| CPA4 | Q9UI42 | 51200 | ENSG00000128510 | 181 |
| CPE | P16870 | 1363 | ENSG00000109472 | 182 |
| CPN1 | P15169 | 1369 | ENSG00000120054 | 183 |
| CPNE1 | Q99829 | 8904 | ENSG00000214078 | 184 |
| CPVL | Q9H3G5 | 54504 | ENSG00000106066 | 185 |
| CPXM1 | Q96SM3 | 56265 | ENSG00000088882 | 186 |
| CPXM2 | Q8N436 | 119587 | ENSG00000121898 | 187 |
| CPZ | Q66K79 | 8532 | ENSG00000109625 | 188 |
| CRIM1 | Q9NZV1 | 51232 | ENSG00000150938 | 189 |
| CRISPLD1 | Q9H336 | 83690 | ENSG00000121005 | 190 |
| CRLF1 | O75462 | 9244 | ENSG00000006016 | 191 |
| CRYL1 | Q9Y2S2 | 51084 | ENSG00000165475 | 192 |
| CS | O75390 | 1431 | ENSG00000062485 | 193 |
| CSDE1 | O75534 | 7812 | ENSG00000009307 | 194 |
| CSF2RA | P15509 | 1438 | ENSG00000198223 | 195 |
| CST1 | P01037 | 1469 | ENSG00000170373 | 196 |
| CST3 | P01034 | 1471 | ENSG00000101439 | 197 |
| CST4 | P01036 | 1472 | ENSG00000101441 | 198 |
| CTGF | Q5M8T4 | 1490 | | 199 |
| CTNNA1 | P35221 | 1495 | ENSG00000044115 | 200 |
| CTSD | P07339 | 1509 | ENSG00000117984 | 201 |
| CTSV | O60911 | 1515 | ENSG00000136943 | 202 |
| CUL2 | Q13617 | 8453 | ENSG00000108094 | 203 |
| CUL3 | Q13618 | 8452 | ENSG00000036257 | 204 |
| CUL4B | Q13620 | 8450 | ENSG00000158290 | 205 |
| CUTA | O60888 | 51596 | ENSG00000112514 | 206 |
| CXADR | P78310 | 1525 | ENSG00000154639 | 207 |
| CXCL12 | P48061 | 6387 | ENSG00000107562 | 208 |
| CYR61 | Q6FI18 | 3491 | | 209 |
| DAG1 | Q14118 | 1605 | ENSG00000173402 | 210 |
| DARS | P14868 | 1615 | ENSG00000115866 | 211 |
| DBNL | Q9UJU6 | 28988 | ENSG00000136279 | 212 |
| DCD | P81605 | 117159 | ENSG00000161634 | 213 |
| DDOST | P39656 | 1650 | ENSG00000244038 | 214 |
| DDR1 | Q08345 | 780 | ENSG00000137332 | 215 |
| DDX17 | Q92841 | 10521 | ENSG00000100201 | 216 |
| DDX39B | Q13838 | 7919 | ENSG00000215425 | 213 |
| DENND5A | Q6IQ26 | 23258 | ENSG00000184014 | 218 |
| DHFR | P00374 | 1719 | ENSG00000228716 | 219 |
| DHX29 | Q72478 | 54505 | ENSG00000067248 | 220 |
| DKK1 | O94907 | 22943 | ENSG00000107984 | 221 |
| DKK3 | Q9UBP4 | 27122 | ENSG00000050165 | 222 |
| DKK4 | Q9UBT3 | 27121 | ENSG00000104371 | 223 |
| DLG3 | Q92796 | 1741 | ENSG00000082458 | 224 |
| DMBT1 | Q9UGM3 | 1755 | ENSG00000187908 | 225 |
| DNAAF5 | Q86Y56 | 54919 | ENSG00000164818 | 226 |
| DNAJB11 | Q9UBS4 | 51726 | ENSG00000090520 | 227 |
| DNAJC3 | Q13217 | 5611 | ENSG00000102580 | 228 |
| DNMT1 | P26358 | 1786 | ENSG00000130816 | 229 |
| DRAXIN | Q8NBI3 | 374946 | ENSG00000162490 | 230 |
| DRG1 | Q9Y295 | 4733 | ENSG00000185721 | 231 |
| DSG2 | Q14126 | 1829 | ENSG00000046604 | 232 |
| ECM1 | Q16610 | 1893 | ENSG00000143369 | 233 |
| EDA | Q92838 | 1896 | ENSG00000158813 | 234 |
| EDIL3 | O43854 | 10085 | ENSG00000164176 | 235 |
| EEF1G | P26641 | 1937 | ENSG00000254772 | 236 |
| EFEMP1 | Q12805 | 2202 | ENSG00000115380 | 237 |
| EFTUD2 | Q15029 | 9343 | ENSG00000108883 | 238 |
| EGFLAM | Q63HQ2 | 133584 | ENSG00000164318 | 239 |
| EIF1AY | O14602 | 9086 | ENSG00000198692 | 240 |
| EIF2B4 | Q9UI10 | 8890 | ENSG00000115211 | 241 |
| EIF2S1 | P05198 | 1965 | ENSG00000134001 | 242 |
| EIF2S2 | P20042 | 8894 | ENSG00000125977 | 243 |
| EIF3A | Q14152 | 8661 | ENSG00000107581 | 244 |
| EIF3C | Q99613 | 8663 | ENSG00000184110 | 245 |
| EIF3F | O00303 | 8665 | ENSG00000175390 | 246 |
| EIF3H | O15372 | 8667 | ENSG00000147677 | 247 |
| EIF3M | Q7L2H7 | 10480 | ENSG00000149100 | 248 |
| EIF5 | P55010 | 1983 | ENSG00000100664 | 249 |
| EIF5B | O60841 | 9669 | ENSG00000158417 | 250 |
| ELAC2 | Q9BQ52 | 60528 | ENSG00000006744 | 251 |
| ELP3 | Q9H9T3 | 55140 | ENSG00000134014 | 252 |
| EMILIN2 | Q9BXX0 | 84034 | ENSG00000132205 | 253 |
| EPHA4 | P54764 | 2043 | ENSG00000116106 | 254 |
| EPHB2 | P29323 | 2048 | ENSG00000133216 | 255 |
| EPHB4 | P54760 | 2050 | ENSG00000196411 | 256 |
| EPRS | P07814 | 2058 | ENSG00000136628 | 257 |
| ERBB3 | P21860 | 2065 | ENSG00000065361 | 258 |
| ERLIN1 | O75477 | 10613 | ENSG00000107566 | 259 |
| ERVMER34-1 | Q9H9K5 | 100288413 | ENSG00000226887 | 260 |
| EXTL2 | Q9UBQ6 | 2135 | ENSG00000162694 | 261 |
| EZR | P15311 | 7430 | ENSG00000092820 | 262 |
| F10 | P00742 | 2159 | ENSG00000126218 | 263 |
| F13A1 | P00488 | 2162 | ENSG00000124491 | 264 |
| F2 | P00734 | 2147 | ENSG00000180210 | 265 |
| F5 | P12259 | 2153 | ENSG00000198734 | 266 |
| FAM129B | Q96TA1 | 64855 | ENSG00000136830 | 267 |
| FAP | Q12884 | 2191 | ENSG00000078098 | 268 |
| FAT1 | Q14517 | 2195 | ENSG00000083857 | 269 |
| FBLN1 | P23142 | 2192 | ENSG00000077942 | 270 |
| FBLN2 | P98095 | 2199 | ENSG00000163520 | 271 |
| FBN1 | P35555 | 2200 | | 272 |
| FBN2 | P35556 | 2201 | ENSG00000138829 | 273 |
| FERMT2 | Q96AC1 | 10979 | ENSG00000073712 | 274 |
| FGB | P02675 | 2244 | ENSG00000171564 | 275 |
| FGF17 | O60258 | 8822 | ENSG00000158815 | 276 |
| FGF2 | P09038 | 2247 | ENSG00000138685 | 277 |
| FGF8 | P55075 | 2253 | ENSG00000107831 | 278 |
| FGF4 | P08620 | 2249 | ENSG00000075388 | 279 |
| FGF6 | P10767 | 2251 | ENSG00000111241 | 280 |
| FGFBP3 | Q8TAT2 | 143282 | ENSG00000174721 | 281 |
| FGFR1 | P11362 | 2260 | ENSG00000077782 | 282 |
| FGFR2 | P21802 | 2263 | ENSG00000066468 | 283 |
| FGFR4 | P22455 | 2264 | ENSG00000160867 | 284 |
| FGFRL1 | Q8N441 | 53834 | ENSG00000127418 | 285 |
| FH | P07954 | 2271 | ENSG00000091483 | 286 |
| FLT1 | P17948 | 2321 | ENSG00000102755 | 287 |
| FN1 | P02751 | 2335 | ENSG00000115414 | 288 |
| FRAS1 | Q86XX4 | 80144 | ENSG00000138759 | 289 |
| FRZB | Q92765 | 2487 | ENSG00000162998 | 290 |
| FST | P19883 | 10468 | ENSG00000134363 | 291 |
| FSTL1 | Q12841 | 11167 | ENSG00000163430 | 292 |
| FUCA2 | Q9BTY2 | 2519 | ENSG00000001036 | 293 |
| FXR1 | P51114 | 8087 | ENSG00000114416 | 294 |
| GALNT1 | Q10472 | 2589 | ENSG00000141429 | 295 |
| GALNT16 | Q8N428 | 57452 | ENSG00000100626 | 296 |
| GALNT2 | Q10471 | 2590 | ENSG00000143641 | 297 |
| GALNT7 | Q86SF2 | 51809 | ENSG00000109586 | 298 |
| GANAB | Q14697 | 23193 | ENSG00000089597 | 299 |
| GARS | P41250 | 2617 | ENSG00000106105 | 300 |
| GBA | P04062 | 2629 | ENSG00000177628 | 301 |
| GC | P02774 | 2638 | ENSG00000145321 | 302 |
| GCNT1 | Q02742 | 2650 | ENSG00000187210 | 303 |
| GDF11 | O95390 | 10220 | ENSG00000135414 | 304 |
| GDF15 | Q99988 | 9518 | ENSG00000130513 | 305 |
| GDF6 | Q6KF10 | 392255 | ENSG00000156466 | 306 |
| GEMIN5 | Q8TEQ6 | 25929 | ENSG00000082516 | 307 |
| GFAP | P14136 | 2670 | ENSG00000131095 | 308 |
| GGH | Q92820 | 8836 | ENSG00000137563 | 309 |
| GLB1 | P16278 | 2720 | ENSG00000170266 | 310 |
| GLG1 | Q92896 | 2734 | ENSG00000090863 | 311 |
| GM2A | P17900 | 2760 | ENSG00000196743 | 312 |
| GNAS | O95467 | 2778 | ENSG00000087460 | 313 |
| GOLM1 | Q8NBJ4 | 51280 | ENSG00000135052 | 314 |
| GOT2 | P00505 | 2806 | ENSG00000125166 | 315 |
| GPC1 | P35052 | 2817 | ENSG00000063660 | 316 |
| GPC3 | P51654 | 2719 | ENSG00000147257 | 317 |
| GPC4 | O75487 | 2239 | ENSG00000076716 | 318 |
| GPI | P06744 | 2821 | ENSG00000105220 | 319 |
| GPRC5B | Q9NZH0 | 51704 | ENSG00000167191 | 320 |
| GPX4 | P36969 | 2879 | ENSG00000167468 | 321 |
| GREM1 | O60565 | 26585 | ENSG00000166923 | 322 |
| GRN | P28799 | 2896 | ENSG00000030582 | 323 |
| GRSF1 | Q12849 | 2926 | ENSG00000132463 | 324 |
| GSN | P06396 | 2934 | ENSG00000148180 | 325 |
| GSPT1 | P15170 | 2935 | ENSG00000103342 | 326 |
| GTF3C3 | Q9Y5Q9 | 9330 | ENSG00000119041 | 327 |
| HABP2 | Q14520 | 3026 | ENSG00000148702 | 328 |
| HADHB | P55084 | 3032 | ENSG00000138029 | 329 |
| HAPLN1 | P10915 | 1404 | ENSG00000145681 | 330 |
| HAPLN3 | Q96S86 | 145864 | | 331 |
| HAPLN4 | Q86UW8 | 404037 | | 332 |
| HARS | P12081 | 3035 | ENSG00000170445 | 333 |
| HBB | P68871 | 3043 | ENSG00000244734 | 334 |
| HBS1L | Q9Y450 | 10767 | ENSG00000112339 | 335 |
| HDGF | P51858 | 3068 | ENSG00000143321 | 336 |
| HDGFL2 | Q7Z4V5 | 84717 | ENSG00000167674 | 337 |
| HDLBP | Q00341 | 3069 | ENSG00000115677 | 338 |
| HGF | P14210 | 3082 | ENSG00000019991 | 339 |
| HGFAC | Q04756 | 3083 | ENSG00000109758 | 340 |
| HIST1H1C | P16403 | 3006 | ENSG00000187837 | 341 |
| HIST1H1E | P10412 | 3008 | ENSG00000168298 | 342 |
| HLA-C | P04222 | | ENSG00000225691 | 343 |
| HMCN1 | Q96RW7 | 83872 | ENSG00000143341 | 344 |
| HMCN2 | Q8NDA2 | | ENSG00000148357 | 345 |
| HMGB1 | P09429 | 3146 | ENSG00000189403 | 346 |
| HMGB2 | P26583 | 3148 | ENSG00000164104 | 347 |
| HMGB3 | O15347 | 3149 | ENSG00000029993 | 348 |
| HMGN1 | P05114 | 3150 | ENSG00000205581 | 349 |
| HMGN5 | P82970 | 79366 | ENSG00000198157 | 350 |
| HNRNPA2B1 | P22626 | 3181 | ENSG00000122566 | 351 |
| HNRNPDL | O14979 | 9987 | ENSG00000152795 | 352 |
| HP | P00738 | 3240 | ENSG00000257017 | 353 |
| HP1BP3 | Q5SSJ5 | 50809 | ENSG00000127483 | 354 |
| HPR | P00739 | 3250 | ENSG00000261701 | 355 |
| HPX | P02790 | 3263 | ENSG00000110169 | 356 |
| HS3ST3A1 | Q9Y663 | 9955 | ENSG00000153976 | 357 |
| HS6ST1 | O60243 | 9394 | ENSG00000136720 | 358 |
| HS6ST2 | Q96MM7 | 90161 | ENSG00000171004 | 359 |
| HSD17B10 | Q99714 | 3028 | ENSG00000072506 | 360 |
| HSD17B4 | P51659 | 3295 | ENSG00000133835 | 361 |
| HSP90AA1 | P07900 | 3320 | ENSG00000080824 | 362 |
| HSP90AB1 | P08238 | 3326 | ENSG00000096384 | 363 |
| HSP90B1 | P14625 | 7184 | ENSG00000166598 | 364 |
| HSPA5 | P11021 | 3309 | ENSG00000044574 | 365 |
| HSPG2 | P98160 | 3339 | ENSG00000142798 | 366 |
| HTRA1 | Q92743 | 5654 | ENSG00000166033 | 367 |
| HYOU1 | Q9Y4L1 | 10525 | ENSG00000149428 | 368 |
| IARS | P41252 | 3376 | ENSG00000196305 | 369 |
| ICAM2 | P13598 | 3384 | ENSG00000108622 | 370 |
| IDE | P14735 | 3416 | ENSG00000119912 | 371 |
| IDH1 | O75874 | 3417 | ENSG00000138413 | 372 |
| IDH2 | P48735 | 3418 | ENSG00000182054 | 373 |
| IGF1 | P05019 | 3479 | ENSG00000017427 | 374 |
| IGF2 | P01344 | 3481 | ENSG00000167244 | 375 |
| IGFBP2 | P18065 | 3485 | ENSG00000115457 | 376 |
| IGFBP3 | P17936 | 3486 | ENSG00000146674 | 377 |
| IGFBP4 | P22692 | 3487 | ENSG00000141753 | 378 |
| IGFBP5 | P24593 | 3488 | ENSG00000115461 | 376 |
| IGFBP6 | P24592 | 3489 | ENSG00000167779 | 380 |
| IGFBP7 | Q16270 | 3490 | ENSG00000163453 | 381 |
| IGFBPL1 | Q8WX77 | 347252 | ENSG00000137142 | 382 |
| IGHA1 | P01876 | | ENSG00000211895 | 383 |
| IGHA2 | P01877 | | ENSG00000211890 | 384 |
| IGHG1 | P01857 | | ENSG00000211896 | 385 |
| IGHG2 | P01859 | | ENSG00000211893 | 386 |
| IGHG4 | P01861 | | ENSG00000211892 | 387 |
| IGHM | P01871 | | ENSG00000211899 | 388 |
| IGKC | P01834 | | | 389 |
| IGKV2-28 | A0A075B6P5 | | ENSG00000244116 | 390 |
| IGKV2D-40 | P01614 | | ENSG00000251039 | 391 |
| IGKV3D-20 | A0A0C4DH25 | | ENSG00000211625 | 392 |
| IGLC2 | P0DOY2 | | ENSG00000211677 | 393 |
| IGLC3 | P0DOY3 | | ENSG00000211679 | 394 |
| IGLV2-11 | P01706 | | ENSG00000211668 | 395 |
| IGSF1 | Q8N6C5 | 3547 | ENSG00000147255 | 396 |
| IGSF10 | Q6WRI0 | 285313 | ENSG00000152580 | 397 |
| ILF2 | Q12905 | 3608 | ENSG00000143621 | 398 |
| INHBA | P08476 | 3624 | ENSG00000122641 | 399 |
| INS | P01308 | 3630 | ENSG00000254647 | 400 |
| INS-IGF2 | F8WCM5 | 723961 | ENSG00000129965 | 401 |
| IPO11 | Q9UI26 | 51194 | ENSG00000086200 | 402 |
| IPO5 | O00410 | 3843 | ENSG00000065150 | 403 |
| IPO8 | O15397 | 10526 | ENSG00000133704 | 404 |
| IQGAP1 | P46940 | 8826 | ENSG00000140575 | 405 |
| ISOC1 | O96CN7 | 51015 | ENSG00000066583 | 406 |
| ITGAL | P20701 | 3683 | ENSG00000005844 | 407 |
| ITIH1 | P19827 | 3697 | ENSG00000055957 | 408 |
| ITIH2 | P19823 | 3698 | ENSG00000151655 | 409 |
| ITIH3 | Q06033 | 3699 | ENSG00000162267 | 410 |
| ITIH4 | Q14624 | 3700 | ENSG00000055955 | 411 |
| ITIH5 | Q86UX2 | 80760 | ENSG00000123243 | 412 |
| ITLN2 | Q8WWU7 | 142683 | ENSG00000158764 | 413 |
| JCHAIN | P01591 | 3512 | ENSG00000132465 | 414 |
| KARS | Q15046 | 3735 | ENSG00000065427 | 415 |
| KDM1A | O60341 | 23028 | ENSG00000004487 | 416 |
| KMT2A | Q03164 | 4297 | ENSG00000118058 | 417 |
| KNG1 | P01042 | 3827 | ENSG00000113889 | 418 |
| KRT10 | P13645 | 3858 | ENSG00000186395 | 419 |
| KRT14 | P02533 | 3861 | ENSG00000186847 | 420 |
| KRT17 | Q04695 | 3872 | ENSG00000128422 | 421 |
| KRT18 | P05783 | 3875 | ENSG00000111057 | 422 |
| KRT8 | P05787 | 3856 | ENSG00000170421 | 423 |
| LACRT | Q9GZZ8 | 90070 | ENSG00000135413 | 424 |
| LAG3 | P18627 | 3902 | ENSG00000089692 | 425 |
| LAMA1 | P25391 | 284217 | ENSG00000101680 | 426 |
| LAMA2 | P24043 | 3908 | ENSG00000196569 | 427 |
| LAMA5 | O15230 | 3911 | ENSG00000130702 | 428 |
| LAMB1 | P07942 | 3912 | ENSG00000091136 | 429 |
| LAMB2 | P55268 | 3913 | ENSG00000172037 | 430 |
| LAMC1 | P11047 | 3915 | ENSG00000135862 | 431 |
| LARS | Q9P2J5 | 51520 | ENSG00000133706 | 432 |
| LCAT | P04180 | 3931 | ENSG00000213398 | 433 |
| LSN1 | P31025 | 3933 | ENSG00000160349 | 434 |
| LDHA | P00338 | 3939 | ENSG00000134333 | 435 |
| LECT2 | O14960 | 3950 | ENSG00000145826 | 436 |
| LEFTY1 | O75610 | 10637 | ENSG00000243709 | 437 |
| LEFTY2 | O00292 | 7044 | ENSG00000143768 | 438 |
| LEFTYA | | | | 439 |
| LENG | Q8NES3 | 3955 | ENSG00000106003 | 440 |
| LGALS3BP | Q08380 | 3959 | ENSG00000108679 | 441 |
| LGALS7 | M0R281 | | ENSG00000205076 | 442 |
| LIG3 | P49916 | 3980 | ENSG00000005156 | 443 |
| LINGO1 | Q96FE5 | 84894 | ENSG00000169783 | 444 |
| LIPG | Q9Y5X9 | 9388 | ENSG00000101670 | 445 |
| LMAN2 | Q12907 | 10960 | ENSG00000169223 | 446 |
| LMNA | P02545 | 4000 | ENSG00000160789 | 447 |
| LOXL1 | Q08397 | 4016 | ENSG00000129038 | 448 |
| LOXL2 | Q9Y4K0 | 4017 | ENSG00000134013 | 449 |
| LOXL3 | P58215 | 84695 | ENSG00000115318 | 450 |
| LPL | P06858 | 4023 | ENSG00000175445 | 451 |
| LRG1 | P02750 | 116844 | ENSG00000171236 | 452 |
| LRP1 | Q07954 | 4035 | ENSG00000123384 | 453 |
| LRPAP1 | P30533 | 4043 | ENSG00000163956 | 454 |
| LRRC59 | Q96AG4 | 55379 | ENSG00000108829 | 455 |
| LRRTM4 | Q86VH4 | 80059 | ENSG00000176204 | 456 |
| LSR | Q86X29 | 51599 | ENSG00000105699 | 457 |
| LTBP1 | Q14766 | 4052 | ENSG00000049323 | 458 |
| LTBP4 | Q8N2S1 | 8425 | ENSG00000090006 | 459 |
| LTF | P02788 | 4057 | ENSG00000012223 | 460 |
| LUM | P51884 | 4060 | ENSG00000139329 | 461 |
| LYAR | Q9NX58 | 55646 | ENSG00000145220 | 462 |
| LYZ | P61626 | 4069 | ENSG00000090382 | 463 |
| MANF | P55145 | 7873 | ENSG00000145050 | 464 |
| MAP2K2 | P36507 | 5605 | ENSG00000126934 | 465 |
| MAP4 | P27816 | 4134 | ENSG00000047849 | 466 |
| MAPK1 | P28482 | 5594 | ENSG00000100030 | 467 |
| MASP1 | P48740 | 5648 | ENSG00000127241 | 468 |
| MATN2 | O00339 | 4147 | ENSG00000132561 | 469 |
| MATN3 | O15232 | 4148 | ENSG00000132031 | 470 |
| MATR3 | P43243 | 9782 | ENSG00000015479 | 471 |
| MAZ | P56270 | 4150 | ENSG00000103495 | 472 |
| MBNL1 | Q9NR56 | 4154 | ENSG00000152601 | 473 |
| MCM5 | P33992 | 4174 | ENSG00000100297 | 474 |
| MDH2 | P40926 | 4191 | ENSG00000146701 | 475 |
| MDK | P21741 | 4192 | ENSG00000110492 | 476 |
| MEGF10 | Q96KG7 | 84466 | ENSG00000145794 | 477 |
| MEGF6 | O75095 | 1953 | ENSG00000162591 | 478 |
| METAP2 | P50579 | 10988 | ENSG00000111142 | 479 |
| METTL14 | Q9HCE5 | 57721 | ENSG00000145388 | 480 |
| MFAP2 | P55001 | 4237 | ENSG00000117122 | 481 |
| MFGE8 | Q08431 | 4240 | ENSG00000140545 | 482 |
| MGAT1 | P26572 | 4245 | ENSG00000131446 | 483 |
| MIF | P14174 | 4282 | ENSG00000240972 | 484 |
| MINPP1 | Q9UNW1 | 9562 | ENSG00000107789 | 485 |
| MMP2 | P08253 | 4313 | ENSG00000087245 | 486 |
| MMP9 | P14780 | 4318 | ENSG00000100985 | 487 |
| MSMB | P08118 | 4477 | ENSG00000263639 | 488 |
| MSN | P26038 | 4478 | ENSG00000147065 | 489 |
| MST1 | P26927 | 4485 | ENSG00000173531 | 490 |
| MST1L | Q2TV78 | 11223 | | 491 |
| MTDH | Q86UE4 | 92140 | ENSG00000147649 | 492 |
| MTHFD1 | P11586 | 4522 | ENSG00000100714 | 493 |
| MTHFD2 | P13995 | 10797 | ENSG00000065911 | 494 |
| MXRA5 | Q9NR99 | 25878 | ENSG00000101825 | 495 |
| MYBBP1A | Q9BQG0 | 10514 | ENSG00000132382 | 496 |
| MYL3 | P08590 | 4634 | ENSG00000160808 | 497 |
| MYL4 | P12829 | 4635 | ENSG00000198336 | 498 |
| NAA15 | Q9BXJ9 | 80155 | ENSG00000164134 | 499 |
| NAMPT | P43490 | 10135 | ENSG00000105835 | 500 |
| NASP | P49321 | 4678 | ENSG00000132780 | 501 |
| NCAM1 | P13591 | 4684 | ENSG00000149294 | 502 |
| NCAN | O14594 | 1463 | ENSG00000130287 | 503 |
| NDNF | Q8TB73 | 79625 | ENSG00000173376 | 504 |
| NDST1 | P52848 | 3340 | ENSG00000070614 | 505 |
| NECTIN1 | Q15223 | 5818 | ENSG00000110400 | 506 |
| NECTIN3 | Q9NQS3 | 25945 | ENSG00000177707 | 507 |
| NELL2 | Q99435 | 4753 | ENSG00000184613 | 508 |
| NID1 | P14543 | 4811 | ENSG00000116962 | 509 |
| NID2 | Q14112 | 22795 | ENSG00000087303 | 510 |
| NIPBL | Q6KC79 | 25836 | ENSG00000164190 | 511 |
| NLGN3 | Q9NZ94 | 54413 | ENSG00000196338 | 512 |
| NLGN4Y | Q8NFZ3 | 22829 | ENSG00000165246 | 513 |
| NME1-NME2 | J3KPD9 | | ENSG00000011052 | 514 |
| NMT1 | P30419 | 4836 | ENSG00000136448 | 515 |
| NOLC1 | Q14978 | 9221 | ENSG00000166197 | 516 |
| NOV | A0A024R9J4 | 4856 | | 517 |
| NPC2 | P61916 | 10577 | ENSG00000119655 | 518 |
| NPM3 | O75607 | 10360 | ENSG00000107833 | 519 |
| NPTX1 | Q15818 | 4884 | | 520 |
| NPTX2 | P47972 | 4885 | ENSG00000106236 | 521 |
| NPTXR | O95502 | 23467 | ENSG00000221890 | 522 |
| NRG1 | Q02297 | 3084 | ENSG00000157168 | 523 |
| NRG2 | O14511 | 9542 | ENSG00000158458 | 524 |
| NRP1 | O14786 | 8829 | ENSG00000099250 | 525 |
| NRP2 | O60462 | 8828 | ENSG00000118257 | 526 |
| NSUN5 | Q96P11 | 55695 | ENSG00000130305 | 527 |
| NTS | P30990 | 4922 | ENSG00000133636 | 528 |
| NUBP2 | Q9Y5Y2 | 10101 | ENSG00000095906 | 529 |
| NUCB1 | Q02818 | 4924 | ENSG00000104805 | 530 |
| NUMA1 | Q14980 | 4926 | ENSG00000137497 | 531 |
| NUP155 | O75694 | 9631 | ENSG00000113569 | 532 |
| OAF | Q86UD1 | 220323 | ENSG00000184232 | 533 |
| OLA1 | Q9NTK5 | 29789 | ENSG00000138430 | 534 |
| OLFM2 | O95897 | 93145 | | 535 |
| OLFML2A | Q68BL7 | 169611 | ENSG00000185585 | 536 |
| OLFML3 | Q9NRN5 | 56944 | ENSG00000116774 | 537 |
| ORM1 | P02763 | 5004 | ENSG00000229314 | 538 |
| ORM2 | P19652 | 5005 | ENSG00000228278 | 539 |
| P4HB | P07237 | 5034 | ENSG00000185624 | 540 |
| PACSIN2 | Q9UNF0 | 11252 | ENSG00000100266 | 541 |
| PAFAH1B1 | P43034 | 5048 | ENSG00000007168 | 542 |
| PAIP1 | Q9H074 | 10605 | ENSG00000172239 | 543 |
| PAM | P19021 | 5066 | ENSG00000145730 | 544 |
| PAMR1 | Q6UXH9 | 25891 | ENSG00000149090 | 545 |
| PAPLN | O95428 | 89932 | ENSG00000100767 | 546 |
| PAPPA | Q13219 | 5069 | ENSG00000182752 | 547 |
| PARP1 | P09874 | 142 | ENSG00000143799 | 548 |
| PC | P11498 | 5091 | ENSG00000173599 | 549 |
| PCDH1 | Q08174 | 5097 | ENSG00000156453 | 550 |
| PCLO | Q9Y6V0 | 27445 | ENSG00000186472 | 551 |
| PCOLCE | Q15113 | 5118 | ENSG00000106333 | 552 |
| PCOLCE2 | Q9UKZ9 | 26577 | ENSG00000163710 | 553 |
| PCSK5 | Q92824 | 5125 | ENSG00000099139 | 554 |
| PCSK9 | Q8NBP7 | 255738 | ENSG00000169174 | 555 |
| PDCD6IP | Q8WUM4 | 10015 | ENSG00000170248 | 556 |
| PDGFD | Q9GZP0 | 80310 | ENSG00000170962 | 557 |
| PDGFRL | Q15198 | 5157 | ENSG00000104213 | 558 |
| PDIA3 | P30101 | 2923 | ENSG00000167004 | 559 |
| PDIA4 | P13667 | 9601 | ENSG00000155660 | 560 |
| PDIA5 | Q14554 | 10954 | ENSG00000065485 | 561 |
| PDIA6 | Q15084 | 10130 | ENSG00000143870 | 562 |
| PFAS | O15067 | 5198 | ENSG00000178921 | 563 |
| PFKP | Q01813 | 5214 | ENSG00000067057 | 564 |
| PFN1 | P07737 | 5216 | ENSG00000108518 | 565 |
| PGD | P52209 | 5226 | ENSG00000142657 | 566 |
| PGLYRP2 | Q96PD5 | 114770 | ENSG00000161031 | 567 |
| PHGDH | O43175 | 26227 | ENSG00000092621 | 568 |
| PI16 | Q6UXB8 | 221476 | ENSG00000164530 | 569 |
| PIGR | P01833 | 5284 | ENSG00000162896 | 570 |
| PIP | P12273 | 5304 | ENSG00000159763 | 571 |
| PKDCC | Q504Y2 | 91461 | ENSG00000162878 | 572 |
| PKM | P14618 | 5315 | ENSG00000067225 | 573 |
| PLAT | P00750 | 5327 | ENSG00000104368 | 574 |
| PLAU | P00749 | 5328 | ENSG00000122861 | 575 |
| PLCB3 | Q01970 | 5331 | ENSG00000149782 | 576 |
| PLEC | Q15149 | 5339 | ENSG00000178209 | 577 |
| PLG | P00747 | 5340 | ENSG00000122194 | 578 |
| PLIN4 | Q96Q06 | 729359 | ENSG00000167676 | 579 |
| PLOD1 | Q02809 | 5351 | ENSG00000083444 | 580 |
| PLOD2 | O00469 | 5352 | ENSG00000152952 | 581 |
| PLOD3 | O60568 | 8985 | ENSG00000106397 | 582 |
| PLTP | P55058 | 5360 | ENSG00000100979 | 583 |
| POLL | Q9UGP5 | 27343 | ENSG00000166169 | 584 |
| POMC | P01189 | 5443 | ENSG00000115138 | 585 |
| POSTN | Q15063 | 10631 | ENSG00000133110 | 586 |
| PPIA | P62937 | 5478 | ENSG00000196262 | 587 |
| PPIB | P23284 | 5479 | ENSG00000166794 | 588 |
| PPP1CA | P62136 | 5499 | ENSG00000172531 | 589 |
| PPP1CC | P36873 | 5501 | ENSG00000186298 | 590 |
| PPP2R1A | P30153 | 5518 | ENSG00000105568 | 591 |
| PPT1 | P50897 | 5538 | ENSG00000131238 | 592 |
| PRB3 | Q04118 | | ENSG00000197870 | 593 |
| PRB4 | P10163 | | | 594 |
| PRCP | P42785 | 5547 | ENSG00000137509 | 595 |
| PRDX2 | P32119 | 7001 | ENSG00000167815 | 596 |
| PRDX4 | Q13162 | 10549 | ENSG00000123131 | 597 |
| PRDX5 | P30044 | 25824 | ENSG00000126432 | 598 |
| PRG4 | Q92954 | 10216 | ENSG00000116690 | 599 |
| PRKDC | P78527 | 5591 | ENSG00000253729 | 600 |
| PRMT1 | Q99873 | 3276 | ENSG00000126457 | 601 |
| PRMT5 | O14744 | 10419 | ENSG00000100462 | 602 |
| PROM1 | O43490 | 8842 | ENSG00000007062 | 603 |
| PRPF19 | Q9UMS4 | 27339 | ENSG00000110107 | 604 |
| PRPF40A | O75400 | 55660 | ENSG00000196504 | 605 |
| PRPF4B | Q13523 | 8899 | ENSG00000112739 | 606 |
| PRPF6 | O94906 | 24148 | ENSG00000101161 | 607 |
| PRPF8 | Q6P209 | 10594 | ENSG00000174231 | 608 |
| PRPSAP2 | O60256 | 5636 | ENSG00000141127 | 609 |
| PRR4 | Q16378 | 11272 | ENSG00000111215 | 610 |
| PRSS2 | P07478 | 5645 | ENSG00000275896 | 611 |
| PRSS23 | O95084 | 11098 | ENSG00000150687 | 612 |
| PRSS3 | P35030 | 5646 | ENSG00000010438 | 613 |
| PRTG | Q2VWP7 | 283659 | ENSG00000166450 | 614 |
| PSIP1 | O75475 | 11168 | ENSG00000164985 | 615 |
| PSMB6 | P28072 | 5694 | ENSG00000142507 | 616 |
| PSMD1 | Q99460 | 5707 | ENSG00000173692 | 617 |
| PSMD2 | Q13200 | 5708 | ENSG00000175166 | 618 |
| PSMD5 | Q16401 | 5711 | ENSG00000095261 | 619 |
| PSMD6 | Q15008 | 9861 | ENSG00000163636 | 620 |
| PSMD8 | P48556 | 5714 | ENSG00000099341 | 621 |
| PSME3 | P61289 | 10197 | ENSG00000131467 | 622 |
| PTK2 | Q05397 | 5747 | ENSG00000169398 | 623 |
| PTK7 | Q13308 | 5754 | ENSG00000112655 | 624 |
| PTN | P21246 | 5764 | ENSG00000105894 | 625 |
| PTPRC | P08575 | 5788 | ENSG00000081237 | 626 |
| PTPRD | P23468 | 5789 | ENSG00000153707 | 627 |
| PTPRF | P10586 | 5792 | ENSG00000142949 | 628 |
| PTPRS | Q13332 | 5802 | ENSG00000105426 | 629 |
| PTPRZ1 | P23471 | 5803 | ENSG00000106278 | 630 |
| PUF60 | Q9UHX1 | 22827 | ENSG00000179950 | 631 |
| PXDN | Q92626 | 7837 | ENSG00000130508 | 632 |
| PZP | P20742 | | ENSG00000126838 | 633 |
| QPRT | Q15274 | 23475 | ENSG00000103485 | 634 |
| QSOX1 | O00391 | 5768 | ENSG00000116260 | 635 |
| RAB7A | P51149 | 7879 | ENSG00000075785 | 636 |
| RACK1 | P63244 | 10399 | ENSG00000204628 | 637 |
| RARRES2 | Q99969 | 5919 | ENSG00000106538 | 638 |
| RBMX | P38159 | 27316 | ENSG00000147274 | 639 |
| RBP4 | P02753 | 5950 | ENSG00000138207 | 640 |
| RCC1 | P18754 | 1104 | ENSG00000180198 | 641 |
| RCOR1 | Q9UKL0 | 23186 | ENSG00000089902 | 642 |
| RECQL | P46063 | 5965 | ENSG00000004700 | 643 |
| RELN | P78509 | 5649 | ENSG00000189056 | 644 |
| RNASE1 | P07998 | 6035 | ENSG00000129538 | 645 |
| RNASE4 | P34096 | 6038 | ENSG00000258818 | 646 |
| ROBO1 | Q9Y6N7 | 6091 | ENSG00000169855 | 647 |
| RPL14 | P50914 | 9045 | ENSG00000188846 | 648 |
| RPL18 | Q07020 | 6141 | ENSG00000063177 | 649 |
| RPL19 | P84098 | 6143 | ENSG00000108298 | 650 |
| RPL23A | P62750 | 6147 | ENSG00000198242 | 651 |
| RPL26 | P61254 | 6154 | ENSG00000161970 | 652 |
| RPL29 | P47914 | 6159 | ENSG00000162244 | 653 |
| RPL3 | P39023 | 6122 | ENSG00000100316 | 654 |
| RPL35 | P42766 | 11224 | ENSG00000136942 | 655 |
| RPL4 | P36578 | 6124 | ENSG00000174444 | 656 |
| RPL9 | A0A2R8Y5Y7 | | ENSG00000163682 | 657 |
| RPLP0 | P05388 | 6175 | ENSG00000089157 | 658 |
| RPLP1 | P05386 | 6176 | ENSG00000137818 | 659 |
| RPLP2 | P05387 | 6181 | ENSG00000177600 | 660 |
| RPN2 | P04844 | 6185 | ENSG00000118705 | 661 |
| RPS13 | P62277 | 6207 | ENSG00000110700 | 662 |
| RPS20 | P60866 | 6224 | ENSG00000008988 | 663 |
| RPS23 | P62266 | 6228 | ENSG00000186468 | 664 |
| RPS27A | P62979 | 6233 | ENSG00000143947 | 665 |
| RPS27L | Q71UM5 | 51065 | ENSG00000185088 | 666 |
| RPS3 | P23396 | 6188 | ENSG00000149273 | 667 |
| RRBP1 | Q9P2E9 | 6238 | ENSG00000125844 | 668 |
| RSF1 | Q96T23 | 51773 | ENSG00000048649 | 669 |
| RSL1D1 | O76021 | 26156 | ENSG00000171490 | 670 |
| RTF1 | Q92541 | 23168 | ENSG00000137815 | 671 |
| RTN4 | Q9NQC3 | 57142 | ENSG00000115310 | 672 |
| RTN4RL2 | Q86UN3 | 349667 | ENSG00000186907 | 673 |
| RUVBL2 | Q9Y230 | 10856 | ENSG00000183207 | 674 |
| S100A12 | P80511 | 6283 | ENSG00000163221 | 675 |
| S100A13 | Q99584 | 6284 | ENSG00000189171 | 676 |
| S100A7 | P31151 | 6278 | ENSG00000143556 | 677 |
| S100A8 | P05109 | 6279 | ENSG00000143546 | 678 |
| S100A9 | P06702 | 6280 | ENSG00000163220 | 679 |
| SAP30 | O75446 | 8819 | ENSG00000164105 | 680 |
| SARS | P49591 | 6301 | ENSG00000031698 | 681 |
| SBSN | Q6UWP8 | 374897 | ENSG00000189001 | 682 |
| SCG3 | Q8WXD2 | 29106 | ENSG00000104112 | 683 |
| SCGB2A2 | Q13296 | 4250 | ENSG00000110484 | 684 |
| SCUBE1 | Q8IWY4 | 80274 | ENSG00000159307 | 685 |
| SCUBE3 | Q8IX30 | 222663 | ENSG00000146197 | 686 |
| SDC1 | P18827 | 6382 | ENSG00000115884 | 687 |
| SDC4 | P31431 | 6385 | ENSG00000124145 | 688 |
| SDCBP | O00560 | 6386 | ENSG00000137575 | 689 |
| SDF4 | Q9BRK5 | 51150 | ENSG00000078808 | 690 |
| SEC13 | P55735 | 6396 | ENSG00000157020 | 691 |
| SELENOP | P49908 | 6414 | ENSG00000250722 | 692 |
| SEMA3A | Q14563 | 10371 | ENSG00000075213 | 693 |
| SEMA3F | Q13275 | 6405 | ENSG00000001617 | 694 |
| SEMA4B | Q9NPR2 | 10509 | ENSG00000185033 | 695 |
| SEMA4D | Q92854 | 10507 | ENSG00000187764 | 696 |
| SEMA5A | Q13591 | 9037 | ENSG00000112902 | 697 |
| SEMA6A | Q9H2E6 | 57556 | ENSG00000092421 | 698 |
| SEMA6D | Q8NFY4 | 80031 | ENSG00000137872 | 699 |
| SEMG1 | P04279 | 6406 | ENSG00000124233 | 700 |
| SEPT9 | Q9UHD8 | 10801 | ENSG00000184640 | 701 |
| SERBP1 | Q8NC51 | 26135 | ENSG00000142864 | 702 |
| SERPINA1 | P01009 | 5265 | ENSG00000197249 | 703 |
| SERPINA3 | P01011 | 12 | ENSG00000196136 | 704 |
| SERPINA5 | P05154 | 5104 | ENSG00000188488 | 705 |
| SERPINA7 | P05543 | 6906 | ENSG00000123561 | 706 |
| SERPINB12 | Q96P63 | 89777 | ENSG00000166634 | 707 |
| SERPINB3 | P29508 | 6317 | ENSG00000057149 | 708 |
| SERPINB9 | P50453 | 5272 | ENSG00000170542 | 709 |
| SERPINC1 | P01008 | 462 | ENSG00000117601 | 710 |
| SERPIND1 | P05546 | 3053 | ENSG00000099937 | 711 |
| SERPINE1 | P05121 | 5054 | ENSG00000106366 | 712 |
| SERPINE2 | P07093 | 5270 | ENSG00000135919 | 713 |
| SERPINF1 | P36955 | 5176 | ENSG00000132386 | 714 |
| SERPINF2 | P08697 | 5345 | ENSG00000167711 | 715 |
| SERPING1 | P05155 | 710 | ENSG00000149131 | 716 |
| SERPINH1 | P50454 | 871 | ENSG00000149257 | 717 |
| SERPINI1 | Q99574 | 5274 | ENSG00000163536 | 718 |
| SF3B1 | O75533 | 23451 | ENSG00000115524 | 719 |
| SF3B2 | Q13435 | 10992 | ENSG00000087365 | 720 |
| SF3B3 | Q15393 | 23450 | ENSG00000189091 | 721 |
| SFPQ | P23246 | 6421 | ENSG00000116560 | 722 |
| SFRP1 | Q8N474 | 6422 | ENSG00000104332 | 723 |
| SFRP2 | Q96HF1 | 6423 | ENSG00000145423 | 724 |
| SH2B1 | Q9NRF2 | 25970 | ENSG00000178188 | 725 |
| SHBG | P04278 | 6462 | ENSG00000129214 | 726 |
| SHMT1 | P34896 | 6470 | ENSG00000176974 | 727 |
| SKIV2L2 | L8E9T8 | | | 728 |
| SLC1A5 | Q15758 | 6510 | ENSG00000105281 | 729 |
| SLC2A14 | Q8TDB8 | 144195 | ENSG00000173262 | 730 |
| SLC39A10 | Q9ULF5 | 57181 | ENSG00000196950 | 731 |
| SLIT2 | O94813 | 9353 | | 732 |
| SLIT3 | O75094 | 6586 | ENSG00000184347 | 733 |
| SLPI | P03973 | 6590 | ENSG00000124107 | 734 |
| SLTM | Q9NWH9 | 79811 | ENSG00000137776 | 735 |
| SLURP1 | P55000 | 57152 | ENSG00000126233 | 736 |
| SMARCC1 | Q92922 | 6599 | ENSG00000173473 | 737 |
| SMARCD1 | Q96GM5 | 6602 | ENSG00000066117 | 738 |
| SMARCD2 | Q92925 | 6603 | ENSG00000108604 | 739 |
| SMC1A | Q14683 | 8243 | ENSG00000072501 | 740 |
| SMC3 | Q9UQE7 | 9126 | ENSG00000108055 | 741 |
| SMOC1 | Q9H4F8 | 64093 | ENSG00000198732 | 742 |
| SMOC2 | Q9H3U7 | 64094 | ENSG00000112562 | 743 |
| SMPDL3B | Q92485 | 27293 | ENSG00000130768 | 744 |
| SMR3B | P02814 | 10879 | ENSG00000171201 | 745 |
| SNRPB | P14678 | 6628 | ENSG00000125835 | 746 |
| SNRPD1 | P62314 | 6632 | ENSG00000167088 | 747 |
| SNRPD3 | P62318 | 6634 | ENSG00000100028 | 748 |
| SOD3 | P08294 | 6649 | ENSG00000109610 | 749 |
| SPARC | P09486 | 6678 | ENSG00000113140 | 750 |
| SPINT1 | O43278 | 6692 | ENSG00000166145 | 751 |
| SPINT2 | O43291 | 10653 | ENSG00000167642 | 752 |
| SPOCK1 | Q08629 | 6695 | ENSG00000152377 | 753 |
| SPON1 | Q9HCB6 | 10418 | ENSG00000262655 | 754 |
| SPP1 | P10451 | 6696 | ENSG00000118785 | 755 |
| SRP14 | P37108 | 6727 | ENSG00000140319 | 756 |
| SRPX | P78539 | 8406 | ENSG00000101955 | 757 |
| SRPX2 | O60687 | 27286 | ENSG00000102359 | 758 |
| SRSF1 | Q07955 | 6426 | ENSG00000136450 | 759 |
| SSB | P05455 | 6741 | ENSG00000138385 | 760 |
| SSC5D | A1L4H1 | 284297 | ENSG00000179954 | 761 |
| ST6GAL1 | P15907 | 6480 | ENSG00000073849 | 762 |
| ST6GAL2 | Q96JF0 | 84620 | ENSG00000144057 | 763 |
| STAG1 | Q8WVM7 | 10274 | ENSG00000118007 | 764 |
| STC1 | P52823 | 6781 | ENSG00000159167 | 765 |
| STC2 | O76061 | 8614 | ENSG00000113739 | 766 |
| SUB1 | P53999 | 10923 | ENSG00000113387 | 767 |
| SULF2 | Q8IWU5 | 55959 | ENSG00000196562 | 768 |
| SUMF2 | Q8NBJ7 | 25870 | ENSG00000129103 | 769 |
| SUPT16H | Q9Y5B9 | 11198 | ENSG00000092201 | 770 |
| SUPT6H | Q7KZ85 | 6830 | ENSG00000109111 | 771 |
| SVEP1 | Q4LDE5 | 79987 | ENSG00000165124 | 772 |
| SYNCRIP | O60506 | 10492 | ENSG00000135316 | 773 |
| TAGLN2 | P37802 | 8407 | ENSG00000158710 | 774 |
| TBL1XR1 | Q9BZK7 | 79718 | ENSG00000177565 | 775 |
| TCN2 | P20062 | 6948 | ENSG00000185339 | 776 |
| TCOF1 | Q13428 | 6949 | ENSG00000070814 | 777 |
| TF | P02787 | 7018 | ENSG00000091513 | 778 |
| TFAM | Q00059 | 7019 | ENSG00000108064 | 779 |
| TFPI | P10646 | 7035 | ENSG00000003436 | 780 |
| TFRC | P02786 | 7037 | ENSG00000072274 | 781 |
| TGFB2 | P61812 | 7042 | ENSG00000092969 | 782 |
| TGFBI | Q15582 | 7045 | ENSG00000120708 | 783 |
| THBS1 | P07996 | 7057 | ENSG00000137801 | 784 |
| THBS2 | P35442 | 7058 | ENSG00000186340 | 785 |
| THBS3 | P49746 | 7059 | ENSG00000169231 | 786 |
| THBS4 | P35443 | 7060 | ENSG00000113296 | 787 |
| THOC3 | Q96J01 | 84321 | ENSG00000051596 | 788 |
| THSD7A | Q9UPZ6 | 221981 | ENSG00000005108 | 789 |
| TIMP1 | P01033 | 7076 | ENSG00000102265 | 790 |
| TIMP2 | P16035 | 7077 | ENSG00000035862 | 791 |
| TIMP3 | P35625 | 7078 | ENSG00000100234 | 792 |
| TINAGL1 | Q9GZM7 | 64129 | ENSG00000142910 | 793 |
| TJP1 | Q07157 | 7082 | ENSG00000104067 | 794 |
| TNC | P24821 | 3371 | ENSG00000041982 | 795 |
| TNN | Q9UQP3 | 63923 | | 796 |
| TNXB | P22105 | 7148 | ENSG00000168477 | 797 |
| TOP1 | P11387 | 7150 | ENSG00000198900 | 798 |
| TPM4 | P67936 | 7171 | ENSG00000167460 | 799 |
| TPP2 | P29144 | 7174 | ENSG00000134900 | 800 |
| TRIM28 | Q13263 | 10155 | ENSG00000130726 | 801 |
| TRIP10 | Q15642 | 9322 | ENSG00000125733 | 802 |
| TRMT1 | Q9NXH9 | 55621 | ENSG00000104907 | 803 |
| TSKU | Q8WUA8 | 25987 | ENSG00000182704 | 804 |
| TTR | P02766 | 7276 | ENSG00000118271 | 805 |
| TUBB4A | P04350 | 10382 | ENSG00000104833 | 806 |
| TUFM | P49411 | 7284 | ENSG00000178952 | 807 |
| TWSG1 | Q9GZX9 | 57045 | ENSG00000128791 | 808 |
| TXN | P10599 | 7295 | ENSG00000136810 | 809 |
| TXNDC16 | Q9P2K2 | 57544 | ENSG00000087301 | 810 |
| TXNDC5 | Q8NBS9 | 81567 | ENSG00000239264 | 811 |
| U2AF2 | P26368 | 11338 | ENSG00000063244 | 812 |
| UBE2O | Q9C0C9 | 63893 | ENSG00000175931 | 813 |
| UBR4 | Q5T4S7 | 23352 | ENSG00000127481 | 814 |
| UCHL1 | P09936 | 7345 | ENSG00000154277 | 815 |
| UCHL3 | P15374 | 7347 | ENSG00000118939 | 816 |
| UFL1 | O94874 | 23376 | ENSG00000014123 | 817 |
| UGP2 | Q16851 | 7360 | ENSG00000169764 | 818 |
| USP11 | P51784 | 8237 | ENSG00000102226 | 819 |
| USP14 | P54578 | 9097 | ENSG00000101557 | 820 |
| USP43 | Q70EL4 | 124739 | ENSG00000154914 | 821 |
| UTP4 | Q969X6 | 84916 | ENSG00000141076 | 822 |
| VARS | P26640 | 7407 | ENSG00000096171 | 823 |
| VASN | Q6EMK4 | 114990 | ENSG00000168140 | 824 |
| VCAN | P13611 | 1462 | ENSG00000038427 | 825 |
| VCP | P55072 | 7415 | ENSG00000165280 | 826 |
| VEGFA | P15692 | 7422 | ENSG00000112715 | 827 |
| VIT | Q6UXI7 | 5212 | ENSG00000205221 | 828 |
| VNN1 | O95497 | 8876 | ENSG00000112299 | 829 |
| VPS35 | Q96QK1 | 55737 | ENSG00000069329 | 830 |
| VTN | P04004 | 7448 | ENSG00000109072 | 831 |
| VWF | P04275 | 7450 | ENSG00000110799 | 832 |
| WDR3 | Q9UNX4 | 10885 | ENSG00000065183 | 833 |
| WDR36 | Q8NI36 | 134430 | ENSG00000134987 | 834 |
| WDR4 | P57081 | 10785 | ENSG00000160193 | 835 |
| WDR43 | Q15061 | 23160 | ENSG00000163811 | 836 |
| WFIKKN1 | Q96NZ8 | 117166 | ENSG00000127578 | 837 |
| WFIKKN2 | Q8TBU8 | 124857 | ENSG00000173714 | 838 |
| XRCC5 | P13010 | 7520 | ENSG00000079246 | 839 |
| XYLT1 | Q86Y38 | 64131 | ENSG00000103489 | 840 |
| XYLT2 | Q9H1B5 | 64132 | ENSG00000015532 | 841 |
| YBX1 | P67809 | 4904 | ENSG00000065978 | 842 |
| YBX3 | P16989 | 8531 | ENSG00000060138 | 843 |
| ZG16B | Q96DA0 | 124220 | ENSG00000162078 | 844 |
| ZNF207 | O43670 | 7756 | ENSG00000010244 | 845 |
| ZNF326 | Q5BKZ1 | 284695 | ENSG00000162664 | 846 |
| ZNF706 | Q9Y5V0 | 51123 | ENSG00000120963 | 847 |
| APLP1 | P51693 | 333 | ENSG00000105290 | 848 |
| APP | P05067 | 351 | ENSG00000142192 | 849 |
| NPNT | Q6UXI9 | 255743 | ENSG00000168743 | 850 |
| RPL22 | Q6UXI9 | 6146 | ENSG00000116251 | 851 |
| FGF19 | O95750 | 9965 | ENSG00000162344 | 852 |
| BTC | P35070 | 685 | ENSG00000174808 | 853 |
| IL13RA2 | Q14627 | 3598 | ENSG00000123496 | 854 |
| CD170 | O15389 | | ENSG00000105501 | 855 |
| IL15 | P40933 | 3600 | ENSG00000164136 | 856 |
| WAP | Q8TEU8 | | ENSG00000173714 | 857 |
| MFRP | Q9BY79 | 83552 | ENSG00000235718 | 858 |
| IL10Ra | Q13651 | 3587 | ENSG00000110324 | 859 |
| ChemR23 | Q99788 | 1240 | ENSG00000174600 | 860 |
| HBEGF | Q99075 | 1839 | ENSG00000113070 | 861 |
| IL16 | Q14005 | 3603 | ENSG00000172349 | 862 |
| IL7Ra | P16871 | 3575 | ENSG00000168685 | 863 |
| TNFSF10C | O14798 | 8794 | ENSG00000173535 | 864 |
| BMP6 | P22004 | 654 | ENSG00000153162 | 865 |
| IL36g | P14778 | 56300 | ENSG00000136688 | 866 |
| IL1RA | P18510 | 3557 | ENSG00000136689 | 867 |
| KREMEN2 | Q8NCW0 | 79412 | | 868 |
| TNFSF10D | Q9UBN6 | 8793 | ENSG00000173530 | 869 |
| CXCCR1 | P49238 | 1524 | ENSG00000168329 | 870 |
| CCL23 | P55773 | 6368 | ENSG00000276114 | 871 |
| Catenin | P35222 | 1499 | ENSG00000168036 | 872 |
| TNFSF10 | P50591 | 8743 | ENSG00000121858 | 873 |
| CCL14 | Q16627 | 6358 | ENSG00000276409 | 874 |
| IL2 | P60568 | 3558 | ENSG00000109471 | 875 |
| FGF1 | P05230 | 2246 | ENSG00000113578 | 876 |

In certain embodiments, the heparin-associated polypeptide comprises one or more of the polypeptides listed in Table 2, Table 1, or a proteoform thereof. In certain embodiments, the heparin-associated polypeptide is at least about 85%, 90%, 95%, 97%, 98%, 99% or 100% identical to a polypeptide listed in Table 2, Table 1, or a proteoform thereof. In certain embodiments, the heparin-associated polypeptide comprises a polypeptide at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAM4A5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, or RPL29, or any combination thereof. In certain embodiments, the heparin-associated polypeptide comprises a polypeptide at least about 85%, 90%, 95%, 97%, 98%, 99%, or 100% identical to VTN, POSTN, FGF7, THBS2, THBS4, THBS1, IL-15, or IGF2, or any combination thereof. In certain embodiments, the heparin-associated polypeptide comprises THBS1. In certain embodiments, the heparin-associated polypeptide comprises THBS2 In certain embodiments, the heparin-associated polypeptide comprises THBS4. In certain embodiments, the heparin-associated polypeptide comprises FGF17. In certain embodiments, the heparin-associated polypeptide comprises VTN. In certain embodiments, the heparin-associated polypeptide comprises POSTN. In certain embodiments, the heparin-associated polypeptide comprises IGF2 In certain embodiments, the heparin-associated polypeptide comprises IL-15. In certain embodiments, described herein, is a composition comprising any 1, 2, 3, 4, 5, 6, 7, 8, 9, 10 or more polypeptides at least about 85%, 90%, 95%, 97%, 98%, 99%, or 100% identical to a polypeptide Table 2, Table 1, ADAMTS12, INS-IGF2, AOC1, SOD3, CLU, ITIH1l, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, a proteoform thereof, or a combination thereof; and a pharmaceutically acceptable excipient, carrier, or diluent. In certain embodiments, the composition comprises a plurality of peptides from Table 2, and optionally a pharmaceutically acceptable excipient, carrier, or diluent. In some cases the plurality comprises 1, 2, 3, 4, 5, 6, 7, 8, 9, 10 or more polypeptides of Table 2. In some cases, one or more of the plurality of polypeptides is at least about 85%, 90%, 95%, 97%, 98%, 99%, or 100% identical to a polypeptide of Table 2. In certain embodiments, the composition comprises a plurality of peptides from Table 1; and optionally a pharmaceutically acceptable excipient, carrier, or diluent. In some cases the plurality comprises 1, 2, 3, 4, 5, 6, 7, 8, 9, 10 or more polypeptides of Table 1. In some cases, one or more of the plurality of polypeptides is at least about 85%, 90%, 95%, 97%, 98%, 99%, or 100% identical to a polypeptide of Table 1. In certain embodiments, the composition comprises THBS1. In certain embodiments, the composition comprises THBS2. In certain embodiments, the composition comprises THBS4. In certain embodiments, the composition comprises FGF17. In certain embodiments, the composition comprises VTN. In certain embodiments, the composition comprises POSTN. In certain embodiments, the composition comprises IGF2. In certain embodiments, the composition comprises IL-15. In certain embodiments, the composition comprises IGF2, THBS2, and THBS4. In certain embodiments, the composition comprises IL-15, TH-2, and THBS4. In certain embodiments, the composition comprises THBS2 and THBS4. In certain embodiments, the composition comprises THBS2, THBS4, and VTN. In certain embodiments, the composition comprises THBS2, THBS4, and ANOS1. In certain embodiments, the composition comprises THBS2, THBS4, and IL-15. In certain embodiments, the composition comprises THBS2, THBS4, and IGF2 In certain embodiments, the composition comprises THBS1 and FGF17. In certain embodiments, the composition comprises THBS2 and VTN. In certain embodiments, the composition comprises THBS1 and VTN. In certain embodiments, the composition comprises THBS1 and THBS2. In certain embodiments, the composition comprises THBS2 and FGF17. In certain embodiments, the composition comprises THBS1 and THBS4. In certain embodiments, the composition comprises VTN and FGF17. In certain embodiments, the composition comprises THBS4 and VTN. In certain embodiments, the composition comprises THBS4 and FGF17. In certain embodiments, described herein, is a composition comprising any 1, 2, 3, 4, 5, 6, 7, 8, 9, 10 or more polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent. In certain embodiments, described herein, is a composition comprising 1, 2, 3, 4, 5, 6, 7, 8, 9, 10 or more polypeptides, wherein one or more the polypeptides are at least about 85%, 90%, 95%, 97%, 98%, 99%, or 100% identical to VTN, POSTN, FGF17, THBS2, THBS1, IGF2, IL-15, or THBS4, and a pharmaceutically acceptable excipient, carrier, or diluent.

In certain embodiments, described herein is a composition comprising a polypeptide comprising at least about 85%, 90%, 95%, 97%, 98%, 99%, or 100% homology or identity to SEQ ID NO: 1 or amino acids 20-478 of SEQ ID NO: 1, and a pharmaceutically acceptable excipient, carrier, or diluent. In some cases, the polypeptide does not comprise a signal sequence of SEQ ID NO 1. In certain embodiments, described herein is a composition comprising a polypeptide comprising at least about 85%, 90%, 95%, 97%, 98%, 99%, or 100% homology or identity to SEQ ID NO: 2, and a pharmaceutically acceptable excipient, carrier, or diluent. In some cases, the polypeptide does not comprise a signal sequence of SEQ ID NO: 2. In certain embodiments, described herein is a composition comprising a polypeptide comprising at least about 85%, 90%, 95%, 97%, 98%, 99%, or 100% homology or identity to SEQ ID NO: 3, and a pharmaceutically acceptable excipient, carrier, or diluent. In some cases, the polypeptide does not comprise a signal sequence of SEQ ID NO: 3. In certain embodiments, described herein is a composition comprising a polypeptide comprising at least about 85%, 90%, 95%, 97%, 98%, 99%, or 100% homology or identity to SEQ ID NO: 4 or amino acids 19-1172 of SEQ ID NO: 4, and a pharmaceutically acceptable excipient, carrier, or diluent. In some cases, the polypeptide does not comprise a signal sequence of SEQ ID NO: 4. In certain embodiments, described herein is a composition comprising a polypeptide comprising at least about 85%, 90%, 95%, 97%, 98%, 99%, or 100% homology or identity to SEQ ID NO: 5, and a pharmaceutically acceptable excipient, carrier, or diluent. In some cases, the polypeptide does not comprise a signal sequence of SEQ ID NO: 5. In certain embodiments, described herein is a composition comprising a polypeptide comprising at least about 85%, 90%, 95%, 97%, 98%, 99%, or 100% homology or identity to SEQ ID NO: 6 or amino acids 22-836 of SEQ ID NO: 6, and a pharmaceutically acceptable excipient, carrier, or diluent. In some cases, the polypeptide does not comprise a signal sequence of SEQ ID NO: 6. In certain embodiments, described herein is a composition comprising a polypeptide comprising at least about 85%, 90%, 95% 97%, 98%, 99%, or 100% homology or identity to SEQ ID NO: 7 or amino acids 23-216 of SEQ ID NO:7, and a pharmaceutically acceptable excipient, carrier, or diluent. In some cases, the polypeptide does not comprise a signal sequence of SEQ ID NO: 7. In certain embodiments, described herein is a composition comprising a polypeptide comprising at least about 85%, 90%, 95%, 97%, 98%, 99%, or 100% homology or identity to SEQ ID NO: 8 or amino acids 27-961 of SEQ ID NO: 8, and a pharmaceutically acceptable excipient, carrier, or diluent. In some cases, the polypeptide does not comprise a signal sequence of SEQ ID NO: 8. In certain embodiments, described herein is a composition comprising a polypeptide comprising at least about 85%, 90%, 95%, 97%, 98%, 99%, or 100% homology or identity to SEQ ID NO: 9 or amino acids 19-1170 of SEQ ID NO: 9, and a pharmaceutically acceptable excipient, carrier, or diluent. In some cases, the polypeptide does not comprise a signal sequence of SEQ ID NO: 9. In certain embodiments, described herein is a composition comprising a polypeptide comprising at least about 85%, 90%, 95%, 97%, 98%, 99%, or 100% homology or identity to SEQ ID NO: 10 or amino acids 49-162 of SEQ ID NO: 10, and a pharmaceutically acceptable excipient, carrier, or diluent. In some cases, the polypeptide does not comprise a signal sequence of SEQ ID NO: 10. In certain embodiments, described herein is a composition comprising a polypeptide comprising at least about 85%, 90%, 95%, 97%, 98%, 99%, or 100% homology or identity to SEQ ID NO: 11 or amino acids 25-91 of SEQ ID NO: 11, and a pharmaceutically acceptable excipient, carrier, or diluent. In some cases, the polypeptide does not comprise a signal sequence of SEQ ID NO: 11.

In certain embodiments, described herein is a composition comprising a polypeptide comprising at least about 85%, 90%, 95%, 97%, 98%, 99%, or 100% homology or identity to SEQ ID NO: 12, and a pharmaceutically acceptable excipient, carrier, or diluent. In some cases, the polypeptide does not comprise a signal sequence of SEQ ID NO: 12. In certain embodiments, described herein is a composition comprising a polypeptide comprising at least about 85%, 90%, 95%, 97%, 98%, 99%, or 100% homology or identity to SEQ ID NO: 13, and a pharmaceutically acceptable excipient, carrier, or diluent. In some cases, the polypeptide does not comprise a signal sequence of SEQ ID NO: 13. In certain embodiments, described herein is a composition comprising a polypeptide comprising at least about 85%, 90%, 95%, 97%, 98%, 99%, or 100% homology or identity to SEQ ID NO: 14, and a pharmaceutically acceptable excipient, carrier, or diluent. In some cases, the polypeptide does not comprise a signal sequence of SEQ ID NO: 14. In certain embodiments, described herein is a composition comprising a polypeptide comprising at least about 85%, 90%, 95%, 97%, 98%, 99%, or 100% homology or identity to SEQ ID NO: 15 and a pharmaceutically acceptable excipient, carrier, or diluent. In some cases, the polypeptide does not comprise a signal sequence of SEQ ID NO: 15 In certain embodiments, described herein is a composition comprising a polypeptide comprising at least about 85%, 90%, 95%, 97%, 98%, 99%, or 100% homology or identity to SEQ ID NO: 16, and a pharmaceutically acceptable excipient, carrier, or diluent. In some cases, the polypeptide does not comprise a signal sequence of SEQ ID NO: 16. In certain embodiments, described herein is a composition comprising a polypeptide comprising at least about 85%, 90%, 95%, 97%, 98%, 99%, or 100% homology or identity to SEQ ID NO: 17, and a pharmaceutically acceptable excipient, carrier, or diluent. In some cases, the polypeptide does not comprise a signal sequence of SEQ ID NO: 17. In certain embodiments, described herein is a composition comprising a polypeptide comprising at least about 85%, 90%, 95%, 97%, 98%, 99%, or 100% homology or identity to SEQ ID NO: 18, and a pharmaceutically acceptable excipient, carrier, or diluent. In some cases, the polypeptide does not comprise a signal sequence of SEQ ID NO: 18. In certain embodiments, described herein is a composition comprising a polypeptide comprising at least about 85%, 90%, 95%, 97%, 98%, 99%, or 100% homology or identity to SEQ ID NO: 19, and a pharmaceutically acceptable excipient, carrier, or diluent. In some cases, the polypeptide does not comprise a signal sequence of SEQ ID NO: 19.

In certain embodiments, described herein is a composition comprising a polypeptide comprising at least about 85%, 90%, 95%, 97%, 98%, 99%, or 100% homology or identity to SEQ ID NO: 20, and a pharmaceutically acceptable excipient, carrier, or diluent. In some cases, the polypeptide does not comprise a signal sequence of SEQ ID NO: 20. In certain embodiments, described herein is a composition comprising a polypeptide comprising at least about 85%, 90%, 95%, 97%, 98%, 99%, or 100% homology or identity to SEQ ID NO: 21, and a pharmaceutically acceptable excipient, carrier, or diluent. In some cases, the polypeptide does not comprise a signal sequence of SEQ ID NO: 21. In certain embodiments, described herein is a composition comprising a polypeptide comprising at least about 85%, 90%, 95%, 97%, 98%, 99%, or 100% homology or identity to SEQ ID NO: 22, and a pharmaceutically acceptable excipient, carrier, or diluent. In some cases, the polypeptide does not comprise a signal sequence of SEQ ID NO: 22. In certain embodiments, described herein is a composition comprising a polypeptide comprising at least about 85%, 90%, 95%, 97%, 98%, 99%, or 100% homology or identity to SEQ ID NO: 23, and a pharmaceutically acceptable excipient, carrier, or diluent. In some cases, the polypeptide does not comprise a signal sequence of SEQ ID NO: 23. In certain embodiments, described herein is a composition comprising a polypeptide comprising at least about 85%, 90%, 95%, 97%, 98%, 99%, or 100% homology or identity to SEQ ID NO: 24, and a pharmaceutically acceptable excipient, carrier, or diluent. In some cases, the polypeptide does not comprise a signal sequence of SEQ ID NO: 24. In certain embodiments, described herein is a composition comprising a polypeptide comprising at least about 85%, 90%, 95%, 97%, 98%, 99%, or 100% homology or identity to SEQ ID NO: 25, and a pharmaceutically acceptable excipient, carrier, or diluent. In some cases, the polypeptide does not comprise a signal sequence of SEQ ID NO: 25. In certain embodiments, described herein is a composition comprising a polypeptide comprising at least about 85%, 90%, 95%, 97%, 98%, 99%, or 100% homology or identity to SEQ ID NO: 26, and a pharmaceutically acceptable excipient, carrier, or diluent. In some cases, the polypeptide does not comprise a signal sequence of SEQ ID NO: 26. In certain embodiments, described herein is a composition comprising a polypeptide comprising at least about 85%, 90%, 95%, 97%, 98%, 99%, or 100% homology or identity to SEQ ID NO: 27, and a pharmaceutically acceptable excipient, carrier, or diluent. In some cases, the polypeptide does not comprise a signal sequence of SEQ ID NO: 27. In certain embodiments, described herein is a composition comprising a polypeptide comprising at least about 85%, 90%, 95%, 97%, 98%, 99%, or 100% homology or identity to SEQ ID NO: 28, and a pharmaceutically acceptable excipient, carrier, or diluent. In some cases, the polypeptide does not comprise a signal sequence of SEQ ID NO: 28. In certain embodiments, described herein is a composition comprising a polypeptide comprising at least about 85%, 90%00, 97%, 98%, 99%, or 100% homology or identity to SEQ ID NO: 29, and a pharmaceutically acceptable excipient, carrier, or diluent in some cases, the polypeptide does not comprise a signal sequence of SEQ ID NO: 29.

In certain embodiments, described herein is a composition comprising a polypeptide comprising at least about 85%, 90%, 95%, 97%, 98%, 99%, or 100% homology or identity to SEQ ID NO: 30, and a pharmaceutically acceptable excipient, carrier, or diluent. In some cases, the polypeptide does not comprise a signal sequence of SEQ ID NO: 30. In certain embodiments, described herein is a composition comprising a polypeptide comprising at least about 85%, 90%, 95%, 97%, 98%, 99%, or 100% homology or identity to SEQ ID NO: 31, and a pharmaceutically acceptable excipient, carrier, or diluent. In some cases, the polypeptide does not comprise a signal sequence of SEQ ID NO: 31. In certain embodiments, described herein is a composition comprising a polypeptide comprising at least about 85%, 90%, 95%, 97%, 98%, 99%, or 100% homology or identity to SEQ ID NO: 32, and a pharmaceutically acceptable excipient, carrier, or diluent. In some cases, the polypeptide does not comprise a signal sequence of SEQ ID NO: 32. In certain embodiments, described herein is a composition comprising a polypeptide comprising at least about 85%, 90%, 95%, 97%, 98%, 99%, or 100% homology or identity to SEQ ID NO: 33, and a pharmaceutically acceptable excipient, carrier, or diluent. In some cases, the polypeptide does not comprise a signal sequence of SEQ ID NO: 33. In certain embodiments, described herein is a composition comprising a polypeptide comprising at least about 85%, 90%, 95%, 97%, 98%, 99%, or 1000% homology or identity to SEQ ID NO: 34, and a pharmaceutically acceptable excipient, carrier, or diluent. In some cases, the polypeptide does not comprise a signal sequence of SEQ ID NO: 34. In certain embodiments, described herein is a composition comprising a polypeptide comprising at least about 85%, 90%, 95%, 97%, 98%, 99%, or 100% homology or identity to SEQ ID NO: 35, and a pharmaceutically acceptable excipient, carrier, or diluent. In some cases, the polypeptide does not comprise a signal sequence of SEQ ID NO: 35. In certain embodiments, described herein is a composition comprising a polypeptide comprising at least about 85%, 90%, 95%, 97%, 98%, 99%, or 100% homology or identity to SEQ ID NO: 36, and a pharmaceutically acceptable excipient, carrier, or diluent. In some cases, the polypeptide does not comprise a signal sequence of SEQ ID NO: 36. In certain embodiments, described herein is a composition comprising a polypeptide comprising at least about 85%, 90%, 95%, 97%, 98%, 99%, or 100% homology or identity to SEQ ID NO: 37, and a pharmaceutically acceptable excipient, carrier, or diluent. In some cases, the polypeptide does not comprise a signal sequence of SEQ ID NO: 37. In certain embodiments, described herein is a composition comprising a polypeptide comprising at least about 85%, 90%, 95%, 97%, 98%, 99%, or 100% homology or identity to SEQ ID NO: 38, and a pharmaceutically acceptable excipient, carrier, or diluent. In some cases, the polypeptide does not comprise a signal sequence of SEQ ID NO: 38. In certain embodiments, described herein is a composition comprising a polypeptide comprising at least about 85%, 90%, 95%, 97%, 98%, 99%, or 1000% homology or identity to SEQ ID NO: 39, and a pharmaceutically acceptable excipient, carrier, or diluent. In some cases, the polypeptide does not comprise a signal sequence of SEQ ID NO: 39.

In certain embodiments, described herein is a composition comprising a polypeptide comprising at least about 85%, 90%, 95%, 97%, 98%, 99%, or 100% homology or identity to SEQ ID NO: 40, and a pharmaceutically acceptable excipient, carrier, or diluent. In some cases, the polypeptide does not comprise a signal sequence of SEQ ID NO: 40. In certain embodiments, described herein is a composition comprising a polypeptide comprising at least about 85%, 90%, 95%, 97%, 98%, 99%, or 100% homology or identity to SEQ ID NO: 41, and a pharmaceutically acceptable excipient, carrier, or diluent. In some cases, the polypeptide does not comprise a signal sequence of SEQ ID NO: 41. In certain embodiments, described herein is a composition comprising a polypeptide comprising at least about 85%, 90%, 95%, 97%, 98%, 99%, or 100% homology or identity to SEQ ID NO: 42, and a pharmaceutically acceptable excipient, carrier, or diluent. In some cases, the polypeptide does not comprise a signal sequence of SEQ ID NO: 42. In certain embodiments, described herein is a composition comprising a polypeptide comprising at least about 85%, 90%, 95%, 97%, 98%, 99%, or 100% homology or identity to SEQ ID NO: 43, and a pharmaceutically acceptable excipient, carrier, or diluent. In some cases, the polypeptide does not comprise a signal sequence of SEQ ID NO: 43. In certain embodiments, described herein is a composition comprising a polypeptide comprising at least about 85%, 90%, 95%, 97%, 98%, 99%, or 100% homology or identity to SEQ ID NO: 44, and a pharmaceutically acceptable excipient, carrier, or diluent. In some cases, the polypeptide does not comprise a signal sequence of SEQ ID NO 44. In certain embodiments, described herein is a composition comprising a polypeptide comprising at least about 85%, 90%, 95%, 97%, 98%, 99%, or 100% homology or identity to SEQ ID NO: 58, and a pharmaceutically acceptable excipient, carrier, or diluent. In some cases, the polypeptide does not comprise a signal sequence of SEQ ID NO: 58. In certain embodiments, described herein is a composition comprising a polypeptide comprising at least about 85%, 90%, 95%, 97%, 98%, 99%, or 100% homology or identity to SEQ ID NO: 59, and a pharmaceutically acceptable excipient, carrier, or diluent. In some cases, the polypeptide does not comprise a signal sequence of SEQ ID NO: 59. In certain embodiments, described herein is a composition comprising a polypeptide comprising at least about 85%, 90%, 95%, 97%, 98%, 99%, or 100% homology or identity to SEQ ID NO: 60, and a pharmaceutically acceptable excipient, carrier, or diluent. In some cases, the polypeptide does not comprise a signal sequence of SEQ ID NO: 60. In certain embodiments, described herein is a composition comprising a polypeptide comprising at least about 85%, 90%, 95%, 97%, 98%, 99%, or 100% homology or identity to SEQ ID NO: 61, and a pharmaceutically acceptable excipient, carrier, or diluent. In some cases, the polypeptide does not comprise a signal sequence of SEQ ID NO: 61. In certain embodiments, described herein is a composition comprising a polypeptide comprising at least about 85%, 90%, 95%, 97%, 98%, 99%, or 1000% homology or identity to SEQ ID NO: 62, and a pharmaceutically acceptable excipient, carrier, or diluent. In some cases, the polypeptide does not comprise a signal sequence of SEQ ID NO: 62. In certain embodiments, described herein is a composition comprising a polypeptide comprising at least about 85%, 90%, 95%, 97%, 98%, 99%, or 100% homology or identity to SEQ ID NO: 63, and a pharmaceutically acceptable excipient, carrier, or diluent. In some cases, the polypeptide does not comprise a signal sequence of SEQ ID NO: 63. In certain embodiments, described herein is a composition comprising a polypeptide comprising at least about 85%, 90%, 95%, 97%, 98%, 99%, or 100% homology or identity to SEQ ID NO: 64, and a pharmaceutically acceptable excipient, carrier, or diluent. In some cases, the polypeptide does not comprise a signal sequence of SEQ ID NO: 64. In certain embodiments, described herein is a composition comprising a polypeptide comprising at least about 85%, 90%, 95%, 97%, 98%, 99%, or 100% homology or identity to SEQ ID NO: 65, and a pharmaceutically acceptable excipient, carrier, or diluent. In some cases, the polypeptide does not comprise a signal sequence of SEQ ID NO: 65. In certain embodiments, described herein is a composition comprising a polypeptide comprising at least about 85%, 90%, 95%, 97%, 98%, 99%, or 100% homology or identity to SEQ ID NO: 66, and a pharmaceutically acceptable excipient, carrier, or diluent. In some cases, the polypeptide does not comprise a signal sequence of SEQ ID NO: 66. In certain embodiments, described herein is a composition comprising a polypeptide comprising at least about 85%, 90%, 95%, 97%, 98%, 99%, or 100% homology or identity to SEQ ID NO: 67, and a pharmaceutically acceptable excipient, carrier, or diluent. In some cases, the polypeptide does not comprise a signal sequence of SEQ ID NO: 67. In certain embodiments, described herein is a composition comprising a polypeptide comprising at least about 85%, 90%, 95%, 97%, 98%, 99%, or 100% homology or identity to SEQ ID NO: 68, and a pharmaceutically acceptable excipient, carrier, or diluent. In some cases, the polypeptide does not comprise a signal sequence of SEQ ID NO: 68. In certain embodiments, described herein is a composition comprising a polypeptide comprising at least about 85%, 90%, 95%, 97% 98%, 99%, or 100% homology or identity to SEQ ID NO: 69, and a pharmaceutically acceptable excipient, carrier, or diluent. In some cases, the polypeptide does not comprise a signal sequence of SEQ ID NO: 69. In certain embodiments, described herein is a composition comprising a polypeptide comprising at least about 85%, 90%, 95%, 97%, 98%, 99%, or 100% homology or identity to SEQ ID NO: 70, and a pharmaceutically acceptable excipient, carrier, or diluent. In some cases, the polypeptide does not comprise a signal sequence of SEQ ID NO: 70.

In certain embodiments, described herein, is a composition comprising a plurality of polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COLSA3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, and combinations thereof; and a pharmaceutically acceptable excipient, carrier, or diluent. In certain embodiments, described herein, is a composition comprising a plurality of polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

In certain embodiments, described herein, is a composition consisting essentially of a plurality of polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, RPL29, and combinations thereof; and a pharmaceutically acceptable excipient, carrier, or diluent. In certain embodiments, described herein, is a composition consisting essentially of a plurality of polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

In certain embodiments, described herein, is a composition consisting essentially of any 1, 2, 3, 4, 5, 6, 7, 8, 9, 10 or more polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COLSA1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, and RPL29, and a pharmaceutically acceptable excipient, carrier, or diluent. In certain embodiments, described herein, is a composition consisting essentially of any 1, 2, 3, 4, 5, 6, 7, 8, 9, 10 or more polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

In certain embodiments, described herein, is a composition consisting of any 1, 2, 3, 4, 5, 6, 7, 8, 9, 10 or more polypeptides at least about 85%, 90%, 95%, 97%, 98%, 99%, or 100% identical to ADAMTS12, INS-IGF2, AOC1, SOD3, CLU, ITIH1, APLP1, THBS1, COCH, ITIH2, APLP2, THBS3, COL11A1, LAMA1, APOB, TNXB, COL12A1, LAMA2, APOE, VEGFA, COL14A1, LAMA5, APOH, VTN, COL18A1, LAMB1, APP, ZNF207, COL1A1, LAMB2, CCDC80, COL1A2, LTF, CFH, COL2A1, MATN2, CLEC3B, COL3A1, MDK, COL25A1, COL5A1, MST1, COL5A3, COL5A2, NID1, CYR61, COL6A1, NPNT, F2, COL6A2, OLFML3, FGF2, COL6A3, PCOLCE, FGFBP3, CTGF, POSTN, FSTL1, DCD, PTN, HDGF, DRAXIN, RARRES2, KNG1, ECM1, RELN, NDNF, FBLN1, SFRP1, NRP1, FBN1, SLIT3, PAFAH1B1, FBN2, SPON1, PCOLCE2, FN1, STC1, PTPRF, FST, STC2, PTPRS, HGFAC, SVEP1, RPL22, IGFBP2, THBS2, and RPL29; and a pharmaceutically acceptable excipient, carrier, or diluent. In certain embodiments, described herein, is a composition consisting of any 1, 2, 3, 4, 5, 6, 7, 8, 9, 10 or more polypeptides at least about 85%, 90%, 95%, 97%, 98%, 99%, or 100% identical to CTGF, THBS1, THBS2, THBS3, HGFAC, IGFBP3, IGFBP5, IGFBP7, IGFBP4, SFRP1, STC1, STC2, IGFBP2; and a pharmaceutically acceptable excipient, carrier, or diluent.

In certain embodiments, compositions comprising heparin-associated polypeptides do not comprise fibroblast growth factors (FGF). In certain embodiments, compositions comprising heparin-associated polypeptides do not comprise fibroblast growth factor 2 (FGF2). In certain embodiments, compositions comprising heparin-associated polypeptides do not comprise FGF19, Angiogenin, BTC, IL-13 R alpha 2, Siglec-5/CD170, IL-15, APJ, IGFBP-2, Chordin-Like 1, GASP-1/WFIKKNRP, MFRP, IL-10 R alpha, Chem R23, HB-EGF, FGF-6, HGF, IL-16, IL-7 R alpha, TRAIL R3/TNFRSF10C, BMP-6, IL-1 F9/IL-1 H1, IL-1 beta, Kremen-2, TRAIL R4/TNFRSF10D, CXCR1/IL-8 RA, Ck beta 8-1/CCL23, Beta-catenin, FGF-13 1B, TRAIL/TNFSF10, CCL14/HCC-1/HCC-3, or FGF-4, or a combination thereof.

In certain aspects, heparin-associated binding polypeptides and compositions of heparin-associated binding polypeptides herein comprise polypeptides that increase the proliferation of muscle cell precursors, and/or increase their differentiation into muscle cells. In certain embodiments, the heparin-associated polypeptides increase proliferation of a muscle cell precursor by at least about 20%, 30%, 40%, 50%, or 100% compared to a muscle cell precursor not treated with the heparin-associated binding polypeptide. In certain embodiments, the heparin-associated polypeptides increase proliferation of a myoblast by at least about 20%, 30%, 40%, 50%, 100%, 200%, or 500% compared to a myoblast not treated with the heparin-associated binding polypeptide. In certain embodiments, the myoblast is a human myoblast cell line. In certain embodiments, the myoblast is a mouse myoblast cell line (e.g., C2C12). Proliferation can be measured by BrdU or EdU incorporation, which can be quantified using suitable methods such as, by way of non-limiting embodiment, microscopy, flow cytometry, or ELISA.

in certain embodiments, the heparin-associated polypeptides increase differentiation and/or fusion of a muscle cell precursor by at least about 50%, 75%, 100%, 200%, or 500% compared to a muscle cell precursor not treated with the heparin-associated binding polypeptide. In certain embodiments, the heparin-associated polypeptides increase differentiation of a myoblast by at least about 50%, 75%, or 100% compared to a myoblast not treated with the heparin-associated binding polypeptide 1n certain embodiments, the myoblast is a human myoblast cell line. In certain embodiments, the myoblast is a mouse myoblast cell line (e.g., C2C12). Differentiation can be measured and/or quantified by eMyHC staining, which detects fusion of a myoblast or muscle cell precursor This staining can be quantified, for example, by microscopy or flow cytometry.

Heparin-associated polypeptides that increase muscle or connective tissue cell precursor proliferation and/or differentiation are useful in methods of treating muscle or connective tissue disorders. These disorders can arise from the normal aging process, injury related to trauma or physical exertion, genetic predispositions, or incident to other disease states.

Heparin-associated binding polypeptides that are useful for increasing muscle cell precursor differentiation or proliferation are described herein, and in certain embodiments comprise Vitronectin (VTN), Stanniocalcin-2 (STC2), Periostin (POSTN), Agrin (AGRN), Fibroblast growth factor (FGF17, also known as Fibroblast growth factor 13 or FGF13), Thrombospondin 2 (THBS2), follistatin (FST), Thrombospondin 4 (THBS4), Thrombospondin 1 (THBS1), Insulin-like growth factor 2 (IGF2), or Interleukin 15 (IL-15), or any combination thereof. In certain embodiments, any one, two, three, four, or five of VTN, STC2, AGRN, THBS2, or FST are present in a composition useful for increasing muscle cell precursor proliferation or muscle cell differentiation. In certain embodiments, any one, two, three, four, five, six, seven, or eight of VTN, POSTN, FGF17, THBS2, THBS1, IL-15, IGF2, and THBS4 are present in a composition useful for increasing muscle cell precursor proliferation or muscle cell differentiation.

In certain embodiments, a heparin-associated binding polypeptide composition comprises Vitronectin (VTN). VTN may be further included in the composition with any one, two, three, four, five, six, seven, eight, nine, or all polypeptides selected from STC2, AGRN, POSTN, FGF17, THBS2, FST, THBS1, IL-15, IGF2, and THBS4. In certain embodiments, the composition comprises VTN and STC2. In certain embodiments, the composition comprises VTN and AGRN In certain embodiments, the composition comprises VTN and THBS2. In certain embodiments, the composition comprises VTN and FST. In certain embodiments, the composition comprises VTN and POSTN. In certain embodiments, the composition comprises VTN and FGF17. In certain embodiments, the composition comprises VTN and THBS4. In certain embodiments, the composition comprises VTN and THBS1. In certain embodiments, the composition comprises VTN and IGF2. In certain embodiments, the composition comprises VTN and IL-15. Human VTN is disclosed in SEQ ID NO: 1. In certain embodiments, the VTN of the heparin-associated binding polypeptide composition comprises an amino acid sequence that is at least about 80%, 85%, 90%, 95%, 97%, 98%, 99%, or 100% identical to SEQ ID NO: 1 or amino acids 20-478 of SEQ ID NO: 1. In certain embodiments, the VTN polypeptide lacks a secretory leader sequence, e.g., amino acids 1-19 of SEQ ID NO: 1 in certain embodiments, the VTN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the VTN polypeptide comprises one or more additional modifications to increase stability. In certain embodiments, the VTN polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the VTN polypeptide is fused with an Fc region of an immunoglobulin or with serum albumin. In certain embodiments, the VTN polypeptide is present in a concatemer with one, two, three, four or more distinct polypeptides selected from STC2, AGRN, THBS2, THBS1, THBS4, FGF17, POSTN, IGF2, IL-15 and FST. In certain embodiments, the VTN polypeptide is present in a concatemer with one, two, three, four or more distinct polypeptides selected from POSTN, FGF17, THBS2, THBS1, IGF2, IL-15, and THBS4. In certain embodiments, the VTN polypeptide is present in a concatemer with one, two, three, four, or more distinct VTN polypeptides. In certain embodiments, the VTN polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the VTN polypeptide is prepared recombinantly in an expression system (e.g., bacteria, yeast, mammalian, insect). In some cases, the VTN polypeptide is prepared by chemical synthesis.

In certain embodiments, a heparin-associated binding polypeptide composition comprises Periostin (POSTN). POSTN may be further included in the composition with any one, two, three, four, five, six, or all polypeptides selected from VTN, FGF17, THBS2, THBS1, IGF2, IL-15, and THBS4. In certain embodiments, the composition comprises POSTN and VTN. In certain embodiments, the composition comprises POSTN and FGF17. In certain embodiments, the composition comprises POSTN and THBS2. In certain embodiments, the composition comprises POSTN and THBS4 in certain embodiments, the composition comprises POSTN and THBS1 In certain embodiments, the composition comprises POSTN and IGF2. In certain embodiments, the composition comprises POSTN and IL-15 Human POSTN is disclosed in SEQ ID NO: 6. In certain embodiments, the POSTN of the heparin-associated binding polypeptide composition comprises an amino acid sequence that is at least about 80%, 85%, 90%, 95%, 97%, 98%, 99%, or 100% identical to SEQ ID NO: 6 or amino acids 22-836 of SEQ ID NO: 6. In certain embodiments, the POSTN polypeptide lacks a secretory leader sequence, e.g., amino acids 1-21 of SEQ ID NO: 6. In certain embodiments, the POSTN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the POSTN polypeptide comprises one or more additional modifications to increase stability. In certain embodiments, the POSTN polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the POSTN polypeptide is fused with an Fc region of an immunoglobulin or with serum albumin. In certain embodiments, the POSTN polypeptide is present in a concatemer with one, two, three, four or more distinct polypeptides selected from VTN, FGF17, THBS2, THBS1, IGF2, IL-15 and THBS4. In certain embodiments, the POSTN polypeptide is present in a concatemer with one, two, three, four, or more distinct POSTN polypeptides. In certain embodiments, the POSTN polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the POSTN polypeptide is prepared recombinantly in an expression system (e.g., bacteria, yeast, mammalian, insect). In some cases, the POSTN polypeptide is prepared by chemical synthesis.

In certain embodiments, a heparin-associated binding polypeptide composition comprises Fibroblast growth factor (FGF17). FGF17 may be further included in the composition with any one, two, three, four, five, six or all polypeptides selected from VTN, POSTN, THBS2, THBS1, IL-15, IGF2, and THBS4 in certain embodiments, the composition comprises FGF17 and VTN. In certain embodiments, the composition comprises FGF17 and POSTN. In certain embodiments, the composition comprises FGF17 and THBS2. In certain embodiments, the composition comprises FGF17 and THBS4. In certain embodiments, the composition comprises FGF17 and THBS1. In certain embodiments, the composition comprises FGF17 and IGF2. In certain embodiments, the composition comprises FGF17 and IL-15. Human FGF17 is disclosed in SEQ ID NO: 7. In certain embodiments, the FGF17 of the heparin-associated binding polypeptide composition comprises an amino acid sequence that is at least about 80%, 85%, 90%, 95%, 97%, 98%, 99%, or 100% identical to SEQ ID NO: 7, or amino acids 23-216 of SEQ ID NO: 7. In certain embodiments, the FGF17 polypeptide lacks a secretory leader sequence, e.g., amino acids 1-22 of SEQ ID NO: 7. In certain embodiments, the FGF17 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. In certain embodiments, the FGF17 polypeptide comprises one or more additional modifications to increase stability. In certain embodiments, the FGF17 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the FGF17 polypeptide is fused with an Fc region of an immunoglobulin or with serum albumin. In certain embodiments, the FGF17 polypeptide is present in a concatemer with one, two, three, four or more distinct polypeptides selected from VTN, POSTN, THBS2, THBS1, IGF2, IL-15, and THBS4. In certain embodiments, the FGF17 polypeptide is present in a concatemer with one, two, three, four, or more distinct FGF17 polypeptides. In certain embodiments, the FGF17 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the FGF17 polypeptide is prepared recombinantly in an expression system (e.g., bacteria, yeast, mammalian, insect). In some cases, the FGF17 polypeptide is prepared by chemical synthesis.

In certain embodiments, a heparin-associated binding polypeptide composition comprises Stanniocalcin-2 (STC2) STC-2 may be further included in the composition with any one, two, three, four or more polypeptides selected from VTN, AGRN, THBS2, THBS1, THBS4, POSTN, FGF17, IGF2, IL-15, and FST. In certain embodiments, the composition comprises STC2 and VTN. In certain embodiments, the composition comprises STC2 and AGRN. In certain embodiments, the composition comprises STC2 and THBS2. In certain embodiments, the composition comprises STC2 and FST Human STC2 is disclosed in SEQ ID NO: 2. In certain embodiments, the STC2 of the heparin-associated binding polypeptide composition comprises an amino acid sequence that is at least about 80%, 85%, 90%, 95%, 97%, 98%, 99%, or 100% identical to SEQ ID NO: 2. In certain embodiments, the STC2 polypeptide lacks a secretory leader sequence. In certain embodiments, the STC2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the STC2 polypeptide comprises one or more additional modifications to increase stability. In certain embodiments, the STC2 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the STC2 polypeptide is fused with an Fc region of an immunoglobulin or with serum albumin. In certain embodiments, the STC2 polypeptide is present in a concatemer with one, two, three, four or more distinct polypeptides selected from VTN, AGRN, THBS2, THBS1, THBS4, FGF17, POSTN, IGF2, IL_15, and FST. In certain embodiments, the STC2 polypeptide is present in a concatemer with one, two, three, four, or more distinct STC2 polypeptides. In certain embodiments, the STC2 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, a heparin-associated binding polypeptide composition comprises Agrin (AGRN). AGRN may be further included in the composition with any one, two, three, four or more polypeptides selected from VTN, STC2, THBS2, THBS1, THBS4, FGF17, POSTN, IGF2, IL-15, and FST. In certain embodiments, the composition comprises AGRN and VTN. In certain embodiments, the composition comprises AGRN and STC2. In certain embodiments, the composition comprises AGRN and THBS2. In certain embodiments, the composition comprises AGRN and FST. Human AGRN is disclosed in SEQ ID NO: 3. In certain embodiments, the AGRN of the heparin-associated binding polypeptide composition comprises an amino acid sequence that is at least about 80%, 85%, 90%, 95%, 97%, 98%, 99%, or 100% identical to SEQ ID NO: 3. In certain embodiments, the AGRN polypeptide lacks a secretory leader sequence. In certain embodiments, the AGRN polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. In certain embodiments, the AGRN polypeptide comprises one or more additional modifications to increase stability. In certain embodiments, the AGRN polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the AGRN polypeptide is fused with an Fc region of an immunoglobulin or with serum albumin. In certain embodiments, the AGRN polypeptide is present in a concatemer with one, two, three, or four other distinct polypeptides selected from VTN, STC2, THBS2, THBS1, THBS4, FGF17, POSTN, IGF2, IL-15, and FST. In certain embodiments, the AGRN polypeptide is present in a concatemer with one, two, three, four, or more distinct AGRN polypeptides. In certain embodiments, the AGRN polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, a heparin-associated binding polypeptide composition comprises Thrombospondin 2 (THBS2) THBS2 may be further included in the composition with any one, two, three, four, five, six or all polypeptides selected from VTN, STC2, AGRN, THBS1, IL-15, IGF2, and FST. In certain embodiments, the composition comprises THBS2 and VTN. In certain embodiments, the composition comprises THBS2 and STC2. In certain embodiments, the composition comprises THBS2 and AGRN. In certain embodiments, the composition comprises THBS2 and FST. In certain embodiments, the composition comprises AGRN and FST. In certain embodiments, the composition comprises THBS2 and THBS1. In certain embodiments, the composition comprises THBS2 and IGF2. In certain embodiments, the composition comprises THBS2 and IL-15. THBS2 may be further included in the composition with any one, two, three, four, five, six, or all polypeptides selected from VTN, POSTN, FGF17, THBS1, IL-15, IGF2, and THBS4. In certain embodiments, the composition comprises THBS2 and VTN. In certain embodiments, the composition comprises THBS2 and POSTN. In certain embodiments, the composition comprises THBS2 and FGF17. In certain embodiments, the composition comprises THBS2 and THBS4. In certain embodiments, the composition comprises FGF17 and THBS4. Human THBS2 is disclosed in SEQ ID NO: 4 In certain embodiments, the THBS2 of the heparin-associated binding polypeptide composition comprises an amino acid sequence that is at least about 80%, 85%, 90%, 95%, 97%, 98%, 99%, or 100% identical to SEQ ID NO: 4 or amino acids 19-1,172 of SEQ ID NO: 4. In certain embodiments, the THBS2 polypeptide lacks a secretory leader sequence, e.g., amino acids 1-18 of SEQ ID NO: 4 In certain embodiments, the THBS2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, or 300 amino acids from the N-terminal or C-terminal end of the polypeptide, including increments therein. In certain embodiments, the THBS2 polypeptide comprises one or more additional modifications to increase stability. In certain embodiments, the THBS2 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the THBS2 polypeptide is fused with an Fc region of an immunoglobulin or with serum albumin. In certain embodiments, the THBS2 polypeptide is present in a concatemer with one, two, three, four or more distinct polypeptides selected from VTN, STC2, AGRN, THBS1, THBS4, FGF17, POSTN, IGF2, IL-15, and FST. In certain embodiments, the THBS2 polypeptide is present in a concatemer with one, two, three, four or more distinct polypeptides selected from VTN, POSTN, THBS1, IGF2, IL-15, FGF17, and THBS4. In certain embodiments, the THBS2 polypeptide is present in a concatemer with one, two, three, four, or more distinct THBS2 polypeptides. In certain embodiments, the THBS2 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the THBS2 polypeptide is prepared recombinantly in an expression system (e.g., bacteria, yeast, mammalian, insect). In some cases, the THBS2 polypeptide is prepared by chemical synthesis.

In certain embodiments, a heparin-associated binding polypeptide composition comprises Thrombospondin 4 (THBS4). THBS4 may be further included in the composition with any one, two, three, four, five, six or all polypeptides selected from VTN, POSTN, FGF17, THBS1, IL-15, IGF2, and THBS2. In certain embodiments, the composition comprises THBS4 and VTN. In certain embodiments, the composition comprises THBS4 and POSTN. In certain embodiments, the composition comprises THBS4 and FGF17. In certain embodiments, the composition comprises THBS4 and THBS2. In certain embodiments, the composition comprises THBS4 and THBS1 In certain embodiments, the composition comprises THBS4 and IL-15. In certain embodiments, the composition comprises THBS4 and IGF2. Human THBS4 is disclosed in SEQ ID NO: 8. In certain embodiments, the THBS4 of the heparin-associated binding polypeptide composition comprises an amino acid sequence that is at least about 80%, 85%, 90%, 95%, 97%, 98%, 99%, or 100% identical to SEQ ID NO: 8 or amino acids 27-961 of SEQ ID NO: 8. In certain embodiments, the THBS4 polypeptide lacks a secretory leader sequence, e.g., amino acids 1-26 of SEQ ID NO: 8. In certain embodiments, the THBS4 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the THBS4 polypeptide comprises one or more additional modifications to increase stability. In certain embodiments, the THBS4 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the THBS4 polypeptide is fused with an Fc region of an immunoglobulin or with serum albumin. In certain embodiments, the THBS4 polypeptide is present in a concatemer with one, two, three, four or more distinct polypeptides selected from VTN, POSTN, FGF17, THBS1, IGF2, IL-15, and THBS2. In certain embodiments, the THBS4 polypeptide is present in a concatemer with one, two, three, four, or more distinct THBS4 polypeptides. In certain embodiments, the THBS4 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the THBS4 polypeptide is prepared recombinantly in an expression system (e.g., bacteria, yeast, mammalian, insect). In some cases, the THBS2 polypeptide is prepared by chemical synthesis.

In certain embodiments, a heparin-associated binding polypeptide composition comprises follistatin (FST). FST may be further included in the composition with any one, two, three, four or more polypeptides selected from VTN, STC2, AGRN, THBS1, THBS4, FGF17, POSTN, IGF2, IL-15, and THBS2. In certain embodiments, the composition comprises FST and VTN. In certain embodiments, the composition comprises FST and STC2. In certain embodiments, the composition comprises FST and AGRN. In certain embodiments, the composition comprises FST and THBS2. Human FST is disclosed in SEQ ID NO: 5. In certain embodiments, the FST of the heparin-associated binding polypeptide composition comprises an amino acid sequence that is at least about 80%, 85%, 90%, 95%, 97%, 98%, 99%, or 100% identical to SEQ ID NO: 5 In certain embodiments, the FST polypeptide lacks a secretory leader sequence. In certain embodiments, the FST polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the FST polypeptide comprises one or more additional modifications to increase stability. In certain embodiments, the FST polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the FST polypeptide is fused with an Fc region of an immunoglobulin or with serum albumin. In certain embodiments, the FST polypeptide is present in a concatemer with one, two, three, four or more distinct polypeptides selected from VTN, STC2, AGRN, THBS1, THBS4, FGF17, POSTN, IGF2, IL-15, and THBS2. In certain embodiments, the FST polypeptide is present in a concatemer with one, two, three, four, or more distinct FST polypeptides. In certain embodiments, the FST polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, a heparin-associated binding polypeptide composition comprises Thrombospondin 1 (THBS1) THSB1 may be further included in the composition with any one, two, three, four, five, six, seven, eight or all polypeptides selected from VTN, STC2, AGRN, THBS4, FGF17, POSTN, IGF2, IL-15, and THBS2 In certain embodiments, the composition comprises THSB1 and VTN. In certain embodiments, the composition comprises THSB1 and STC2. In certain embodiments, the composition comprises THSB1 and AGRN. In certain embodiments, the composition comprises THSB1 and THBS2. In certain embodiments, the composition comprises THSB1 and THBS4. In certain embodiments, the composition comprises THSB1 and FGF17 In certain embodiments, the composition comprises THSB1 and POSTN. In certain embodiments, the composition comprises THSB1 and IGF2. In certain embodiments, the composition comprises THSB1 and IL-15. Human THSB1 is disclosed in SEQ ID NO: 9. In certain embodiments, the THSB1 of the heparin-associated binding polypeptide composition comprises an amino acid sequence that is at least about 80%, 85%, 90%, 95%, 97%, 98%, 99%, or 100% identical to SEQ ID NO: 9 or amino acids 19-1170 of SEQ ID NO: 9. In certain embodiments, the THSB1 polypeptide lacks a secretory leader sequence. In certain embodiments, the THSB1 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the THSB1 polypeptide comprises one or more additional modifications to increase stability. In certain embodiments, the THSB1 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the THSB1 polypeptide is fused with an Fc region of an immunoglobulin or with serum albumin. In certain embodiments, the THSB1 polypeptide is present in a concatemer with one, two, three, four or more distinct polypeptides selected from VTN, STC2, AGRN, THBS4, FGF17, POSTN, IGF2, IL-15 and THBS2. In certain embodiments, the THSB1 polypeptide is present in a concatemer with one, two, three, four, or more distinct THSB1 polypeptides. In certain embodiments, the THSB1 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the THBS1 polypeptide is prepared recombinantly in an expression system (e.g., bacteria, yeast, mammalian, insect). In some cases, the TBS1 polypeptide is prepared by chemical synthesis.

In certain embodiments, a heparin-associated binding polypeptide composition comprises Interleukin-15 (IL-15). IL-15 may be further included in the composition with any one, two, three, four or more polypeptides selected from VTN, STC2, AGRN, THBS1, THBS4, IGF2, POSTN, FGF17, and THBS2. In certain embodiments, the composition comprises IL-15 and VTN. In certain embodiments, the composition comprises IL-15 and STC2. In certain embodiments, the composition comprises IL-15 and AGRN. In certain embodiments, the composition comprises IL-15 and THBS2. In certain embodiments, the composition comprises IL-15 and THBS1 In certain embodiments, the composition comprises IL-15 and THBS4. In certain embodiments, the composition comprises IL-15 and IGF2. In certain embodiments, the composition comprises IL-15 and POSTN. In certain embodiments, the composition comprises IL-15 and FGF17. In certain embodiments, the composition comprises IL-15 and THBS1. Human IL-15 is disclosed in SEQ ID NO: 10. In certain embodiments, the IL-15 of the heparin-associated binding polypeptide composition comprises an amino acid sequence that is at least about 80%, 85%, 90%, 95%, 97%, 98%, 99%, or 100% identical to SEQ ID NO: 10 or amino acids 49-162 of SEQ ID NO: 10 In certain embodiments, the IL-15 polypeptide lacks a secretory leader sequence. In certain embodiments, the IL-15 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the IL-15 polypeptide comprises one or more additional modifications to increase stability. In certain embodiments, the IL-15 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the IL-15 polypeptide is fused with an Fc region of an immunoglobulin or with serum albumin. In certain embodiments, the IL-15 polypeptide is present in a concatemer with one, two, three, four or more distinct polypeptides selected from VTN, STC2, AGRN, THBS1, THBS4, FGF17, POSTN, IGF2, and THBS2 In certain embodiments, the IL-15 polypeptide is present in a concatemer with one, two, three, four, or more distinct IL-15 polypeptides. In certain embodiments, the IL-15 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(DL-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the IL-15 polypeptide is prepared recombinantly in an expression system (e.g., bacteria, yeast, mammalian, insect). In some cases, the THBS1 polypeptide is prepared by chemical synthesis.

In certain embodiments, a heparin-associated binding polypeptide composition comprises Insulin-like growth factor 2 (IGF2). IGF2 may be further included in the composition with any one, two, three, four or more polypeptides selected from VTN, STC2, AGRN, THBS1, THBS4, POSTN, FGF17, IL-15 and THBS2. In certain embodiments, the composition comprises IGF2 and VTN. In certain embodiments, the composition comprises IGF2 and STC2. In certain embodiments, the composition comprises IGF2 and AGRN. In certain embodiments, the composition comprises IGF2 and THBS2. In certain embodiments, the composition comprises IGF2 and THBS1 In certain embodiments, the composition comprises IGF2 and THBS4. In certain embodiments, the composition comprises IGF2 and IL-15. In certain embodiments, the composition comprises IGF2 and POSTN. In certain embodiments, the composition comprises IGF2 and FGF17. Human IGF2 is disclosed in SEQ ID NO: 11. In certain embodiments, the IGF2 of the heparin-associated binding polypeptide composition comprises an amino acid sequence that is at least about 80%, 85%, 90%, 95%, 97%, 98%, 99%, or 100% identical to SEQ ID NO: 10 or amino acids 25-91 of SEQ ID NO: 11. In certain embodiments, the IGF2 polypeptide lacks a secretory leader sequence. In certain embodiments, the IGF2 polypeptide is modified by a deletion of 1, 2, 3, 4, 5, 6, 7, 8, 9, 10, 15, 20, 25, 30, 35, 40, 45, 50, 55, 60, 65, 70, 75, 80, 85, 90, 95, or 100 amino acids from the N-terminal or C-terminal end of the polypeptide, including increments therein. In certain embodiments, the IGF2 polypeptide comprises one or more additional modifications to increase stability. In certain embodiments, the IGF2 polypeptide is covalently conjugated to polyethylene glycol, polypropylene glycol, polyoxyalkylenes, or copolymers of polyethylene glycol, polysialic acid, glycolic acid, or polypropylene glycol. In certain embodiments, the heparin-associated polypeptide is fused or conjugated to another protein to increase stability and or bioavailability. In certain embodiments, the IGF2 polypeptide is fused with an Fc region of an immunoglobulin or with serum albumin. In certain embodiments, the IGF2 polypeptide is present in a concatemer with one, two, three, four or more distinct polypeptides selected from VTN, STC2, AGRN, THBS1, THBS4, FGF17, POSTN, IL-15, and THBS2 In certain embodiments, the IGF2 polypeptide is present in a concatemer with one, two, three, four, or more distinct IGF2 polypeptides. In certain embodiments, the IGF2 polypeptide is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA). In some cases, the IGF2 polypeptide is prepared recombinantly in an expression system (e.g., bacteria, yeast, mammalian, insect). In some cases, the THBS1 polypeptide is prepared by chemical synthesis.

In certain embodiments, the heparin-associated binding polypeptide composition comprises any two polypeptides selected from VTN, POSTN, FGF17, THBS2, THBS1, IGF2, IL-15, and THBS4 In certain embodiments, the composition comprises THBS1 In certain embodiments, the composition comprises THBS2. In certain embodiments, the composition comprises THBS4. In certain embodiments, the composition comprises FGF17. In certain embodiments, the composition comprises VTN. In certain embodiments, the composition comprises POSTN. In certain embodiments, the composition comprises IGF2. In certain embodiments, the composition comprises IL-15. In certain embodiments, the composition comprises VTN and POSTN. In certain embodiments, the composition comprises VTN and FGF17. In certain embodiments, the composition comprises VTN and THBS2 In certain embodiments, the composition comprises VTN and THBS1. In certain embodiments, the composition comprises VTN and IGF2. In certain embodiments, the composition comprises VTN and IL-15. In certain embodiments, the composition comprises VTN and THBS4. In certain embodiments, the composition comprises POSTN and FGF17. In certain embodiments, the composition comprises POSTN and THBS2. In certain embodiments, the composition comprises POSTN and THBS1. In certain embodiments, the composition comprises POSTN and IGF2. In certain embodiments, the composition comprises POSTN and IL-15. In certain embodiments, the composition comprises POSTN and THBS4. In certain embodiments, the composition comprises FGF17 and THBS2. In certain embodiments, the composition comprises FGF17 and THBS1. In certain embodiments, the composition comprises FGF17 and IGF2. In certain embodiments, the composition comprises FGF17 and IL-15. In certain embodiments, the composition comprises FGF17 and THBS4. In certain embodiments, the composition comprises THBS2 and THBS1. In certain embodiments, the composition comprises THBS2 and IGF2. In certain embodiments, the composition comprises THBS2 and IL-15. In certain embodiments, the composition comprises THBS2 and THBS4. In certain embodiments, the composition comprises THBS1 and IGF2. In certain embodiments, the composition comprises THBS1 and IL-15. In certain embodiments, the composition comprises THBS11 and THBS4. In certain embodiments, the composition comprises IGF2 and IL-15. In certain embodiments, the composition comprises IGF2 and THBS4. In certain embodiments, the composition comprises IL-15 and THBS4.

In certain embodiments, the heparin-associated binding polypeptide composition comprising any two polypeptides selected from VTN, POSTN, FGF17, THBS2, THBS1, IGF2, IL-15, and THBS4, comprises one or more additional modifications to increase stability. In certain embodiments, one or more of the heparin-associated polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the heparin-associated polypeptides is fused or conjugated to another protein to increase stability and or bioavailability. In certain embodiments, one or more of the heparin-associated polypeptides is fused with an Fc region of an immunoglobulin or with serum albumin. In certain embodiments, one or more of the heparin-associated polypeptides is present in a concatemer with one, two, three, or four other distinct polypeptides selected from THBS2, VTN, POSTN, FGF17, THBS1, IGF2, IL-15, and THBS4. In certain embodiments, one or more of the heparin-associated polypeptides is present in a concatemer with one, two, three, four, or more distinct polypeptides. In certain embodiments, one or more of the heparin-associated polypeptides is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA) in certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, the heparin-associated binding polypeptide composition comprises any three polypeptides selected from VTN, POSTN, FGF17, THBS2, THBS1, IGF2, IL-15, and THBS4. In certain embodiments, the composition comprises THBS1. In certain embodiments, the composition comprises THBS2. In certain embodiments, the composition comprises THBS4 In certain embodiments, the composition comprises FGF17. In certain embodiments, the composition comprises VTN. In certain embodiments, the composition comprises POSTN. In certain embodiments, the composition comprises IGF2. In certain embodiments, the composition comprises IL-15. In certain embodiments, the composition comprises IGF2, THBS2, and THBS4. In certain embodiments, the composition comprises IL-15, THBS2, and THBS4 in certain embodiments, the composition comprises THBS2 and THBS4. In certain embodiments, the composition comprises THBS2. THBS4, and VTN. In certain embodiments, the composition comprises THBS2, THBS4, and ANOS1. In certain embodiments, the composition comprises THBS2, THBS4, and IL-15. In certain embodiments, the composition comprises THBS2, THBS4, and IGF2. In certain embodiments, the composition comprises THBS1 and FGF17. In certain embodiments, the composition comprises THBS2 and VTN. In certain embodiments, the composition comprises THBS1 and VTN in certain embodiments, the composition comprises THBS1 and THBS2. In certain embodiments, the composition comprises THBS2 and FGF17. In certain embodiments, the composition comprises THBS1 and THBS4. In certain embodiments, the composition comprises VTN and FGF17 In certain embodiments, the composition comprises THBS4 and VTN. In certain embodiments, the composition comprises THBS4 and FGF17.

In certain embodiments, the composition comprises VTN, POSTN, and FGF17. In certain embodiments, the composition comprises VTN, POSTN, and THBS2. In certain embodiments, the composition comprises VTN, POSTN, and FGF17. In certain embodiments, the composition comprises VTN, POSTN, THBS2. In certain embodiments, the composition comprises VTN, POSTN, and THBS1 In certain embodiments, the composition comprises VTN, POSTN, IGF2 In certain embodiments, the composition comprises VTN, POSTN, and IL-15. In certain embodiments, the composition comprises VTN, POSTN, and THBS4.

In certain embodiments, the composition comprises VTN, FGF17, and THBS2. In certain embodiments, the composition comprises VTN, FGF17, and POSTN. In certain embodiments, the composition comprises VTN, FGF17, and THBS2. In certain embodiments, the composition comprises VTN, FGF17, and THBS1. In certain embodiments, the composition comprises VTN, FGF17, and IGF2. In certain embodiments, the composition comprises VTN, FGF17, and IL-15 In certain embodiments, the composition comprises VTN, FGF17, and THBS4.

In certain embodiments, the composition comprises VTN, THBS2, and POSTN. In certain embodiments, the composition comprises VTN, THBS2, and FGF17 In certain embodiments, the composition comprises VTN, THBS2, and THBS1. In certain embodiments, the composition comprises VTN, THBS2, and IGF2. In certain embodiments, the composition comprises VTN, THBS2, and IL-15. In certain embodiments, the composition comprises VTN, THBS2, and THBS4.

In certain embodiments, the composition comprises VTN, THBS1, and POSTN. In certain embodiments, the composition comprises VTN, THBS1, and FGF17. In certain embodiments, the composition comprises VTN, THBS1, and THBS2. In certain embodiments, the composition comprises VTN, THBS1, and IGF2. In certain embodiments, the composition comprises VTN, THBS1, and IL-15. In certain embodiments, the composition comprises VTN, THBS1, and THBS4.

In certain embodiments, the composition comprises VTN, IGF2, and POSTN. In certain embodiments, the composition comprises VTN, IGF2, and FGF17. In certain embodiments, the composition comprises VTN, IGF2, and THBS2. In certain embodiments, the composition comprises VTN, IGF2, and THBS1. In certain embodiments, the composition comprises VTN, IGF2, and IL-15 In certain embodiments, the composition comprises VTN, IGF2, and THBS4.

In certain embodiments, the composition comprises VTN, IL-15, and POSTN. In certain embodiments, the composition comprises VTN, IL-15, and FGF17. In certain embodiments, the composition comprises VTN, IL-15, and THBS2. In certain embodiments, the composition comprises VTN, IL-15, and THBS1. In certain embodiments, the composition comprises VTN, IL-15, and IGF2 In certain embodiments, the composition comprises VTN, IL-15, and THBS4.

In certain embodiments, the composition comprises VTN, TBHS4, and POSTN. In certain embodiments, the composition comprises VTN, TBHS4, and FGF17 In certain embodiments, the composition comprises VTN, TBHS4, and THBS2. In certain embodiments, the composition comprises VTN, TBHS4, and THBS1. In certain embodiments, the composition comprises VTN, TBHS4, and IGF2. In certain embodiments, the composition comprises VTN, TBHS4, and IL-15.

In certain embodiments, the composition comprises POSTN, FGF17, and VTN. In certain embodiments, the composition comprises POSTN, FGF17, and TBHS2. In certain embodiments, the composition comprises POSTN, FGF17, and THBS1. In certain embodiments, the composition comprises POSTN, FGF17, and IGF2. In certain embodiments, the composition comprises POSTN, FGF17, and IL-15 In certain embodiments, the composition comprises POSTN, FGF17, and THBS4.

In certain embodiments, the composition comprises POSTN, THBS2, and VTN. In certain embodiments, the composition comprises POSTN, THBS2, and FGF17. In certain embodiments, the composition comprises POSTN, THBS2, and THBS1. In certain embodiments, the composition comprises POSTN, THBS2, and IGF2. In certain embodiments, the composition comprises POSTN, THBS2, and IL-15. In certain embodiments, the composition comprises POSTN, THBS2, and THBS4.

In certain embodiments, the composition comprises POSTN, THBS1, and VTN. In certain embodiments, the composition comprises POSTN, THBS1, and FGF17. In certain embodiments, the composition comprises POSTN, THBS1, and THBS2. In certain embodiments, the composition comprises POSTN, THBS1, and IGF2. In certain embodiments, the composition comprises POSTN, THBS1, and IL-15. In certain embodiments, the composition comprises POSTN, THBS1, and THBS4.

In certain embodiments, the composition comprises POSTN, IGF2, and VTN. In certain embodiments, the composition comprises POSTN, IGF2, and FGF17. In certain embodiments, the composition comprises POSTN, IGF2, and THBS2. In certain embodiments, the composition comprises POSTN, IGF2, and THBS1. In certain embodiments, the composition comprises POSTN, IGF2, and IL-15. In certain embodiments, the composition comprises POSTN, IGF2, and THBS4.

In certain embodiments, the composition comprises POSTN, IL-15, and VTN. In certain embodiments, the composition comprises POSTN, IL-15, and FGF17. In certain embodiments, the composition comprises POSTN, IL-15, and THBS2. In certain embodiments, the composition comprises POSTN, IL-15, and THBS1. In certain embodiments, the composition comprises POSTN. IL-15, and IGF2. In certain embodiments, the composition comprises POSTN, IL-15, and THBS4.

In certain embodiments, the composition comprises POSTN, THBS4, and VTN. In certain embodiments, the composition comprises POSTN, THBS4, and FGF17. In certain embodiments, the composition comprises POSTN, THBS4, and THBS2. In certain embodiments, the composition comprises POSTN, THBS4, and THBS1. In certain embodiments, the composition comprises POSTN, THBS4, and IGF2. In certain embodiments, the composition comprises POSTN, THBS4, and IL-15.

In certain embodiments, the composition comprises FGF17, THBS2, and VTN. In certain embodiments, the composition comprises FGF17, THBS2, and POSTN. In certain embodiments, the composition comprises FGF17, THBS2, and THBS1. In certain embodiments, the composition comprises FGF17, THBS2, and IGF2. In certain embodiments, the composition comprises FGF17, THBS2, and IL-15. In certain embodiments, the composition comprises FGF17, THBS2, and THBS4.

In certain embodiments, the composition comprises FGF17, THBS1, and VTN. In certain embodiments, the composition comprises FGF17, THBS1, and POSTN. In certain embodiments, the composition comprises FGF17, THBS1, and THBS2. In certain embodiments, the composition comprises FGF17, THBS1, and IGF2. In certain embodiments, the composition comprises FGF17, THBS1, and IL-15. In certain embodiments, the composition comprises FGF17, THBS1, and THBS4.

In certain embodiments, the composition comprises FGF17, IGF2, and VTN. In certain embodiments, the composition comprises FGF17, IGF2, and POSTN. In certain embodiments, the composition comprises FGF17, IGF2, and THBS2. In certain embodiments, the composition comprises FGF17, IGF2, and THBS1. In certain embodiments, the composition comprises FGF17, IGF2, and IL-15. In certain embodiments, the composition comprises FGF17, IGF2, and THBS4.

In certain embodiments, the composition comprises FGF17, IL-15, and VIN. In certain embodiments, the composition comprises FGF7, IL-15, and POSTN. In certain embodiments, the composition comprises FGF17, IL-15, and THBS2. In certain embodiments, the composition comprises FGF17, IL-15, and THBS1. In certain embodiments, the composition comprises FGF17, IL-5, and IGF2. In certain embodiments, the composition comprises FGF17, IL-15, and THBS4.

In certain embodiments, the composition comprises FGF17, THBS4, and VTN. In certain embodiments, the composition comprises FGF17, THBS4, and POSTN. In certain embodiments, the composition comprises FGF17, THBS4, and THBS2. In certain embodiments, the composition comprises FGF17, THBS4, and THBS1 In certain embodiments, the composition comprises FGF17, THBS4, and IGF2. In certain embodiments, the composition comprises FGF17, THBS4, and IL-15.

In certain embodiments, the composition comprises THBS2. THBS1, and VTN. In certain embodiments, the composition comprises THBS2, THBS1, and POSTN. In certain embodiments, the composition comprises THBS2. THBS1, and FGF17. In certain embodiments, the composition comprises THBS2, THBS1, and IGF2 In certain embodiments, the composition comprises THBS2, THBS1, and IL-15. In certain embodiments, the composition comprises THBS2, THBS1, and THBS4.

In certain embodiments, the composition comprises THBS2, IGF2, and VTN. In certain embodiments, the composition comprises THBS2. IGF2, and POSTN. In certain embodiments, the composition comprises THBS2, IGF2, and FGF17. In certain embodiments, the composition comprises THBS2. IGF2, and THBS1 In certain embodiments, the composition comprises THBS2, IGF2, and IL-15 In certain embodiments, the composition comprises THBS2, IGF2, and THBS4.

In certain embodiments, the composition comprises THBS2, IL-15, and VTN. In certain embodiments, the composition comprises THBS2, IL-15, and POSTN. In certain embodiments, the composition comprises THBS2, IL-15, and FGF17. In certain embodiments, the composition comprises THBS2, IL-15, and THBS1 In certain embodiments, the composition comprises THBS2, IL-15, and IGF2. In certain embodiments, the composition comprises THBS2, IL-15, and THBS4.

In certain embodiments, the composition comprises THBS2, THBS4, and VTN. In certain embodiments, the composition comprises THBS2, THBS4, and POSTN. In certain embodiments, the composition comprises THBS2, THBS4, and FGF17. In certain embodiments, the composition comprises THBS2, THBS4, and THBS1. In certain embodiments, the composition comprises THBS2, THBS4, and IGF2. In certain embodiments, the composition comprises THBS2, THBS4, and IL-15.

In certain embodiments, the composition comprises THBS1, IGF2, and VTN. In certain embodiments, the composition comprises THBS1, IGF2, and POSTN. In certain embodiments, the composition comprises THBS1, IGF2, and FGF17. In certain embodiments, the composition comprises THBS1, IGF2, and THBS2. In certain embodiments, the composition comprises THBS1, IGF2, and IL-15. In certain embodiments, the composition comprises THBS1, IGF2, and THBS4.

In certain embodiments, the composition comprises THBS1, IL-15, and VTN. In certain embodiments, the composition comprises THBS1, IL-15, and POSTN. In certain embodiments, the composition comprises THBS1. IL-15, and FGF17. In certain embodiments, the composition comprises THBS1, IL-15, and THBS2. In certain embodiments, the composition comprises THBS1, IL-15, and IGF2. In certain embodiments, the composition comprises THBS1, IL-15, and THBS4.

In certain embodiments, the composition comprises THBS1, and THBS4, and VTN. In certain embodiments, the composition comprises THBS1, and THBS4, and POSTN. In certain embodiments, the composition comprises THBS1, and THBS4, and FGF17. In certain embodiments, the composition comprises THBS1, and THBS4, and THBS2. In certain embodiments, the composition comprises THBS1, and THBS4, and IGF2. In certain embodiments, the composition comprises THBS1, and THBS4, and IL-15.

In certain embodiments, the composition comprises IGF2, IL-15, and VTN. In certain embodiments, the composition comprises IGF2. IL-15, and POSTN. In certain embodiments, the composition comprises IGF2, IL-15, and FGF17. In certain embodiments, the composition comprises IGF2, IL-15, and THBS2. In certain embodiments, the composition comprises IGF2, IL-15, and THBS1 In certain embodiments, the composition comprises IGF2, IL-15, and THBS4.

In certain embodiments, the composition comprises IGF2, THBS4, and VTN. In certain embodiments, the composition comprises IGF2, THBS4, and POSTN. In certain embodiments, the composition comprises IGF2, THBS4, and FGF17. In certain embodiments, the composition comprises IGF2, THBS4, and THBS2 In certain embodiments, the composition comprises IGF2, THBS4, and THBS1. In certain embodiments, the composition comprises IGF2, THBS4, and IL-15.

In certain embodiments, the composition comprises IL-15, and THBS4, and VTN. In certain embodiments, the composition comprises IL-15, and THBS4, and POSTN. In certain embodiments, the composition comprises IL-15, and THBS4, and FGF17. In certain embodiments, the composition comprises IL-15, and THBS4, and THBS2. In certain embodiments, the composition comprises IL-15, and THBS4, and THBS1. In certain embodiments, the composition comprises IL-15, and THBS4, and IGF2.

In certain embodiments, the composition comprises VTN, POSTN, and FGF17. In certain embodiments, the composition comprises VTN, POSTN, and THBS2 In certain embodiments, the composition comprises VTN, POSTN, and THBS4 In certain embodiments, the composition comprises VTN, FGF17, and POSTN. In certain embodiments, the composition comprises VTN, FGF17, and THBS2 In certain embodiments, the composition comprises VTN, FGF17, and THBS4. In certain embodiments, the composition comprises VTN, THBS2, and POSTN. In certain embodiments, the composition comprises VTN, THBS2, and FGF17. In certain embodiments, the composition comprises VTN, THBS2, and THBS4. In certain embodiments, the composition comprises VTN, THBS4, and POSTN. In certain embodiments, the composition comprises VTN, THBS4, and FGF17 In certain embodiments, the composition comprises VTN, THBS4, and THBS2. In certain embodiments, the composition comprises POSTN, VTN, and THBS4. In certain embodiments, the composition comprises POSTN, VTN, and FGF17. In certain embodiments, the composition comprises POSTN, VTN, and THBS2. In certain embodiments, the composition comprises POSTN, FGF17, and THBS4. In certain embodiments, the composition comprises POSTN, FGF17, and VTN. In certain embodiments, the composition comprises POSTN, FGF17, and THBS2. In certain embodiments, the composition comprises POSTN, THBS2, and THBS4. In certain embodiments, the composition comprises POSTN, THBS2, and VTN. In certain embodiments, the composition comprises POSTN, THBS2, and FGF17. In certain embodiments, the composition comprises POSTN, THBS4, and THBS2. In certain embodiments, the composition comprises POSTN, THBS4, and VTN. In certain embodiments, the composition comprises POSTN, THBS4, and FGF17. In certain embodiments, the composition comprises FGF17, VTN, and THBS2. In certain embodiments, the composition comprises FGF17, VTN, and THBS4. In certain embodiments, the composition comprises FGF17, VTN, and POSTN. In certain embodiments, the composition comprises FGF17, POSTN, and THBS2. In certain embodiments, the composition comprises FGF17, POSTN, and THBS4. In certain embodiments, the composition comprises FGF17, POSTN, and VTN. In certain embodiments, the composition comprises FGF17, THBS2, and POSTN. In certain embodiments, the composition comprises FGF17, THBS2, and THBS4. In certain embodiments, the composition comprises FGF17, THBS2, and VTN. In certain embodiments, the composition comprises FGF17, THBS4, and POSTN. In certain embodiments, the composition comprises FGF17, THBS4, and THBS2. In certain embodiments, the composition comprises FGF17, THBS4, and VTN. In certain embodiments, the composition comprises THBS2. VTN, and POSTN. In certain embodiments, the composition comprises THBS2, VTN, and FGF17 In certain embodiments, the composition comprises THBS2. VTN, and THBS4. In certain embodiments, the composition comprises THBS2, POSTN, and VTN. In certain embodiments, the composition comprises THBS2, POSTN, and FGF17. In certain embodiments, the composition comprises THBS2, POSTN, and THBS4. In certain embodiments, the composition comprises THBS2, FGF17, and VTN. In certain embodiments, the composition comprises THBS2, FGF17, and POSTN. In certain embodiments, the composition comprises THBS2, FGF17, and THBS4. In certain embodiments, the composition comprises THBS2, THBS4, and VTN. In certain embodiments, the composition comprises THBS2, THBS4, and POSTN. In certain embodiments, the composition comprises THBS2, THBS4, and FGF17. In certain embodiments, the composition comprises THBS4, VTN, and THBS2. In certain embodiments, the composition comprises THBS4, VTN, and POSTN. In certain embodiments, the composition comprises THBS4, VTN, and FGF17. In certain embodiments, the composition comprises THBS4, POSTN, and THBS2. In certain embodiments, the composition comprises THBS4. POSTN, and VTN. In certain embodiments, the composition comprises THBS4, POSTN, and FGF17. In certain embodiments, the composition comprises THBS4. FGF17, and THBS2. In certain embodiments, the composition comprises THBS4, FGF17, and VTN. In certain embodiments, the composition comprises THBS4, FGF17, and POSTN. In certain embodiments, the composition comprises THBS4, THBS2, and FGF17. In certain embodiments, the composition comprises THBS4, THBS2, and VTN. In certain embodiments, the composition comprises THBS4, THBS2, and POSTN.

In certain embodiments, the heparin-associated binding polypeptide composition comprising any three polypeptides selected from VTN, POSTN, FGF17, THBS2, THBS1, IGF2, IL-15, and THBS4, comprises one or more additional modifications to increase stability. In certain embodiments, one or more of the heparin-associated polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the heparin-associated polypeptides is fused or conjugated to another protein to increase stability and or bioavailability. In certain embodiments, one or more of the heparin-associated polypeptides is fused with an Fc region of an immunoglobulin or with serum albumin. In certain embodiments, one or more of the heparin-associated polypeptides is present in a concatemer with one, two, three, or four other distinct polypeptides selected from THBS2, VTN, POSTN, FGF17, THBS1, IGF2, IL-15, and THBS4. In certain embodiments, one or more of the heparin-associated polypeptides is present in a concatemer with one, two, three, four, or more distinct polypeptides. In certain embodiments, one or more of the heparin-associated polypeptides is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, the heparin-associated binding polypeptide composition comprises any four polypeptides selected from VTN, POSTN, FGF17, THBS1, IGF2, IL-15, THBS2, and THBS4. In certain embodiments, the composition comprises VTN, POSTN, FGF17, and THBS2. In certain embodiments, the composition comprises VTN, POSTN, FGF17, and THBS4 In certain embodiments, the composition comprises VTN, POSTN, THBS2, and FGF17. In certain embodiments, the composition comprises VTN, POSTN, THBS2, and THBS4. In certain embodiments, the composition comprises VTN, POSTN, THBS4, and FGF17. In certain embodiments, the composition comprises VTN, POSTN, THBS4, and THBS2. In certain embodiments, the composition comprises VTN, FGF17, POSTN, and THBS4. In certain embodiments, the composition comprises VTN, FGF17, POSTN, and THBS2. In certain embodiments, the composition comprises VTN, FGF17, THBS2, and THBS4. In certain embodiments, the composition comprises VTN, FGF17, THBS2, and POSTN. In certain embodiments, the composition comprises VTN, FGF17, THBS4, and THBS2. In certain embodiments, the composition comprises VTN, FGF17, THBS4, and POSTN. In certain embodiments, the composition comprises VTN, THBS2, POSTN, and FGF17. In certain embodiments, the composition comprises VTN, THBS2, POSTN, and THBS4. In certain embodiments, the composition comprises VTN, THBS2, FGF17, and POSTN. In certain embodiments, the composition comprises VTN, THBS2, FGF17, and THBS4 In certain embodiments, the composition comprises VTN, THBS2, THBS4, and POSTN. In certain embodiments, the composition comprises VTN, THBS2, THBS4, and FGF17. In certain embodiments, the composition comprises VTN, THBS4, POSTN, and THBS2. In certain embodiments, the composition comprises VTN, THBS4, POSTN, and FGF17. In certain embodiments, the composition comprises VTN, THBS4, FGF17, and THBS2 In certain embodiments, the composition comprises VTN, THBS4, FGF17, and POSTN. In certain embodiments, the composition comprises VTN, THBS4, THBS2, and FGF17. In certain embodiments, the composition comprises VTN, THBS4, THBS2, and POSTN. In certain embodiments, the composition comprises POSTN, VTN, THBS4, and FGF17. In certain embodiments, the composition comprises POSTN, VTN, THBS4, and THBS2. In certain embodiments, the composition comprises POSTN, VTN, FGF17, and THBS4. In certain embodiments, the composition comprises POSTN, VTN, FGF17, and THBS2. In certain embodiments, the composition comprises POSTN, VTN, THBS2, and THBS4. In certain embodiments, the composition comprises POSTN, VTN, THBS2, and FGF17. In certain embodiments, the composition comprises POSTN, FGF17, THBS4, and THBS2. In certain embodiments, the composition comprises POSTN, FGF17, THBS4, and VTN. In certain embodiments, the composition comprises POSTN, FGF17, VTN, and THBS2. In certain embodiments, the composition comprises POSTN, FGF17, VTN, and THBS4. In certain embodiments, the composition comprises POSTN, FGF17, THBS2, and VTN. In certain embodiments, the composition comprises POSTN, FGF17, THBS2, and THBS4. In certain embodiments, the composition comprises POSTN, THBS2, THBS4, and VTN. In certain embodiments, the composition comprises POSTN, THBS2, THBS4, and FGF17. In certain embodiments, the composition comprises POSTN, THBS2, VTN, and THBS4. In certain embodiments, the composition comprises POSTN, THBS2, VTN, and FGF17. In certain embodiments, the composition comprises POSTN, THBS2, FGF17, and THBS4. In certain embodiments, the composition comprises POSTN, THBS2, FGF17, and VTN. In certain embodiments, the composition comprises POSTN, THBS4, THBS2, and FGF17. In certain embodiments, the composition comprises POSTN, THBS4, THBS2, and VTN. In certain embodiments, the composition comprises POSTN, THBS4, VTN, and FGF17. In certain embodiments, the composition comprises POSTN, THBS4, VTN, and THBS2. In certain embodiments, the composition comprises POSTN, THBS4, FGF17, and VTN. In certain embodiments, the composition comprises POSTN, THBS4, FGF17, and THBS2 In certain embodiments, the composition comprises FGF17, VTN, THBS2, and THBS4. In certain embodiments, the composition comprises FGF17, VTN, THBS2, and POSTN. In certain embodiments, the composition comprises FGF17, VTN, THBS4, and THBS2. In certain embodiments, the composition comprises FGF17, VTN, THBS4, and POSTN. In certain embodiments, the composition comprises FGF17, VTN, POSTN, and THBS2. In certain embodiments, the composition comprises FGF17, VTN, POSTN, and THBS4. In certain embodiments, the composition comprises FGF17, POSTN, THBS2, and VTN. In certain embodiments, the composition comprises FGF17, POSTN, THBS2, and THBS4. In certain embodiments, the composition comprises FGF17, POSTN, THBS4, and VTN. In certain embodiments, the composition comprises FGF17, POSTN, THBS4, and THBS2 In certain embodiments, the composition comprises FGF17, POSTN, VTN, and THBS4. In certain embodiments, the composition comprises FGF17, POSTN, VTN, and THBS2. In certain embodiments, the composition comprises FGF17, THBS2, POSTN, and THBS4. In certain embodiments, the composition comprises FGF17, THBS2, POSTN, and VTN. In certain embodiments, the composition comprises FGF17, THBS2, THBS4, and POSTN. In certain embodiments, the composition comprises FGF17, THBS2, THBS4, and VTN. In certain embodiments, the composition comprises FGF17, THBS2, VTN, and POSTN. In certain embodiments, the composition comprises FGF17, THBS2, VTN, and THBS4. In certain embodiments, the composition comprises FGF17, THBS4, POSTN, and VTN. In certain embodiments, the composition comprises FGF17, THBS4, POSTN, and THBS2. In certain embodiments, the composition comprises FGF17, THBS4, THBS2, and VTN. In certain embodiments, the composition comprises FGF17, THBS4, THBS2, and POSTN. In certain embodiments, the composition comprises FGF17, THBS4, VTN, and THBS2 In certain embodiments, the composition comprises FGF17, THBS4, VTN, and POSTN. In certain embodiments, the composition comprises THBS2, VTN, POSTN, and FGF17. In certain embodiments, the composition comprises THBS2, VTN, POSTN, and THBS4. In certain embodiments, the composition comprises THBS2, VTN, FGF17, and POSTN. In certain embodiments, the composition comprises THBS2, VTN, FGF17, and THBS4 In certain embodiments, the composition comprises THBS2, VTN, THBS4, and POSTN. In certain embodiments, the composition comprises THBS2, VTN, THBS4, and FGF17. In certain embodiments, the composition comprises THBS2, POSTN, VTN, and THBS4. In certain embodiments, the composition comprises THBS2, POSTN, VTN, and FGF17. In certain embodiments, the composition comprises THBS2, POSTN, FGF17, and THBS4. In certain embodiments, the composition comprises THBS2, POSTN, FGF17, and VTN. In certain embodiments, the composition comprises THBS2, POSTN, THBS4, and FGF17. In certain embodiments, the composition comprises THBS2, POSTN, THBS4, and VTN. In certain embodiments, the composition comprises THBS2, FGF17, VTN, and POSTN. In certain embodiments, the composition comprises THBS2, FGF17, VTN, and THBS4. In certain embodiments, the composition comprises THBS2, FGF17, POSTN, and VTN. In certain embodiments, the composition comprises THBS2, FGF17, POSTN, and THBS4. In certain embodiments, the composition comprises THBS2, FGF17, THBS4, and VTN. In certain embodiments, the composition comprises THBS2, FGF17, THBS4, and POSTN. In certain embodiments, the composition comprises THBS2, THBS4, VTN, and FGF17. In certain embodiments, the composition comprises THBS2, THBS4, VTN, and POSTN. In certain embodiments, the composition comprises THBS2, THBS4, POSTN, and FGF17. In certain embodiments, the composition comprises THBS2, THBS4, POSTN, and VTN. In certain embodiments, the composition comprises THBS2, THBS4, FGF17, and POSTN. In certain embodiments, the composition comprises THBS2, THBS4, FGF17, and VTN. In certain embodiments, the composition comprises THBS4, VTN, THBS2, and POSTN. In certain embodiments, the composition comprises THBS4, VTN, THBS2, and FGF17. In certain embodiments, the composition comprises TBS4, VTN, POSTN, and THBS2. In certain embodiments, the composition comprises THBS4, VTN, POSTN, and FGF17. In certain embodiments, the composition comprises THBS4, VTN, FGF17, and THBS2. In certain embodiments, the composition comprises THBS4, VIN, FGF17, and POSTN. In certain embodiments, the composition comprises THBS4, POSTN, THBS2, and FGF17 In certain embodiments, the composition comprises THBS4, POSTN, THBS2, and VTN. In certain embodiments, the composition comprises THBS4, POSTN, VTN, and FGF17. In certain embodiments, the composition comprises THBS4, POSTN, VTN, and THBS2. In certain embodiments, the composition comprises THBS4, POSTN, FGF17, and VTN. In certain embodiments, the composition comprises TBS4, POSTN, FGF17, and THBS2 In certain embodiments, the composition comprises THBS4, FGF17, THBS2, and VTN. In certain embodiments, the composition comprises THBS4, FGF17, THBS2, and POSTN. In certain embodiments, the composition comprises THBS4, FGF17, VTN, and THBS2. In certain embodiments, the composition comprises THBS4, FGF17, VTN, and POSTN. In certain embodiments, the composition comprises THBS4, FGF17, POSTN, and THBS2. In certain embodiments, the composition comprises THBS4, FGF17, POSTN, and VTN. In certain embodiments, the composition comprises THBS4, THBS2, FGF17, and POSTN. In certain embodiments, the composition comprises THBS4, THBS2, FGF17, and VTN. In certain embodiments, the composition comprises THBS4, THBS2, VTN, and POSTN. In certain embodiments, the composition comprises THBS4, THBS2, VTN, and FGF17. In certain embodiments, the composition comprises THBS4, THBS2, POSTN, and VTN. In certain embodiments, the composition comprises THBS4, THBS2, POSTN, and FGF17.

In certain embodiments, the composition comprises VTN, POSTN, FGF17, and a polypeptide comprising VTN, POSTN, FGF17, THBS2, THBS1, IGF2, IL-15, or THBS4. In certain embodiments, the composition comprises VTN, POSTN, THBS2, and a polypeptide comprising VTN, POSTN, FGF17, THBS2, THBS1, IGF2, IL-15, or THBS4. In certain embodiments, the composition comprises VTN, POSTN, FGF17, and a polypeptide comprising VTN, POSTN, FGF17, THBS2, THBS1, IGF2, IL-15, or THBS4 in certain embodiments, the composition comprises VTN, POSTN, THBS2, and a polypeptide comprising VTN, POSTN, FGF17, THBS2, THBS1, IGF2, IL-15, or THBS4. In certain embodiments, the composition comprises VTN, POSTN, THBS1, and a polypeptide comprising VTN, POSTN, FGF17, THBS2, THBS1, IGF2, IL-15, or THBS4 In certain embodiments, the composition comprises VTN, POSTN, IGF2, and a polypeptide comprising VTN, POSTN, FGF17, THBS2, THBS1, IGF2, IL-15, or THBS4. In certain embodiments, the composition comprises VTN, POSTN, IL-15, and a polypeptide comprising VTN, POSTN, FGF17, THBS2, THBS1, IGF2, IL-15, or THBS4. In certain embodiments, the composition comprises VTN, POSTN, THBS4, and a polypeptide comprising VIN, POSTN, FGF17, THBS2, THBS1, IGF2, IL-15, or THBS4.

In certain embodiments, the composition comprises VTN, FGF17, THBS2, and a polypeptide comprising VTN, POSTN, FGF17, THBS2, THBS1, IGF2, IL-15, or THBS4. In certain embodiments, the composition comprises VTN, FGF17, POSTN, and a polypeptide comprising VTN, POSTN, FGF17, THBS2, THBS1, IGF2, IL-15, or THBS4. In certain embodiments, the composition comprises VTN, FGF17, THBS2, and a polypeptide comprising VTN, POSTN, FGF17, THBS2, THBS1, IGF2, IL-15, or THBS4. In certain embodiments, the composition comprises VTN, FGF17, THBS1, and a polypeptide comprising VTN, POSTN, FGF17, THBS2, THBS1, IGF2, IL-15, or THBS4. In certain embodiments, the composition comprises VTN. FGF17, IGF2, and a polypeptide comprising VTN, POSTN, FGF17, THBS2, THBS1, IGF2, IL-15, or THBS4. In certain embodiments, the composition comprises VTN, FGF17, IL-15, and a polypeptide comprising VTN, POSTN, FGF17, THBS2, THBS1, IGF2, IL-15, or THBS4. In certain embodiments, the composition comprises VTN, FGF17, THBS4, and a polypeptide comprising VTN, POSTN, FGF17, THBS2, THBS1, IGF2, IL-15, or THBS4.

In certain embodiments, the composition comprises VTN, THBS2, POSTN, and a polypeptide comprising VTN, POSTN, FGF17, THBS2, THBS1, IGF2, IL-15, or THBS4. In certain embodiments, the composition comprises VTN, THBS2, FGF17, and a polypeptide comprising VTN, POSTN, FGF17, THBS2, THBS1, IGF2, IL-15, or THBS4. In certain embodiments, the composition comprises VTN, THBS2, THBS1, and a polypeptide comprising VTN, POSTN, FGF17, THBS2, THBS1, IGF2, IL-15, or THBS4. In certain embodiments, the composition comprises VTN, THBS2, IGF2, and a polypeptide comprising VTN, POSTN, FGF17, THBS2, THBS1, IGF2, IL-15, or THBS4. In certain embodiments, the composition comprises VTN, THBS2, IL-15, and a polypeptide comprising VTN, POSTN, FGF17, THBS2, THBS1, IGF2, IL-15, or THBS4 In certain embodiments, the composition comprises VTN, THBS2, THBS4, and a polypeptide comprising VTN, POSTN, FGF17, THBS2, THBS1, IGF2, IL-15, or THBS4.

In certain embodiments, the composition comprises VTN, THBS1, POSTN, and a polypeptide comprising VTN, POSTN, FGF17, THBS2, THBS1, IGF2, IL-15, or THBS4 In certain embodiments, the composition comprises VTN, THBS1, FGF17, and a polypeptide comprising VTN, POSTN, FGF17, THBS2, THBS1, IGF2, IL-15, or THBS4. In certain embodiments, the composition comprises VTN, THBS1, THBS2, and a polypeptide comprising VTN, POSTN, FGF17, THBS2, THBS1, IGF2, IL-15, or THBS4. In certain embodiments, the composition comprises VTN, THBS1, IGF2, and a polypeptide comprising VTN, POSTN, FGF17, THBS2, THBS1, IGF2, IL-15, or THBS4. In certain embodiments, the composition comprises VTN, THBS1, IL-15, and a polypeptide comprising VTN, POSTN, FGF17, THBS2, THBS1, IGF2, IL-15, or THBS4. In certain embodiments, the composition comprises VTN, THBS1, THBS4, and a polypeptide comprising VTN, POSTN, FGF17, THBS2, THBS1, IGF2, IL-15, or THBS4.

In certain embodiments, the composition comprises VTN, IGF2, POSTN, and a polypeptide comprising VTN, POSTN, FGF17, THBS2, THBS1, IGF2, IL-15, or THBS4. In certain embodiments, the composition comprises VTN, IGF2, FGF17, and a polypeptide comprising VTN, POSTN, FGF17, THBS2, THBS1, IGF2, IL-15, or THBS4. In certain embodiments, the composition comprises VTN, IGF2, THBS2, and a polypeptide comprising VTN, POSTN, FGF17, THBS2, THBS1, IGF2, IL-15, or THBS4. In certain embodiments, the composition comprises VTN, IGF2, THBS1, and a polypeptide comprising VTN, POSTN, FGF17, THBS2, THBS1, IGF2, IL-15, or THBS4. In certain embodiments, the composition comprises VTN, IGF2, IL-15, and a polypeptide comprising VTN, POSTN, FGF17, THBS2, THBS1, IGF2, IL-15, or THBS4. In certain embodiments, the composition comprises VTN, IGF2, THBS4, and a polypeptide comprising VTN, POSTN, FGF17, THBS2, THBS1, IGF2, IL-15, or THBS4.

In certain embodiments, the composition comprises VTN, IL-15, POSTN, and a polypeptide comprising VTN, POSTN, FGF17, THBS2, THBS1, IGF2, IL-15, or THBS4 in certain embodiments, the composition comprises VTN, IL-15, FGF17, and a polypeptide comprising VTN, POSTN, FGF17, THBS2, THBS1, IGF2, IL-15, or THBS4. In certain embodiments, the composition comprises VTN, IL-15, THBS2, and a polypeptide comprising VTN, POSTN, FGF17, THBS2, THBS1, IGF2, IL-15, or THBS4. In certain embodiments, the composition comprises VTN, IL-15, THBS1, and a polypeptide comprising VTN, POSTN, FGF117, THBS2, THBS1, IGF2, IL-15, or THBS4. In certain embodiments, the composition comprises VTN, IL-15, IGF2, and a polypeptide comprising VTN, POSTN, FGF17, THBS2, THBS1, IGF2, IL-15, or THBS4. In certain embodiments, the composition comprises VTN, IL-15, THBS4, and a polypeptide comprising VTN, POSTN, FGF17, THBS2, THBS1, IGF2, IL-15, or THBS4.

In certain embodiments, the composition comprises VTN, TBHS4, POSTN, and a polypeptide comprising VTN, POSTN, FGF17, THBS2, THBS1, IGF2, IL-15, or THBS4. In certain embodiments, the composition comprises VTN, TBHS4, FGF17, and a polypeptide comprising VTN, POSTN, FGF17, THBS2, THBS1, IGF2, IL-15, or THBS4. In certain embodiments, the composition comprises VTN, TBHS4, THBS2, and a polypeptide comprising VTN, POSTN, FGF17, THBS2, THBS1, IGF2, IL-15, or THBS4. In certain embodiments, the composition comprises VTN, TBHS4, THBS1, and a polypeptide comprising VTN, POSTN, FGF17, THBS2, THBS1, IGF2, IL-15, or THBS4. In certain embodiments, the composition comprises VTN, TBHS4, IGF2, and a polypeptide comprising VTN, POSTN, FGF17, THBS2, THBS1, IGF2, IL-15, or THBS4. In certain embodiments, the composition comprises VTN, TBHS4, IL-15, and a polypeptide comprising VTN, POSTN, FGF17, THBS2, THBS1, IGF2, IL-15, or THBS4.

In certain embodiments, the composition comprises POSTN, FGF17, VTN, and a polypeptide comprising VTN, POSTN, FGF17, THBS2, THBS1, IGF2, IL-15, or THBS4. In certain embodiments, the composition comprises POSTN, FGF17, TBHS2, and a polypeptide comprising VTN, POSTN, FGF17, THBS2, THBS1, IGF2, IL-15, or THBS4. In certain embodiments, the composition comprises POSTN, FGF17, THBS1, and a polypeptide comprising VTN, POSTN, FGF17, THBS2, THBS1, IGF2, IL-15, or THBS4. In certain embodiments, the composition comprises POSTN, FGF17, IGF2, and a polypeptide comprising VTN, POSTN, FGF17, THBS2, THBS1, IGF2, IL-15, or THBS4. In certain embodiments, the composition comprises POSTN, FGF17, IL-15, and a polypeptide comprising VTN, POSTN, FGF17, THBS2, THBS1, IGF2, IL-15, or THBS4. In certain embodiments, the composition comprises POSTN, FGF17, THBS4, and a polypeptide comprising VTN, POSTN, FGF17, THBS2, THBS1, IGF2, IL-15, or THBS4.

In certain embodiments, the composition comprises POSTN, THBS2, VTN, and a polypeptide comprising VTN, POSTN, FGF17, THBS2, THBS1, IGF2, IL-15, or THBS4. In certain embodiments, the composition comprises POSTN, THBS2, FGF17, and a polypeptide comprising VTN, POSTN, FGF17, THBS2, THBS1, IGF2, IL-15, or THBS4. In certain embodiments, the composition comprises POSTN, THBS2, THBS1, and a polypeptide comprising VTN, POSTN, FGF17, THBS2, THBS1, IGF2, IL-15, or THBS4. In certain embodiments, the composition comprises POSTN, THBS2, IGF2, and a polypeptide comprising VTN, POSTN, FGF17, THBS2, THBS1, IGF2, IL-15, or THBS4. In certain embodiments, the composition comprises POSTN, THBS2, IL-15, and a polypeptide comprising VTN, POSTN, FGF17, THBS2, THBS1, IGF2, IL-15, or THBS4. In certain embodiments, the composition comprises POSTN, THBS2, THBS4, and a polypeptide comprising VTN, POSTN, FGF17, THBS2, THBS1, IGF2, IL-15, or THBS4.

In certain embodiments, the composition comprises POSTN, THBS1, VTN, and a polypeptide comprising VTN, POSTN, FGF17, THBS2, THBS1, IGF2, IL-15, or THBS4. In certain embodiments, the composition comprises POSTN, THBS1, FGF17, and a polypeptide comprising VTN, POSTN, FGF17, THBS2, THBS1, IGF2, IL-15, or THBS4. In certain embodiments, the composition comprises POSTN, THBS1, THBS2, and a polypeptide comprising VTN, POSTN, FGF17, THBS2, THBS1, IGF2, IL-15, or THBS4. In certain embodiments, the composition comprises POSTN, THBS1, IGF2, and a polypeptide comprising VTN, POSTN, FGF17, THBS2, THBS1, IGF2, IL-15, or THBS4. In certain embodiments, the composition comprises POSTN, THBS1, IL-15, and a polypeptide comprising VTN, POSTN, FGF17, THBS2, THBS1, IGF2, IL-15, or THBS4. In certain embodiments, the composition comprises POSTN, THBS1, THBS4, and a polypeptide comprising VTN, POSTN, FGF17, THBS2, THBS1, IGF2, IL-15, or THBS4.

In certain embodiments, the composition comprises POSTN, IGF2, VTN, and a polypeptide comprising VTN, POSTN, FGF17, THBS2, THBS1, IGF2, IL-15, or THBS4 in certain embodiments, the composition comprises POSTN, IGF2, FGF17, and a polypeptide comprising VTN, POSTN, FGF17, THBS2, THBS1, IGF2, IL-15, or THBS4. In certain embodiments, the composition comprises POSTN, IGF2, THBS2, and a polypeptide comprising VTN, POSTN, FGF17, THBS2, THBS1, IGF2, IL-15, or THBS4. In certain embodiments, the composition comprises POSTN, IGF2, THBS1, and a polypeptide comprising VTN, POSTN, FGF17, THBS2, THBS1, IGF2, IL-15, or THBS4. In certain embodiments, the composition comprises POSTN, IGF2, IL-15, and a polypeptide comprising VTN, POSTN, FGF17, THBS2, THBS1, IGF2, IL-15, or THBS4. In certain embodiments, the composition comprises POSTN, IGF2, THBS4, and a polypeptide comprising VTN, POSTN, FGF17, THBS2, THBS1, IGF2, IL-15, or THBS4.

In certain embodiments, the composition comprises POSTN, IL-15, VTN, and a polypeptide comprising VTN, POSTN, FGF17, THBS2, THBS1, IGF2, IL-15, or THBS4. In certain embodiments, the composition comprises POSTN, IL-15, FGF17, and a polypeptide comprising VTN, POSTN, FGF17, THBS2, THBS1, IGF2, IL-15, or THBS4. In certain embodiments, the composition comprises POSTN, IL-15, THBS2, and a polypeptide comprising VTN, POSTN, FGF17, THBS2, THBS1, IGF2, IL-15, or THBS4. In certain embodiments, the composition comprises POSTN, IL-15, THBS1, and a polypeptide comprising VTN, POSTN, FGF17, THBS2, THBS1, IGF2, IL-15, or THBS4. In certain embodiments, the composition comprises POSTN, IL-15, IGF2, and a polypeptide comprising VTN, POSTN, FGF17, THBS2, THBS1, IGF2, IL-15, or THBS4. In certain embodiments, the composition comprises POSTN, IL-15, THBS4, and a polypeptide comprising VTN, POSTN, FGF17, THBS2, THBS1, IGF2, IL-15, or THBS4.

In certain embodiments, the composition comprises POSTN, THBS4, VTN, and a polypeptide comprising VTN, POSTN, FGF17, THBS2, THBS1, IGF2, IL-15, or THBS4. In certain embodiments, the composition comprises POSTN, THBS4, FGF17, and a polypeptide comprising VTN, POSTN, FGF17, THBS2, THBS1, IGF2, IL-15, or THBS4. In certain embodiments, the composition comprises POSTN, THBS4, THBS2, and a polypeptide comprising VTN, POSTN, FGF17, THBS2, THBS1, IGF2, IL-15, or THBS4. In certain embodiments, the composition comprises POSTN, THBS4, THBS1, and a polypeptide comprising VTN, POSTN, FGF17, THBS2, THBS1, IGF2, IL-15, or THBS4. In certain embodiments, the composition comprises POSTN, THBS4, IGF2, and a polypeptide comprising VTN, POSTN, FGF17, THBS2, THBS1, IGF2, IL-15, or THBS4. In certain embodiments, the composition comprises POSTN, THBS4, IL-15, and a polypeptide comprising VTN, POSTN, FGF17, THBS2, THBS1, IGF2, IL-15, or THBS4.

In certain embodiments, the composition comprises FGF17, THBS2, VTN, and a polypeptide comprising VTN, POSTN, FGF17, THBS2, THBS1, IGF2, IL-15, or THBS4. In certain embodiments, the composition comprises FGF17, THBS2, POSTN, and a polypeptide comprising VTN, POSTN, FGF17, THBS2, THBS1, IGF2, IL-15, or THBS4. In certain embodiments, the composition comprises FGF17, THBS2, THBS1, and a polypeptide comprising VTN, POSTN, FGF17, THBS2, THBS1, IGF2, IL-15, or THBS4. In certain embodiments, the composition comprises FGF17, THBS2, IGF2, and a polypeptide comprising VTN, POSTN, FGF17, THBS2, THBS1, IGF2, IL-15, or THBS4. In certain embodiments, the composition comprises FGF17, THBS2, IL-15, and a polypeptide comprising VTN, POSTN, FGF17, THBS2, THBS1, IGF2, IL-15, or THBS4. In certain embodiments, the composition comprises FGF17, THBS2, THBS4, and a polypeptide comprising VTN, POSTN, FGF17, THBS2, THBS1, IGF2, IL-15, or THBS4.

In certain embodiments, the composition comprises FGF17, THBS1, VTN, and a polypeptide comprising VTN, POSTN, FGF17, THBS2, THBS1, IGF2, IL-15, or THBS4. In certain embodiments, the composition comprises FGF17, THBS1, POSTN, and a polypeptide comprising VTN, POSTN, FGF17, THBS2, THBS1, IGF2, IL-15, or THBS4. In certain embodiments, the composition comprises FGF17, THBS1, THBS2, and a polypeptide comprising VTN, POSTN, FGF17, THBS2, THBS1, IGF2, IL-15, or THBS4. In certain embodiments, the composition comprises FGF17, THBS1, IGF2, and a polypeptide comprising VTN, POSTN, FGF17, THBS2, THBS1, IGF2, IL-15, or THBS4. In certain embodiments, the composition comprises FGF17, THBS1, IL-15, and a polypeptide comprising VTN, POSTN, FGF17, THBS2, THBS1, IGF2, IL-15, or THBS4. In certain embodiments, the composition comprises FGF17, THBS1, THBS4, and a polypeptide comprising VTN, POSTN, FGF17, THBS2, THBS1, IGF2, IL-15, or THBS4.

In certain embodiments, the composition comprises FGF17, IGF2, VTN, and a polypeptide comprising VTN, POSTN, FGF17, THBS2, THBS1, IGF2, IL-15, or THBS4 In certain embodiments, the composition comprises FGF17, IGF2, POSTN, and a polypeptide comprising VTN, POSTN, FGF17, THBS2, THBS1, IGF2, IL-15, or THBS4. In certain embodiments, the composition comprises FGF17, IGF2, THBS2, and a polypeptide comprising VTN, POSTN, FGF17, THBS2, THBS1, IGF2, IL-15, or THBS4. In certain embodiments, the composition comprises FGF17, IGF2, THBS1, and a poly peptide comprising VTN, POSTN, FGF17, THBS2, THBS1, IGF2, IL-15, or THBS4. In certain embodiments, the composition comprises FGF17, IGF2, IL-15, and a polypeptide comprising VTN, POSTN, FGF17, THBS2, THBS1, IGF2, IL-15, or THBS4. In certain embodiments, the composition comprises FGF17, IGF2, THBS4, and a polypeptide comprising VTN, POSTN, FGF17, THBS2, THBS1, IGF2, IL-15, or THBS4.

In certain embodiments, the composition comprises FGF17, IL-15, VTN, and a polypeptide comprising VTN, POSTN, FGF17, THBS2, THBS1, IGF2, IL-15, or THBS4. In certain embodiments, the composition comprises FGF17, IL-15, POSTN, and a polypeptide comprising VTN, POSTN, FGF17, THBS2, THBS1, IGF2, IL-15, or THBS4. In certain embodiments, the composition comprises FGF17, IL-15, THBS2, and a polypeptide comprising VTN, POSTN, FGF17, THBS2, THBS1, IGF2, IL-15, or THBS4. In certain embodiments, the composition comprises FGF17, IL-15, THBS1, and a polypeptide comprising VTN, POSTN, FGF17, THBS2, THBS1, IGF2, IL-15, or THBS4. In certain embodiments, the composition comprises FGF17, IL-15, IGF2, and a polypeptide comprising VTN, POSTN, FGF17, THBS2, THBS1, IGF2, IL-15, or THBS4. In certain embodiments, the composition comprises FGF17, IL-15, THBS4, and a polypeptide comprising VTN, POSTN, FGF17, THBS2, THBS1, IGF2, IL-15, or THBS4.

In certain embodiments, the composition comprises FGF17, THBS4, VTN, and a polypeptide comprising VTN, POSTN, FGF17, THBS2, THBS1, IGF2, IL-15, or THBS4. In certain embodiments, the composition comprises FGF17, THBS4, POSTN, and a polypeptide comprising VTN, POSTN, FGF17, THBS2, THBS1, IGF2, IL-15, or THBS4. In certain embodiments, the composition comprises FGF17, THBS4, THBS2, and a polypeptide comprising VTN, POSTN, FGF17, THBS2, THBS1, IGF2, IL-15, or THBS4. In certain embodiments, the composition comprises FGF17, THBS4, THBS1, and a polypeptide comprising VTN, POSTN, FGF17, THBS2, THBS1, IGF2, IL-15, or THBS4. In certain embodiments, the composition comprises FGF17, THBS4, IGF2, and a polypeptide comprising VTN, POSTN, FGF17, THBS2, THBS1, IGF2, IL-15, or THBS4. In certain embodiments, the composition comprises FGF17, THBS4, IL-15, and a polypeptide comprising VTN, POSTN, FGF17, THBS2, THBS1, IGF2, IL-15, or THBS4.

In certain embodiments, the composition comprises THBS2, THBS1, VTN, and a polypeptide comprising VTN, POSTN, FGF17, THBS2, THBS1, IGF2, IL-15, or THBS4. In certain embodiments, the composition comprises THBS2, THBS1, POSTN, and a polypeptide comprising VTN, POSTN, FGF17, THBS2, THBS1, IGF2, IL-15, or THBS4. In certain embodiments, the composition comprises THBS2, THBS1, FGF17, and a polypeptide comprising VTN, POSTN, FGF17, THBS2, THBS1, IGF2, IL-15, or THBS4. In certain embodiments, the composition comprises THBS2, THBS1, IGF2, and a polypeptide comprising VTN, POSTN, FGF17, THBS2, THBS1, IGF2, IL-15, or THBS4. In certain embodiments, the composition comprises THBS2, THBS1, IL-15, and a polypeptide comprising VTN, POSTN, FGF17, THBS2, THBS1, IGF2, IL-15, or THBS4. In certain embodiments, the composition comprises THBS2, THBS1, THBS4, and a polypeptide comprising VTN, POSTN, FGF17, THBS2, THBS1, IGF2, IL-15, or THBS4.

In certain embodiments, the composition comprises THBS2, IGF2, VTN, and a polypeptide comprising VTN, POSTN, FGF17, THBS2, THBS1, IGF2, IL-15, or THBS4. In certain embodiments, the composition comprises THBS2, IGF2, POSTN, and a polypeptide comprising VTN, POSTN, FGF17, THBS2, THBS1, IGF2, IL-15, or THBS4. In certain embodiments, the composition comprises THBS2, IGF2, FGF17, and a polypeptide comprising VTN, POSTN, FGF17, THBS2, THBS1, IGF2, IL-15, or THBS4 In certain embodiments, the composition comprises THBS2, IGF2, THBS1, and a polypeptide comprising VTN, POSTN, FGF17, THBS2, THBS1, IGF2, IL-15, or THBS4 In certain embodiments, the composition comprises THBS2, IGF2, IL-15, and a polypeptide comprising VTN, POSTN, FGF17, THBS2, THBS1, IGF2, IL-15, or THBS4. In certain embodiments, the composition comprises THBS2, IGF2, THBS4, and a polypeptide comprising VTN, POSTN, FGF17, THBS2, THBS1, IGF2, IL-15, or THBS4.

In certain embodiments, the composition comprises THBS2, IL-15, VTN, and a polypeptide comprising VTN, POSTN, FGF17, THBS2, THBS1, IGF2, IL-15, or THBS4 In certain embodiments, the composition comprises THBS2, IL-15, POSTN, and a polypeptide comprising VTN, POSTN, FGF17, THBS2, THBS1, IGF2, IL-15, or THBS4. In certain embodiments, the composition comprises THBS2, IL-15, FGF17, and a polypeptide comprising VTN, POSTN, FGF17, THBS2, THBS1, IGF2, IL-15, or THBS4. In certain embodiments, the composition comprises THBS2, IL-15, THBS1, and a polypeptide comprising VTN, POSTN, FGF17, THBS2, THBS1, IGF2, IL-15, or THBS4. In certain embodiments, the composition comprises THBS2, IL-15, IGF2, and a polypeptide comprising VTN, POSTN, FGF117, THBS2, THBS1, IGF2, IL-15, or THBS4. In certain embodiments, the composition comprises THBS2, IL-15, THBS4, and a polypeptide comprising VTN, POSTN, FGF17, THBS2, THBS1, IGF2, IL-15, or THBS4.

In certain embodiments, the composition comprises THBS2, THBS4, VTN, and a polypeptide comprising VTN, POSTN, FGF17, THBS2, THBS1, IGF2, IL-15, or THBS4. In certain embodiments, the composition comprises THBS2, THBS4, POSTN, and a polypeptide comprising VTN, POSTN, FGF17, THBS2, THBS1, IGF2, IL-15, or THBS4. In certain embodiments, the composition comprises THBS2, THBS4, FGF17, and a polypeptide comprising VTN, POSTN, FGF17, THBS2, THBS1, IGF2, IL-15, or THBS4. In certain embodiments, the composition comprises THBS2, THBS4, THBS1, and a polypeptide comprising VTN, POSTN, FGF17, THBS2, THBS1, IGF2, IL-15, or THBS4. In certain embodiments, the composition comprises THBS2, THBS4, IGF2, and a polypeptide comprising VTN, POSTN, FGF17, THBS2, THBS1, IGF2, IL-15, or THBS4. In certain embodiments, the composition comprises THBS2, THBS4, IL-15, and a polypeptide comprising VTN, POSTN, FGF17, THBS2, THBS1, IGF2, IL-15, or THBS4.

In certain embodiments, the composition comprises THBS1, IGF2, VTN, and a polypeptide comprising VTN, POSTN, FGF17, THBS2, THBS1, IGF2, IL-15, or THBS4. In certain embodiments, the composition comprises THBS1, IGF2, POSTN, and a polypeptide comprising VTN, POSTN, FGF17, THBS2, THBS1, IGF2, IL-15, or THBS4. In certain embodiments, the composition comprises THBS1, IGF2, FGF17, and a polypeptide comprising VTN, POSTN, FGF17, THBS2, THBS1, IGF2, IL-15, or THBS4. In certain embodiments, the composition comprises THBS1, IGF2, THBS2, and a polypeptide comprising VTN, POSTN, FGF17, THBS2, THBS1, IGF2, IL-15, or THBS4. In certain embodiments, the composition comprises THBS1, IGF2, IL-15, and a polypeptide comprising VTN, POSTN, FGF17, THBS2, THBS1, IGF2, IL-15, or THBS4. In certain embodiments, the composition comprises THBS1, IGF2, THBS4, and a polypeptide comprising VTN, POSTN, FGF17, THBS2, THBS1, IGF2, IL-15, or THBS4.

In certain embodiments, the composition comprises THBS1, IL-15, VTN, and a polypeptide comprising VTN, POSTN, FGF17, THBS2, THBS1, IGF2, IL-15, or THBS4. In certain embodiments, the composition comprises THBS1, IL-15, POSTN, and a polypeptide comprising VTN, POSTN, FGF17, THBS2, THBS1, IGF2, IL-15, or THBS4. In certain embodiments, the composition comprises THBS1, IL-15, FGF17, and a polypeptide comprising VTN, POSTN, FGF17, THBS2, THBS1, IGF2, IL-15, or THBS4. In certain embodiments, the composition comprises THBS1, IL-15, THBS2, and a polypeptide comprising VTN, POSTN, FGF17, THBS2, THBS1, IGF2, IL-15, or THBS4. In certain embodiments, the composition comprises THBS1, IL-15, IGF2, and a polypeptide comprising VTN, POSTN, FGF17, THBS2, THBS1, IGF2, IL-15, or THBS4. In certain embodiments, the composition comprises THBS1, IL-15, THBS4, and a polypeptide comprising VTN, POSTN, FGF17, THBS2, THBS1, IGF2, IL-15, or THBS4.

In certain embodiments, the composition comprises THBS1, and THBS4, VTN, and a polypeptide comprising VTN, POSTN, FGF17, THBS2, THBS1, IGF2, IL-15, or THBS4. In certain embodiments, the composition comprises THBS1, THBS4, POSTN, and a polypeptide comprising VTN, POSTN, FGF17, THBS2, THBS1, IGF2, IL-15, or THBS4. In certain embodiments, the composition comprises THBS1, THBS4, FGF17, and a polypeptide comprising VTN, POSTN, FGF17, THBS2, THBS1, IGF2, IL-15, or THBS4. In certain embodiments, the composition comprises THBS1, THBS4, THBS2, and a polypeptide comprising VTN, POSTN, FGF17, THBS2, THBS1, IGF2, IL-15, or THBS4. In certain embodiments, the composition comprises THBS1, THBS4, IGF2, and a polypeptide comprising VTN, POSTN, FGF17, THBS2, THBS1, IGF2, IL-15, or THBS4. In certain embodiments, the composition comprises THBS1, THBS4, IL-15, and a polypeptide comprising VTN, POSTN, FGF17, THBS2, THBS1, IGF2, IL-15, or THBS4.

In certain embodiments, the composition comprises IGF2, IL-15, VTN, and a polypeptide comprising VTN, POSTN, FGF17, THBS2, THBS1, IGF2, IL-15, or THBS4. In certain embodiments, the composition comprises IGF2, IL-15, POSTN, and a polypeptide comprising VTN, POSTN, FGF17, THBS2, THBS1, IGF2, IL-15, or THBS4. In certain embodiments, the composition comprises IGF2, IL-15, FGF17, and a polypeptide comprising VTN, POSTN, FGF17, THBS2, THBS1, IGF2, IL-15, or THBS4. In certain embodiments, the composition comprises IGF2, IL-15, THBS2, and a polypeptide comprising VTN, POSTN, FGF17, THBS2, THBS1, IGF2, IL-15, or THBS4 In certain embodiments, the composition comprises IGF2, IL-15, THBS1, and a polypeptide comprising VTN, POSTN, FGF17, THBS2, THBS1, IGF2, IL-15, or THBS4. In certain embodiments, the composition comprises IGF2, IL-15, THBS4, and a polypeptide comprising VTN, POSTN, FGF17, THBS2, THBS1, IGF2, IL-15, or THBS4.

In certain embodiments, the composition comprises IGF2, THBS4, VTN, and a polypeptide comprising VTN, POSTN, FGF17, THBS2, THBS1, IGF2, IL-15, or THBS4. In certain embodiments, the composition comprises IGF2, THBS4, POSTN, and a polypeptide comprising VTN, POSTN, FGF17, THBS2, THBS1, IGF2, IL-15, or THBS4. In certain embodiments, the composition comprises IGF2, THBS4, FGF17, and a polypeptide comprising VTN, POSTN, FGF17, THBS2, THBS1, IGF2, IL-15, or THBS4. In certain embodiments, the composition comprises IGF2, THBS4, THBS2, and a polypeptide comprising VTN, POSTN, FGF17, THBS2, THBS1, IGF2, IL-15, or THBS4. In certain embodiments, the composition comprises IGF2, THBS4, THBS1, and a polypeptide comprising VTN, POSTN, FGF17, THBS2, THBS1, IGF2, IL-15, or THBS4 in certain embodiments, the composition comprises IGF2, THBS4, IL-15, and a polypeptide comprising VTN, POSTN, FGF17, THBS2, THBS1, IGF2, IL-15, or THBS4.

In certain embodiments, the composition comprises IL-15, THBS4, VTN, and a polypeptide comprising VTN, POSTN, FGF17, THBS2, THBS1, IGF2, IL-15, or THBS4. In certain embodiments, the composition comprises IL-15, THBS4, POSTN, and a polypeptide comprising VTN, POSTN, FGF17, THBS2, THBS1, IGF2, IL-15, or THBS4. In certain embodiments, the composition comprises IL-15, THBS4, FGF17, and a poly peptide comprising VTN, POSTN, FGF17, THBS2, THBS1, IGF2, IL-15, or THBS4. In certain embodiments, the composition comprises IL-15, THBS4, THBS2, and a polypeptide comprising VTN, POSTN, FGF17, THBS2, THBS1, IGF2, IL-15, or THBS4. In certain embodiments, the composition comprises IL-15, THBS4, THBS1, and a polypeptide comprising VTN, POSTN, FGF17, THBS2, THBS1, IGF2, IL-15, or THBS4. In certain embodiments, the composition comprises IL-15, THBS4, IGF2, and a polypeptide comprising VTN, POSTN, FGF17, THBS2, THBS1, IGF2, IL-15, or THBS4.

In certain embodiments, the heparin-associated binding polypeptide composition comprising any four polypeptides selected from VTN, POSTN, FGF17, THBS2, THBS1, IGF2, IL-15, and THBS4, comprises one or more additional modifications to increase stability. In certain embodiments, one or more of the heparin-associated polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the heparin-associated polypeptides is fused or conjugated to another protein to increase stability and or bioavailability. In certain embodiments, one or more of the heparin-associated polypeptides is fused with an Fc region of an immunoglobulin or with serum albumin. In certain embodiments, one or more of the heparin-associated polypeptides is present in a concatemer with one, two, three, four or more distinct polypeptides selected from THBS2, VTN, POSTN, FGF17, THBS1, IL-15, IGF2, and THBS4 In certain embodiments, one or more of the heparin-associated polypeptides is present in a concatemer with one, two, three, four, or more distinct polypeptides. In certain embodiments, one or more of the heparin-associated polypeptides is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, the heparin-associated binding polypeptide composition comprises any three polypeptides selected from VTN, STC2, AGRN, THBS2, and FST. In certain embodiments, the composition comprises VTN, STC2, and AGRN. In certain embodiments, the composition comprises VTN, STC2, and THBS2. In certain embodiments, the composition comprises VTN, STC2, and FST. In certain embodiments, the composition comprises VTN, AGRN, and STC2 In certain embodiments, the composition comprises VTN, AGRN, and THBS2. In certain embodiments, the composition comprises VTN, AGRN, and FST. In certain embodiments, the composition comprises VTN, THBS2, and STC2. In certain embodiments, the composition comprises VTN, THBS2, and AGRN. In certain embodiments, the composition comprises VTN, THBS2, and FST. In certain embodiments, the composition comprises VTN, FST, and STC2. In certain embodiments, the composition comprises VTN, FST, and AGRN. In certain embodiments, the composition comprises VTN, FST, and THBS2. In certain embodiments, the composition comprises STC2, VTN, and FST. In certain embodiments, the composition comprises STC2, VTN, and AGRN. In certain embodiments, the composition comprises STC2, VTN, and THBS2. In certain embodiments, the composition comprises STC2, AGRN, and FST. In certain embodiments, the composition comprises STC2, AGRN, and VTN. In certain embodiments, the composition comprises STC2, AGRN, and THBS2. In certain embodiments, the composition comprises STC2, THBS2, and FST. In certain embodiments, the composition comprises STC2, THBS2, and VTN. In certain embodiments, the composition comprises STC2, THBS2, and AGRN. In certain embodiments, the composition comprises STC2, FST, and THBS2. In certain embodiments, the composition comprises STC2, FST, and VTN. In certain embodiments, the composition comprises STC2, FST, and AGRN. In certain embodiments, the composition comprises AGRN, VTN, and THBS2. In certain embodiments, the composition comprises AGRN, VTN, and FST. In certain embodiments, the composition comprises AGRN, VTN, and STC2. In certain embodiments, the composition comprises AGRN, STC2, and THBS2. In certain embodiments, the composition comprises AGRN, STC2, and FST. In certain embodiments, the composition comprises AGRN, STC2, and VTN. In certain embodiments, the composition comprises AGRN, THBS2, and STC2. In certain embodiments, the composition comprises AGRN, THBS2, and FST. In certain embodiments, the composition comprises AGRN, THBS2, and VTN. In certain embodiments, the composition comprises AGRN, FST, and STC2. In certain embodiments, the composition comprises AGRN, FST, and THBS2 In certain embodiments, the composition comprises AGRN, FST, and VTN. In certain embodiments, the composition comprises THBS2, VTN, and STC2. In certain embodiments, the composition comprises THBS2, VTN, and AGRN. In certain embodiments, the composition comprises THBS2, VTN, and FST. In certain embodiments, the composition comprises THBS2. STC2, and VTN. In certain embodiments, the composition comprises THBS2, STC2, and AGRN. In certain embodiments, the composition comprises THBS2, STC2, and FST. In certain embodiments, the composition comprises THBS2, AGRN, and VTN. In certain embodiments, the composition comprises THBS2, AGRN, and STC2 In certain embodiments, the composition comprises THBS2, AGRN, and FST. In certain embodiments, the composition comprises THBS2, FST, and VTN. In certain embodiments, the composition comprises THBS2, FST, and STC2. In certain embodiments, the composition comprises THBS2, FST, and AGRN. In certain embodiments, the composition comprises FST, VTN, and THBS2. In certain embodiments, the composition comprises FST, VTN, and STC2. In certain embodiments, the composition comprises FST, VTN, and AGRN. In certain embodiments, the composition comprises FST, STC2, and THBS2. In certain embodiments, the composition comprises FST, STC2, and VTN. In certain embodiments, the composition comprises FST, STC2, and AGRN. In certain embodiments, the composition comprises FST, AGRN, and THBS2 In certain embodiments, the composition comprises FST, AGRN, and VTN. In certain embodiments, the composition comprises FST, AGRN, and STC2. In certain embodiments, the composition comprises FST, THBS2, and AGRN. In certain embodiments, the composition comprises FST, THBS2, and VTN. In certain embodiments, the composition comprises FST. THBS2, and STC2. In certain embodiments, one or more of the heparin-associated polypeptides comprise one or more additional modifications to increase stability. In certain embodiments, one or more of the heparin-associated polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the heparin-associated polypeptide is fused or conjugated to another protein to increase stability and or bioavailability. In certain embodiments, one or more of the heparin-associated polypeptides is fused with an Fc region of an immunoglobulin or with serum albumin. In certain embodiments, one or more of the heparin-associated polypeptides is present in a concatemer with one, two, three, four or more distinct polypeptides selected from THBS2, VTN, STC2, AGRN, or FST. In certain embodiments, one or more of the heparin-associated polypeptides is present in a concatemer with one, two, three, four, or more distinct polypeptides. In certain embodiments, one or more of the heparin-associated polypeptides is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, the heparin-associated binding polypeptide composition comprises any four polypeptides selected from VTN, STC2, AGRN, THBS2, and FST. In certain embodiments, the composition comprises VTN, STC2, AGRN, and THBS2 In certain embodiments, the composition comprises VTN, STC2, AGRN, and FST. In certain embodiments, the composition comprises VTN, STC2, THBS2, and AGRN. In certain embodiments, the composition comprises VTN, STC2, THBS2, and FST. In certain embodiments, the composition comprises VTN, STC2, FST, and AGRN. In certain embodiments, the composition comprises VTN, STC2, FST, and THBS2. In certain embodiments, the composition comprises VTN, AGRN, STC2, and FST. In certain embodiments, the composition comprises VTN, AGRN, STC2, and THBS2. In certain embodiments, the composition comprises VTN, AGRN, THBS2, and FST. In certain embodiments, the composition comprises VTN, AGRN, THBS2, and STC2. In certain embodiments, the composition comprises VTN, AGRN, FST, and THBS2. In certain embodiments, the composition comprises VTN, AGRN, FST, and STC2. In certain embodiments, the composition comprises VTN, THBS2, STC2, and AGRN. In certain embodiments, the composition comprises VTN, THBS2, STC2, and FST. In certain embodiments, the composition comprises VTN, THBS2, AGRN, and STC2. In certain embodiments, the composition comprises VTN, THBS2, AGRN, and FST. In certain embodiments, the composition comprises VTN, THBS2, FST, and STC2. In certain embodiments, the composition comprises VTN, THBS2, FST, and AGRN. In certain embodiments, the composition comprises VTN, FST, STC2, and THBS2. In certain embodiments, the composition comprises VTN, FST, STC2, and AGRN. In certain embodiments, the composition comprises VTN, FST, AGRN, and THBS2. In certain embodiments, the composition comprises VTN, FST, AGRN, and STC2. In certain embodiments, the composition comprises VTN, FST, THBS2, and AGRN. In certain embodiments, the composition comprises VTN, FST, THBS2, and STC2. In certain embodiments, the composition comprises STC2, VTN, FST, and AGRN. In certain embodiments, the composition comprises STC2, VTN, FST, and THBS2. In certain embodiments, the composition comprises STC2, VTN, AGRN, and FST. In certain embodiments, the composition comprises STC2, VTN, AGRN, and THBS2. In certain embodiments, the composition comprises STC2, VTN, THBS2, and FST. In certain embodiments, the composition comprises STC2, VTN, THBS2, and AGRN. In certain embodiments, the composition comprises STC2, AGRN, FST, and THBS2. In certain embodiments, the composition comprises STC2, AGRN, FST, and VTN. In certain embodiments, the composition comprises STC2, AGRN, VTN, and THBS2 In certain embodiments, the composition comprises STC2, AGRN, VTN, and FST. In certain embodiments, the composition comprises STC2, AGRN, THBS2, and VTN. In certain embodiments, the composition comprises STC2, AGRN, THBS2, and FST. In certain embodiments, the composition comprises STC2, THBS2, FST, and VTN. In certain embodiments, the composition comprises STC2, THBS2, FST, and AGRN. In certain embodiments, the composition comprises STC2, THBS2, VTN, and FST. In certain embodiments, the composition comprises STC2, THBS2, VTN, and AGRN. In certain embodiments, the composition comprises STC2, THBS2, AGRN, and FST. In certain embodiments, the composition comprises STC2, THBS2, AGRN, and VTN. In certain embodiments, the composition comprises STC2, FST, THBS2, and AGRN. In certain embodiments, the composition comprises STC2, FST, THBS2, and VTN. In certain embodiments, the composition comprises STC2, FST, VTN, and AGRN. In certain embodiments, the composition comprises STC2, FST, VTN, and THBS2. In certain embodiments, the composition comprises STC2, FST, AGRN, and VTN. In certain embodiments, the composition comprises STC2, FST, AGRN, and THBS2. In certain embodiments, the composition comprises AGRN, VTN, THBS2, and FST. In certain embodiments, the composition comprises AGRN, VTN, THBS2, and STC2. In certain embodiments, the composition comprises AGRN, VTN, FST, and THBS2. In certain embodiments, the composition comprises AGRN, VTN, FST, and STC2. In certain embodiments, the composition comprises AGRN, VTN, STC2, and THBS2. In certain embodiments, the composition comprises AGRN, VTN, STC2, and FST. In certain embodiments, the composition comprises AGRN, STC2, THBS2, and VTN. In certain embodiments, the composition comprises AGRN, STC2, THBS2, and FST. In certain embodiments, the composition comprises AGRN, STC2, FST, and VTN. In certain embodiments, the composition comprises AGRN, STC2, FST, and THBS2 In certain embodiments, the composition comprises AGRN, STC2, VTN, and FST. In certain embodiments, the composition comprises AGRN, STC2, VTN, and THBS2. In certain embodiments, the composition comprises AGRN, THBS2, STC2, and FST. In certain embodiments, the composition comprises AGRN, THBS2, STC2, and VTN. In certain embodiments, the composition comprises AGRN, THBS2, FST, and STC2. In certain embodiments, the composition comprises AGRN, THBS2, FST, and VTN. In certain embodiments, the composition comprises AGRN, THBS2, VTN, and STC2. In certain embodiments, the composition comprises AGRN, THBS2, VTN, and FST. In certain embodiments, the composition comprises AGRN, FST, STC2, and VTN. In certain embodiments, the composition comprises AGRN, FST, STC2, and THBS2. In certain embodiments, the composition comprises AGRN, FST, THBS2, and VTN. In certain embodiments, the composition comprises AGRN, FST, THBS2, and STC2. In certain embodiments, the composition comprises AGRN, FST, VTN, and THBS2 In certain embodiments, the composition comprises AGRN, FST, VTN, and STC2. In certain embodiments, the composition comprises THBS2, VTN, STC2, and AGRN. In certain embodiments, the composition comprises THBS2, VTN, STC2, and FST. In certain embodiments, the composition comprises THBS2, VTN, AGRN, and STC2. In certain embodiments, the composition comprises THBS2, VTN, AGRN, and FST. In certain embodiments, the composition comprises THBS2, VIN, FST, and STC2. In certain embodiments, the composition comprises THBS2, VTN, FST, and AGRN. In certain embodiments, the composition comprises THBS2, STC2, VTN, and FST. In certain embodiments, the composition comprises THBS2, STC2, VTN, and AGRN. In certain embodiments, the composition comprises THBS2, STC2, AGRN, and FST. In certain embodiments, the composition comprises THBS2, STC2, AGRN, and VTN. In certain embodiments, the composition comprises THBS2, STC2, FST, and AGRN. In certain embodiments, the composition comprises THBS2, STC2, FST, and VTN. In certain embodiments, the composition comprises THBS2, AGRN. VTN, and STC2. In certain embodiments, the composition comprises THBS2, AGRN, VTN, and FST. In certain embodiments, the composition comprises THBS2, AGRN, STC2, and VTN. In certain embodiments, the composition comprises THBS2, AGRN, STC2, and FST. In certain embodiments, the composition comprises THBS2, AGRN, FST, and VTN. In certain embodiments, the composition comprises THBS2, AGRN, FST, and STC2. In certain embodiments, the composition comprises THBS2, FST. VTN, and AGRN. In certain embodiments, the composition comprises THBS2, FST, VTN, and STC2. In certain embodiments, the composition comprises THBS2, FST, STC2, and AGRN. In certain embodiments, the composition comprises THBS2, FST, STC2, and VTN. In certain embodiments, the composition comprises THBS2, FST, AGRN, and STC2. In certain embodiments, the composition comprises THBS2, FST, AGRN, and VTN. In certain embodiments, the composition comprises FST, VTN, THBS2, and STC2. In certain embodiments, the composition comprises FST, VTN, THBS2, and AGRN. In certain embodiments, the composition comprises FST, VTN, STC2, and THBS2. In certain embodiments, the composition comprises FST, VTN, STC2, and AGRN. In certain embodiments, the composition comprises FST, VTN, AGRN, and THBS2. In certain embodiments, the composition comprises FST, VTN, AGRN, and STC2. In certain embodiments, the composition comprises FST, STC2, THBS2, and AGRN. In certain embodiments, the composition comprises FST, STC2, THBS2, and VTN. In certain embodiments, the composition comprises FST, STC2, VTN, and AGRN. In certain embodiments, the composition comprises FST, STC2, VTN, and THBS2. In certain embodiments, the composition comprises FST, STC2, AGRN, and VTN. In certain embodiments, the composition comprises FST, STC2, AGRN, and THBS2. In certain embodiments, the composition comprises FST, AGRN, THBS2, and VTN. In certain embodiments, the composition comprises FST, AGRN, THBS2, and STC2. In certain embodiments, the composition comprises FST, AGRN, VTN, and THBS2. In certain embodiments, the composition comprises FST, AGRN, VTN, and STC2. In certain embodiments, the composition comprises FST, AGRN, STC2, and THBS2. In certain embodiments, the composition comprises FST, AGRN, STC2, and VTN. In certain embodiments, the composition comprises FST, THBS2, AGRN, and STC2. In certain embodiments, the composition comprises FST, THBS2, AGRN, and VTN. In certain embodiments, the composition comprises FST, THBS2, VTN, and STC2. In certain embodiments, the composition comprises FST, THBS2, VTN, and AGRN. In certain embodiments, the composition comprises FST, THBS2, STC2, and VTN. In certain embodiments, the composition comprises FST, THBS2, STC2, and AGRN. In certain embodiments, one or more of the heparin-associated polypeptides comprise one or more additional modifications to increase stability. In certain embodiments, one or more of the heparin-associated polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the heparin-associated polypeptides is fused or conjugated to another protein to increase stability and or bioavailability. In certain embodiments, one or more of the heparin-associated polypeptides is fused with an Fc region of an immunoglobulin or with serum albumin. In certain embodiments, one or more of the heparin-associated polypeptides is present in a concatemer with one, two, three, or four other distinct polypeptides selected from THBS2, VTN, STC2, AGRN, or FST. In certain embodiments, one or more of the heparin-associated polypeptides is present in a concatemer with one, two, three, four, or more distinct polypeptides. In certain embodiments, one or more of the heparin-associated polypeptides is included in a composition comprising a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In some embodiments, a composition herein comprises polypeptide 1 and one or more polypeptides from Table 2. In some embodiments, a composition herein comprises polypeptide 2 and one or more polypeptides from Table 2. In some embodiments, a composition herein comprises polypeptide 3 and one or more polypeptides from Table 2. In some embodiments, a composition herein comprises polypeptide 4 and one or more polypeptides from Table 2 In some embodiments, a composition herein comprises polypeptide 5 and one or more polypeptides from Table 2. In some embodiments, a composition herein comprises polypeptide 6 and one or more polypeptides from Table 2. In some embodiments, a composition herein comprises polypeptide 7 and one or more polypeptides from Table 2. In some embodiments, a composition herein comprises polypeptide 8 and one or more polypeptides from Table 2. In some embodiments, a composition herein comprises polypeptide 9 and one or more polypeptides from Table 2. In some embodiments, a composition herein comprises polypeptide 10 and one or more polypeptides from Table 2. In some embodiments, a composition herein comprises polypeptide 11 and one or more polypeptides from Table 2. In some embodiments, a composition herein comprises polypeptide 12 and one or more polypeptides from Table 2. In some embodiments, a composition herein comprises polypeptide 13 and one or more polypeptides from Table 2 In some embodiments, a composition herein comprises polypeptide 14 and one or more polypeptides from Table 2. In some embodiments, a composition herein comprises polypeptide 15 and one or more polypeptides from Table 2. In some embodiments, a composition herein comprises polypeptide 16 and one or more polypeptides from Table 2 In some embodiments, a composition herein comprises polypeptide 17 and one or more polypeptides from Table 2. In some embodiments, a composition herein comprises polypeptide 18 and one or more polypeptides from Table 2. In some embodiments, a composition herein comprises polypeptide 19 and one or more polypeptides from Table 2. In some embodiments, a composition herein comprises polypeptide 21 and one or more polypeptides from Table 2. In some embodiments, a composition herein comprises polypeptide 22 and one or more polypeptides from Table 2. In some embodiments, a composition herein comprises polypeptide 23 and one or more polypeptides from Table 2. In some embodiments, a composition herein comprises polypeptide 24 and one or more polypeptides from Table 2. In some embodiments, a composition herein comprises polypeptide 25 and one or more polypeptides from Table 2. In some embodiments, a composition herein comprises polypeptide 26 and one or more polypeptides from Table 2 In some embodiments, a composition herein comprises polypeptide 27 and one or more polypeptides from Table 2. In some embodiments, a composition herein comprises polypeptide 28 and one or more polypeptides from Table 2. In some embodiments, a composition herein comprises polypeptide 29 and one or more polypeptides from Table 2 In some embodiments, a composition herein comprises polypeptide 30 and one or more polypeptides from Table 2. In some embodiments, a composition herein comprises polypeptide 31 and one or more polypeptides from Table 2. In some embodiments, a composition herein comprises polypeptide 32 and one or more polypeptides from Table 2. In some embodiments, a composition herein comprises polypeptide 33 and one or more polypeptides from Table 2. In some embodiments, a composition herein comprises polypeptide 34 and one or more polypeptides from Table 2. In some embodiments, a composition herein comprises polypeptide 35 and one or more polypeptides from Table 2. In some embodiments, a composition herein comprises polypeptide 36 and one or more polypeptides from Table 2. In some embodiments, a composition herein comprises polypeptide 37 and one or more polypeptides from Table 2. In some embodiments, a composition herein comprises polypeptide 38 and one or more polypeptides from Table 2 In some embodiments, a composition herein comprises polypeptide 39 and one or more polypeptides from Table 2. In some embodiments, a composition herein comprises polypeptide 40 and one or more polypeptides from Table 2. In some embodiments, a composition herein comprises polypeptide 41 and one or more polypeptides from Table 2. In some embodiments, a composition herein comprises polypeptide 42 and one or more polypeptides from Table 2. In some embodiments, a composition herein comprises polypeptide 43 and one or more polypeptides from Table 2. In some embodiments, a composition herein comprises polypeptide 44 and one or more polypeptides from Table 2. In some embodiments, a composition herein comprises polypeptide 45 and one or more polypeptides from Table 2. In some embodiments, a composition herein comprises polypeptide 46 and one or more polypeptides from Table 2. In some embodiments, a composition herein comprises polypeptide 47 and one or more polypeptides from Table 2. In some embodiments, a composition herein comprises polypeptide 48 and one or more polypeptides from Table 2. In some embodiments, a composition herein comprises polypeptide 49 and one or more polypeptides from Table 2. In some embodiments, a composition herein comprises polypeptide 50 and one or more polypeptides from Table 2 In some embodiments, a composition herein comprises polypeptide 51 and one or more polypeptides from Table 2. In some embodiments, a composition herein comprises polypeptide 52 and one or more polypeptides from Table 2. In some embodiments, a composition herein comprises polypeptide 53 and one or more polypeptides from Table 2. In some embodiments, a composition herein comprises polypeptide 54 and one or more polypeptides from Table 2. In some embodiments, a composition herein comprises polypeptide 55 and one or more polypeptides from Table 2. In some embodiments, a composition herein comprises polypeptide 56 and one or more polypeptides from Table 2. In some embodiments, a composition herein comprises polypeptide 57 and one or more polypeptides from Table 2. In some embodiments, a composition herein comprises polypeptide 58 and one or more polypeptides from Table 2. In some embodiments, a composition herein comprises polypeptide 59 and one or more polypeptides from Table 2. In some embodiments, a composition herein comprises polypeptide 60 and one or more polypeptides from Table 2. In some embodiments, a composition herein comprises polypeptide 61 and one or more polypeptides from Table 2. In some embodiments, a composition herein comprises polypeptide 62 and one or more polypeptides from Table 2. In some embodiments, a composition herein comprises polypeptide 63 and one or more polypeptides from Table 2. In some embodiments, a composition herein comprises polypeptide 64 and one or more polypeptides from Table 2. In some embodiments, a composition herein comprises polypeptide 65 and one or more polypeptides from Table 2. In some embodiments, a composition herein comprises polypeptide 66 and one or more polypeptides from Table 2. In some embodiments, a composition herein comprises polypeptide 67 and one or more polypeptides from Table 2. In some embodiments, a composition herein comprises polypeptide 68 and one or more polypeptides from Table 2. In some embodiments, a composition herein comprises polypeptide 69 and one or more polypeptides from Table 2. In some embodiments, a composition herein comprises polypeptide 70 and one or more polypeptides from Table 2. In some embodiments, a composition herein comprises polypeptide 71 and one or more polypeptides from Table 2. In some embodiments, a composition herein comprises polypeptide 72 and one or more polypeptides from Table 2. In some embodiments, a composition herein comprises polypeptide 73 and one or more polypeptides from Table 2. In some embodiments, a composition herein comprises polypeptide 74 and one or more polypeptides from Table 2. In some embodiments, a composition herein comprises polypeptide 75 and one or more polypeptides from Table 2. In some embodiments, a composition herein comprises polypeptide 76 and one or more polypeptides from Table 2. In some embodiments, a composition herein comprises polypeptide 77 and one or more polypeptides from Table 2. In some embodiments, a composition herein comprises polypeptide 78 and one or more polypeptides from Table 2. In some embodiments, a composition herein comprises polypeptide 79 and one or more polypeptides from Table 2. In some embodiments, a composition herein comprises polypeptide 80 and one or more polypeptides from Table 2. In some embodiments, a composition herein comprises polypeptide 81 and one or more polypeptides from Table 2. In some embodiments, a composition herein comprises polypeptide 82 and one or more polypeptides from Table 2. In some embodiments, a composition herein comprises polypeptide 83 and one or more polypeptides from Table 2. In some embodiments, a composition herein comprises polypeptide 84 and one or more polypeptides from Table 2. In some embodiments, a composition herein comprises polypeptide 85 and one or more polypeptides from Table 2. In some embodiments, a composition herein comprises polypeptide 86 and one or more polypeptides from Table 2. In some embodiments, a composition herein comprises polypeptide 87 and one or more polypeptides from Table 2. In some embodiments, a composition herein comprises polypeptide 88 and one or more polypeptides from Table 2. In some embodiments, a composition herein comprises polypeptide 89 and one or more polypeptides from Table 2. In some embodiments, a composition herein comprises polypeptide 90 and one or more polypeptides from Table 2. In some embodiments, a composition herein comprises polypeptide 91 and one or more polypeptides from Table 2. In some embodiments, a composition herein comprises polypeptide 92 and one or more polypeptides from Table 2. In some embodiments, a composition herein comprises polypeptide 93 and one or more polypeptides from Table 2. In some embodiments, a composition herein comprises polypeptide 94 and one or more polypeptides from Table 2. In some embodiments, a composition herein comprises polypeptide 95 and one or more polypeptides from Table 2 In some embodiments, a composition herein comprises polypeptide 96 and one or more polypeptides from Table 2. In some embodiments, a composition herein comprises polypeptide 97 and one or more polypeptides from Table 2. In some embodiments, a composition herein comprises polypeptide 98 and one or more polypeptides from Table 2. In some embodiments, a composition herein comprises polypeptide 99 and one or more polypeptides from Table 2. In some embodiments, a composition herein comprises polypeptide 100 and one or more polypeptides from Table 2.

in some embodiments, a composition herein comprises polypeptide 101 and one or more polypeptides from Table 2. In some embodiments, a composition herein comprises polypeptide 102 and one or more polypeptides from Table 2. In some embodiments, a composition herein comprises polypeptide 103 and one or more polypeptides from Table 2. In some embodiments, a composition herein comprises polypeptide 104 and one or more polypeptides from Table 2. In some embodiments, a composition herein comprises polypeptide 105 and one or more polypeptides from Table 2. In some embodiments, a composition herein comprises polypeptide 106 and one or more polypeptides from Table 2. In some embodiments, a composition herein comprises polypeptide 107 and one or more polypeptides from Table 2. In some embodiments, a composition herein comprises polypeptide 108 and one or more polypeptides from Table 2. In some embodiments, a composition herein comprises polypeptide 109 and one or more polypeptides from Table 2. In some embodiments, a composition herein comprises polypeptide 110 and one or more polypeptides from Table 2. In some embodiments, a composition herein comprises polypeptide 111 and one or more polypeptides from Table 2. In some embodiments, a composition herein comprises polypeptide 112 and one or more polypeptides from Table 2. In some embodiments, a composition herein comprises polypeptide 113 and one or more polypeptides from Table 2. In some embodiments, a composition herein comprises polypeptide 114 and one or more polypeptides from Table 2 in some embodiments, a composition herein comprises polypeptide 115 and one or more polypeptides from Table 2. In some embodiments, a composition herein comprises polypeptide 116 and one or more polypeptides from Table 2. In some embodiments, a composition herein comprises polypeptide 117 and one or more polypeptides from Table 2. In some embodiments, a composition herein comprises polypeptide 118 and one or more polypeptides from Table 2. In some embodiments, a composition herein comprises polypeptide 119 and one or more polypeptides from Table 2. In some embodiments, a composition herein comprises polypeptide 121 and one or more polypeptides from Table 2. In some embodiments, a composition herein comprises polypeptide 122 and one or more polypeptides from Table 2. In some embodiments, a composition herein comprises polypeptide 123 and one or more polypeptides from Table 2. In some embodiments, a composition herein comprises polypeptide 124 and one or more polypeptides from Table 2. In some embodiments, a composition herein comprises polypeptide 125 and one or more polypeptides from Table 2. In some embodiments, a composition herein comprises polypeptide 126 and one or more polypeptides from Table 2. In some embodiments, a composition herein comprises polypeptide 127 and one or more polypeptides from Table 2. In some embodiments, a composition herein comprises polypeptide 128 and one or more polypeptides from Table 2. In some embodiments, a composition herein comprises polypeptide 129 and one or more polypeptides from Table 2. In some embodiments, a composition herein comprises polypeptide 130 and one or more polypeptides from Table 2 In some embodiments, a composition herein comprises polypeptide 131 and one or more polypeptides from Table 2. In some embodiments, a composition herein comprises polypeptide 132 and one or more polypeptides from Table 2 In some embodiments, a composition herein comprises polypeptide 133 and one or more polypeptides from Table 2. In some embodiments, a composition herein comprises polypeptide 134 and one or more polypeptides from Table 2. In some embodiments, a composition herein comprises polypeptide 135 and one or more polypeptides from Table 2. In some embodiments, a composition herein comprises polypeptide 136 and one or more polypeptides from Table 2. In some embodiments, a composition herein comprises polypeptide 137 and one or more polypeptides from Table 2. In some embodiments, a composition herein comprises polypeptide 138 and one or more polypeptides from Table 2. In some embodiments, a composition herein comprises polypeptide 139 and one or more polypeptides from Table 2. In some embodiments, a composition herein comprises polypeptide 140 and one or more polypeptides from Table 2. In some embodiments, a composition herein comprises polypeptide 141 and one or more polypeptides from Table 2. In some embodiments, a composition herein comprises polypeptide 142 and one or more polypeptides from Table 2. In some embodiments, a composition herein comprises polypeptide 143 and one or more polypeptides from Table 2. In some embodiments, a composition herein comprises polypeptide 144 and one or more polypeptides from Table 2. In some embodiments, a composition herein comprises polypeptide 145 and one or more polypeptides from Table 2. In some embodiments, a composition herein comprises polypeptide 146 and one or more polypeptides from Table 2. In some embodiments, a composition herein comprises polypeptide 147 and one or more polypeptides from Table 2 In some embodiments, a composition herein comprises polypeptide 148 and one or more polypeptides from Table 2. In some embodiments, a composition herein comprises polypeptide 149 and one or more polypeptides from Table 2. In some embodiments, a composition herein comprises polypeptide 150 and one or more polypeptides from Table 2. In some embodiments, a composition herein comprises polypeptide 151 and one or more polypeptides from Table 2. In some embodiments, a composition herein comprises polypeptide 152 and one or more polypeptides from Table 2. In some embodiments, a composition herein comprises polypeptide 153 and one or more polypeptides from Table 2. In some embodiments, a composition herein comprises polypeptide 154 and one or more polypeptides from Table 2. In some embodiments, a composition herein comprises polypeptide 155 and one or more polypeptides from Table 2. In some embodiments, a composition herein comprises polypeptide 156 and one or more polypeptides from Table 2. In some embodiments, a composition herein comprises polypeptide 157 and one or more polypeptides from Table 2. In some embodiments, a composition herein comprises polypeptide 158 and one or more polypeptides from Table 2. In some embodiments, a composition herein comprises polypeptide 159 and one or more polypeptides from Table 2. In some embodiments, a composition herein comprises polypeptide 160 and one or more polypeptides from Table 2. In some embodiments, a composition herein comprises polypeptide 161 and one or more polypeptides from Table 2. In some embodiments, a composition herein comprises polypeptide 162 and one or more polypeptides from Table 2 in some embodiments, a composition herein comprises polypeptide 163 and one or more polypeptides from Table 2. In some embodiments, a composition herein comprises polypeptide 164 and one or more polypeptides from Table 2. In some embodiments, a composition herein comprises polypeptide 165 and one or more polypeptides from Table 2 In some embodiments, a composition herein comprises polypeptide 166 and one or more polypeptides from Table 2. In some embodiments, a composition herein comprises polypeptide 167 and one or more polypeptides from Table 2. In some embodiments, a composition herein comprises polypeptide 168 and one or more polypeptides from Table 2. In some embodiments, a composition herein comprises polypeptide 169 and one or more polypeptides from Table 2. In some embodiments, a composition herein comprises polypeptide 170 and one or more polypeptides from Table 2. In some embodiments, a composition herein comprises polypeptide 171 and one or more polypeptides from Table 2. In some embodiments, a composition herein comprises polypeptide 172 and one or more polypeptides from Table 2. In some embodiments, a composition herein comprises polypeptide 173 and one or more polypeptides from Table 2. In some embodiments, a composition herein comprises polypeptide 174 and one or more polypeptides from Table 2. In some embodiments, a composition herein comprises polypeptide 175 and one or more polypeptides from Table 2. In some embodiments, a composition herein comprises polypeptide 176 and one or more polypeptides from Table 2. In some embodiments, a composition herein comprises polypeptide 177 and one or more polypeptides from Table 2 in some embodiments, a composition herein comprises polypeptide 178 and one or more polypeptides from Table 2. In some embodiments, a composition herein comprises polypeptide 179 and one or more polypeptides from Table 2. In some embodiments, a composition herein comprises polypeptide 180 and one or more polypeptides from Table 2 In some embodiments, a composition herein comprises polypeptide 181 and one or more polypeptides from Table 2. In some embodiments, a composition herein comprises polypeptide 182 and one or more polypeptides from Table 2. In some embodiments, a composition herein comprises polypeptide 183 and one or more polypeptides from Table 2. In some embodiments, a composition herein comprises polypeptide 184 and one or more polypeptides from Table 2. In some embodiments, a composition herein comprises polypeptide 185 and one or more polypeptides from Table 2. In some embodiments, a composition herein comprises polypeptide 186 and one or more polypeptides from Table 2. In some embodiments, a composition herein comprises polypeptide 187 and one or more polypeptides from Table 2. In some embodiments, a composition herein comprises polypeptide 188 and one or more polypeptides from Table 2. In some embodiments, a composition herein comprises polypeptide 189 and one or more polypeptides from Table 2. In some embodiments, a composition herein comprises polypeptide 190 and one or more polypeptides from Table 2. In some embodiments, a composition herein comprises polypeptide 191 and one or more polypeptides from Table 2. In some embodiments, a composition herein comprises polypeptide 192 and one or more polypeptides from Table 2. In some embodiments, a composition herein comprises polypeptide 193 and one or more polypeptides from Table 2. In some embodiments, a composition herein comprises polypeptide 194 and one or more polypeptides from Table 2. In some embodiments, a composition herein comprises polypeptide 195 and one or more polypeptides from Table 2 in some embodiments, a composition herein comprises polypeptide 196 and one or more polypeptides from Table 2. In some embodiments, a composition herein comprises polypeptide 197 and one or more polypeptides from Table 2. In some embodiments, a composition herein comprises polypeptide 198 and one or more polypeptides from Table 2. In some embodiments, a composition herein comprises polypeptide 199 and one or more polypeptides from Table 2. In some embodiments, a composition herein comprises polypeptide 200 and one or more polypeptides from Table 2.

In some embodiments, a composition herein comprises polypeptide 201 and one or more polypeptides from Table 2. In some embodiments, a composition herein comprises polypeptide 202 and one or more polypeptides from Table 2. In some embodiments, a composition herein comprises polypeptide 203 and one or more polypeptides from Table 2. In some embodiments, a composition herein comprises polypeptide 204 and one or more polypeptides from Table 2. In some embodiments, a composition herein comprises polypeptide 205 and one or more polypeptides from Table 2. In some embodiments, a composition herein comprises polypeptide 206 and one or more polypeptides from Table 2. In some embodiments, a composition herein comprises polypeptide 207 and one or more polypeptides from Table 2. In some embodiments, a composition herein comprises polypeptide 208 and one or more polypeptides from Table 2. In some embodiments, a composition herein comprises polypeptide 209 and one or more polypeptides from Table 2. In some embodiments, a composition herein comprises polypeptide 210 and one or more polypeptides from Table 2. In some embodiments, a composition herein comprises polypeptide 211 and one or more polypeptides from Table 2. In some embodiments, a composition herein comprises polypeptide 212 and one or more polypeptides from Table 2. In some embodiments, a composition herein comprises polypeptide 213 and one or more polypeptides from Table 2. In some embodiments, a composition herein comprises polypeptide 214 and one or more polypeptides from Table 2 in some embodiments, a composition herein comprises polypeptide 215 and one or more polypeptides from Table 2. In some embodiments, a composition herein comprises polypeptide 216 and one or more polypeptides from Table 2. In some embodiments, a composition herein comprises polypeptide 217 and one or more polypeptides from Table 2. In some embodiments, a composition herein comprises polypeptide 218 and one or more polypeptides from Table 2. In some embodiments, a composition herein comprises polypeptide 219 and one or more polypeptides from Table 2. In some embodiments, a composition herein comprises polypeptide 221 and one or more polypeptides from Table 2. In some embodiments, a composition herein comprises polypeptide 222 and one or more polypeptides from Table 2. In some embodiments, a composition herein comprises polypeptide 223 and one or more polypeptides from Table 2. In some embodiments, a composition herein comprises polypeptide 224 and one or more polypeptides from Table 2. In some embodiments, a composition herein comprises polypeptide 225 and one or more polypeptides from Table 2. In some embodiments, a composition herein comprises polypeptide 226 and one or more polypeptides from Table 2. In some embodiments, a composition herein comprises polypeptide 227 and one or more polypeptides from Table 2. In some embodiments, a composition herein comprises polypeptide 228 and one or more polypeptides from Table 2. In some embodiments, a composition herein comprises polypeptide 229 and one or more polypeptides from Table 2. In some embodiments, a composition herein comprises polypeptide 230 and one or more polypeptides from Table 2 in some embodiments, a composition herein comprises polypeptide 231 and one or more polypeptides from Table 2. In some embodiments, a composition herein comprises polypeptide 232 and one or more polypeptides from Table 2 In some embodiments, a composition herein comprises polypeptide 233 and one or more polypeptides from Table 2. In some embodiments, a composition herein comprises polypeptide 234 and one or more polypeptides from Table 2. In some embodiments, a composition herein comprises polypeptide 235 and one or more polypeptides from Table 2. In some embodiments, a composition herein comprises polypeptide 236 and one or more polypeptides from Table 2. In some embodiments, a composition herein comprises polypeptide 237 and one or more polypeptides from Table 2. In some embodiments, a composition herein comprises polypeptide 238 and one or more polypeptides from Table 2. In some embodiments, a composition herein comprises polypeptide 239 and one or more polypeptides from Table 2. In some embodiments, a composition herein comprises polypeptide 240 and one or more polypeptides from Table 2. In some embodiments, a composition herein comprises polypeptide 241 and one or more polypeptides from Table 2. In some embodiments, a composition herein comprises polypeptide 242 and one or more polypeptides from Table 2. In some embodiments, a composition herein comprises polypeptide 243 and one or more polypeptides from Table 2. In some embodiments, a composition herein comprises polypeptide 244 and one or more polypeptides from Table 2. In some embodiments, a composition herein comprises polypeptide 245 and one or more polypeptides from Table 2. In some embodiments, a composition herein comprises polypeptide 246 and one or more polypeptides from Table 2. In some embodiments, a composition herein comprises polypeptide 247 and one or more polypeptides from Table 2 In some embodiments, a composition herein comprises polypeptide 248 and one or more polypeptides from Table 2. In some embodiments, a composition herein comprises polypeptide 249 and one or more polypeptides from Table 2. In some embodiments, a composition herein comprises polypeptide 250 and one or more polypeptides from Table 2. In some embodiments, a composition herein comprises polypeptide 251 and one or more polypeptides from Table 2. In some embodiments, a composition herein comprises polypeptide 252 and one or more polypeptides from Table 2. In some embodiments, a composition herein comprises polypeptide 253 and one or more polypeptides from Table 2. In some embodiments, a composition herein comprises polypeptide 254 and one or more polypeptides from Table 2. In some embodiments, a composition herein comprises polypeptide 255 and one or more polypeptides from Table 2. In some embodiments, a composition herein comprises polypeptide 256 and one or more polypeptides from Table 2. In some embodiments, a composition herein comprises polypeptide 257 and one or more polypeptides from Table 2. In some embodiments, a composition herein comprises polypeptide 258 and one or more polypeptides from Table 2. In some embodiments, a composition herein comprises polypeptide 259 and one or more polypeptides from Table 2. In some embodiments, a composition herein comprises polypeptide 260 and one or more polypeptides from Table 2. In some embodiments, a composition herein comprises polypeptide 261 and one or more polypeptides from Table 2. In some embodiments, a composition herein comprises polypeptide 262 and one or more polypeptides from Table 2 in some embodiments, a composition herein comprises polypeptide 263 and one or more polypeptides from Table 2. In some embodiments, a composition herein comprises polypeptide 264 and one or more polypeptides from Table 2. In some embodiments, a composition herein comprises polypeptide 265 and one or more polypeptides from Table 2 In some embodiments, a composition herein comprises polypeptide 266 and one or more polypeptides from Table 2. In some embodiments, a composition herein comprises polypeptide 267 and one or more polypeptides from Table 2. In some embodiments, a composition herein comprises polypeptide 268 and one or more polypeptides from Table 2. In some embodiments, a composition herein comprises polypeptide 269 and one or more polypeptides from Table 2. In some embodiments, a composition herein comprises polypeptide 270 and one or more polypeptides from Table 2. In some embodiments, a composition herein comprises polypeptide 271 and one or more polypeptides from Table 2. In some embodiments, a composition herein comprises polypeptide 272 and one or more polypeptides from Table 2. In some embodiments, a composition herein comprises polypeptide 273 and one or more polypeptides from Table 2. In some embodiments, a composition herein comprises polypeptide 274 and one or more polypeptides from Table 2. In some embodiments, a composition herein comprises polypeptide 275 and one or more polypeptides from Table 2. In some embodiments, a composition herein comprises polypeptide 276 and one or more polypeptides from Table 2. In some embodiments, a composition herein comprises polypeptide 277 and one or more polypeptides from Table 2 in some embodiments, a composition herein comprises polypeptide 278 and one or more polypeptides from Table 2. In some embodiments, a composition herein comprises polypeptide 279 and one or more polypeptides from Table 2. In some embodiments, a composition herein comprises polypeptide 280 and one or more polypeptides from Table 2 In some embodiments, a composition herein comprises polypeptide 281 and one or more polypeptides from Table 2. In some embodiments, a composition herein comprises polypeptide 282 and one or more polypeptides from Table 2. In some embodiments, a composition herein comprises polypeptide 283 and one or more polypeptides from Table 2. In some embodiments, a composition herein comprises polypeptide 284 and one or more polypeptides from Table 2. In some embodiments, a composition herein comprises polypeptide 285 and one or more polypeptides from Table 2. In some embodiments, a composition herein comprises polypeptide 286 and one or more polypeptides from Table 2. In some embodiments, a composition herein comprises polypeptide 287 and one or more polypeptides from Table 2. In some embodiments, a composition herein comprises polypeptide 288 and one or more polypeptides from Table 2. In some embodiments, a composition herein comprises polypeptide 289 and one or more polypeptides from Table 2. In some embodiments, a composition herein comprises polypeptide 290 and one or more polypeptides from Table 2. In some embodiments, a composition herein comprises polypeptide 291 and one or more polypeptides from Table 2. In some embodiments, a composition herein comprises polypeptide 292 and one or more polypeptides from Table 2. In some embodiments, a composition herein comprises polypeptide 293 and one or more polypeptides from Table 2. In some embodiments, a composition herein comprises polypeptide 294 and one or more polypeptides from Table 2. In some embodiments, a composition herein comprises polypeptide 295 and one or more polypeptides from Table 2 in some embodiments, a composition herein comprises polypeptide 296 and one or more polypeptides from Table 2. In some embodiments, a composition herein comprises polypeptide 297 and one or more polypeptides from Table 2. In some embodiments, a composition herein comprises polypeptide 298 and one or more polypeptides from Table 2. In some embodiments, a composition herein comprises polypeptide 299 and one or more polypeptides from Table 2. In some embodiments, a composition herein comprises polypeptide 300 and one or more polypeptides from Table 2.

In some embodiments, a composition herein comprises polypeptide 401 and one or more polypeptides from Table 2. In some embodiments, a composition herein comprises polypeptide 402 and one or more polypeptides from Table 2. In some embodiments, a composition herein comprises polypeptide 403 and one or more polypeptides from Table 2. In some embodiments, a composition herein comprises polypeptide 404 and one or more polypeptides from Table 2. In some embodiments, a composition herein comprises polypeptide 405 and one or more polypeptides from Table 2. In some embodiments, a composition herein comprises polypeptide 406 and one or more polypeptides from Table 2. In some embodiments, a composition herein comprises polypeptide 407 and one or more polypeptides from Table 2. In some embodiments, a composition herein comprises polypeptide 408 and one or more polypeptides from Table 2. In some embodiments, a composition herein comprises polypeptide 409 and one or more polypeptides from Table 2. In some embodiments, a composition herein comprises polypeptide 410 and one or more polypeptides from Table 2. In some embodiments, a composition herein comprises polypeptide 411 and one or more polypeptides from Table 2 in some embodiments, a composition herein comprises polypeptide 412 and one or more polypeptides from Table 2. In some embodiments, a composition herein comprises polypeptide 413 and one or more polypeptides from Table 2. In some embodiments, a composition herein comprises polypeptide 414 and one or more polypeptides from Table 2 In some embodiments, a composition herein comprises polypeptide 415 and one or more polypeptides from Table 2. In some embodiments, a composition herein comprises polypeptide 416 and one or more polypeptides from Table 2. In some embodiments, a composition herein comprises polypeptide 417 and one or more polypeptides from Table 2. In some embodiments, a composition herein comprises polypeptide 418 and one or more polypeptides from Table 2. In some embodiments, a composition herein comprises polypeptide 419 and one or more polypeptides from Table 2. In some embodiments, a composition herein comprises polypeptide 421 and one or more polypeptides from Table 2. In some embodiments, a composition herein comprises polypeptide 422 and one or more polypeptides from Table 2. In some embodiments, a composition herein comprises polypeptide 423 and one or more polypeptides from Table 2. In some embodiments, a composition herein comprises polypeptide 424 and one or more polypeptides from Table 2. In some embodiments, a composition herein comprises polypeptide 425 and one or more polypeptides from Table 2. In some embodiments, a composition herein comprises polypeptide 426 and one or more polypeptides from Table 2. In some embodiments, a composition herein comprises polypeptide 427 and one or more polypeptides from Table 2. In some embodiments, a composition herein comprises polypeptide 428 and one or more polypeptides from Table 2. In some embodiments, a composition herein comprises polypeptide 429 and one or more polypeptides from Table 2. In some embodiments, a composition herein comprises polypeptide 430 and one or more polypeptides from Table 2 in some embodiments, a composition herein comprises polypeptide 431 and one or more polypeptides from Table 2. In some embodiments, a composition herein comprises polypeptide 432 and one or more polypeptides from Table 2. In some embodiments, a composition herein comprises polypeptide 433 and one or more polypeptides from Table 2. In some embodiments, a composition herein comprises polypeptide 434 and one or more polypeptides from Table 2. In some embodiments, a composition herein comprises polypeptide 435 and one or more polypeptides from Table 2. In some embodiments, a composition herein comprises polypeptide 436 and one or more polypeptides from Table 2. In some embodiments, a composition herein comprises polypeptide 437 and one or more polypeptides from Table 2. In some embodiments, a composition herein comprises polypeptide 438 and one or more polypeptides from Table 2. In some embodiments, a composition herein comprises polypeptide 439 and one or more polypeptides from Table 2. In some embodiments, a composition herein comprises polypeptide 440 and one or more polypeptides from Table 2. In some embodiments, a composition herein comprises polypeptide 441 and one or more polypeptides from Table 2. In some embodiments, a composition herein comprises polypeptide 442 and one or more polypeptides from Table 2. In some embodiments, a composition herein comprises polypeptide 443 and one or more polypeptides from Table 2. In some embodiments, a composition herein comprises polypeptide 444 and one or more polypeptides from Table 2. In some embodiments, a composition herein comprises polypeptide 445 and one or more polypeptides from Table 2 In some embodiments, a composition herein comprises polypeptide 446 and one or more polypeptides from Table 2. In some embodiments, a composition herein comprises polypeptide 447 and one or more polypeptides from Table 2 In some embodiments, a composition herein comprises polypeptide 448 and one or more polypeptides from Table 2. In some embodiments, a composition herein comprises polypeptide 449 and one or more polypeptides from Table 2. In some embodiments, a composition herein comprises polypeptide 450 and one or more polypeptides from Table 2. In some embodiments, a composition herein comprises polypeptide 451 and one or more polypeptides from Table 2. In some embodiments, a composition herein comprises polypeptide 452 and one or more polypeptides from Table 2. In some embodiments, a composition herein comprises polypeptide 453 and one or more polypeptides from Table 2. In some embodiments, a composition herein comprises polypeptide 454 and one or more polypeptides from Table 2. In some embodiments, a composition herein comprises polypeptide 455 and one or more polypeptides from Table 2. In some embodiments, a composition herein comprises polypeptide 456 and one or more polypeptides from Table 2. In some embodiments, a composition herein comprises polypeptide 457 and one or more polypeptides from Table 2. In some embodiments, a composition herein comprises polypeptide 458 and one or more polypeptides from Table 2. In some embodiments, a composition herein comprises polypeptide 459 and one or more polypeptides from Table 2. In some embodiments, a composition herein comprises polypeptide 460 and one or more polypeptides from Table 2. In some embodiments, a composition herein comprises polypeptide 461 and one or more polypeptides from Table 2. In some embodiments, a composition herein comprises polypeptide 462 and one or more polypeptides from Table 2 In some embodiments, a composition herein comprises polypeptide 463 and one or more polypeptides from Table 2. In some embodiments, a composition herein comprises polypeptide 464 and one or more polypeptides from Table 2. In some embodiments, a composition herein comprises polypeptide 465 and one or more polypeptides from Table 2. In some embodiments, a composition herein comprises polypeptide 466 and one or more polypeptides from Table 2. In some embodiments, a composition herein comprises polypeptide 467 and one or more polypeptides from Table 2. In some embodiments, a composition herein comprises polypeptide 468 and one or more polypeptides from Table 2. In some embodiments, a composition herein comprises polypeptide 469 and one or more polypeptides from Table 2. In some embodiments, a composition herein comprises polypeptide 470 and one or more polypeptides from Table 2. In some embodiments, a composition herein comprises polypeptide 471 and one or more polypeptides from Table 2. In some embodiments, a composition herein comprises polypeptide 472 and one or more polypeptides from Table 2. In some embodiments, a composition herein comprises polypeptide 473 and one or more polypeptides from Table 2. In some embodiments, a composition herein comprises polypeptide 474 and one or more polypeptides from Table 2. In some embodiments, a composition herein comprises polypeptide 475 and one or more polypeptides from Table 2. In some embodiments, a composition herein comprises polypeptide 476 and one or more polypeptides from Table 2. In some embodiments, a composition herein comprises polypeptide 477 and one or more polypeptides from Table 2 in some embodiments, a composition herein comprises polypeptide 478 and one or more polypeptides from Table 2. In some embodiments, a composition herein comprises polypeptide 479 and one or more polypeptides from Table 2. In some embodiments, a composition herein comprises polypeptide 480 and one or more polypeptides from Table 2 In some embodiments, a composition herein comprises polypeptide 481 and one or more polypeptides from Table 2. In some embodiments, a composition herein comprises polypeptide 482 and one or more polypeptides from Table 2. In some embodiments, a composition herein comprises polypeptide 483 and one or more polypeptides from Table 2. In some embodiments, a composition herein comprises polypeptide 484 and one or more polypeptides from Table 2. In some embodiments, a composition herein comprises polypeptide 485 and one or more polypeptides from Table 2. In some embodiments, a composition herein comprises polypeptide 486 and one or more polypeptides from Table 2. In some embodiments, a composition herein comprises polypeptide 487 and one or more polypeptides from Table 2. In some embodiments, a composition herein comprises polypeptide 488 and one or more polypeptides from Table 2. In some embodiments, a composition herein comprises polypeptide 489 and one or more polypeptides from Table 2. In some embodiments, a composition herein comprises polypeptide 490 and one or more polypeptides from Table 2. In some embodiments, a composition herein comprises polypeptide 491 and one or more polypeptides from Table 2. In some embodiments, a composition herein comprises polypeptide 492 and one or more polypeptides from Table 2 in some embodiments, a composition herein comprises polypeptide 493 and one or more polypeptides from Table 2. In some embodiments, a composition herein comprises polypeptide 494 and one or more polypeptides from Table 2. In some embodiments, a composition herein comprises polypeptide 495 and one or more polypeptides from Table 2 In some embodiments, a composition herein comprises polypeptide 496 and one or more polypeptides from Table 2. In some embodiments, a composition herein comprises polypeptide 497 and one or more polypeptides from Table 2. In some embodiments, a composition herein comprises polypeptide 498 and one or more polypeptides from Table 2. In some embodiments, a composition herein comprises polypeptide 499 and one or more polypeptides from Table 2. In some embodiments, a composition herein comprises polypeptide 500 and one or more polypeptides from Table 2.

In some embodiments, a composition herein comprises polypeptide 501 and one or more polypeptides from Table 2. In some embodiments, a composition herein comprises polypeptide 502 and one or more polypeptides from Table 2. In some embodiments, a composition herein comprises polypeptide 503 and one or more polypeptides from Table 2. In some embodiments, a composition herein comprises polypeptide 504 and one or more polypeptides from Table 2. In some embodiments, a composition herein comprises polypeptide 505 and one or more polypeptides from Table 2. In some embodiments, a composition herein comprises polypeptide 506 and one or more polypeptides from Table 2. In some embodiments, a composition herein comprises polypeptide 507 and one or more polypeptides from Table 2. In some embodiments, a composition herein comprises polypeptide 508 and one or more polypeptides from Table 2. In some embodiments, a composition herein comprises polypeptide 509 and one or more polypeptides from Table 2. In some embodiments, a composition herein comprises polypeptide 510 and one or more polypeptides from Table 2. In some embodiments, a composition herein comprises polypeptide 511 and one or more polypeptides from Table 2 in some embodiments, a composition herein comprises polypeptide 512 and one or more polypeptides from Table 2. In some embodiments, a composition herein comprises polypeptide 513 and one or more polypeptides from Table 2. In some embodiments, a composition herein comprises polypeptide 514 and one or more polypeptides from Table 2 In some embodiments, a composition herein comprises polypeptide 515 and one or more polypeptides from Table 2. In some embodiments, a composition herein comprises polypeptide 516 and one or more polypeptides from Table 2. In some embodiments, a composition herein comprises polypeptide 517 and one or more polypeptides from Table 2. In some embodiments, a composition herein comprises polypeptide 518 and one or more polypeptides from Table 2. In some embodiments, a composition herein comprises polypeptide 519 and one or more polypeptides from Table 2. In some embodiments, a composition herein comprises polypeptide 521 and one or more polypeptides from Table 2. In some embodiments, a composition herein comprises polypeptide 522 and one or more polypeptides from Table 2. In some embodiments, a composition herein comprises polypeptide 523 and one or more polypeptides from Table 2. In some embodiments, a composition herein comprises polypeptide 524 and one or more polypeptides from Table 2. In some embodiments, a composition herein comprises polypeptide 525 and one or more polypeptides from Table 2. In some embodiments, a composition herein comprises polypeptide 526 and one or more polypeptides from Table 2. In some embodiments, a composition herein comprises polypeptide 527 and one or more polypeptides from Table 2. In some embodiments, a composition herein comprises polypeptide 528 and one or more polypeptides from Table 2. In some embodiments, a composition herein comprises polypeptide 529 and one or more polypeptides from Table 2. In some embodiments, a composition herein comprises polypeptide 530 and one or more polypeptides from Table 2 in some embodiments, a composition herein comprises polypeptide 531 and one or more polypeptides from Table 2. In some embodiments, a composition herein comprises polypeptide 532 and one or more polypeptides from Table 2. In some embodiments, a composition herein comprises polypeptide 533 and one or more polypeptides from Table 2. In some embodiments, a composition herein comprises polypeptide 534 and one or more polypeptides from Table 2. In some embodiments, a composition herein comprises polypeptide 535 and one or more polypeptides from Table 2. In some embodiments, a composition herein comprises polypeptide 536 and one or more polypeptides from Table 2. In some embodiments, a composition herein comprises polypeptide 537 and one or more polypeptides from Table 2. In some embodiments, a composition herein comprises polypeptide 538 and one or more polypeptides from Table 2. In some embodiments, a composition herein comprises polypeptide 539 and one or more polypeptides from Table 2. In some embodiments, a composition herein comprises polypeptide 540 and one or more polypeptides from Table 2. In some embodiments, a composition herein comprises polypeptide 541 and one or more polypeptides from Table 2. In some embodiments, a composition herein comprises polypeptide 542 and one or more polypeptides from Table 2. In some embodiments, a composition herein comprises polypeptide 543 and one or more polypeptides from Table 2. In some embodiments, a composition herein comprises polypeptide 544 and one or more polypeptides from Table 2. In some embodiments, a composition herein comprises polypeptide 545 and one or more polypeptides from Table 2 in some embodiments, a composition herein comprises polypeptide 546 and one or more polypeptides from Table 2. In some embodiments, a composition herein comprises polypeptide 547 and one or more polypeptides from Table 2 In some embodiments, a composition herein comprises polypeptide 548 and one or more polypeptides from Table 2. In some embodiments, a composition herein comprises polypeptide 549 and one or more polypeptides from Table 2. In some embodiments, a composition herein comprises polypeptide 550 and one or more polypeptides from Table 2. In some embodiments, a composition herein comprises polypeptide 551 and one or more polypeptides from Table 2. In some embodiments, a composition herein comprises polypeptide 552 and one or more polypeptides from Table 2. In some embodiments, a composition herein comprises polypeptide 553 and one or more polypeptides from Table 2. In some embodiments, a composition herein comprises polypeptide 554 and one or more polypeptides from Table 2. In some embodiments, a composition herein comprises polypeptide 555 and one or more polypeptides from Table 2. In some embodiments, a composition herein comprises polypeptide 556 and one or more polypeptides from Table 2. In some embodiments, a composition herein comprises polypeptide 557 and one or more polypeptides from Table 2. In some embodiments, a composition herein comprises polypeptide 558 and one or more polypeptides from Table 2. In some embodiments, a composition herein comprises polypeptide 559 and one or more polypeptides from Table 2. In some embodiments, a composition herein comprises polypeptide 560 and one or more polypeptides from Table 2. In some embodiments, a composition herein comprises polypeptide 561 and one or more polypeptides from Table 2. In some embodiments, a composition herein comprises polypeptide 562 and one or more polypeptides from Table 2 In some embodiments, a composition herein comprises polypeptide 563 and one or more polypeptides from Table 2. In some embodiments, a composition herein comprises polypeptide 564 and one or more polypeptides from Table 2. In some embodiments, a composition herein comprises polypeptide 565 and one or more polypeptides from Table 2. In some embodiments, a composition herein comprises polypeptide 566 and one or more polypeptides from Table 2. In some embodiments, a composition herein comprises polypeptide 567 and one or more polypeptides from Table 2. In some embodiments, a composition herein comprises polypeptide 568 and one or more polypeptides from Table 2. In some embodiments, a composition herein comprises polypeptide 569 and one or more polypeptides from Table 2. In some embodiments, a composition herein comprises polypeptide 570 and one or more polypeptides from Table 2. In some embodiments, a composition herein comprises polypeptide 571 and one or more polypeptides from Table 2. In some embodiments, a composition herein comprises polypeptide 572 and one or more polypeptides from Table 2. In some embodiments, a composition herein comprises polypeptide 573 and one or more polypeptides from Table 2. In some embodiments, a composition herein comprises polypeptide 574 and one or more polypeptides from Table 2. In some embodiments, a composition herein comprises polypeptide 575 and one or more polypeptides from Table 2. In some embodiments, a composition herein comprises polypeptide 576 and one or more polypeptides from Table 2. In some embodiments, a composition herein comprises polypeptide 577 and one or more polypeptides from Table 2 in some embodiments, a composition herein comprises polypeptide 578 and one or more polypeptides from Table 2. In some embodiments, a composition herein comprises polypeptide 579 and one or more polypeptides from Table 2. In some embodiments, a composition herein comprises polypeptide 580 and one or more polypeptides from Table 2 In some embodiments, a composition herein comprises polypeptide 581 and one or more polypeptides from Table 2. In some embodiments, a composition herein comprises polypeptide 582 and one or more polypeptides from Table 2. In some embodiments, a composition herein comprises polypeptide 583 and one or more polypeptides from Table 2. In some embodiments, a composition herein comprises polypeptide 584 and one or more polypeptides from Table 2. In some embodiments, a composition herein comprises polypeptide 585 and one or more polypeptides from Table 2. In some embodiments, a composition herein comprises polypeptide 586 and one or more polypeptides from Table 2. In some embodiments, a composition herein comprises polypeptide 587 and one or more polypeptides from Table 2. In some embodiments, a composition herein comprises polypeptide 588 and one or more polypeptides from Table 2. In some embodiments, a composition herein comprises polypeptide 589 and one or more polypeptides from Table 2. In some embodiments, a composition herein comprises polypeptide 590 and one or more polypeptides from Table 2. In some embodiments, a composition herein comprises polypeptide 591 and one or more polypeptides from Table 2. In some embodiments, a composition herein comprises polypeptide 592 and one or more polypeptides from Table 2 in some embodiments, a composition herein comprises polypeptide 593 and one or more polypeptides from Table 2. In some embodiments, a composition herein comprises polypeptide 594 and one or more polypeptides from Table 2. In some embodiments, a composition herein comprises polypeptide 595 and one or more polypeptides from Table 2 In some embodiments, a composition herein comprises polypeptide 596 and one or more polypeptides from Table 2. In some embodiments, a composition herein comprises polypeptide 597 and one or more polypeptides from Table 2. In some embodiments, a composition herein comprises polypeptide 598 and one or more polypeptides from Table 2. In some embodiments, a composition herein comprises polypeptide 599 and one or more polypeptides from Table 2. In some embodiments, a composition herein comprises polypeptide 600 and one or more polypeptides from Table 2.

In some embodiments, a composition herein comprises polypeptide 701 and one or more polypeptides from Table 2. In some embodiments, a composition herein comprises polypeptide 702 and one or more polypeptides from Table 2. In some embodiments, a composition herein comprises polypeptide 703 and one or more polypeptides from Table 2. In some embodiments, a composition herein comprises polypeptide 704 and one or more polypeptides from Table 2. In some embodiments, a composition herein comprises polypeptide 705 and one or more polypeptides from Table 2. In some embodiments, a composition herein comprises polypeptide 706 and one or more polypeptides from Table 2. In some embodiments, a composition herein comprises polypeptide 707 and one or more polypeptides from Table 2. In some embodiments, a composition herein comprises polypeptide 708 and one or more polypeptides from Table 2. In some embodiments, a composition herein comprises polypeptide 709 and one or more polypeptides from Table 2. In some embodiments, a composition herein comprises polypeptide 710 and one or more polypeptides from Table 2. In some embodiments, a composition herein comprises polypeptide 711 and one or more polypeptides from Table 2 In some embodiments, a composition herein comprises polypeptide 712 and one or more polypeptides from Table 2. In some embodiments, a composition herein comprises polypeptide 713 and one or more polypeptides from Table 2. In some embodiments, a composition herein comprises polypeptide 714 and one or more polypeptides from Table 2 In some embodiments, a composition herein comprises polypeptide 715 and one or more polypeptides from Table 2. In some embodiments, a composition herein comprises polypeptide 716 and one or more polypeptides from Table 2. In some embodiments, a composition herein comprises polypeptide 717 and one or more polypeptides from Table 2. In some embodiments, a composition herein comprises polypeptide 718 and one or more polypeptides from Table 2. In some embodiments, a composition herein comprises polypeptide 719 and one or more polypeptides from Table 2. In some embodiments, a composition herein comprises polypeptide 721 and one or more polypeptides from Table 2. In some embodiments, a composition herein comprises polypeptide 722 and one or more polypeptides from Table 2. In some embodiments, a composition herein comprises polypeptide 723 and one or more polypeptides from Table 2. In some embodiments, a composition herein comprises polypeptide 724 and one or more polypeptides from Table 2. In some embodiments, a composition herein comprises polypeptide 725 and one or more polypeptides from Table 2. In some embodiments, a composition herein comprises polypeptide 726 and one or more polypeptides from Table 2. In some embodiments, a composition herein comprises polypeptide 727 and one or more polypeptides from Table 2 In some embodiments, a composition herein comprises polypeptide 728 and one or more polypeptides from Table 2. In some embodiments, a composition herein comprises polypeptide 729 and one or more polypeptides from Table 2. In some embodiments, a composition herein comprises polypeptide 730 and one or more polypeptides from Table 2 In some embodiments, a composition herein comprises polypeptide 731 and one or more polypeptides from Table 2. In some embodiments, a composition herein comprises polypeptide 732 and one or more polypeptides from Table 2. In some embodiments, a composition herein comprises polypeptide 733 and one or more polypeptides from Table 2. In some embodiments, a composition herein comprises polypeptide 734 and one or more polypeptides from Table 2. In some embodiments, a composition herein comprises polypeptide 735 and one or more polypeptides from Table 2. In some embodiments, a composition herein comprises polypeptide 736 and one or more polypeptides from Table 2. In some embodiments, a composition herein comprises polypeptide 737 and one or more polypeptides from Table 2. In some embodiments, a composition herein comprises polypeptide 738 and one or more polypeptides from Table 2. In some embodiments, a composition herein comprises polypeptide 739 and one or more polypeptides from Table 2. In some embodiments, a composition herein comprises polypeptide 740 and one or more polypeptides from Table 2. In some embodiments, a composition herein comprises polypeptide 741 and one or more polypeptides from Table 2. In some embodiments, a composition herein comprises polypeptide 742 and one or more polypeptides from Table 2. In some embodiments, a composition herein comprises polypeptide 743 and one or more polypeptides from Table 2. In some embodiments, a composition herein comprises polypeptide 744 and one or more polypeptides from Table 2. In some embodiments, a composition herein comprises polypeptide 745 and one or more polypeptides from Table 2 in some embodiments, a composition herein comprises polypeptide 746 and one or more polypeptides from Table 2. In some embodiments, a composition herein comprises polypeptide 747 and one or more polypeptides from Table 2. In some embodiments, a composition herein comprises polypeptide 748 and one or more polypeptides from Table 2. In some embodiments, a composition herein comprises polypeptide 749 and one or more polypeptides from Table 2. In some embodiments, a composition herein comprises polypeptide 750 and one or more polypeptides from Table 2. In some embodiments, a composition herein comprises polypeptide 751 and one or more polypeptides from Table 2. In some embodiments, a composition herein comprises polypeptide 752 and one or more polypeptides from Table 2. In some embodiments, a composition herein comprises polypeptide 753 and one or more polypeptides from Table 2. In some embodiments, a composition herein comprises polypeptide 754 and one or more polypeptides from Table 2. In some embodiments, a composition herein comprises polypeptide 755 and one or more polypeptides from Table 2. In some embodiments, a composition herein comprises polypeptide 756 and one or more polypeptides from Table 2. In some embodiments, a composition herein comprises polypeptide 757 and one or more polypeptides from Table 2. In some embodiments, a composition herein comprises polypeptide 758 and one or more polypeptides from Table 2. In some embodiments, a composition herein comprises polypeptide 759 and one or more polypeptides from Table 2. In some embodiments, a composition herein comprises polypeptide 760 and one or more polypeptides from Table 2 In some embodiments, a composition herein comprises polypeptide 761 and one or more polypeptides from Table 2. In some embodiments, a composition herein comprises polypeptide 762 and one or more polypeptides from Table 2 In some embodiments, a composition herein comprises polypeptide 763 and one or more polypeptides from Table 2. In some embodiments, a composition herein comprises polypeptide 764 and one or more polypeptides from Table 2. In some embodiments, a composition herein comprises polypeptide 765 and one or more polypeptides from Table 2. In some embodiments, a composition herein comprises polypeptide 766 and one or more polypeptides from Table 2. In some embodiments, a composition herein comprises polypeptide 767 and one or more polypeptides from Table 2. In some embodiments, a composition herein comprises polypeptide 768 and one or more polypeptides from Table 2. In some embodiments, a composition herein comprises polypeptide 769 and one or more polypeptides from Table 2. In some embodiments, a composition herein comprises polypeptide 770 and one or more polypeptides from Table 2. In some embodiments, a composition herein comprises polypeptide 771 and one or more polypeptides from Table 2. In some embodiments, a composition herein comprises polypeptide 772 and one or more polypeptides from Table 2. In some embodiments, a composition herein comprises polypeptide 773 and one or more polypeptides from Table 2. In some embodiments, a composition herein comprises polypeptide 774 and one or more polypeptides from Table 2. In some embodiments, a composition herein comprises polypeptide 775 and one or more polypeptides from Table 2. In some embodiments, a composition herein comprises polypeptide 776 and one or more polypeptides from Table 2. In some embodiments, a composition herein comprises polypeptide 777 and one or more polypeptides from Table 2 In some embodiments, a composition herein comprises polypeptide 778 and one or more polypeptides from Table 2. In some embodiments, a composition herein comprises polypeptide 779 and one or more polypeptides from Table 2. In some embodiments, a composition herein comprises polypeptide 780 and one or more polypeptides from Table 2. In some embodiments, a composition herein comprises polypeptide 781 and one or more polypeptides from Table 2. In some embodiments, a composition herein comprises polypeptide 782 and one or more polypeptides from Table 2. In some embodiments, a composition herein comprises polypeptide 783 and one or more polypeptides from Table 2. In some embodiments, a composition herein comprises polypeptide 784 and one or more polypeptides from Table 2. In some embodiments, a composition herein comprises polypeptide 785 and one or more polypeptides from Table 2. In some embodiments, a composition herein comprises polypeptide 786 and one or more polypeptides from Table 2. In some embodiments, a composition herein comprises polypeptide 787 and one or more polypeptides from Table 2. In some embodiments, a composition herein comprises polypeptide 788 and one or more polypeptides from Table 2. In some embodiments, a composition herein comprises polypeptide 789 and one or more polypeptides from Table 2. In some embodiments, a composition herein comprises polypeptide 790 and one or more polypeptides from Table 2. In some embodiments, a composition herein comprises polypeptide 791 and one or more polypeptides from Table 2. In some embodiments, a composition herein comprises polypeptide 792 and one or more polypeptides from Table 2 in some embodiments, a composition herein comprises polypeptide 793 and one or more polypeptides from Table 2. In some embodiments, a composition herein comprises polypeptide 794 and one or more polypeptides from Table 2. In some embodiments, a composition herein comprises polypeptide 795 and one or more polypeptides from Table 2 In some embodiments, a composition herein comprises polypeptide 796 and one or more polypeptides from Table 2. In some embodiments, a composition herein comprises polypeptide 797 and one or more polypeptides from Table 2. In some embodiments, a composition herein comprises polypeptide 798 and one or more polypeptides from Table 2. In some embodiments, a composition herein comprises polypeptide 799 and one or more polypeptides from Table 2. In some embodiments, a composition herein comprises polypeptide 800 and one or more polypeptides from Table 2.

In some embodiments, a composition herein comprises polypeptide 801 and one or more polypeptides from Table 2. In some embodiments, a composition herein comprises polypeptide 802 and one or more polypeptides from Table 2. In some embodiments, a composition herein comprises polypeptide 803 and one or more polypeptides from Table 2. In some embodiments, a composition herein comprises polypeptide 804 and one or more polypeptides from Table 2. In some embodiments, a composition herein comprises polypeptide 805 and one or more polypeptides from Table 2. In some embodiments, a composition herein comprises polypeptide 806 and one or more polypeptides from Table 2. In some embodiments, a composition herein comprises polypeptide 807 and one or more polypeptides from Table 2. In some embodiments, a composition herein comprises polypeptide 808 and one or more polypeptides from Table 2. In some embodiments, a composition herein comprises polypeptide 809 and one or more polypeptides from Table 2. In some embodiments, a composition herein comprises polypeptide 810 and one or more polypeptides from Table 2. In some embodiments, a composition herein comprises polypeptide 811 and one or more polypeptides from Table 2 in some embodiments, a composition herein comprises polypeptide 812 and one or more polypeptides from Table 2. In some embodiments, a composition herein comprises polypeptide 813 and one or more polypeptides from Table 2. In some embodiments, a composition herein comprises polypeptide 814 and one or more polypeptides from Table 2 In some embodiments, a composition herein comprises polypeptide 815 and one or more polypeptides from Table 2. In some embodiments, a composition herein comprises polypeptide 816 and one or more polypeptides from Table 2. In some embodiments, a composition herein comprises polypeptide 817 and one or more polypeptides from Table 2. In some embodiments, a composition herein comprises polypeptide 818 and one or more polypeptides from Table 2. In some embodiments, a composition herein comprises polypeptide 819 and one or more polypeptides from Table 2. In some embodiments, a composition herein comprises polypeptide 821 and one or more polypeptides from Table 2. In some embodiments, a composition herein comprises polypeptide 822 and one or more polypeptides from Table 2. In some embodiments, a composition herein comprises polypeptide 823 and one or more polypeptides from Table 2. In some embodiments, a composition herein comprises polypeptide 824 and one or more polypeptides from Table 2. In some embodiments, a composition herein comprises polypeptide 825 and one or more polypeptides from Table 2. In some embodiments, a composition herein comprises polypeptide 826 and one or more polypeptides from Table 2. In some embodiments, a composition herein comprises polypeptide 827 and one or more polypeptides from Table 2 in some embodiments, a composition herein comprises polypeptide 828 and one or more polypeptides from Table 2. In some embodiments, a composition herein comprises polypeptide 829 and one or more polypeptides from Table 2. In some embodiments, a composition herein comprises polypeptide 830 and one or more polypeptides from Table 2 In some embodiments, a composition herein comprises polypeptide 831 and one or more polypeptides from Table 2. In some embodiments, a composition herein comprises polypeptide 832 and one or more polypeptides from Table 2. In some embodiments, a composition herein comprises polypeptide 833 and one or more polypeptides from Table 2. In some embodiments, a composition herein comprises polypeptide 834 and one or more polypeptides from Table 2. In some embodiments, a composition herein comprises polypeptide 835 and one or more polypeptides from Table 2. In some embodiments, a composition herein comprises polypeptide 836 and one or more polypeptides from Table 2. In some embodiments, a composition herein comprises polypeptide 837 and one or more polypeptides from Table 2. In some embodiments, a composition herein comprises polypeptide 838 and one or more polypeptides from Table 2. In some embodiments, a composition herein comprises polypeptide 839 and one or more polypeptides from Table 2. In some embodiments, a composition herein comprises polypeptide 840 and one or more polypeptides from Table 2. In some embodiments, a composition herein comprises polypeptide 841 and one or more polypeptides from Table 2. In some embodiments, a composition herein comprises polypeptide 842 and one or more polypeptides from Table 2. In some embodiments, a composition herein comprises polypeptide 843 and one or more polypeptides from Table 2. In some embodiments, a composition herein comprises polypeptide 844 and one or more polypeptides from Table 2. In some embodiments, a composition herein comprises polypeptide 845 and one or more polypeptides from Table 2 in some embodiments, a composition herein comprises polypeptide 846 and one or more polypeptides from Table 2. In some embodiments, a composition herein comprises polypeptide 847 and one or more polypeptides from Table 2. In some embodiments, a composition herein comprises polypeptide 848 and one or more polypeptides from Table 2. In some embodiments, a composition herein comprises polypeptide 849 and one or more polypeptides from Table 2. In some embodiments, a composition herein comprises polypeptide 850 and one or more polypeptides from Table 2. In some embodiments, a composition herein comprises polypeptide 851 and one or more polypeptides from Table 2. In some embodiments, a composition herein comprises polypeptide 852 and one or more polypeptides from Table 2. In some embodiments, a composition herein comprises polypeptide 853 and one or more polypeptides from Table 2. In some embodiments, a composition herein comprises polypeptide 854 and one or more polypeptides from Table 2. In some embodiments, a composition herein comprises polypeptide 855 and one or more polypeptides from Table 2. In some embodiments, a composition herein comprises polypeptide 856 and one or more polypeptides from Table 2. In some embodiments, a composition herein comprises polypeptide 857 and one or more polypeptides from Table 2. In some embodiments, a composition herein comprises polypeptide 858 and one or more polypeptides from Table 2. In some embodiments, a composition herein comprises polypeptide 859 and one or more polypeptides from Table 2. In some embodiments, a composition herein comprises polypeptide 860 and one or more polypeptides from Table 2 in some embodiments, a composition herein comprises polypeptide 861 and one or more polypeptides from Table 2. In some embodiments, a composition herein comprises polypeptide 862 and one or more polypeptides from Table 2 In some embodiments, a composition herein comprises polypeptide 863 and one or more polypeptides from Table 2. In some embodiments, a composition herein comprises polypeptide 864 and one or more polypeptides from Table 2. In some embodiments, a composition herein comprises polypeptide 865 and one or more polypeptides from Table 2. In some embodiments, a composition herein comprises polypeptide 866 and one or more polypeptides from Table 2. In some embodiments, a composition herein comprises polypeptide 867 and one or more polypeptides from Table 2. In some embodiments, a composition herein comprises polypeptide 868 and one or more polypeptides from Table 2. In some embodiments, a composition herein comprises polypeptide 869 and one or more polypeptides from Table 2. In some embodiments, a composition herein comprises polypeptide 870 and one or more polypeptides from Table 2. In some embodiments, a composition herein comprises polypeptide 871 and one or more polypeptides from Table 2. In some embodiments, a composition herein comprises polypeptide 872 and one or more polypeptides from Table 2. In some embodiments, a composition herein comprises polypeptide 873 and one or more polypeptides from Table 2. In some embodiments, a composition herein comprises polypeptide 874 and one or more polypeptides from Table 2. In some embodiments, a composition herein comprises polypeptide 875 and one or more polypeptides from Table 2. In some embodiments, a composition herein comprises polypeptide 876 and one or more polypeptides from Table 2.

In some cases, the one or more polypeptides from Table 2 comprises IL-15. In some cases, the one or more polypeptides from Table 2 comprises THBS4. In some cases, the one or more polypeptides from Table 2 comprises POSTN. In some cases, the one or more polypeptides from Table 2 comprises THBS1 In some cases, the one or more polypeptides from Table 2 comprises THBS2 In some cases, the the one or more polypeptides from Table 2 comprises VTN. In some cases, the one or more polypeptides from Table 2 comprises FGF17. In some cases, the one or more polypeptides from Table 2 comprises IGF2. In some cases, the one or more polypeptides from Table 2 comprises polypeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, or 876, In some cases, one or a plurality of the polypeptides of the composition are heparin-associated polypolypeptides in some cases, one or a plurality of the polypeptides of the composition are mitogenic and/or fusion promoting polypolypeptides. In certain embodiments, one or more of the polypeptides of the composition comprise one or more additional modifications to increase stability. In certain embodiments, one or more of the polypeptides is covalently conjugated to polyethylene glycol, polypropylene glycol, polyoxyalkylenes, or copolymers of polyethylene glycol, polysialic acid, glycolic acid, or polypropylene glycol. In certain embodiments, one or more of the polypeptides is fused or conjugated to another protein to increase stability and or bioavailability in certain embodiments, one or more of the polypeptides is fused with an Fc region of an immunoglobulin or with serum albumin in certain embodiments, one or more of the polypeptides is present in a concatemer with one, two, three, four or more distinct polypeptides selected from Table 2 and/or Table 1. In certain embodiments, one or more of the polypeptides is present in a concatemer with one, two, three, four, or more distinct polypeptides. In certain embodiments, one or more of the polypeptides is included in the composition with a biodegradable or bioabsorbable carrier that promotes polypeptide stability. In certain embodiments, the biodegradable or bioabsorable carrier comprises polylactic acid (PLA), polyglycolic acid (PGA), or Poly(D,L-lactic-coglycolic-acid) (PLGA). In certain embodiments, the biodegradable or bioabsorable carrier comprises polylactic acid (PLA). In certain embodiments, the biodegradable or bioabsorable carrier comprises polyglycolic acid (PGA). In certain embodiments, the biodegradable or bioabsorable carrier comprises Poly(D,L-lactic-coglycolic-acid) (PLGA).

In certain embodiments, the heparin-associated polypeptides increase the mitogenic (e.g., proliferative capacity) of a somatic cell that is a tissue cell or a tissue precursor, such as, a muscle cell, a muscle precursor cell, a tenocyte, a tenocyte precursor cell, a chondrocyte, a chondrocyte precursor, a mesenchymal stem cell, or a fibroblast. The cell can be a precursor cell derived from any mammal, such as, monkeys, apes, dogs, cats, horses, rats, mice, or humans. In certain embodiments, the precursor cell is a human precursor cell. In certain embodiments, the heparin-associated polypeptides increase the proliferative capacity of a mouse myoblast by at least about 1.5-fold, about 2-fold, about 3-fold, or about 4-fold as measured by BrdU or EdU incorporation.

### Therapeutic Indications

In certain aspects, heparin-associated polypeptides and compositions comprising heparin-associated polypeptides, described herein, are useful for treating diseases and disorders that involve soft-tissue injury, degradation, or destruction Aging disorders that result in the deterioration and loss of muscle tissue are such soft-tissue disorders. Sarcopenia, for example, is the degenerative loss of skeletal muscle mass quality, and strength associated with aging. Injuries that result in acute muscle damage are other such disorders. The disorders include muscle ruptures, strains, and contusions. A rupture is a separating of the muscle tissues. Muscle strains are contraction-induced injuries in which muscle fibers tear due to extensive mechanical stress, and can be classified as a grade I, II, or III. Muscle contusions are muscle hematomas. Muscle injury can also be caused by non-mechanical stresses such as cachexia. Cachexia may be caused by malnutrition, cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), Crohn's disease, untreated/severe type 1 diabetes mellitus, anorexia nervosa, chemotherapy, muscular dystrophy or other genetic diseases which cause immobility, and hormonal deficiencies. Certain disorders that are weaknesses of specific muscles such as dysphagia or facioscapulohumeral muscular dystrophy may also be treated by the polypeptides described herein. Additional soft-tissues disorders that may be treated using the heparin-associated polypeptides described herein are those that inflict injury to the tendons, ligaments or cartilage. In certain embodiments, the muscle wasting disease is a muscular dystrophy. In certain embodiments, the muscular dystrophy comprises myotonic muscular dystrophy, Duchenne muscular dystrophy, Becker muscular dystrophy, Limb-girdle muscular dystrophy, facioscapulohumeral muscular dystrophy, congenital, muscular dystrophy, oculopharyngeal muscular dystrophy, or distal muscular dystrophy. In certain embodiments, the muscular dystrophy is Becker muscular dystrophy. In certain embodiments, the heparin-associated polypeptides useful for treating a soft-tissue disorder comprise any one, two, three, four, or five heparin-associated polypeptides selected from Vitronectin (VTN), Periostin (POSTN), Fribroblast growth factor (FGF17), Thrombospondin 2 (THBS2), Thrombospondin 4 (THBS4), Thrombospondin 1 (THBS1), Insulin-like growth factor 2 (IGF2), and Interleukin 15 (IL-15). In certain embodiments, the heparin-associated polypeptides useful for treating a soft-tissue disorder comprise any one, two, three, four, or five heparin-associated polypeptides selected from Vitronectin (VTN), Stanniocalcin-2 (STC2), Agrin (AGRN), Thrombospondin 2 (THBS2), follistatin (FST), Periostin (POSTN), Fribroblast growth factor (FGF17), Thrombospondin 4 (THBS4), Thrombospondin 1 (THBS1), Insulin-like growth factor 2 (IGF2), and Interleukin 15 (IL-15).

In certain embodiments, the heparin-associated polypeptides and compositions comprising heparin-associated polypeptides, described herein, are for use in treating an individual with an aging disorder, a muscle wasting disorder, a muscle injury, an injury to a connective tissue, or an injury to a non-muscle soft-tissue, or any combination thereof. In certain embodiments, the aging disorder is sarcopenia. In certain embodiments, the muscle wasting disorder is cachexia. In certain embodiments, the cachexia is a result of a cancer. In certain embodiments, the cachexia is a result of AIDS. In certain embodiments, the injury is a muscle injury. In certain embodiments, the muscle wasting is atrophy do to limb immobilization or disuse. In certain embodiments, the muscle injury is a strain or a tear in certain embodiments, the muscle injury is a Grade lit strain. In certain embodiments, sarcopenia contributes to the incidence of the muscle injury. In certain embodiments, the injury is ligament damage. In certain embodiments, the ligament damage is a rupture or a tear. In certain embodiments, the injury is tendon damage. In certain embodiments, the tendon damage is a rupture or a tear. In certain embodiments, the injury is cartilage damage. In certain embodiments, the heparin-associated polypeptides comprise any one, two, three, four, five or more heparin-associated polypeptides selected from Vitronectin (VTN), Periostin (POSTN), Fribroblast growth factor (FGF17), Thrombospondin 2 (THBS2), Thrombospondin 4 (THBS4), Thrombospondin 1 (THBS1), Insulin-like growth factor 2 (IGF2), and Interleukin 15 (IL-15). In certain embodiments, the heparin-associated polypeptides comprise any one, two, three, four, five or more heparin-associated polypeptides selected from Vitronectin (VTN), Stanniocalcin-2 (STC2), Agrin (AGRN), Thrombospondin 2 (THBS2), and follistatin (FST).

In certain embodiments, the heparin-associated polypeptides and compositions comprising heparin-associated polypeptides, described herein, are for use in a method of treating myositis. In certain embodiments, the myositis comprises dermatomyositis, polymyositis, necrotizing myopathy (also called necrotizing autoimmune myopathy or immune-mediated necrotizing myopathy), juvenile myositis, or sporadic inclusion-body myositis. In certain embodiments, the heparin-associated polypeptides comprise any one, two, three, four, five or more heparin-associated polypeptides selected from Vitronectin (VTN), Periostin (POSTN), Fribroblast growth factor (FGF17), Thrombospondin 2 (THBS2), Thrombospondin 4 (THBS4), Thrombospondin 1 (THBS1), Insulin-like growth factor 2 (IGF2), and Interleukin 15 (IL-15). In certain embodiments, the heparin-associated polypeptides comprise any one, two, three, four, or five heparin-associated polypeptides selected from Vitronectin (VTN), Stanniocalcin-2 (STC2), Agrin (AGRN), Thrombospondin 2 (THBS2), and follistatin (FST).

In certain embodiments, the heparin-associated polypeptides and compositions comprising heparin-associated polypeptides, described herein, are for use in a method of increasing proliferation or promoting survival of a cell associated with soft-tissue damage. In certain embodiments, the heparin-associated polypeptides described herein are useful in a method of increasing proliferation or promoting survival of any one or more of a muscle cell, a muscle precursor cell, a tenocyte, a tenocyte precursor cell, a chondrocyte, a chondrocyte precursor cell, a mesenchymal stem cell, or a fibroblast. In certain embodiments, the heparin-associated polypeptides comprise any one, two, three, four, five or more heparin-associated polypeptides selected from Vitronectin (VTN), Periostin (POSTN), Fribroblast growth factor (FGF17), Thrombospondin 2 (THBS2), Thrombospondin 4 (THBS4), Thrombospondin 1 (THBS1), Insulin-like growth factor 2 (IGF2), and Interleukin 15 (IL-15). In certain embodiments, the heparin-associated polypeptides comprise any one, two, three, four, or five heparin-associated polypeptides selected from Vitronectin (VTN), Stanniocalcin-2 (STC2), Agrin (AGRN), Thrombospondin 2 (THBS2), and follistatin (FST).

The heparin-associated polypeptides compositions described herein can be administered separately or as a mixture of 2, 3, 4, 5, 6, 7, 8, 9, 10 or more heparin-binding or heparin-associated polypeptides for the treatment of any disorder associated with muscle or soft-tissue.

In certain aspects, a method of treating a disease or condition, such as those described herein, in a subject in need thereof comprises administering to the subject a composition comprising a polypeptide of Table 2. In some embodiments, the polypeptide of Table 2 is a polypeptide of Table 1 In some embodiments, the composition comprises a pharmaceutically acceptable excipient, such as described herein. In some embodiments, the disease or condition comprises an aging disorder, muscle wasting disorder, muscle injury, or injury to connective tissue, or a combination thereof. In some embodiments, the aging disorder comprises sarcopenia. In some embodiments, the muscle wasting disorder comprises muscular dystrophy. In some embodiments, the muscle wasting is a result of obesity. In certain embodiments, the muscle wasting is the result of a metabolic disorder. In some cases the metabolic disorder is diabetes. In some cases the diabetes is Type 2 Diabetes. In some embodiments, muscle wasting is a result of disease progression. In some embodiments, muscle wasting is a result of therapeutic treatment. In some embodiments, the muscle wasting is cachexia. In some embodiments, the therapeutic polypeptide promotes fusion of myocytes.

In some embodiments, the polypeptide is a heparin-associated binding polypeptide as described herein. In some embodiments, the polypeptide is a mitogenic and/or fusion promoting polypeptide as described herein. In some embodiments, a composition comprising a plurality of heparin-associated binding polypeptides as described herein is administered. In some embodiments, a composition comprising a plurality of mitogenic and/or fusion promoting polypeptides as described herein is administered.

In some embodiments, the polypeptide has been recombinantly produced. In some embodiments, the polypeptide has been produced in a mammalian cell culture. In certain embodiments, the polypeptide has been produced in a mammalian cell and the mammalian cell is a human cell. In some cases the human cell is a human embryonic kidney-derived epithelial cell (e.g., HEK293 cells). In some embodiments, the mammalian cell culture is a mouse myeloma cell culture. In some embodiments, the mammalian cell culture is a Chinese Hamster Ovary (CHO) cell culture. In some embodiments, the polypeptide has been produced in a non-mammalian cell culture, e.g., in bacteria, yeast, or insect cells. In certain embodiments, the polypeptide has been purified from a human biological sample. In some cases, the human biological sample is human plasma. In some embodiments, the composition is formulated for administration by injection to the subject. In some embodiments, the composition comprises one or more polypeptides having at least about 90% homology to a sequence selected from SEQ ID NOS: 1-44, 55, 56, and 58-70. In some embodiments, the composition comprises polypeptide 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, or 876, or any combination thereof.

In some embodiments, the polypeptide comprises VTN. In some cases, the VTN comprises a polypeptide comprising at least about 90% homology or identity to SEQ ID NO: 1. In some cases, the VTN comprises a polypeptide comprising at least about 90% homology or identity to amino acids 20-478 of SEQ ID NO: 1. In some cases, the VTN is purified from human plasma.

In some embodiments, the polypeptide comprises POSTN. In some cases, the POSTN comprises a polypeptide comprising at least about 90% homology or identity to SEQ ID NO: 6. In some cases, the POSTN comprises a polypeptide comprising at least about 90% homology or identity to amino acids 22-836 of SEQ ID NO: 6. In some cases, the POSTN is expressed in a mouse myeloma cell line.

In some embodiments, the polypeptide comprises FGF17. In some cases, the FGF17 comprises a polypeptide comprising at least about 90% homology or identity to SEQ ID NO: 7. In some cases, the FGF17 comprises a polypeptide comprising at least about 90% homology or identity to amino acids 23-216 of SEQ ID NO: 7. In some cases, the FGF17 is expressed in a bacterial cell. In some embodiments, the bacterial cell is E. coli.

In some embodiments, the polypeptide comprises THBS2. In some cases, the THBS2 comprises a polypeptide comprising at least about 90% homology or identity to SEQ ID NO: 4. In some cases, the THBS2 comprises a polypeptide comprising at least about 90% homology or identity to amino acids 19-1172 of SEQ ID NO: 4. In some cases, the THBS2 is expressed in a mouse myeloma cell line.

In some embodiments, the polypeptide comprises THBS4. In some cases, the THBS4 comprises a polypeptide comprising at least about 90% homology or identity to SEQ ID NO: 8. In some cases, the THBS4 comprises a polypeptide comprising at least about 90% homology or identity to amino acids 27-961 of SEQ ID NO: 8 In some cases, the THBS4 is expressed in Chinese hamster ovary cell.

In some embodiments, the polypeptide comprises IGF2. In some cases, the IGF2 comprises a polypeptide comprising at least about 90% homology or identity to SEQ ID NO: 11. In some cases, the IGF2 comprises a polypeptide comprising at least about 90% homology or identity to amino acids 25-91 of SEQ ID NO: 11. In some cases, the IGF2 is expressed in a bacterial cell. In some embodiments, the bacterial cell is E. coli.

In some embodiments, the polypeptide comprises IL-15. In some cases, the IL-15 comprises a polypeptide comprising at least about 90% homology or identity to SEQ ID NO: 10. In some cases, the IL-15 comprises a polypeptide comprising at least about 90% homology or identity to amino acids 49-162 of SEQ ID NO: 10. In some cases, the IL-15 is expressed in a bacterial cell. In some embodiments, the bacterial cell is E. coli.

In some embodiments, the polypeptide comprises THBS1. In some cases, the THBS1 comprises a polypeptide comprising at least about 90% homology or identity to SEQ ID NO: 9. In some cases, the THBS1 comprises a polypeptide comprising at least about 90% homology or identity to amino acids 19-1170 of SEQ ID NO: 9. In some cases, the THBS1 is expressed in a mouse myeloma cell line.

In some embodiments, the polypeptide comprises IL-15, and the composition further comprises THBS2. In some embodiments, the polypeptide comprises IL-15, and the composition further comprises THBS4 In some embodiments, the polypeptide comprises THBS4, and the composition further comprises THBS2. In some embodiments, the polypeptide comprises IL-15, and the composition further comprises THBS2 and THBS4.

In some embodiments, the polypeptide comprises IGF2, and the composition further comprises THBS2. In some embodiments, the polypeptide comprises IGF2, and the composition further comprises THBS4. In some embodiments, the polypeptide comprises THBS4, and the composition further comprises THBS2 In some embodiments, the polypeptide comprises IGF2, and the composition further comprises THBS2 and THBS4.

### Schedules Routes of Administration and Amounts

In certain embodiments, the heparin-associated polypeptides can be administered by any suitable route such as, for example, subcutaneous, intravenous, or intramuscular. In certain embodiments, the heparin-associated polypeptides are administered on a suitable dosage schedule, for example, weekly, twice weekly, monthly, twice monthly, once every three weeks, or once every four weeks. The heparin-associated polypeptides can be administered in any therapeutically effective amount. In certain embodiments, the therapeutically acceptable amount is about 0.001 mg/kg to about 1 mg/kg. In certain embodiments, the therapeutically acceptable amount is about 0.001 mg/kg to about 0.002 mg/kg, about 0.001 mg/kg to about 0.005 mg/kg, about 0.001 mg/kg to about 0.01 mg/kg, about 0.001 mg/kg to about 0.02 mg/kg, about 0.001 mg/kg to about 0.05 mg/kg, about 0.001 mg/kg to about 0.1 mg/kg, about 0.001 mg/kg to about 0.2 mg/kg, about 0.001 mg/kg to about 0.5 mg/kg, about 0.001 mg/kg to about 1 mg/kg, about 0.002 mg/kg to about 0.005 mg/kg, about 0.002 mg/kg to about 0.01 mg/kg, about 0.002 mg/kg to about 0.02 mg/kg, about 0.002 mg/kg to about 0.05 mg/kg, about 0.002 mg/kg to about 0.1 mg/kg, about 0.002 mg/kg to about 0.2 mg/kg, about 0.002 mg/kg to about 0.5 mg/kg, about 0.002 mg/kg to about 1 mg/kg, about 0.005 mg/kg to about 0.01 mg/kg, about 0.005 mg/kg to about 0.02 mg/kg, about 0.005 mg/kg to about 0.05 mg/kg, about 0.005 mg/kg to about 0.1 mg/kg, about 0.005 mg/kg to about 0.2 mg/kg, about 0.005 mg/kg to about 0.5 mg/kg, about 0.005 mg/kg to about 1 mg/kg, about 0.01 mg/kg to about 0.02 mg/kg, about 0.01 mg/kg to about 0.05 mg/kg, about 0.01 mg/kg to about 0.1 mg/kg, about 0.01 mg/kg to about 0.2 mg/kg, about 0.01 mg/kg to about 0.5 mg/kg, about 0.01 mg/kg to about 1 mg/kg, about 0.02 mg/kg to about 0.05 mg/kg, about 0.02 mg/kg to about 0.1 mg/kg, about 0.02 mg/kg to about 0.2 mg/kg, about 0.02 mg/kg to about 0.5 mg/kg, about 0.02 mg/kg to about 1 mg/kg, about 0.05 mg/kg to about 0.1 mg/kg, about 0.05 mg/kg to about 0.2 mg/kg, about 0.05 mg/kg to about 0.5 mg/kg, about 0.05 mg/kg to about 1 mg/kg, about 0.1 mg/kg to about 0.2 mg/kg, about 0.1 mg/kg to about 0.5 mg/kg, about 0.1 mg/kg to about 1 mg/kg, about 0.2 mg/kg to about 0.5 mg/kg, about 0.2 mg/kg to about 1 mg/kg, or about 0.5 mg/kg to about 1 mg/kg. In certain embodiments, the therapeutically acceptable amount is about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. In certain embodiments, the therapeutically acceptable amount is at least about 0.001 mg/kg, about 0.002 mg/kg, about 0.005 mg/kg, about 0.01 mg/kg, about 0.02 mg/kg, about 0.05 mg/kg, about 0.1 mg/kg, about 0.2 mg/kg, or about 0.5 mg/kg. In certain embodiments, the therapeutically acceptable amount is at most about 0.002 mg/kg, about 0.005 mg/kg, about 0.01 mg/kg, about 0.02 mg/kg, about 0.05 mg/kg, about 0.1 mg/kg, about 0.2 mg/kg, about 0.5 mg/kg, or about 1 mg/kg. In certain embodiments, the therapeutically acceptable amount is about 0.1 mg/kg to about 50 mg/kg. In certain embodiments, the therapeutically acceptable amount is about 0.1 mg/kg to about 0.2 mg/kg, about 0.1 mg/kg to about 0.5 mg/kg, about 0.1 mg/kg to about 1 mg/kg, about 0.1 mg/kg to about 2 mg/kg, about 0.1 mg/kg to about 5 mg/kg, about 0.1 mg/kg to about 10 mg/kg, about 0.1 mg/kg to about 20 mg/kg, about 0.1 mg/kg to about 50 mg/kg, about 0.2 mg/kg to about 0.5 mg/kg, about 0.2 mg/kg to about 1 mg/kg, about 0.2 mg/kg to about 2 mg/kg, about 0.2 mg/kg to about 5 mg/kg, about 0.2 mg/kg to about 10 mg/kg, about 0.2 mg/kg to about 20 mg/kg, about 0.2 mg/kg to about 50 mg/kg, about 0.5 mg/kg to about 1 mg/kg, about 0.5 mg/kg to about 2 mg/kg, about 0.5 mg/kg to about 5 mg/kg, about 0.5 mg/kg to about 10 mg/kg, about 0.5 mg/kg to about 20 mg/kg, about 0.5 mg/kg to about 50 mg/kg, about 1 mg/kg to about 2 mg/kg, about 1 mg/kg to about 5 mg/kg, about 1 mg/kg to about 10 mg/kg, about 1 mg/kg to about 20 mg/kg, about 1 mg/kg to about 50 mg/kg, about 2 mg/kg to about 5 mg/kg, about 2 mg/kg to about 10 mg/kg, about 2 mg/kg to about 20 mg/kg, about 2 mg/kg to about 50 mg/kg, about 5 mg/kg to about 10 mg/kg, about 5 mg/kg to about 20 mg/kg, about 5 mg/kg to about 50 mg/kg, about 10 mg/kg to about 20 mg/kg, about 10 mg/kg to about 50 mg/kg, or about 20 mg/kg to about 50 mg/kg. In certain embodiments, the therapeutically acceptable amount is about 0.1 mg/kg, about 0.2 mg/kg, about 0.5 mg/kg, about 1 mg/kg, about 2 mg/kg, about 5 mg/kg, about 10 mg/kg, about 20 mg/kg, or about 50 mg/kg. In certain embodiments, the therapeutically acceptable amount is at least about 0.1 mg/kg, about 0.2 mg/kg, about 0.5 mg/kg, about 1 mg/kg, about 2 mg/kg, about 5 mg/kg, about 10 mg/kg, or about 20 mg/kg. In certain embodiments, the therapeutically acceptable amount is at most about 0.2 mg/kg, about 0.5 mg/kg, about 1 mg/kg, about 2 mg/kg, about 5 mg/kg, about 10 mg/kg, about 20 mg/kg, or about 50 mg/kg.

### Nucleic Acids

TABLE 3
Nucleic acid sequences.

| | | | Protein | mRNA | | |
|---|---|---|---|---|---|---|
| Protein Name | Size (AA) | UniProtKB | accession# | accession# | Gene ID | SEQ ID |
| FGF17 | 216 | O60258 1 | NP_003858.1 | NM_003867.4 | 8822 | 45 |
| THBS1 | 1170 | P07996.2 | NP_003237.2 | NM_003246.4 | 7057 | 46 |
| THBS2 | 1172 | P35442.2 | NP_003238.2 | NM_003247.3 | 7058 | 47 |
| THBS4 | 961 | P35443.2 | NP_003239.2 | NM_003248.6 | 7060 | 48 |
| IGF2 | 180 | P01344.1 | NP_000603.1 | NM_000612.6 | 3481 | 49 |
| IL15 | 162 | P40933.1 | NP_000576.1 | NM_000585.5 | 3600 | 50 |
| IGFBP7 | 282 | Q16270.1 | NP_001544.1 | NM_001553.3 | 3490 | 51 |
| VTN | 478 | P04004 | NP_000629.3 | NM_000638.4 | 7448 | 52 |
| POSTN | 836 | Q15063.2 | NP_006466.2 | NM_006475.3 | 10631 | 53 |
| PDGFRL | 375 | Q15198.1 | NP_006198.1 | NM_006207.2 | 5157 | 54 |
| ANOS1 | 3730 | P23352 | NP_000207.2 | NM_000216.4 | 3730 | 57 |

In certain embodiments, described herein, are nucleic acids that encode the heparin-associated polypeptides described herein. In certain embodiments, the nucleic acids are exogenous. In certain embodiments, the nucleic acid is a plasmid. In certain embodiments, the nucleic acid is a viral vector In certain embodiments, the viral vector is an adenovirus, lentivirus, retrovirus, adeno-associated virus, or vaccinia virus. In certain embodiments, the nucleic acid comprises RNA. In certain embodiments, the nucleic acid encodes any of the polypeptides listed in Table 1 or Table 2, or VTN, STC2, AGRN, POSTN, FGF17, THBS2, FST, THBS1, IL-15, IGF2, or THBS4. In certain embodiments, the nucleic acid encodes any one or more polypeptide embodiments described herein. Nucleic acids according to this description can comprise additional nucleic acid sequences sufficient to propagate the vector or express a polypeptide encoded by the vector. In certain embodiments, the nucleic acid comprises a universal promoter, such as the CMV promoter, or an inducible promoter system such as a TET_{ON}, TET_{OFF} or GAL4. In certain embodiments, the nucleic acid is expressed via a tissue specific promoter or one compatible with a eukaryotic or prokaryotic cellular expression system. The nucleic acid can further comprise a sequence encoding a suitable purification tag (e.g., HIS-tag, V5, FLAG, MYC).

### Production of Heparin-Associated or Heparin-Binding Polypeptides

Once a polypeptide is determined as a heparin-associated or heparin-binding polypeptide it can be purified or synthesized in any suitable manner. A nucleic acid encoding the polypeptide can be cloned into a suitable vector and expressed in a suitable cellular system. In certain embodiments, the cellular system is a prokaryotic cell system. In certain embodiments, the cellular system is a eukaryotic cell system. In certain embodiments, the cellular system is a mammalian cell system. The supernatants from such an expression system can be subjected to one or more purification steps involving centrifugation, ultracentrifugation, filtration, diafiltration, tangential-flow filtration, dialysis, chromatography (e.g., cation exchange, ion exchange, hydrophobic interaction, reverse phase, affinity, or size exclusion). The polypeptides can be purified to an extent suitable for human administration Additionally, polypeptides can be synthesized for inclusion in a formulation to be administered to a human subject. In certain embodiments, the polypeptides can be produced by a suitable peptide synthesis method, such as solid-phase synthesis.

### Master Cell Bank and Transgenic Cells

In a certain embodiment, described herein is a master cell bank comprising a cell that comprises a nucleic acid encoding one or more heparin-associated polypeptides integrated into its genome creating a transgenic cell-line. In some embodiments, the master cell bank comprises a plurality of cells that each comprise a nucleic acid encoding a heparin-associated polypeptide. In certain embodiments, the nucleic acid is maintained extrachromosomally on a plasmid or yeast artificial chromosome. In certain embodiments, the nucleic acid is integrated into a chromosomal location. In certain embodiments, the cell is a yeast cell. In certain embodiments, the yeast is Pichia pastoris or Saccharomyces cerevisiae. In certain embodiments, the cell is a mammalian cell. In certain embodiments, the mammalian cell is a 293T cell or derivative thereof (e.g., 293T-Rex). In certain embodiments, the cell is a bacterial cell.

In certain embodiments, the transgenic mammalian, yeast, or bacterial cell is a master cell bank that comprises a cryopreservative suitable for freezing to at least about −80′ or below. In certain embodiments, the master cell bank comprises glycerol at between about 10 and about 30%, and is suitable for long-term storage at about −80′ or below. In certain embodiments, the master cell bank can preserve a transgenic mammalian, yeast, or bacterial strain for at least 1, 2, 3, 4, 5, 6, 7, 8, 9, 10 or more years.

### Pharmaceutically Acceptable Excipients, Carriers, and Diluents

The heparin-associated polypeptide(s) described herein can be administered in a pharmaceutical composition that comprises one or more pharmaceutically acceptable excipients, carriers, or diluents. The exact components can differ based upon the preferred route of administration. The excipients used in a pharmaceutical composition can provide additional function to the polypeptide by making the polypeptide suitable for a particular route of administration (e.g., intravenous, topical, subcutaneous, or intramuscular), increasing polypeptide stability, increasing penetration of a desired tissue (e.g., muscle or skin), increasing residence time at particular site, increasing solubility, enhancing the efficacy of the polypeptide, and/or reducing inflammatory reactions coincident with administration.

In certain embodiments, the heparin-associated polypeptide(s) described herein are included in a pharmaceutical composition with a solubilizing emulsifying, or dispersing agent. In certain embodiments, the solubilizing agent can allow high-concentration solutions of heparin-associated polypeptides that exceed at least about 2 mg/mL, 5 mg/mL, 10 mg/mL, 15 mg/mL, or 20 mg/mL. Carbomers in an aqueous pharmaceutical composition serve as emulsifying agents and viscosity modifying agents. In certain embodiments, the pharmaceutically acceptable excipient comprises or consists of a carbomer. In certain embodiments, the carbomer comprises or consists of carbomer 910, carbomer 934, carbomer 934P, carbomer 940, carbomer 941, carbomer 1342, or combinations thereof. Cyclodextrins in an aqueous pharmaceutical composition serve as solubilizing and stabilizing agents agents. In certain embodiments, the pharmaceutically acceptable excipient comprises or consists of a cyclodextrin. In certain embodiments, the cyclodextrin comprises or consists of alpha cyclodextrin, beta cyclodextrin, gamma cyclodextrin, or combinations thereof. Lecithin in a pharmaceutical composition may serve as a solubilizing agent. In certain embodiments, the solubilizing agent comprises or consists of lecithin. Poloxamers in a pharmaceutical composition serve as emulsifying agents, solubilizing agents, and dispersing agents. In certain embodiments, the pharmaceutically acceptable excipient comprises or consists of a poloxamer. In certain embodiments, the poloxamer comprises or consists of poloxamer 124, poloxamer 188, poloxamer 237, poloxamer 338, poloxamer 407, or combinations thereof. Polyoxyethylene sorbitan fatty acid esters in a pharmaceutical composition serve as emulsifying agents, solubilizing agents, surfactants, and dispersing agents. In certain embodiments, the pharmaceutically acceptable excipient comprises or consists of a polyoxyethylene sorbitan fatty acid ester. In certain embodiments, the polyoxyethylene sorbitan fatty acid ester comprises or consists of polysorbate 20, polysorbate 21, polysorbate 40, polysorbate 60, polysorbate 61, polysorbate 65, polysorbate 80, polysorbate 81, polysorbate 85, polysorbate 120, or combinations thereof. Polyoxyethylene stearates in a pharmaceutical composition serve as emulsifying agents, solubilizing agents, surfactants, and dispersing agents. In certain embodiments, the pharmaceutically acceptable excipient comprises or consists of a polyoxyethylene stearate. In certain embodiments, the polyoxyethylene stearate comprises or consists of polyoxyl 2 stearate, polyoxyl 4 stearate, polyoxyl 6 stearate, polyoxyl 8 stearate, polyoxyl 12 stearate, polyoxyl 20 stearate, polyoxyl 30 stearate, polyoxyl 40 stearate, polyoxyl 50 stearate, polyoxyl 100 stearate, polyoxyl 150 stearate, polyoxyl 4 distearate, polyoxyl 8 distearate, polyoxyl 12 distearate, polyoxyl 32 distearate, polyoxyl 150 distearate, or combinations thereof. Sorbitan esters in a pharmaceutical composition serve as emulsifying agents, solubilizing agents, and non-ionic surfactants, and dispersing agents. In certain embodiments, the pharmaceutically acceptable excipient comprises or consists of a sorbitan ester. In certain embodiments, the sorbitan ester comprises or consists of sorbitan laurate, sorbitan oleate, sorbitan palmitate, sorbitan stearate, sorbitan trioleate, sorbitan sesquioleate, or combinations thereof. In certain embodiments, solubility can be achieved with a protein carrier. In certain embodiments the protein carrier comprises recombinant human albumin.

In certain embodiments, the heparin-associated polypeptide(s) of the current disclosure are formulated to increase stability. Polypeptides in aqueous formulations may require stabilization to prevent degradation. In certain embodiments, the stabilizer comprises pH buffers, salts, amino acids, polyols/disaccharides/polysaccharides, liposomes, surfactants, antioxidants, reducing agents, or chelating agents. In certain embodiments, the stabilizer comprises or consists of a polyol/non-reducing sugar. In certain embodiments, the non-reducing sugar comprises or consists of sucrose, mannitol, trehalose, raffinose, stachyose, xylitol, starch, verbascose, or combinations thereof. Polypeptides can be encapsulated in liposomes to increase stability. In certain embodiments, the stabilizer comprises or consists of liposomes. In certain embodiments, the liposomes comprise or consists of ipalmitoylphosphatidylcholine (DPPC) liposomes, phosphatidylcholine:cholesterol (PC:Chol) (70:30) liposomes, or dipalmitoylphosphatidylcholine:dipalmitoylphosphatidylserine (DPPC:DPPS) liposomes (70:30). Non-ionic surfactants can increase the stability of a polypeptide. In certain embodiments, the stabilizer comprises or consists of a non-ionic surfactant. In certain embodiments, the non-ionic surfactant comprises or consists of polysorbates (e.g., poly sorbate 80, poly sorbate 20), alkylsaccharides alkyl ethers and alkyl glyceryl ethers, polyoxyethylene (4) lauryl ether; polyoxyethylene cetyl ethers, polyoxyethylene stearyl ethers, sorbitan fatty acid esters, polyoxyethylene fatty acid esters, or combinations thereof. In certain embodiments, the polypeptide is formulated with a protein surfactant, such as recombinant human serum albumin as a stabilizer. Antioxidants or reducing agents can increase the stability of a polypeptide. In certain embodiments, the stabilizer comprises or consists of an antioxidant or reducing agent. In certain embodiments, the reducing agent comprises or consists of dithiothreitol, ethylenediaminetetraacetic acid, 2-Mercaptoethanol, Tris(2-carboxyethyl)phosphine hydrochloride, Tris(hydroxypropyl)phosphine, or combinations thereof. In certain embodiments, the antioxidant comprises or consists of methionine, ascorbic acid, citric acid, alpha tocopherol, sodium bisulfite, ascorbyl palmitate, erythorbic acid, or combinations thereof. Chelating agents can stabilize polypeptides by reducing the activity of proteases. In certain embodiments, the stabilizer comprises or consists of a chelating agent. In certain embodiments, the chelating agent comprises or consists of ethylenediaminetetraacetic acid (EDTA), ethylene glycol-bis(β-aminoethyl ether)-N,N,N′,N′-tetraacetic acid (EGTA), metal complexes (e.g. Zn-protein complexes), or combinations thereof. Buffer agents can stabilize polypeptides by reducing the acid hydrolysis of polypeptides. In certain embodiments, the stabilizer comprises or consists of a buffer agent. In certain embodiments, the buffer agent comprises or consists sucrose octa-sulfate, ammonium carbonate, ammonium phosphate, boric acid, sodium citrate, potassium citrate, lactic acid, 3-(N-morpholino)propanesulfonic acid (MOPS), 2-(N-morpholino)ethanesulfonic acid (MES), hydroxymethylaminomethane (Tris), calcium carbonate, calcium phosphate or combinations thereof.

The heparin-associated polypeptide(s) also may be entrapped in or associated with microcapsules prepared, for example, by coacervation techniques or by interfacial polymerization (for example, hydroxymethylcellulose or gelatin-microcapsules and poly-(methylmethacylate) microcapsules, respectively), in colloidal delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

The heparin-associated polypeptide(s) of the current disclosure may be formulated or delivered with an anti-inflammatory agent. In certain embodiments, the anti-inflammatory agent comprises or consists of a corticosteroid. In certain embodiments, the corticosteroid comprises or consists of hydrocortisone, cortisone, ethamethasoneb (Celestone), prednisone (Prednisone Intensol), prednisolone (Orapred, Prelone), triamcinolone (Aristospan Intra-Articular, Aristospan intralesional, Kenalog), methylprednisolone (Medrol, Depo-Medrol, Solu-Medrol), or dexamethasone (Dexamethasone Intensol). In certain embodiments, the anti-inflammatory comprises or consists of a non-steroidal anti-inflammatory (NSAID). In certain embodiments, the NSAID comprises or consists of aspirin, celecoxib, diclofenac, diflunisal, etodolac, ibuprofen, indomethacin, ketoprofen, ketorolac, nabumetone, naproxen, oxaprozin, piroxicam, salsalate, sulindac, or tolmetin.

In certain embodiments, the heparin-associated polypeptide(s) of the current disclosure are included in a pharmaceutical composition suitable for intravenous administration comprising one or more pharmaceutically acceptable excipients, carriers, and diluents. In certain embodiments, the polypeptides of the current disclosure are administered suspended in a sterile solution. In certain embodiments, the solution is one commonly used for administration of biological formulations, and comprises, for example, about 0.9% NaCl or about 5% dextrose. In certain embodiments, the solution further comprises one or more of: buffers, for example, acetate, citrate, histidine, succinate, phosphate, potassium phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, histidine, leucine, or arginine, antioxidants, for example, ascorbic acid, methionine; or chelating agents, for example, EDTA, or EGTA.

In certain embodiments, the heparin-associated polypeptide(s) of the current disclosure are included in a pharmaceutical composition suitable for intramuscular or subcutaneous administration comprising one or more pharmaceutically acceptable excipients, carriers, and diluents. Formulations suitable for intramuscular or subcutaneous injection can include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include ethanol, polyols (inositol, propyleneglycol, polyethylene-glycol, glycerol, cremophor and the like) and suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants Formulations suitable for subcutaneous injection also contain optional additives such as preserving, wetting, emulsifying, and dispensing agents.

In certain embodiments, the heparin-associated polypeptide(s) of the current disclosure are formulated for topical administration as a cream, gel, paste, ointment, or emulsion. Excipients in a cream, gel, paste, ointment, or emulsion can comprise gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethylcellulose phthalate, noncrystalline cellulose, magnesium aluminum silicate, triethanolamine, polyvinyl alcohol, polyvinylpyrrolidone, sugars, and starches.

The excipient used with the heparin-associated polypeptide(s) described herein will allow for storage, formulation, or administration of highly concentrated formulations. In certain embodiments, a highly concentrated heparin-associated polypeptide(s) comprises at least about 1, 2, 3, 4, 5, 6, 7, 8, 9, 10, 15, 20, 25, 20, 25, 40, 45, 50 or more milligrams per milliliter.

In certain embodiments, the polypeptides of the current disclosure are shipped/stored lyophilized and reconstituted before administration. In certain embodiments, lyophilized heparin-associated polypeptide formulations comprise a bulking agent such as, mannitol, sorbitol, sucrose, trehalose, and dextran 40. The lyophilized formulation can be contained in a vial comprised of glass. The heparin-associated polypeptides when formulated, whether reconstituted or not, can be buffered at a certain pH, generally less than 7.0 in certain embodiments, the pH can be between 4.5 and 6.5, 4.5 and 6.0, 4.5 and 5.5, 4.5 and 5.0, or 5.0 and 6.0.

### Further Embodiments

1. A composition comprising a mitogenic and/or fusion promoting polypeptide, wherein the polypeptide is a heparin-associated polypeptide secreted from a stem cell or a transformed cell line, wherein the heparin-associated polypeptide possesses mitogenic and/or fusion promoting activity. 2. The composition of embodiment 1, comprising a plurality of mitogenic and/or fusion promoting polypeptides. 3. The composition of embodiment 2, wherein the plurality comprises three, four, five, six, seven, eight, nine, ten or more mitogenic and/or fusion promoting polypeptides. 4. The composition of embodiment 1 to 3, wherein the mitogenic and/or fusion promoting polypeptide or plurality of mitogenic and/or fusion promoting polypeptides are isolated and purified. 5. The composition of embodiment 1 to 3, wherein the mitogenic and/or fusion promoting polypeptide or plurality of mitogenic and/or fusion promoting polypeptides are recombinantly produced. 6. The composition of embodiment 1 to 3, wherein the mitogenic and/or fusion promoting polypeptide or plurality of mitogenic and/or fusion promoting polypeptides are secreted from a stem cell. 7. The composition of embodiment 1 to 3, wherein the stem cell is a pluripotent stem cell. 8. The composition of embodiment 1 to 3, wherein the stem cell is an induced pluripotent stem cell. 9. The composition of embodiment 1 to 3, wherein the mitogenic activity comprises the ability to increase proliferation in a somatic cell or increase the ability of another heparin-associated polypeptide to increase proliferation in a somatic cell 10. The composition of embodiment 1 to 3, wherein the fusion promoting activity comprises the ability to increase fusion of a plurality of somatic cells or increase the ability of another heparin-associated polypeptide to increase fusion in a plurality of somatic cells. 11. The composition of embodiment 10, wherein the somatic cell is a muscle, muscle progenitor cell, tenocyte, or tenocyte precursor. 12 The composition of embodiment 11, wherein the somatic cell is a mammalian cell. 13. The composition of embodiment 11, wherein the somatic cell is a human cell. 14 The composition of any one of embodiments 1 to 13, wherein any one of the mitogenic and/or fusion promoting polypeptides or plurality of mitogenic and/or fusion promoting polypeptides are produced in a heterologous cellular production system. 15. The composition of any one of embodiments 1 to 13, wherein any one of the mitogenic and/or fusion promoting polypeptides or plurality of mitogenic and/or fusion promoting polypeptides are synthetically produced. 16. The composition of any one of embodiments 1 to 15, wherein the mitogenic and/or fusion promoting polypeptide or plurality of mitogenic and/or fusion promoting polypeptides comprise one or more modifications to improve activity, stability, or increase polypeptide yield from a heterologous cellular production system. 17. The composition of embodiment 16, wherein the modification is an alteration of one or more amino acids in the polypeptide sequence of the mitogenic and/or fusion promoting polypeptide compared to the wildtype polypeptide sequence of the mitogenic polypeptide. 18. A nucleic acid encoding at least one mitogenic and/or fusion promoting polypeptide of any one of embodiments 1 to 17, 19. A cell line comprising the nucleic acid of embodiment 18, 20. The cell line of embodiment 19, wherein the cell line is a eukaryotic cell line. 21. The composition comprising the mitogenic and/or fusion promoting polypeptide or plurality of mitogenic and/or fusion promoting polypeptides of any one of embodiments 1 to 17, and a pharmaceutically acceptable excipient, carrier, or diluent 22. The composition of any one of embodiments 1 to 17 or 21, formulated in injectable form. 23. Use of the composition of any one of embodiments 1 to 17, 21, or 22 in a method of treating an aging disorder, a muscle wasting disorder, a muscle injury, or an injury to a connective tissue 24. The use of embodiment 23, wherein the aging disorder is sarcopenia. 25. The use of embodiment 23, wherein the muscle wasting disorder is a muscular dystrophy. 26. A method of treating an individual with aging disorder, a muscle wasting disorder, a muscle injury, or an injury to a connective tissue comprising administering to the individual the composition of any one of embodiments 1 to 17, 21, or 22, 27. The method of embodiment 26, wherein the muscle wasting disorder is sarcopenia. 28. The method of embodiment 26, wherein the aging disorder is a muscular dystrophy. 29. A method of producing a composition suitable for the treatment of an aging disorder comprising admixing a pharmaceutically acceptable excipient, carrier, or diluent with the mitogenic and/or fusion promoting polypeptide or plurality of mitogenic and/or fusion promoting polypeptides of any one of embodiments 1 to 17, 30. A method of producing a mitogenic and/or fusion promoting polypeptide comprising culturing the cell line of embodiment 19 under conditions sufficient to produce the mitogenic and/or fusion promoting polypeptide.

31. A composition comprising a mitogenic and/or fusion promoting polypeptide, wherein the mitogenic and/or fusion promoting polypeptide is identified by: a) identifying at least one polypeptide in a mixture of a plurality of polypeptides secreted from a stem cell or a transformed cell line that binds heparin; and b) determining the mitogenic and/or fusion promoting activity of the at least one polypeptide for a somatic cell, wherein the at least one polypeptide is identified as a mitogenic and/or fusion promoting polypeptide if the at least one polypeptide exhibits mitogenic and/or fusion promoting activity 32. The composition of embodiment 31, wherein the composition comprises a mixture of a plurality of mitogenic and/or fusion promoting polypeptides. 33. The composition of embodiment 32, wherein the plurality of mitogenic and/or fusion promoting polypeptides comprises three, four, five, six, seven, eight, nine, ten or more polypeptides. 34. The composition of any one of embodiments 31 to 33, wherein the mitogenic and/or fusion promoting polypeptide or plurality of mitogenic and/or fusion promoting polypeptides are isolated and purified. 35. The composition of any one of embodiments 31 to 33, wherein the mitogenic and/or fusion promoting polypeptide or plurality of mitogenic and/or fusion promoting polypeptides are recombinantly produced. 36. The composition of any one of embodiments 31 to 33, wherein the mitogenic and/or fusion promoting polypeptide or plurality of mitogenic and/or fusion promoting polypeptides are secreted from a stem cell. 37. The composition of any one of embodiments 31 to 33, wherein the stern cell is a pluripotent stem cell. 38. The composition of any one of embodiments 31 to 33, wherein the stem cell is an induced pluripotent stem cell. 39. The composition of any one of embodiments 31 to 33, wherein the mitogenic activity comprises the ability to increase proliferation in a somatic cell or increase the ability of another heparin-associated polypeptide to increase proliferation in a somatic cell. 40. The composition of any one of embodiments 31 to 33, wherein the fusion promoting activity comprises the ability to increase fusion of a plurality of somatic cells or increase the ability of another heparin-associated polypeptide to increase fusion in a plurality of somatic cells 41. The composition of embodiment 40, wherein the somatic cell is a muscle, muscle progenitor cell, tenocyte, or tenocyte precursor. 42. The composition of embodiment 41, wherein the somatic cell is a mammalian cell. 43. The composition of embodiment 41, wherein the somatic cell is a human cell. 44. The composition of any one of embodiments 31 to 43, wherein any one of the mitogenic and/or fusion promoting polypeptides or plurality of mitogenic and/or fusion promoting polypeptides are produced in a heterologous cellular production system. 45. The composition of any one of embodiments 31 to 43, wherein any one of the mitogenic and/or fusion promoting polypeptides or plurality of mitogenic and/or fusion promoting polypeptides are synthetically produced. 46. The composition of any one of embodiments 31 to 43, wherein the mitogenic and/or fusion promoting polypeptide or plurality of mitogenic and/or fusion promoting polypeptides comprise one or more modifications to improve activity, stability, or increase polypeptide yield from a heterologous cellular production system. 47. The composition of embodiment 46, wherein the modification is an alteration of one or more amino acids in the polypeptide sequence of the mitogenic and/or fusion promoting polypeptide compared to the wildtype polypeptide sequence of the mitogenic and/or fusion promoting polypeptide. 48. A nucleic acid encoding at least one mitogenic and/or fusion promoting polypeptide of any one of embodiments 31 to 47, 49. A cell line comprising the nucleic acid of embodiment 48, 50 The cell line of embodiment 49, wherein the cell line is a eukaryotic cell line. 51. The composition comprising the mitogenic and/or fusion promoting polypeptide or plurality of mitogenic and/or fusion promoting polypeptides of any one of embodiments 31 to 47, and a pharmaceutically acceptable excipient, carrier, or diluent. 52. The composition of any one of embodiments 31 to 47 or 51, formulated in injectable form. 53. Use of the composition of any one of embodiments 31 to 47, 51, or 52 in a method of treating an aging disorder, a muscle wasting disorder, a muscle injury, or an injury to a connective tissue 54. The use of embodiment 53, wherein the aging disorder is sarcopenia. 55. The use of embodiment 53, wherein the muscle wasting disorder is muscular dystrophy. 56. A method of treating an individual with an aging disorder, a muscle wasting disorder, a muscle injury, or an injury to a connective tissue comprising administering to the individual the composition of any one of embodiments 31 to 47, 51, or 52, 57. The method of embodiment 56, wherein the aging disorder is sarcopenia. 58. The method of embodiment 56, wherein the muscle wasting disorder is a muscular dystrophy. 59. A method of producing a composition suitable for the treatment of an aging disorder, a muscle wasting disorder, a muscle injury, or an injury to a connective tissue comprising admixing a pharmaceutically acceptable excipient, carrier, or diluent with the mitogenic and/or fusion promoting polypeptide or plurality of mitogenic and/or fusion promoting polypeptides of any one of embodiments 31 to 47. 60. A method of producing a mitogenic and/or fusion promoting polypeptide comprising culturing the cell line of embodiment 59 under conditions sufficient to produce the mitogenic and/or fusion promoting polypeptide. 61. A composition comprising a mitogenic and/or fusion promoting polypeptide, wherein the mitogenic and/or fusion promoting polypeptide comprises a protein listed in Table 2, and combinations thereof. 62. The composition of embodiment 61, wherein the mitogenic and/or fusion promoting polypeptide comprises VTN, POSTN, FGF17, THBS2, or THBS4. 63. The composition of embodiment 61, wherein the mitogenic and/or fusion promoting polypeptide comprises VTN 64. The composition of embodiment 61, wherein the mitogenic and/or fusion promoting polypeptide comprises POSTN. 65. The composition of embodiment 61, wherein the mitogenic and/or fusion promoting polypeptide comprises FGF17. 66. The composition of embodiment 61, wherein the mitogenic and/or fusion promoting polypeptide comprises THBS2. 67. The composition of embodiment 61, wherein the mitogenic and/or fusion promoting polypeptide comprises THBS4. 68. The composition of any one of embodiments 61 to 67, wherein the composition comprises a mixture of a plurality of mitogenic and/or fusion promoting polypeptides. 69. The composition of embodiment 68, wherein the plurality of mitogenic and/or fusion promoting polypeptides comprises three, four, five, six, seven, eight, nine, ten or more mitogenic and/or fusion promoting polypeptides. 70. The composition of any one of embodiments 61 to 69, wherein the mitogenic and/or fusion promoting polypeptide or plurality of mitogenic and/or fusion promoting polypeptides are isolated and purified. 71. The composition of any one of embodiments 61 to 69, wherein the mitogenic and/or fusion promoting polypeptide or plurality of mitogenic and/or fusion promoting polypeptides are recombinantly produced. 72. The composition of any one of embodiments 61 to 69, wherein the mitogenic and/or fusion promoting polypeptide or plurality of mitogenic and/or fusion promoting polypeptides are secreted from a stem cell. 73. The composition of any one of embodiments 61 to 69, wherein the stern cell is a pluripotent stem cell. 74. The composition of any one of embodiments 61 to 69, wherein the stem cell is an induced pluripotent stem cell. 75. The composition of any one of embodiments 61 to 69, wherein the mitogenic activity comprises the ability to increase proliferation in a somatic cell or increase the ability of another heparin-associated polypeptide to increase proliferation in a somatic cell. 76. The composition of any one of embodiments 61 to 69, wherein the fusion promoting activity comprises the ability to increase fusion of a plurality of somatic cells or increase the ability of another heparin-associated polypeptide to increase fusion in a plurality of somatic cells 77. The composition of embodiment 76, wherein the somatic cell is a muscle, muscle progenitor cell, tenocyte, or tenocyte precursor. 78. The composition of embodiment 77, wherein the somatic cell is a mammalian cell. 79. The composition of embodiment 77, wherein the somatic cell is a human cell. 80. The composition of any one of embodiments 61 to 79, wherein any one of the mitogenic and/or fusion promoting polypeptide or plurality of mitogenic and/or fusion promoting polypeptides are produced in a heterologous cellular production system 81. The composition of any one of embodiments 61 to 79, wherein any one of the mitogenic and/or fusion promoting polypeptide or plurality of mitogenic and/or fusion promoting polypeptides are synthetically produced. 82. The composition of any one of embodiments 61 to 79, wherein the mitogenic and/or fusion promoting polypeptide or plurality of mitogenic and/or fusion promoting polypeptides comprise one or more modifications to improve activity, stability, or increase polypeptide yield from a heterologous cellular production system. 83. The composition of embodiment 82, wherein the modification is an alteration of one or more amino acids in the polypeptide sequence of the mitogenic and/or fusion promoting polypeptide compared to the wildtype polypeptide sequence of the mitogenic and/or fusion promoting polypeptide. 84. A nucleic acid encoding at least one mitogenic and/or fusion promoting polypeptide of any one of embodiments 61 to 83. 85. A cell line comprising the nucleic acid of embodiment 84. 86 The cell line of embodiment 85, wherein the cell line is a eukaryotic cell line. 87. The composition comprising the mitogenic and/or fusion promoting polypeptide or plurality of mitogenic and/or fusion promoting polypeptides of any one of embodiments 61 to 83, and a pharmaceutically acceptable excipient, carrier, or diluent. 88. The composition of any one of embodiments 61 to 83, formulated in injectable form 89. Use of the composition of any one of embodiments 61 to 83 in a method of treating an aging disorder, a muscle wasting disorder, a muscle injury, or an injury to a connective tissue. 90. The use of embodiment 89, wherein the aging disorder is sarcopenia. 91. The use of embodiment 89, wherein the muscle wasting disorder is muscular dystrophy. 92. A method of treating an individual with an aging disorder, a muscle wasting disorder, a muscle injury, or an injury to a connective tissue comprising administering to the individual the composition of any one of embodiments 61 to 83. 93 The method of embodiment 92, wherein the aging disorder is sarcopenia. 94 The method of embodiment 92, wherein the muscle wasting disorder is a muscular dystrophy. 95. A method of producing a composition suitable for the treatment of an aging disorder, a muscle wasting disorder, a muscle injury, or an injury to a connective tissue comprising admixing a pharmaceutically acceptable excipient, carrier, or diluent with the mitogenic and/or fusion promoting polypeptide or plurality of mitogenic and/or fusion promoting polypeptides of any one of embodiments 61 to 83. 96. A method of producing a mitogenic and/or fusion promoting polypeptide comprising culturing the cell line of embodiment 95 under conditions sufficient to produce the mitogenic and/or fusion promoting polypeptide.

97. A composition comprising a mitogenic and/or fusion promoting polypeptide, wherein the mitogenic and/or fusion promoting polypeptide comprises VTN, POSTN, FGF17, THBS2, or THBS4. 98. The composition of embodiment 97, wherein the mitogenic and/or fusion promoting polypeptide comprises VTN. 99. The composition of embodiment 97, wherein the mitogenic and/or fusion promoting polypeptide comprises POSTN. 100. The composition of embodiment 97, wherein the mitogenic and/or fusion promoting polypeptide comprises FGF17. 101. The composition of embodiment 97, wherein the mitogenic and/or fusion promoting polypeptide comprises THBS2. 102. The composition of embodiment 97, wherein the mitogenic and/or fusion promoting polypeptide comprises THBS4. 103 The composition of any one of embodiments 97 to 102, wherein the composition comprises a mixture of a plurality of different mitogenic and/or fusion promoting polypeptides. 104. The composition of embodiment 103, wherein the plurality of different mitogenic and/or fusion promoting polypeptides comprise three, four, or five different mitogenic and/or fusion promoting polypeptides 105. The composition of any one of embodiments 97 to 104, wherein the mitogenic and/or fusion promoting polypeptide or plurality of mitogenic and/or fusion promoting polypeptides are isolated and purified 106. The composition of any one of embodiments 97 to 105, wherein the mitogenic and/or fusion promoting polypeptide or plurality of mitogenic and/or fusion promoting polypeptides are recombinantly or synthetically produced. 107. The composition of any one of embodiments 97 to 105, wherein the mitogenic and/or fusion promoting polypeptide or plurality of mitogenic and/or fusion promoting polypeptides are secreted from a stem cell. 108. The composition embodiment 107, wherein the stem cell is a pluripotent stem cell 109. The composition of embodiment 108, wherein the stem cell is an induced pluripotent stem cell. 110. The composition of any one of embodiments 97 to 109, wherein the mitogenic activity comprises the ability to increase proliferation in a somatic cell or increase the ability of another heparin-associated polypeptide to increase proliferation in a somatic cell 111. The composition of any one of embodiments 97 to 109, wherein the fusion promoting activity comprises the ability to increase fusion of a plurality of somatic cells or increase the ability of another heparin-associated polypeptide to increase fusion in a plurality of somatic cells. 112. The composition of embodiment 111, wherein the muscle cell precursor is a myoblast. 113. The composition of embodiment 112, wherein the somatic cell is a mammalian cell. 114. The composition of embodiment 112, wherein the somatic cell is a human cell. 115. The composition of any one of embodiments 97 to 114, wherein the mitogenic and/or fusion promoting polypeptide or plurality of mitogenic and/or fusion promoting polypeptides comprise one or more modifications to improve activity, stability, or increase polypeptide yield from a heterologous cellular production system. 116. The composition of embodiment 115, wherein the modification is an alteration of one or more amino acids in the polypeptide sequence of the mitogenic and/or fusion promoting polypeptide compared to the wildtype polypeptide sequence of the mitogenic and/or fusion promoting polypeptide. 117. The composition of embodiment 115, wherein the modification is a fusion of a mitogenic and/or fusion promoting polypeptide to a non-mitogenic and/or fusion promoting polypeptide. 118. The composition of embodiment 117, wherein the non-mitogenic and/or fusion promoting polypeptide comprises an immunoglobulin Fc region or serum albumin. 119 The composition of embodiment 117 or 108, wherein the non-mitogenic or fusion promoting polypeptide is a human polypeptide. 120. The composition of any one of embodiments 97 to 114, wherein the mitogenic and/or fusion promoting polypeptide or plurality of mitogenic and/or fusion promoting polypeptides are concatemerized. 121. The composition of embodiment 120, wherein the concatemerized polypeptides are separated by a polypeptide linker. 122. The composition of embodiment 120 or 121, wherein the concatemerized polypeptides are the same polypeptide. 123. The composition of embodiment 120 or 121, wherein the concatemerized polypeptides are different polypeptides. 124. The composition of embodiment 120 or 123, wherein the concatemerized polypeptides are covalently concatemerized through a non-peptide linkage. 125. The composition of embodiment 120 or 123, wherein the concatemerized polypeptides are non-covalently concatemerized. 126. A nucleic acid encoding a mitogenic and/or fusion promoting polypeptide of any one of embodiments 97 to 125. 127. A cell line comprising the nucleic acid of embodiment 126. 128. The cell line of embodiment 127, wherein the cell line is a eukaryotic cell line. 129. The composition comprising the mitogenic and/or fusion promoting polypeptide or plurality of mitogenic and/or fusion promoting polypeptides of any one of embodiments 97 to 125, and a pharmaceutically acceptable excipient, carrier, or diluent. 130 The composition comprising the mitogenic and/or fusion promoting polypeptide or plurality of mitogenic and/or fusion promoting polypeptides of embodiment 129, wherein the pharmaceutically acceptable excipient, carrier, or diluent increases the function of the mitogenic and/or fusion promoting polypeptide or plurality of mitogenic and/or fusion promoting polypeptides. 131. The composition comprising the mitogenic and/or fusion promoting polypeptide or plurality of mitogenic and/or fusion promoting polypeptides of embodiment 130, wherein the function comprises increasing proliferation in a muscle cell precursor, increasing stability of the mitogenic and/or fusion promoting polypeptide or plurality of mitogenic and/or fusion promoting polypeptides, or increasing bioavailability of the mitogenic and/or fusion promoting polypeptide or plurality of mitogenic and/or fusion promoting polypeptides. 132. The composition of any one of embodiments 97 to 131, formulated for subcutaneous, intravenous, intramuscular, or topical administration. 133. Use of the composition of any one of embodiments 97 to 132, in a method of treating an aging disorder, a muscle wasting disorder, a muscle injury, or an injury to a connective tissue. 134. The use of embodiment 133, wherein the aging disorder is sarcopenia. 135. The use of embodiment 133, wherein the muscle wasting disorder is a muscular dystrophy 136. A method of treating an individual with an aging disorder, a muscle wasting disorder, a muscle injury, or an injury to a connective tissue comprising administering to the individual the composition of any one of embodiments 97 to 132. 137. A method of increasing proliferation of a muscle cell precursor or connective tissue cell precursor in an individual comprising administering to the individual the composition of any one of embodiments 97 to 132. 138. The method of embodiment 137, wherein the individual is afflicted with or suspected of being afflicted with an aging disorder, a muscle wasting disorder, a muscle injury, or an injury to a connective tissue 139. The method of embodiment 138, wherein the aging disorder is sarcopenia. 140. The method of embodiment 138, wherein the muscle wasting disorder is a muscular dystrophy. 141. A method of producing a composition suitable for the treatment of an aging disorder, a muscle wasting disorder, a muscle injury, or an injury to a connective tissue comprising admixing a pharmaceutically acceptable excipient, carrier, or diluent with the mitogenic and/or fusion promoting polypeptide or plurality of mitogenic and/or fusion promoting polypeptides of any one of embodiments 97 to 125. 142. A method of producing a mitogenic and/or fusion promoting polypeptide comprising culturing the cell line of embodiment 127, under conditions sufficient to produce the mitogenic and/or fusion promoting polypeptide.

### EXAMPLES

The following illustrative examples are representative of embodiments of the compositions and methods described herein and are not meant to be limiting in any way.

### Example 1—Isolation of Heparin-Associated Polypeptides

hESC secretome collection (differentiated vs undifferentiated): Human embryonic or induced pluripotent stem cells (H1, H9, H7 lines, and 2 iPSC lines derived from 1 healthy young adult female (18-25 years) and 1 aged female (greater than 65 year) donor), were cultured in triplicate on 10 cm plates on diluted Matrigel (1-30), in mTeSR-1 (Stem Cell Technologies), for a total media volume of 10 mL per plate. Another triplicate set of hPSCs/iPSCs were cultured on 10 cm plates and differentiated after plating in mTeSR-1 by changing the medium to DMEM/F12 with 10% Bovine Growth Serum (Hyclone), and culturing for an additional 7 days, hPSCs/iPSCs and differentiated hPSCs/iPSCs (6 plates in total) were washed twice with Opti-MEM (Gibco) and then cultured in Opti-MEM for 16 hours. 10 ml media was then collected per plate as hPSCs/iPSCs-secretome or differentiated hPSCs/iPSCs-secretome containing media. Media was spun for 5 min at 1000 g and transferred to new tubes to remove cell debris, aliquoted and flash frozen at 2 mL per plate as 0.5 mL aliquots and stored at −80C; remaining 8 mL/plate was used immediately for heparin-associated protein purification.

### Heparin-Associated Protein Purification

10 mL of Heparin-Agarose Type I Beads (H 6508, Sigma Aldrich) was washed with molecular grade water and preconditioned in 1 mL OptiMEM as recommended by manufacturer. 8-9 ml secretome containing media was incubated with 1 ml Heparin-Agarose Beads for 2 hours shaking at 4° C. to allow binding. Remaining heparin-depleted hPSCs/iPSCs-conditioned medium or differentiated hPSCs/iPSCs-conditioned medium was aliquoted in 15 mL tubes, flash frozen and stored at −80C to serve as negative controls for efficacy testing. Protein bound heparin beads were washed twice via a 10 minute incubation at 4° C. in 1 mL sterile PBS+0.05% Tween. Proteins were eluted twice for 15 minutes at 4° C. in 400 μl of elution buffer A (Eluted-A) (0.01M HEPES pH 7.5+1.5M NaCl+0.1% BSA) per 10 cm plate for the first two plates, or elution buffer B (Eluted-B) lacking BSA (0.01M HEPES pH 7.5+1.5M NaCl)** for the 3rd 10 cm plate, to collect proteins in a total of 800 μL of elute per original plate. The proteins were desalted by diffusion dialysis (3500 MWCO) (by a 2 hour dialysis shaking at 4C in 50 mil McCoy's 5A Medium or similar tissue culture medium (Gibco) followed by overnight (not more than 16 hours) dialysis shaking at 4C in 200 ml OptiMEM (Gibco). The collected eluate was aliquoted in appropriately capped tubes, flash frozen and stored at −80 C.

### Secretome Heparin-Associated Fraction Validation Assays:

BCA assay (Pierce) was performed for total protein yield in the eluate using 2 ul per sample in triplicate according to manufacturer's instructions from each Elution A sample and Elution B.

SDS-PAGE Silver Stain/SDS-PAGE Coomassie was performed for protein integrity and rough MW analysis (loading <5-10 ug per lane for each sample).

### Mouse Myoblast Proliferation Assay

Reduced regeneration from an individual's tissue progenitor cells is a hallmark of aging, therefore assays that measure mitogenic capacity in tissue progenitor cells serve as a read-out for potential success of any given heparin-associated polypeptide as a regenerative factor. Measuring the increased proliferation rate of treated mouse or human muscle progenitor cells will provide good basis for potentially therapeutic regenerative factors for treating individuals who have suffered illness, injury, or who possess genetic or developmental defects leading to premature tissue loss, wasting, or weakening. As a control, the assay will also be performed on proteins purified from differentiated cells, which result in no in myoblast proliferation, cultured in medium conditioned by differentiated cells, or purified heparin-associated fractions.

Mouse muscle progenitor cells (early passage myoblasts) were cultured and expanded in mouse growth medium: Ham's F-10 (Gibco), 20% Bovine Growth Serum (Hyclone), 5 ng/mL FGF2 and 1% penicillin-streptomycin on Matrigel coated plates (1:300 matrigel:PBS), at 37° C. and 5% CO2. For experimental conditions, cells were plated at 40,000 cells/well on Matrigel coated 8-well chamber slides in 250-500 uL medium per well (1:100 matrigel:PBS) in mouse fusion medium. DMEM (Gibco)+2% horse serum (Hyclone) One hour after plating, mouse myoblasts were treated with 500% respective medias:

TABLE 4
8-well Chamber Slide A: Eluted Heparin-associated
Proteins from H9/H7 hPSCs and 2 iPSC lines -
4 slides total, 1 for each cell line tested.

| | 50% FM/50% | 50% FM/50% | 50% FM/50% |
|---|---|---|---|
| | Eloted-A | Differentiated | Differentiated |
| Fusion Media | Heparin- | hPSC- | hPSC- |
| (FM) | associated | conditioned | conditioned |
| (250 uL) | proteins | OptiMem | OptiMem |
| 50% FM (125 ul)/ | 50% FM/50% | 50% FM/50% | 50% FM/50% |
| 50% Growth | hPSC- | Heparin- | Eluted-B |
| Media | conditioned | depleted | Heparin- |
| (125 uL) | OptiMEM | hESC- | associated |
| | | conditioned | proteins |
| | | OptiMEM | (no BSA) |

TABLE 5
Assay for Eluted heparin-associated proteins purified from
Differentiated hPSSs/iPSCs (Control). 50% FM/50% OptiMEM

| | | |
|---|---|---|
| 50% FM/50% | 50% FM/50% | 50% FM/50% |
| Eluted-A | Differentiated | Eluted-B |
| Heparin- | hPSC- | Heparin- |
| associated | conditioned | associated |
| proteins | OptiMem | proteins |
| | | (no BSA) |

TABLE 6
8-well Chamber Slide B: Eluted Heparin-
associated Protein Serial Dilution.

| | 50% FM/50% | 75% FM/25% | 75% FM/12.5% |
|---|---|---|---|
| | Eluted-A | Eluted-A | Eluted-A |
| Fusion | Heparin- | Heparin- | Heparin- |
| Media (FM) | associated | associated | associated |
| (250 uL) | protein | protein | protein |
| 81.25% FM/ | 84.375% FM/ | 98.44% FM/ | 75% FM/25% |
| 6.25% | 3.125% | 1.56% | hPSC- |
| Eluted-A | Eluted-A | Eluted-A | conditioned |
| Heparin- | Heparin- | Heparin- | OptiMEM |
| associated | associated | associated | |
| protein | protein | protein | |

Mouse Myoblasts were cultured for 24 hours in the above conditions, at 37° C. in 10% CO₂ incubator. BrdU (300 μM) in DMSO was added for 2 hours prior to fixation with cold 70% ethanol and stored at 4° C. until staining.

### Quantifying Regenerative Index

Following permeabilization in PBS+0.25% Triton X-100, antigen retrieval was performed via a 10 minute 4 N HCl treatment followed by PBS washes. Primary staining was performed overnight at 4° C. in PBS+2% FBS. Primary antibodies include: a species-specific monoclonal antibody for mouse anti-embryonic Myosin Heavy Chain (eMyHC, hybridoma clone 1.652, Developmental Studies Hybridoma Bank) and Rat-anti-BrdU (Abcam Inc. ab6326) Secondary staining with fluorophore-conjugated, species-specific antibodies (Donkey anti-Rat-488, #712-485-150; Donkey anti-Mouse-488, #715485-150; all secondary antibodies from Jackson ImmunoResearch) was performed for 1 hour at room temperature at a 1.500 dilution in PBS+2% FBS. Nuclei are visualized by Hoechst staining. For cell quantification, 5 images per well were collected at 20× in each of the channels as well as DIC to achieve at least 2000 imaged cells per condition. Using the Hoechst stain to tally cell number, the percent of cells positive for BrdU and eMyHC were tabulated and reported.

Human muscle progenitor cells (myoblasts) were similarly activated to proliferate when conditioned with hPSC-secreted heparin-associated proteins. Proliferation assays were performed on human myoblasts to test protein candidate factors for enhanced precursor cell activity in an in vitro screening assay. Conditions for culturing human muscle cells were optimized to reflect the slower rate of growth and differentiation of human muscle cells, where early passage human myoblasts were cultured for 72 hours with daily medium changes rather 24 hours, and pulsed for 4 hours with BrdU instead of 2 hours.

### Example 2—Characterization of the Protein Components of the Heparin Head Binding hPSC Secretomes

### Protein Quantification

The protein concentration in the eluted sample was determined using the bicinchoninic acid (BCA) protein assay (Thermo Fisher Scientific, Waltham, MA). The protocol was performed as follows: A volume containing 100 ug protein was extracted and disulfide bonds were reduced with 5 mM tris-(2-carboxyethyl)-phosphine (TCEP), at room temperature for 25 min, and alkylated with 10 mM iodoacetamide at room temperature for 30 min in the dark) Excess iodoacetamide was quenched with 15 mM dithiothreitol (room temperature, 15 min in the dark). At this point the sample were split, with 20 μg analyzed immediately via SDS-PAGE Silver Stain, 20 μg saved for SDS-PAGE Coomassie stained gel band analysis, and 60 ug proceeded to in-solution mass spectrometry sample preparation.

### Quantify the Size Distribution of Proteins

Silver staining provides a sensitive, rapid, low cost way to survey the complexity and general molecular weight distribution of the proteins in a complex mixture. By running a matched sample treated to remove glycans, the presence of this PTM common secreted proteins can be determined by the resulting shift in apparent molecular weight. Additional rounds of selective glycosylation reactions can then be run to gain insight into the identity and structure of glycan modifications on proteins of interest. Five micrograms of sample can be removed and treated with Protein Deglycosylation Mix II (NEB) to remove all N-linked and simple O-linked glycans as well as some complex O-linked glycans, which can be visualized by molecular weight shifts relative to an untreated control on a silver stained SDS-PAGE gel.

A 4-12% acrylamide gel (BioRad) in 1×MOPS buffer was loaded gel with samples (>0.20 ug/lane) and ladder (as per manufacturer's instructions), run at 200V for 45 minutes or until sample front neared the bottom of the gel, and incubated in 50% methanol/50% LC grade water >1 hour. Stain solution was prepared adding a solution of 0.8 g AgNO3 in 4 mL LC grade H₂O dropwise into a solution of 1 mL 0.36% NaOH+1.4 mL 14.8M ammonium hydroxide under constant stirring followed by the addition of LC grade water to a final volume of 100 mL. Gel staining proceeded by incubating gel in stain solution for 15 minutes, before washing twice with LC grade water, allowing 5-8 minutes of incubation per wash step. The silver stain was developed by incubation in a solution of 0.25 mL 1% citric acid+25 uL 37% formaldehyde in 50 mL LC grade water for 10-15 minutes in the dark (or until desired density was achieved). Developer solution was removed and the gel washed with LC grade water to slow development for an imaging series, or development was stopped by incubation in a solution of 45% methanol, 10% acetic acid.

### In-Solution Mass Spectrometry Sample Preparation

Methanol-chloroform precipitation was performed prior to protease digestion (a standard trichloroacetic acid-based precipitation protocol would be substituted here if protein yield from the heparin head eluates are below 25 ug total). In brief, four parts neat methanol was added to each sample and vortexed, one part chloroform was added to the sample and vortexed, and three parts water was added to the sample and vortexed. The sample was centrifuged at 4,000 RPM for 15 min at room temperature and subsequently washed twice with 100% methanol, prior to air-drying. Samples were resuspended in 50 mM HEPES pH 8.5 and digested at room temperature for 12 hrs with LysC protease at a 100:1 protein-to-protease ratio. Trypsin was then added at a 100:1 protein-to-protease ratio and the reaction was incubated 6 hours at 37° C. Peptide concentrations in the digests were measured using the Quantitative Colorometric Peptide assay kit (Pierce). From each sample 10 ug of peptide digestion solution was taken and enzymatic activity quenched with formic acid to a final pH of <2 before de-salting via C-18 Stagetips, using a standard formic acid/acetonitrile buffer system. Stagetips were eluted directly into autosampler vials in a buffer of 70% acetonitrile and 1% formic acid, dried in a vacuum concentrator, and stored at −80C until being resuspended to ˜1 ug/μl of Buffer A (typically ˜0.2% formic acid, 5% acetonitrile) for mass spectrometry analysis.

### SDS-PAGE Coomassie and In-Gel Band Mass Spectrometry Sample Preparation

A gel based sample preparation pipeline may be employed if the abundance distribution of the sample is heavily skewed, or where only a few species of proteins account for a substantial majority of the molecules in the sample. This size based separation method has been shown to effectively improve depth of proteomic coverage in biochemically purified protein mixtures.

Briefly, the protocol begins by running >20 ug per lane of sample out on an SDS-PAGE as in the Silver Stain method above, staining and destaining by Coomassie as per manufacturer's instructions, excising sections of the gel containing potentially interesting proteins, and cutting excised gel sections into 1 mm×1 mm squares. Ensure gel pieces are at neutral pH by adding 50-100 μl 100 mM Ammonium bicarbonate, let sit for 10 minutes and discard. Wash gel pieces with 100-150 μl 50 mM Ammonium bicarbonate/50% acetonitrile for 10 minutes, vortexing every 5 minutes to dehydrate Depending on intensity of stain, repeat step 9 until the gel pieces are clear. Discard solution phase and dry samples in speed vac for 5-10 minutes. To digest proteins add 5 pmol sequencing grade trypsin (Promega Corp.) in 50 mM Ammonium bicarbonate and 0.02% Protease Max to each sample and incubate overnight in 37° C. on a shaking heatblock. Spin down samples at 1000G for 2 minutes, pull off all liquid, and transfer to a glass autosampler vial. Add 40-50 μl 1% formic acid, 66% acetonitrile 33% 100 mM Ammonium bicarbonate and incubate for 10 minutes at 37° C. to increase peptide release from gel. Spin at 10,000G for 2 minutes to pellet insoluble protein or detergent degradation production. Extract all solution being sure to avoid pellet areas and combine into autosampler vial. Speed vac total combined extracts to dryness and store at −80C until being resuspended to ˜1 ug/μl of Buffer A (typically ˜0.2% formic acid, 5% acetonitrile) for mass spectrometry analysis.

nHPLC-MS2 Instrumentation and Analysis

Two, 3-hr gradients were collected per sample using an Orbitrap Fusion instrument coupled to a Waters liquid chromatography (LC) pump (Thermo Fisher Scientific). Peptides are fractionated on a 100 μm inner diameter microcapillary column packed with ˜25 cm of Accucore 150 resin (1.2 μm, 150 Å, ThermoFisher Scientific). For each analysis, 1 μg per sample was loaded onto the column. Peptides were separated using a 3 hr gradient of 6 to 46% acetonitrile in 0.2% formic acid at a flow rate of ˜400 nL/min. Instrument settings for the Orbitrap fusion were as follows: FTMS1 resolution (120,000), ITMS2 isolation window (0.4 m/z), ITMS2 max ion time (120 ms), ITMS2 AGC (2E4), ITMS2 CID energy (35%), dynamic exclusion window (90 sec). A TOP10 method was used where each FTMS1 scan was used to select up to 10 FTMS2 precursors for interrogation by HCD-MS2 with readout in the orbitrap.

### Data Analysis

Resulting mass spectra were searched using commercially available analysis software (e.g., Byonic) against a human database publicly available from Uniprot which was concatenated with common contaminants and reversed sequences of the human and contaminant proteins as decoys for FDR determination. Searches restricted the precursor ion tolerance to 20 ppm, and product ion tolerance window was set to 0.5 m/z. Searches allowed up to two missed cleavages, including static carbamidomethylation of cysteine residues (+57.021 Da) and variable oxidation of methionine residues (+15.995 Da). Additional variable modifications may be included, particularly glycosylations, based on the results of the gel shift assay following de-glycosylation treatment or the preview search PTM scan. Results were filtered to a 1% FDR at the protein level per sample.

### Example 3—In Vitro Screening of Stem Cell Secreted Factors

A deeper understanding of a given protein factor's contribution to the regenerative effects of the pool of heparin-associated hPSC secretome will be gained by screening against an established panel of assays for cellular age Assays include measurements of reactive oxygen species (ROS) production or tolerance, cytoplasmically and in the mitochondria, telomerase activity, measurements of proteostasis capacity via lysosomal, autophagy, and proteasomal routes, epigenetic re-patterning, and cellular energy balance (e.g., ATP/ADP and NAD/NADH ratios). Many of these assay leverage the high-throughput automated microscopy described above to make these measurements in a variety of cell types, including fibroblast, endothelial cells, mesenchymal stem cells, and chondrocytes. Collectively these metrics can inform both the pathway and the mechanisms by which the heparin-associated hPSC secretome or its individual components enact their regenerative effects.

To begin screening and validating potential regenerative protein factors protein coding sequences will be collected from a publicly available source, such as used for the proteomics analysis (e.g., UniProt). The sequence for each of the proteins will be used to construct a DNA sequence encoding the proteins. The sequences are then each cloned into a plasmid vector system tailored for inducible or constitutive high-copy expression (in mammalian or prokaryotic settings). Alternatively such a plasmid vector system may be designed in silico. Such a plasmid can be transformed into a pool of cells where the encoded protein was transiently expressed from the plasmid. Alternatively the gene of interest could be incorporated in the genomes of a pool of cells (e.g. lentiviral transduction for mammalian cells or homologous recombination for S. cerevisiae) to create stable cell lines for recombinant protein production.

To de-bulk the target factor list and validate recombinant expression for factor production, a viable route would be to express the construct in a human cell line (like 293T-REx), which exploits: 1) that proteins of interest naturally purify themselves during the secretion process, and 2) will be processed in a natural context, potentially preserving important post translational processing steps. T-REx cells will be grown on 10 cm plates to ˜50% confluence in DMEM with 10% Bovine Growth Serum (Hyclone), 2 mM L-glutamine, and 1% Pen-Strep before initiating translation of a target protein of interest for 48 hr. The media would be collected, spun at 2,000 g to purify, and the supernatant used for heparin-associated enrichment of target factors in mouse myoblast regeneration assays.

### Machine Learning Classifier

By combining and statistically comparing the information from the Regenerative index assay, the Panel of Cellular Age Makers, the Proteomics we can create deep feature vectors for each protein factor, the pool of all factors (from each repeat of the assays), and the negative control pool (from each repeat of the assays). Treating the pool of all factors (or known factors such as FGF-2) as True Positives, and the negative control pool (or known non-functional proteins such as BSA) as True Negatives a supervised clustering algorithm can be trained to classify protein factors. Using a standard 10-fold cross validation scheme to assess the relative accuracy, recall, and confusion matrix graphs of the output of various algorithms' outputs (eg, Naive Bayes, Support Vector Machine, Linear Regression, or Random Forest) trained classifier most likely to successfully distinguish proteins with regenerative potential from the set of target factors can be selected Target factors (or tested combinations) can then be rank ordered by the probability they derive from the regenerative set compared to the null set. A number of the top scoring target factors (or tested combinations) will then be selected for GMP-grade production for in vivo and in vitro validation.

Based on the complexity of the original heparin-associated fraction of the hPSC secretome and the limits to which individual proteins can recapitulate the activity of the whole pool, we will test combinations of factors as well. In the simplest approach, we would combine the 293T-REx secretome containing media from two or more cell lines each producing a given factor, and test their combined regenerative efficacy across a range of concentrations in an isobologram analysis using the regenerative index from the Myoblast Regeneration assay.

### Example 4—In Vivo Testing of Stem Cell Secreted Factors

There are two main aspects of muscle degeneration with aging, acute loss following trauma and chronic wasting (sarcopenia), and both of them will be tested. As the therapeutic approach to each case is expected to be different two arms for the in vivo validation is envisioned to specifically test each use-case for the factors as therapeutics in humans. The following Acute Injury Model and Sarcopenia/Chronic Administration Model for the most promising proteins emerging from the machine learning classifier can be carried out

### Acute Injury Model

Animals were kept under standard animal husbandry condition Animals were fed standard chow, and have ad libitum access to food and water. Temperature were kept at 22° C. and 12 h light/12 h dark cycles Animals were acclimated prior to study initiation. The experimental design was: C57BL/6J male mice, N=18; Young. 12-13 week old (3-month-old) mice, n=6; Aged: 77-78 week old (18-month-old) mice, n=12. This design was used to test any single factor identified and validated in in vitro assays or complex mixtures of 2 or more factors

TABLE 7
Experimental design of acute injury model.

| | | | | Dose | | Blood | Tissue |
|---|---|---|---|---|---|---|---|
| No | Group | n | Test | (mg/kg) | Administration | collection | collection |
| 1 | Young, | 6 | vehicle | n/a | i.m, q.d; into | terminal | TA/GA muscles |
| | Vehicle | | | | injured muscle | | into 25% |
| 2 | Aged, | 6 | vehicle | n/a | site on day 0, | | sucrose; Brain, |
| | Vehicle | | | | and 2 | | liver, heart, |
| 3 | Aged, | 6 | Test | 0.1 | | | and lung into |
| | Test | | factor | | | | 4% PFA |
| | factor | | | | | | |

On Day 0, mice were weighed and underwent muscle injury with focal injection of barium chloride (BaCl, 10 μL, 1.2% w/v in saline, Sigma-Aldrich) in the Tibialis anterior (TA; Day 0) of both the right and left hindlegs. Injections of vehicle or factor A (0.1 mg/kg) were co-administered intramuscularly (i.m) following the BaCl into the TA injured hindleg sites, and again 48 hours later on day 2 (i.m.) into the TA injured hindleg sites. Also on day 2, BaCl (Ctx; 10 μL, 1.2% w/v in saline, Sigma-Aldrich) was injected into the Gastrocnemius (GA, Day 2, i.m.) muscles of both right and left hind legs. Injections of vehicle or a factor were sequentially administered (i.m.) following the BaCl into the TA hindleg sites post-injury, and again 48 hours later on day 4 (i.m.) into the GA injured hind leg sites Bromodeoxyuridine (BrdU) was be administered (100 mg/kg, i.p) once daily for 3 days, day 2-4, before sacrifice to label proliferating cells.

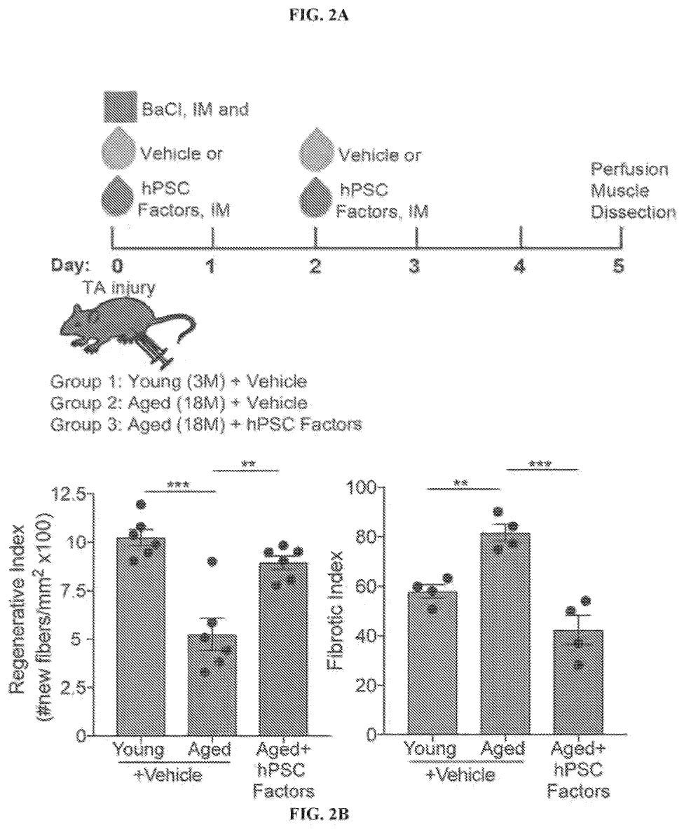

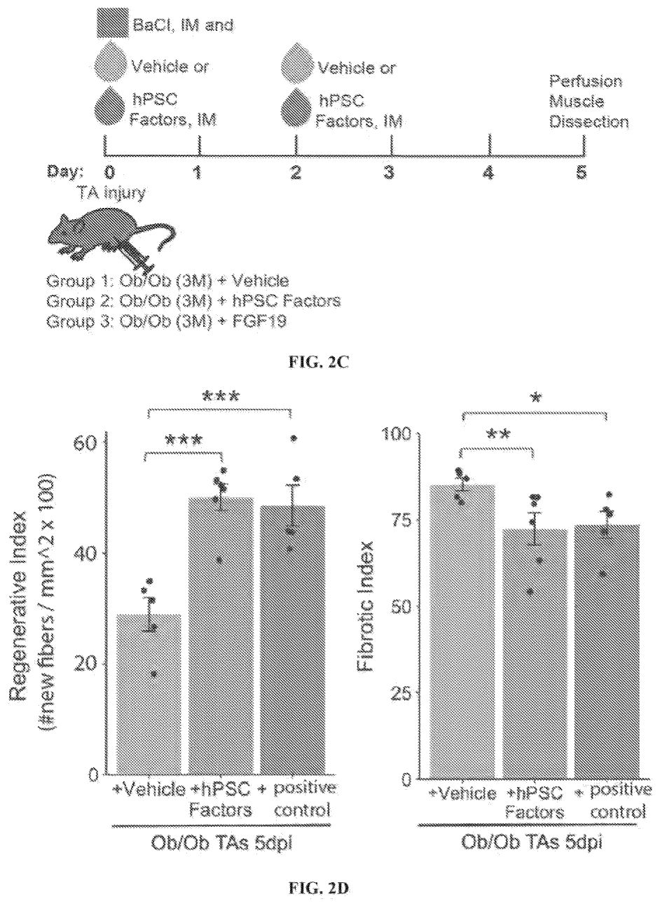

On day 5, animals were sacrificed and animal weight recorded followed by collecting 0.5 ml of terminal blood via cardiac puncture which was processed to plasma and stored at 80° C. We then perfused the animal with 1×PBS, carefully dissected the skin from the GA/TA muscles of each hind leg and took photos (prior to excision). After excision of exclusively the GA or TA muscle, excised tissue was photographed, weighed, then placed into 25% sucrose in PBS at 4° C. for 4 hr rinsed in 1×PBS, immersed in Tissue-TEK OCT and rapidly frozen before storing the muscles tissues frozen at 80° C. Cryosectioning and H&E were performed to ensure muscle injury site was appropriately visualized. A diagram of the time points for these experiments can be seen in FIG. 2A. Muscle tissue composition from new skeletal muscle fibers, fibrotic tissue, and adipose (fat), were measured. Muscle regeneration, as defined as the number of number of new myofibers with centrally located nuclei per millimeter, fibrosis as defined as the area of fibrotic scarring, size of the fibers, as defined as the width and area, adipose tissue, as defined by the amount of fat surrounding the muscle, were measured to assess level of regeneration.

Young muscle regeneration after acute focal injury had the highest regenerative index (measured as the number of new myofibers with centrally located nuclei per millimeter squared). Aged muscle regeneration following acute injuries performed on average 30-50% less than young muscle regeneration, in part due to the reduction of mitogenic muscle precursor/progenitor cell activity (FIG. 2B). It was predicted that systemic treatment of aged animals, injured animals, animals with genetic diseases causing muscle wasting, or animals injured by radiation or other tissue damaging treatment, the heparin-associated polypeptides, individually or in various combinations, will improve skeletal muscle regeneration by 20-50%, approaching comparable levels to young healthy animals. It was also predicted that the composition of fibrous scar tissue and fat tissue will be reduced to levels comparable to younger animals by treatment with the heparin-associated polypeptides. And indeed, treatment with the heparin-associated polypeptides did improve the skeletal muscle regeneration of sarcopenic mice to level indistinguishable from the young by both the number of new myoblasts and the reduction in fibrous scar tissue (FIG. 2B).

### Sarcopenia/Chronic Administration Model

After arrival, animals will be kept under standard animal husbandry condition. Animals will be fed standard chow, and have ad libitum access to food and water. Temperature will be kept at 22° C. and 12 h light/12 h dark cycles. Animals will be acclimated prior to study initiation, including any in vivo assay acclimation, if necessary. The experimental design was C57BL/6J male mice, N=18; Young. 12-13 week old (3-month-old) mice, n=6; Aged: 77-78 week old (18-month-old) mice, n=12. This design can be used to test any single factor identified and validated in in vitro assays or complex mixtures of 2 or more factors.

TABLE 8
Design of sarcopenia/chronic administration model.

| | | | | Dose | | | Blood | Tissue |
|---|---|---|---|---|---|---|---|---|
| No | Group | n | Test | (mg/kg) | Administration | In vivo assay | collection | collection |
| 1 | Young, | 6 | vehicle | n/a | i.p, q.d; on | 2 sets of: Animal | terminal | TA/GA muscles |
| | Vehicle | | | | day −8 to +5 | weight, grip | | into 25% |
| 2 | Aged, | 6 | vehicle | n/a | | strength, running | | sucrose; Brain, |
| | Vehicle | | | | | wheel performance; | | liver, heart, |
| 3 | Aged, | 6 | Test | 0.1 | | horizontal bar | | and lung into |
| | Test | | factor | | | 1 set of: | | 4% PFA |
| | factor | | | | | Incapacitance | | |

On Day −10, mice will have the following in vivo healthspan measurements will be performed over 1 day as a baseline for age-based parameters: Weight, running wheel performance, grip strength, and horizontal bar. Each assay should be run for 4 trials per assay per animal. These healthspan assays will be repeated on day −1. After one day of rest on day −9, mice will begin 1× daily injections (0.1 mg/kg) of vehicle or factor A for the remainder of the experiment until sacrifice (days −8 to +5, 13 days of dosing). On day −4, 6 days after dosing begins, mice will undergo a repeat of the healthspan assays. On day 0, 5 days prior to sacrifice, mice will undergo muscle injury with focal injection of cardiotoxin (Ctx; 10 μg, Sigma-Aldrich) in the Tibialis anterior (TA; Day 0) of the right hindleg only. On day 2, the Gastrocnemius (GA; Day 2) muscle of the right hind leg will then receive cardiotoxin (Ctx; 10 g, Sigma-Aldrich). BrdU will be administered (100 mg/kg, i.p.) once daily for 3 days, day 2-4, before sacrifice. On day +5, prior to take-down, the animals will have an in vivo incapacitance assay run. On day +5, animals will be sacrificed and animal weight recorded. Collect 0.5 ml of blood via cardiac puncture, process to plasma and store plasma samples at 80° C. The animals will then be perfused with 1×PBS. Carefully dissect the skin from the GA/TA muscles of each hind leg and take photos (prior to excision). After excision of exclusively the GA or TA muscle, weigh the muscles, then place muscles into 25% sucrose in PBS at 4° C. for 4 hr. Then rinse the muscles in 1×PBS, adding Tissue-TEK OCT and storing the muscles tissues frozen at 80° C. Perform cryosectioning and H&E, ensuring muscle injury site is appropriately visualized. Carefully excise the inguinal white adipose tissue (WAT) and weigh tissue Discard WAT post-weighing. Collected brain, liver, heart and lung can be post-fixed in 4% PFA for 72 hours, after 72 hours, transferred into 30% sucrose in 1×PBS and stored at −4° C. (brain, liver, heart, lung).

Muscle tissue composition, from new skeletal muscle fibers, fibrotic tissue, and adipose (fat), will be measured. Muscle regeneration, as defined as the number of number of new myofibers with centrally located nuclei per millimeter, fibrosis, as defined as the area of fibrotic scarring, size of the fibers, as defined as the width and area, adipose tissue, as defined by the amount of fat surrounding the muscle, will be measured to assess level of regeneration. Weights of the animals during the duration of treatment with heparin-associated polypeptide(s), as well as healthspan assays including performance on a running wheel (speed, distance, duration), grip strength, and performance on a horizontal bar will take into account the phenotypic outcomes of treatment of the aged animals systemically with the heparin-associated polypeptides for sarcopenia.

The horizontal bar test is performed as described previously (Malinowska et al. 2010) at 8 months (n=6 WT, n=7 MPS 1103) and 10 months (n=3 WT, nt=4 MPS 11B) of age. In brief, a 300-mm metal wire, 2 mm in diameter, was secured between two posts 320 mm above a padded surface. The mouse was allowed to grip the center of the wire and the time to fall or reach the side was recorded, and after 2 min the test was stopped. Crossing the bar in x seconds was scored as 240-x, remaining on the bar was scored as 120, and falling off the bar after y seconds was recorded as the value of y. The test was repeated three times as a practice run followed by a 10-min rest prior to three tests where the score was recorded.

Young muscle regeneration after acute focal injury has the highest regenerative index (measured as the number of new myofibers with centrally located nuclei per millimeter squared). Aged muscle regeneration following acute injuries performs on average 30-50% less than young muscle regeneration, in part due to the reduction of mitogenic muscle precursor/progenitor cell activity. We predict systemic treatment of aged animals, injured animals, animals with genetic diseases causing muscle wasting, or animals injured by radiation or other tissue damaging treatment, the heparin-associated polypeptides, individually or in various combinations, will improve skeletal muscle regeneration by 20-50%, approaching comparable levels to young healthy animals. We also predict the composition of fibrous scar tissue and fat tissue will be reduced to levels comparable to younger animals by treatment with the heparin-associated polypeptides.

Animals will also have better healthspan outcomes: reduced weight, fat composition, scar tissue around muscles, increased running speed, duration, and distance, increased grip strength, and enhanced performance on the horizontal bar test.

### Example 5—Additional Tests for Pro-Regenerative Factors

Mechanistic insight into a given heparin-associated polypeptide factor's pathway of action will be gained by establishing and screening against a panel of assays for cellular age. Assays include measurements of reactive oxygen species (ROS) production or tolerance cytoplasmically and in the mitochondria, telomerase activity, measurements of proteostasis capacity via lysosomal, autophagy, and proteasomal routes, epigenetic re-patterning, and cellular energy balance (e.g., ATP/ADP and NAD/NADH ratios). Many of these assay leverage high-throughput automated microscopy to make these measurements in a variety of cell types, including fibroblast, endothelial cells, mesenchymal stem cells, and chondrocytes. Collectively these metrics can inform both the pathway and the mechanisms by which the heparin-associated hPSC secretome or its individual components enact their regenerative effects. These deep profile vectors can be crucial for approaching combinations of factors rationally, and for machine learning predictions.

To test the cellular effects of secretomes toward reversing the hallmarks of aging, high-throughput automated imaging and quantification of single cells to achieve deep population level statistical power can be employed. Cellular component state profiles of Young, Aged, and Aged+Treatment in human fibroblasts and epithelial cells, myoblasts, mesenchymal stem cells, chondrocytes, and neural progenitor cells will be compared. Some examples of tests and methods include.
1) Epigenetic reprogramming: repressive mark H3K9me3, the heterochromatin-associated protein HP1γ, nuclear lamina support protein LAP2α
2) Nuclear membrane Folding/Blebbing: immunofluorescence of the nuclear membrane protein Lamin A/C
3) Proteolytic Activity: Cleavage of fluorescent-tagged chymotrypsin like substrate corresponds to proteasome 20S core particle activity. Wells are first stained with PrestoBlue Cell Viability dye (Life Technologies) for 10 minutes. Well signals are read using a TECAN fluorescence plate reader as a measure of cell count. Then cells are washed with HBSS/Ca/Mg before switching to original media containing the chymotrypsin like fluorogenic substrate LLVY-R110 (Sigma) which is cleaved by the proteasome 20S core particle. Cells are then incubated at 37° C. in 5% CO2 for 2 hours before signals are again read on the TECAN fluorescence plate reader Readings are then normalized by PrestoBlue cell count.
4) Formation of autophagosomes: Autophagosome number and volume are measured by staining with CellTracker Deep Red (Sigma). The cells are then incubated at 37° C. in 5% CO2 for 20 minutes, washed 2 times using HBSS/Ca/Mg, and stained for 15 minutes using CellTracker Deep Red cell labeling dye. Cells are then switched to HBSS/Ca/Mg for single cell imaging using the Operetta High Content Imaging System (Perkin Elmer).
5) Energy Metabolism: ATP in the cells is measured using colorimetric assay using an ATP assay kit (ab83355; Abcam, Cambridge, MA) following manufacturer's instructions. Cells are washed in cold phosphate buffered saline and homogenized and centrifuged to collect the supernatant. The samples are loaded with assay butTer in triplicate. ATP reaction mix and background control (50 μL) is added to the wells and incubated for 30 min in dark. The plate is read at OD 570 nm using SpectraMax M2e (Molecular Devices, Sunnyvale, CA). The mean optical density is used to estimate of the intracellular ATP concentration relative to the standard curve.
6) Mitochondrial Activity: To measure Mitochondria Membrane Potential, cells are washed twice with Ham's F10 (no serum or pen/strep). Subsequently, MuSCs are stained with MitoTracker Green FM (ThermoFisher, M7514) and DAPI for 30 minutes at 37° C., washed three times with Ham's F10, and analyzed using a BD FACSAria III flow cytometer. To measure
7) Mitochondrial ROS Measurement. Cells are washed with HBSS/Ca/Mg and then switched to HBSS/Ca/Mg containing MitoSOX (Thermo), a live cell permeant flurogenic dye that is selectively targeted to mitochondria and fluoresces when oxidized by superoxide. Cells are incubated for 10 minutes at 37° C. in 5% CO2. Cells are then washed twice with HBSS/Ca/Mg, and stained for 15 minutes using CellTracker Deep Red. Finally, cells are imaged in fresh I-IBSS/Ca/Mg using the Operetta High Content Imaging System (Perkin Elmer).
8) Deregulated Nutrient Sensing: levels of SIRT1 are measured.
9) Senescence: Senescence-associated beta-galactosidase staining is measured in cells washed twice with PBS then fixed with 15% Paraformaldehyde in PBS for 6 minutes. Cells are rinsed 3 times with PBS before staining with X-gal chromogenic substrate, which is cleaved by endogenous Beta galactosidase. Plates are kept in the staining solution, Parafilmed, to prevent from drying out, and incubated overnight at 37° C. with ambient CO2. The next day, cells are washed again with PBS before switching to a 70% glycerol solution for imaging under a Leica brightfield microscope
10) Secretome of the cells: Mass-Spec or O-Link for inflammatory cytokines profiles Soft Tissue Deposition: Immunofluorescence for SOX9, MMP3, MMP13, and COL2A1 expression, the decrease of which is characterized by cartilage loss, pain, cleft-lip, and joint destruction.

### Example 6—Identification of Pro-Regenerative Factors by Mass Spectroscopy

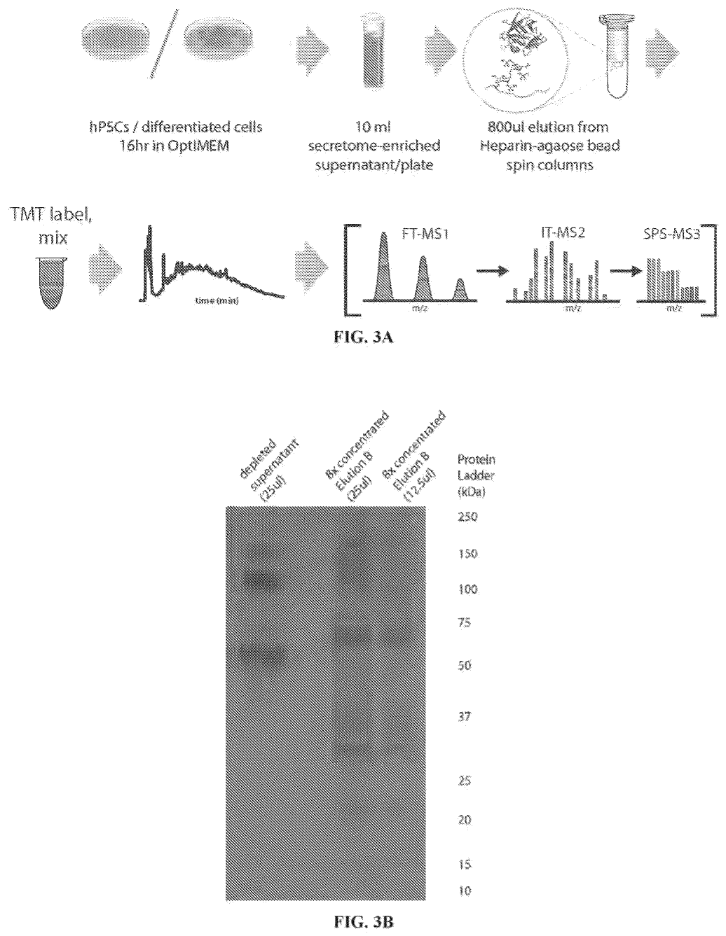

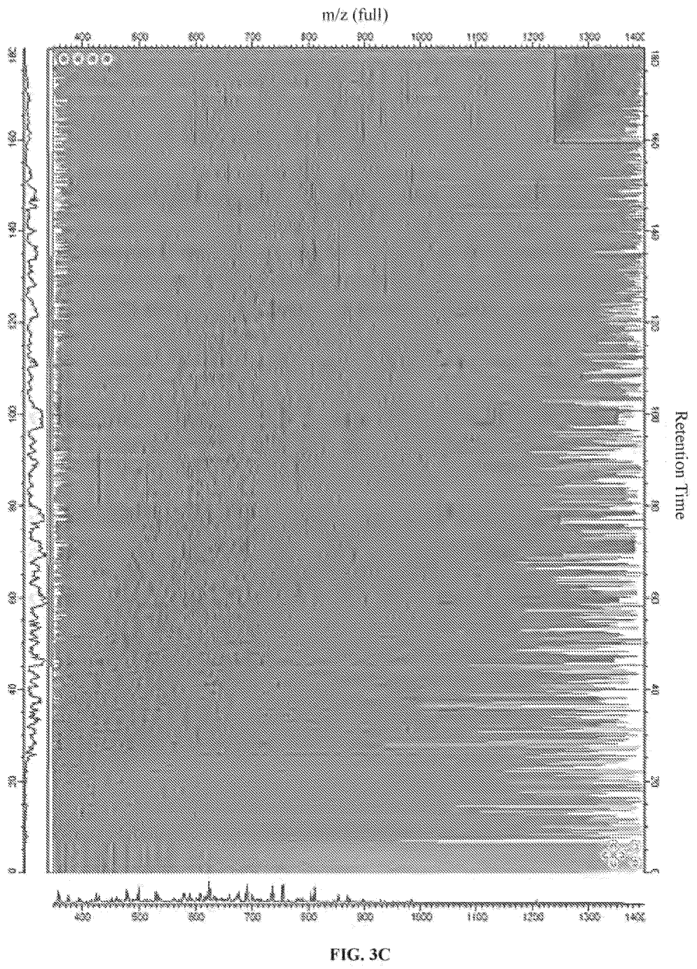

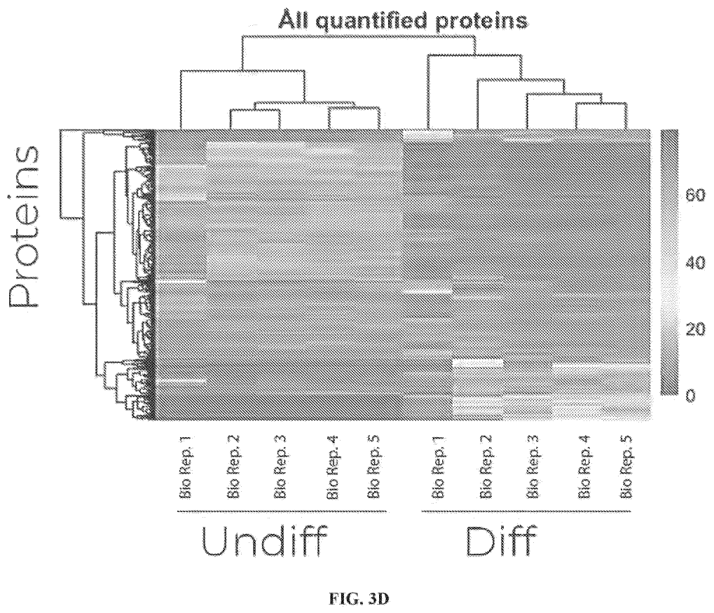

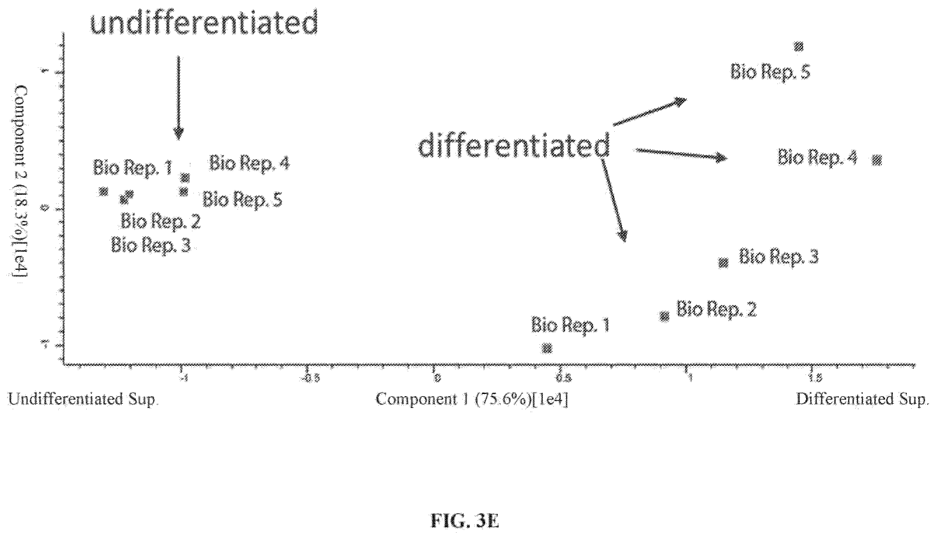

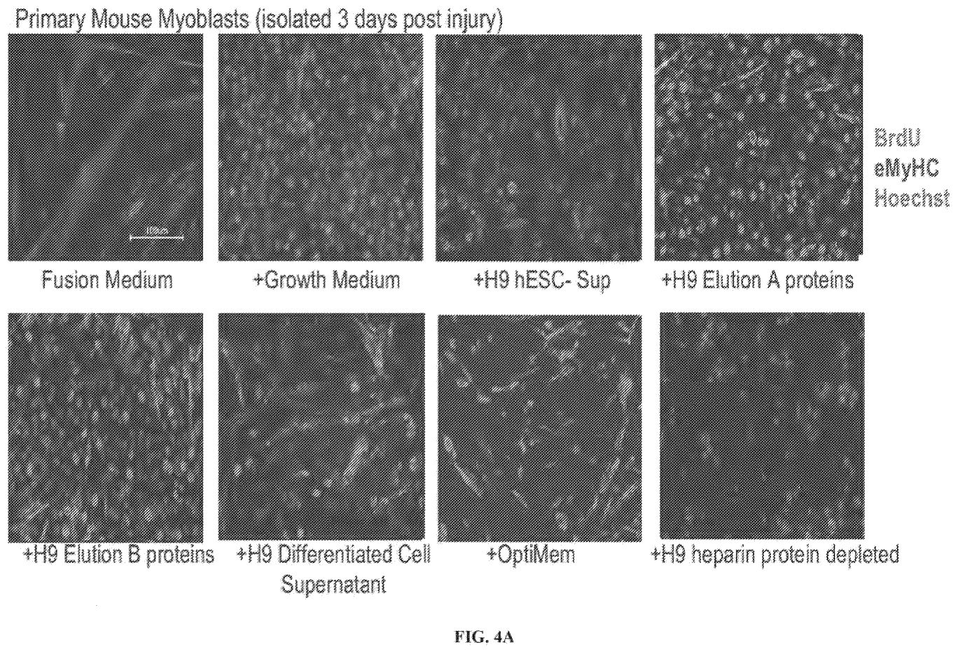

Factors enriched in the secretome of undifferentiated hPSCs can be determined by Mass spectroscopy A schematic of a type of mass spectroscopy experiment employed herein is shown in FIG. 3A.

Five confluent, 15 cm plates of cells per biological replicate were washed with OptiMEM—a basal, synthetic medium—, and then incubated in OptiMEM for 16 hours, yielding roughly 100 ml of media. The media, now containing secreted factors, was collected, cells and cell debris removed by centrifugation, and flash frozen for storage at −80C until processing. The target factors were enriched via affinity purification for heparin binding using heparin-agarose bead columns Heparin-agarose beads (Sigma) were washed with water twice, and once with OptiMEM (minus phenol red), before incubating with factor containing culture media for 2 h at 4° C. shaking at 100 rpm. The ratio of bead slurry (˜50% beads) to media can be effective at 1:10, 1:20, 1:30, 1:40, and 1:50. Heparin-agarose beads were then collected into a column by centrifugation in an Amicon Pro Purification System column set in a 50 ml conical tube at 1000 g for 5 min, washed with 10× column volumes of PBS 0.05% tween at 4° C. twice. Factors were eluted via two repeats of the following: addition of a high salt solution (1.5M NaCl, 0.01M HEPES, pH 7.2, at ratio of 0.4 ml elution buffer per milliliter of bead slurry), incubated at 4° C. for 10 minutes at 100 rpm, and centrifugation at 1000 g for 5 min into a fresh collection tube.

Protein concentration in the eluted fraction was assayed by silver stain densitometry as shown in FIG. 3B, and a BCA assay against standard curves for bovine serum albumin. Protein disulfide bonds were reduced by incubation in 5 mM tris-(2-carboxyethyl)-phosphine (TCEP) for 25 min, and the free cysteines alkylated with 10 mM iodoacetamide at room temperature for 30 min in the dark. Excess iodoacetamide was quenched with 15 mM dithiotreitol during a 15 min incubation. The eluates from all samples were then further purified by protein precipitation using trichloroacetic acid, prior to resuspending in digest buffer and 16 hr of digestion using a mixture of modified Trypsin and Lys-C to yield peptides predominantly with terminal arginine or lysine residues. The resulting peptide concentration were measured using a quantitative colorimetric peptide assay (Promega), and equimolar amounts of peptides from each biological replicate labeled at their free amines with tandem mass tags (TMT) using manufacturer recommended conditions before mixing the peptides. The mixed sample was desalted via reverse phase separation on a C18 StageTip prior to analysis via nHPLC-SPSMS3 on a Fusion Lumos (Thermo Fisher) A TOP10 method was used to select up to 10 MS2 precursors for identification by CID-MS2 analyzed in the ion trap. For synchronous precursor selection of up to 10 ion windows, the FTMS3 isolation window was 0.4 m/z, max ion time 150 ms, automatic gain control 1.5E5, and FTMS3 resolution was 50,000. Resulting spectra were searched using commercial MS analysis software against the Uniprot human database (2018) protein sequences (Swiss-Prot and TrEMBL) concatenated with their reversed sequences as decoys for FDR determination, appended to common contaminant sequences Searches restricted the precursor ion tolerance to 50 ppm and the product ion tolerance window to 0.9 m/z (or 50 ppm), allowed no more than two missed cleavages, included static modification of lysine residues, arginine residues and peptide N-termini with TMT tags (+229.163 Da), static carbamidomethylation of cysteine residues (+57.021 Da), and variable oxidation of methionine residues (+15.995 Da).

Results were filtered to a 1% FDR at the peptide and then protein level using the target-decoy strategy. Peptides were assigned to protein groups, and individual proteins by the parsimony principle Proteins were quantified by summing reporter ion intensities across all PSMs with greater than 70% of their spectral intensity deriving from matched ions and a summed signal to noise intensity greater than 200, normalizing channel level intensities, and computing the percent contribution of a given channel to the total signal. These values were then used for additional statistical modelling of differential abundance.

Heparin-associated proteins from undifferentiated and differentiated supernatants generated distinct sets of secreted factors as indicated by FIGS. 3C, 3D and 3E. Combined results from such experiments are summarized in Table 2 shown previously herein, by the gene name, UniProt ID, Entrez Gene ID, and Ensembl ID.

### Example 7—Validation of Pro-Regenerative Factors In Vitro Using High-Throughput Imaging

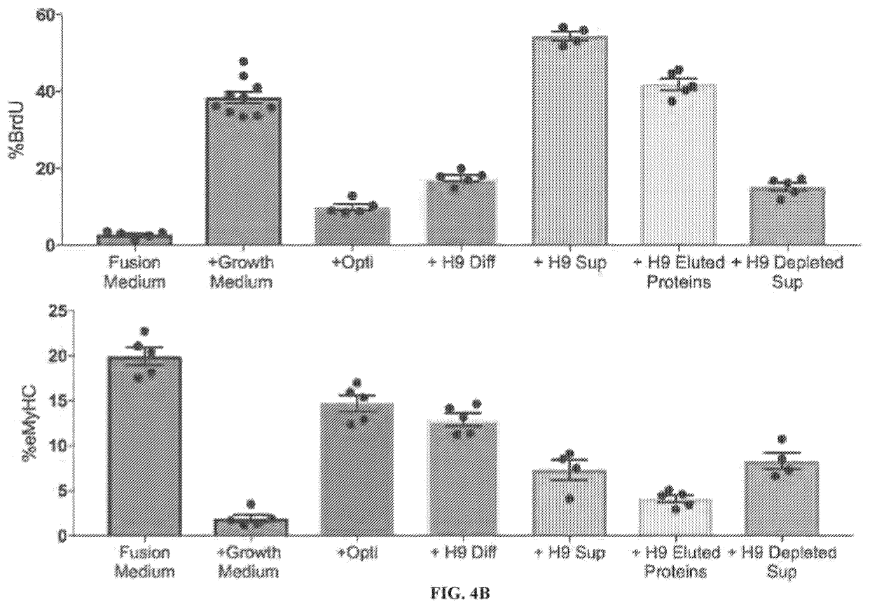

Mass spectroscopy can define candidate pro-regenerative factors, however, as shown in example 6, these experiments can generate large amounts of data that need to be further validated in relevant in vitro and in vivo models. The use of high-throughput imaging can help define individual factors and mixtures of factors that possess regenerative potential. Mouse muscle progenitor cells can be cultured with BrdU or Edu, in the presence or absence of specific potential pro-regenerative factors, and the degree of proliferation determined using high-throughput microscopy. BrdU or Edu staining indicates proliferation, while embryonic Myosin Heavy-Chain (eMyHC) staining indicates terminal differentiation of the progenitor cells. FIGS. 4A and 4B shows an example of data generated using high-throughput imaging.

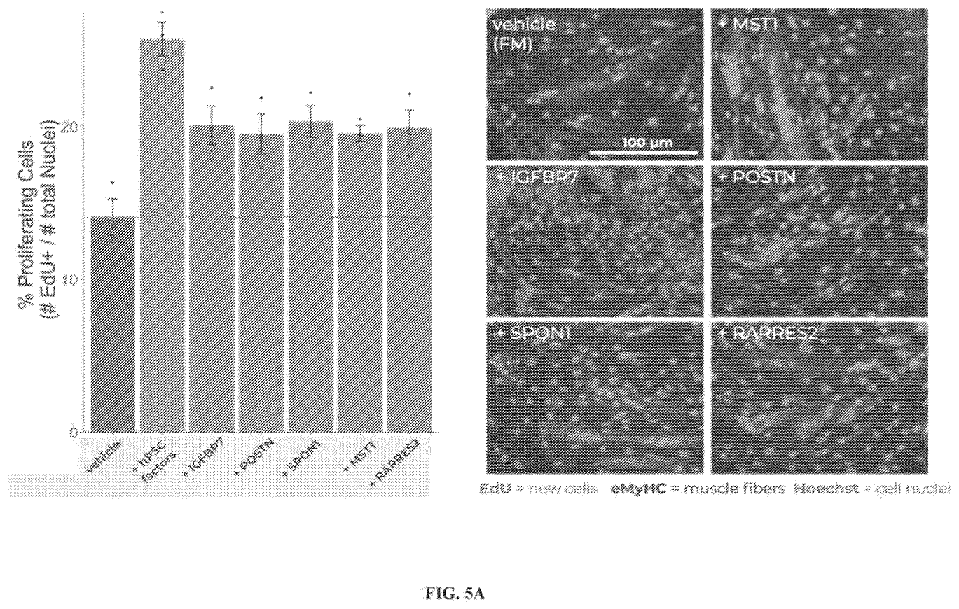

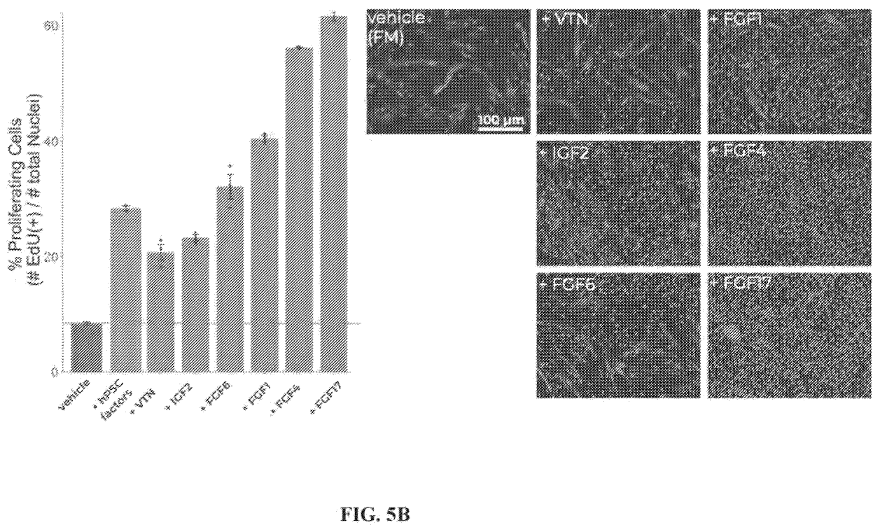

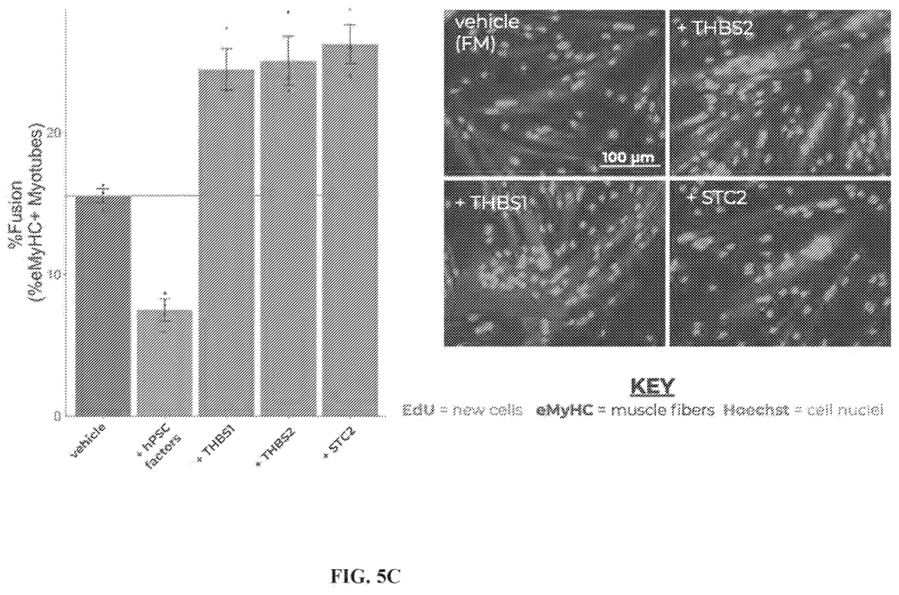

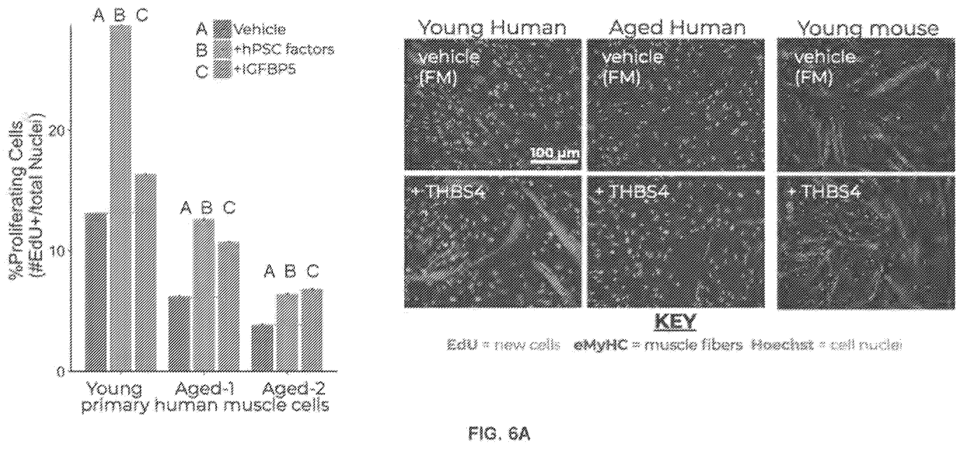

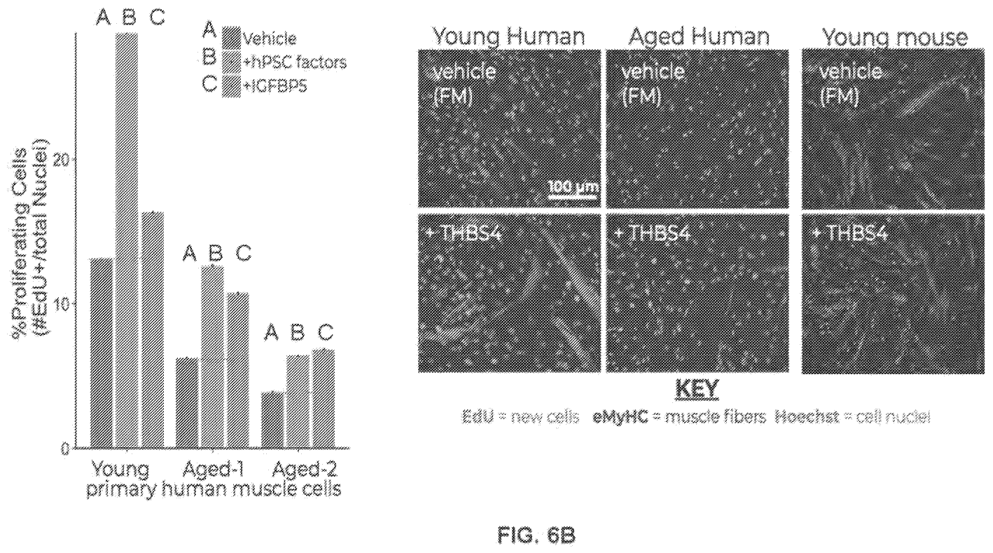

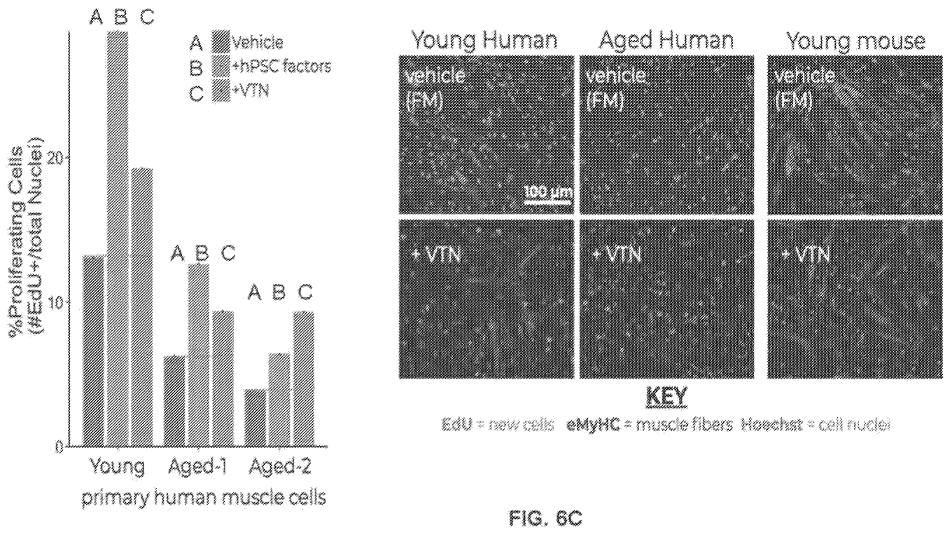

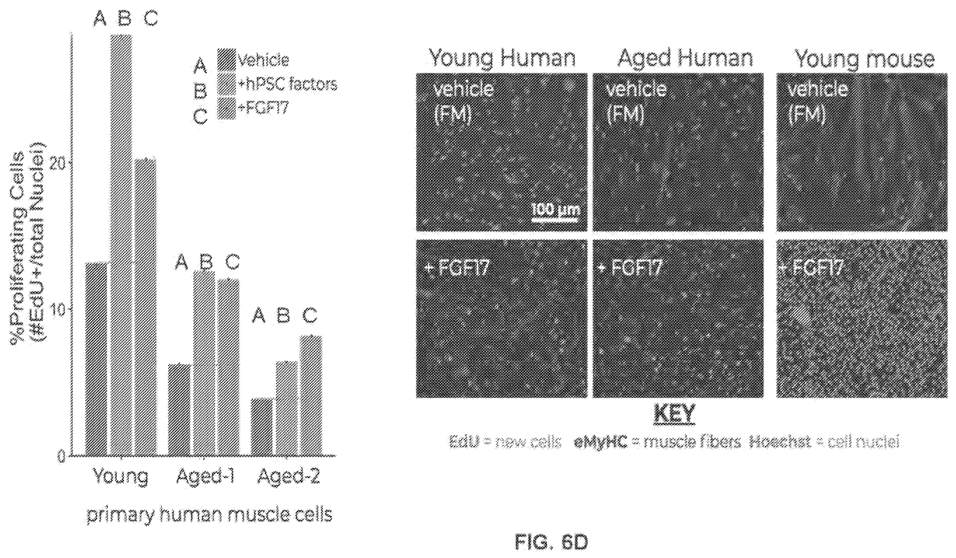

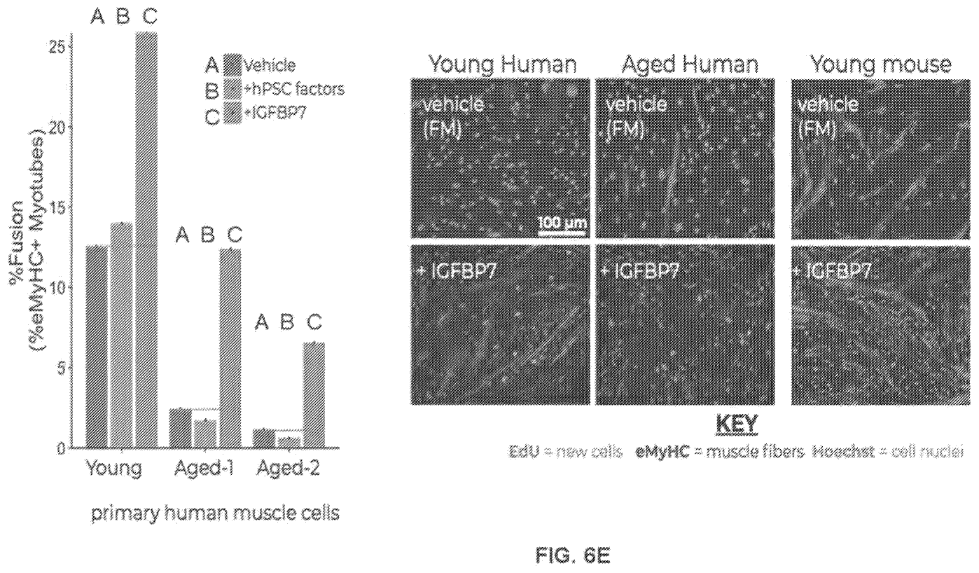

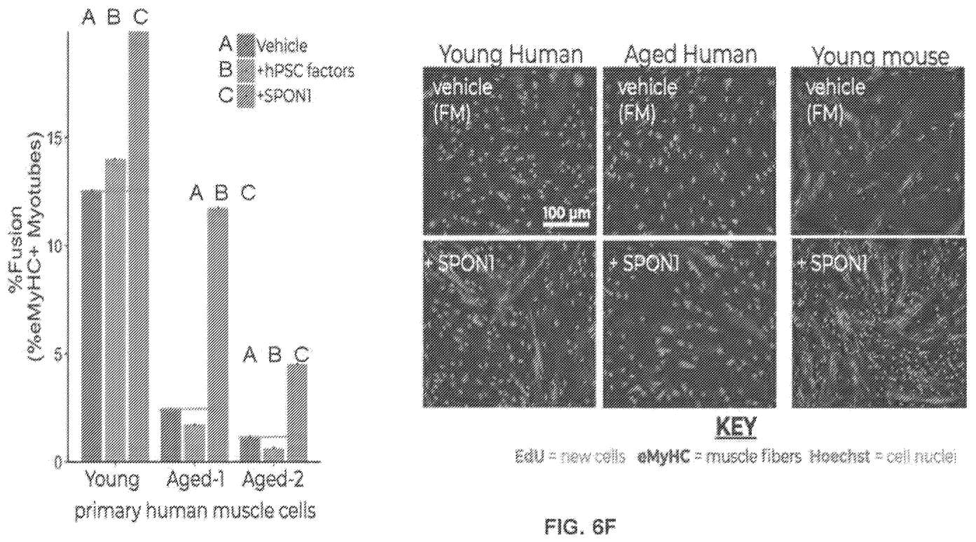

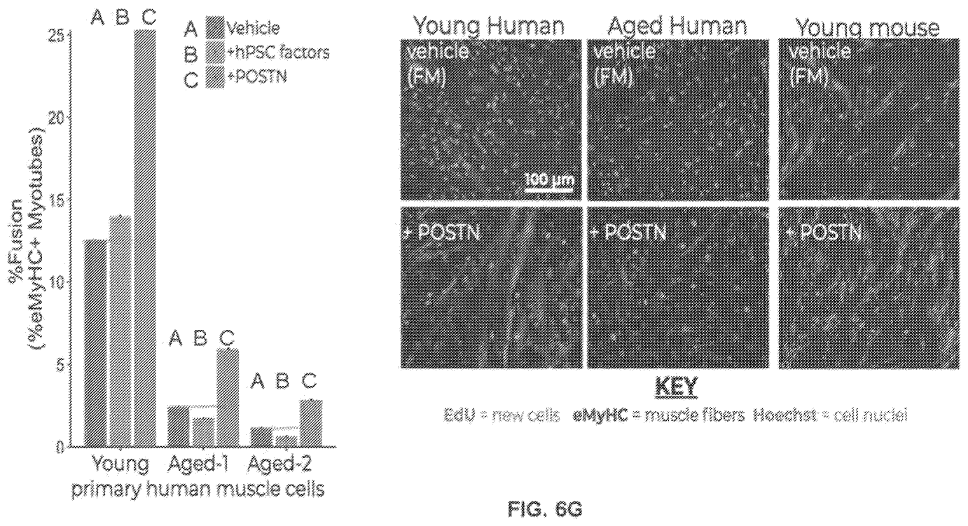

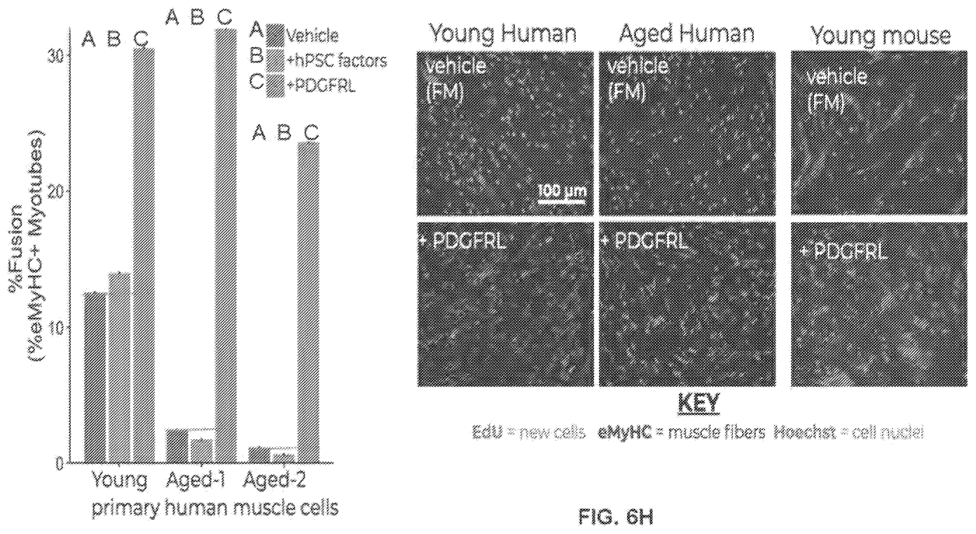

Many of the factors detailed in Table 2 were tested individually for their ability to promote proliferation and/or fusion of mouse muscle progenitor cells in vitro FIG. 5 and FIG. 6.

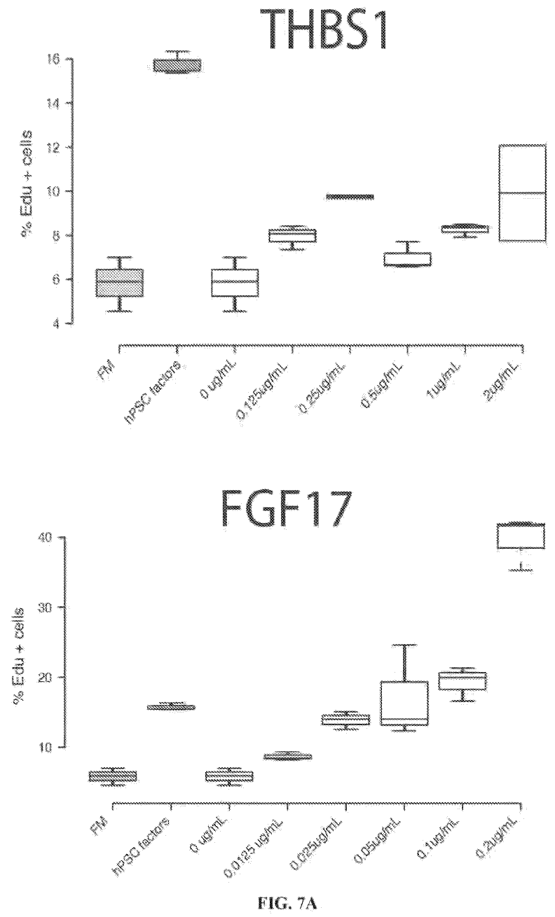

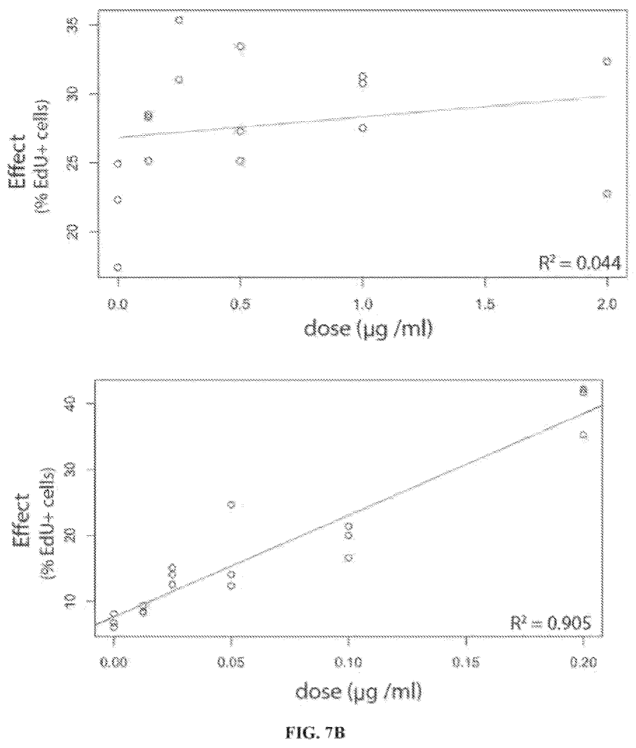

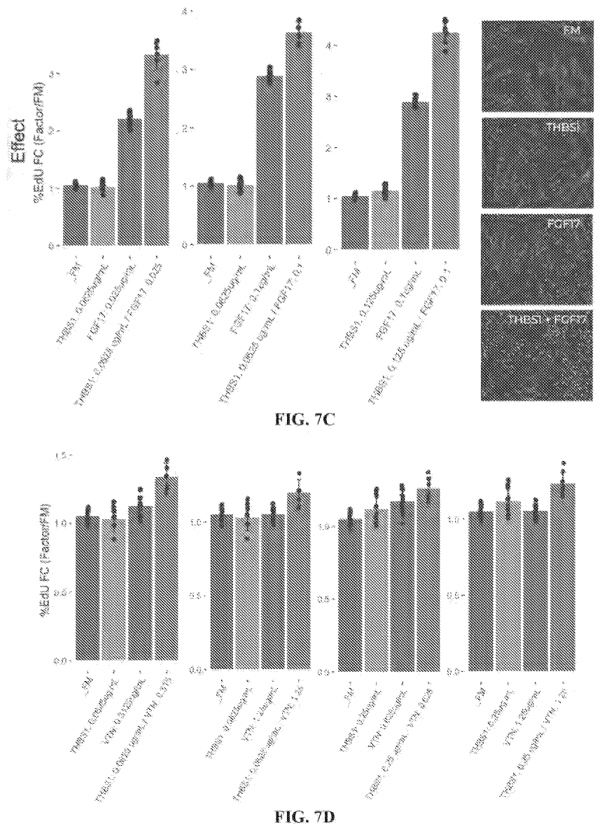

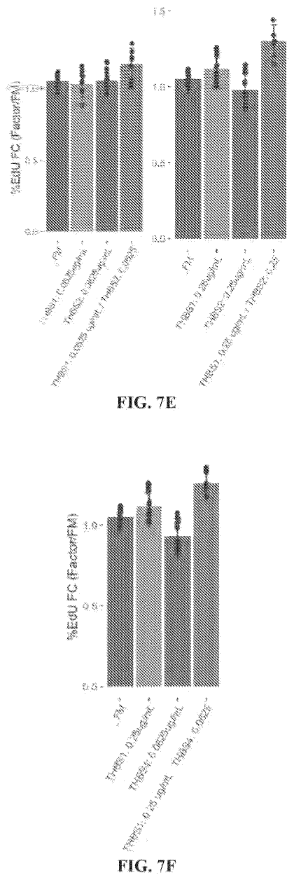

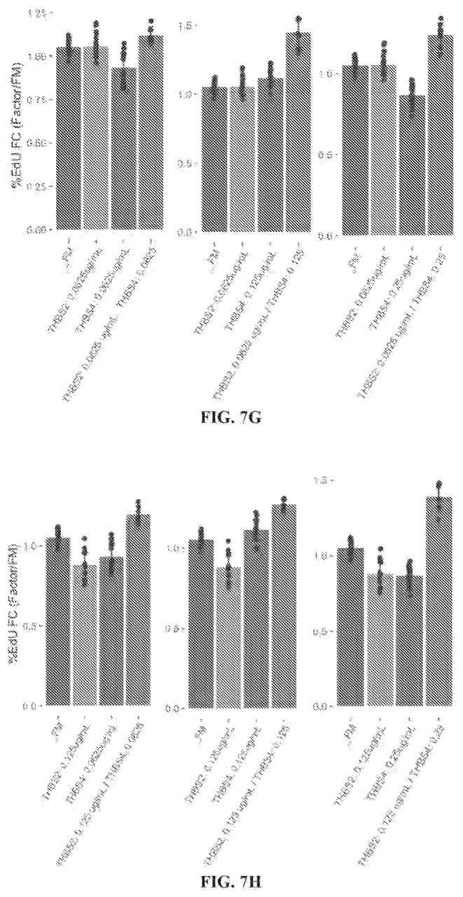

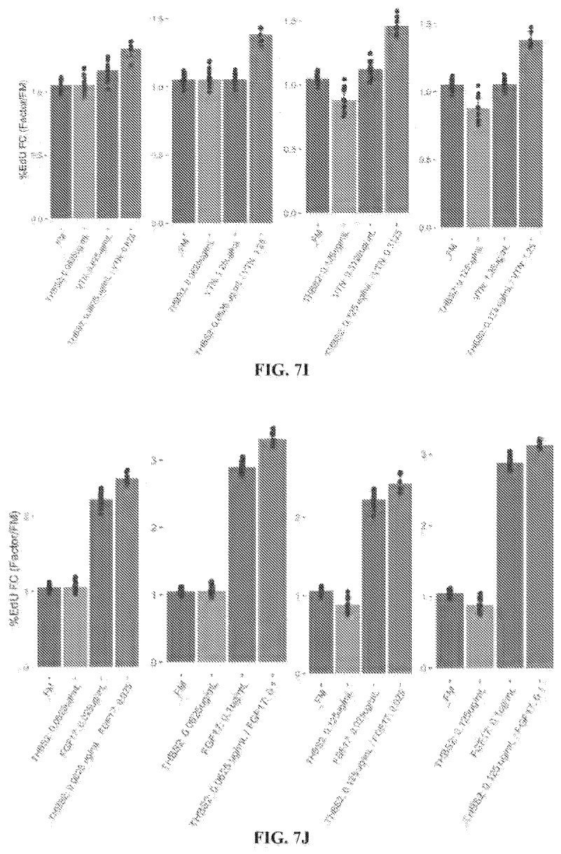

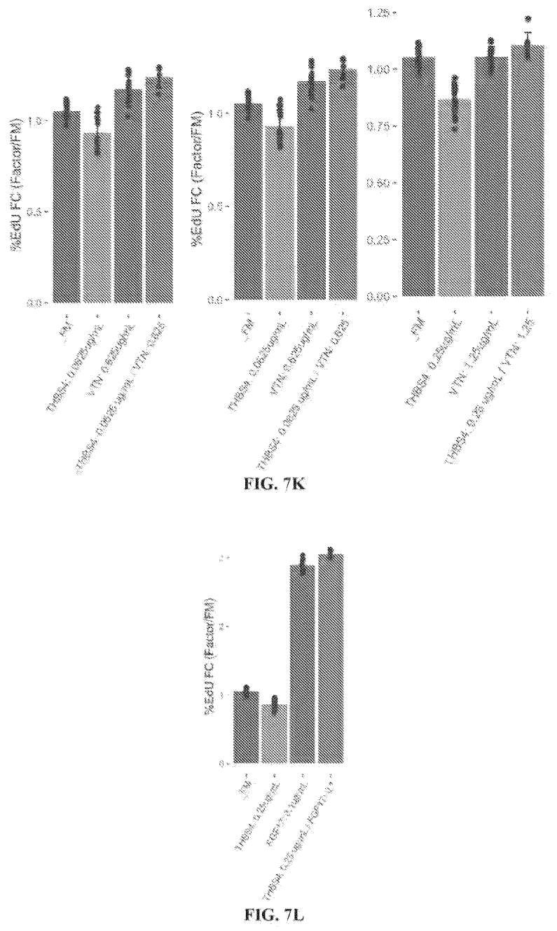

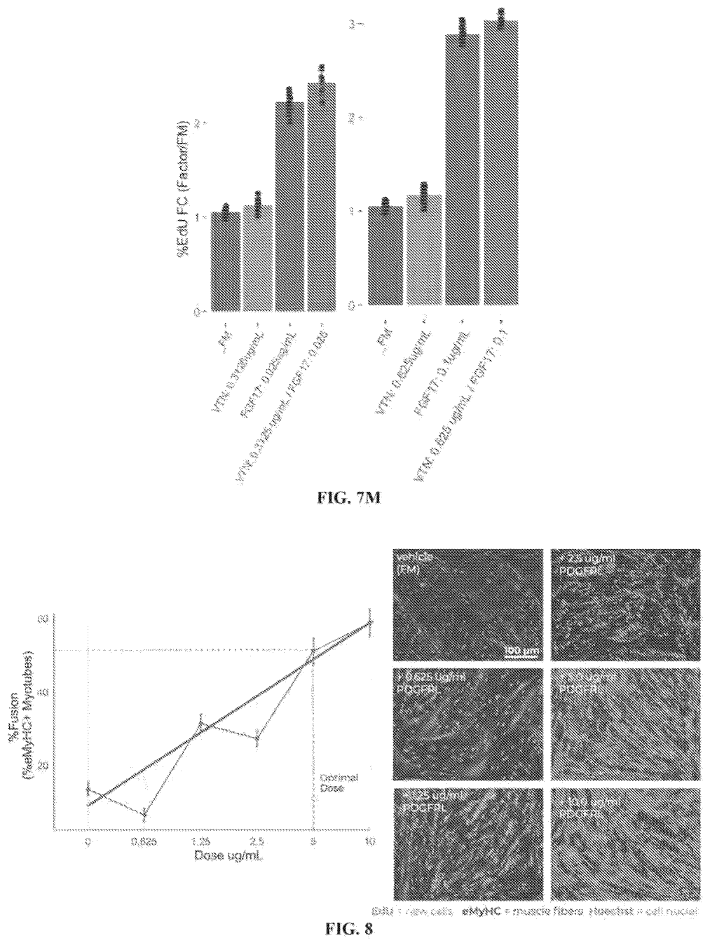

The effect of the candidate factors on myogenic activity was assayed in biological triplicate across a range of concentrations centered around expected physiological levels by adding each factor to mouse myoblasts for 48 hours or human myoblasts for 72 hours with daily media changes (DMEM+2% horse serum) and a second pulse of factors After 24 hours, cells were pulsed for 2-5 hours with EdU (30 uM), ethanol fixed, stained with Hoescht 3342, immunostained for proliferation—as measured by the percent of cells staining positive for EdU (% EdU)—, and immunostained for differentiation—as measured by the increase in cellular area staining positive for embryonic myosin heavy chain (% eMyHC) relative to the negative controls, which received media and vehicle only. Wells were imaged on a Keyence BZ-100 at 4×, the images quantified in Cell Profiler, and the statistics were computed in R. FIGS. 7A-7B show an example of the proliferation dose response for two of the factors tested. Results for additional factors are summarized below in Table 9.

TABLE 9
Effect of individual factors on mouse myoblast growth and fusion

| | | Proliferation (% EdU) | | Fusion (% eMyHC) | |
|---|---|---|---|---|---|
| | | | Statistical | | Statistical |
| | Concen- | Effect Size | Signif- | Effect Size | Signif- |
| Factor | tration | (% relative | icance | (% relative | icance |
| Name | (ug/mL) | to - control) | (p-value) | to - control) | (p-value) |
| FST | 0.33 | 72% | 0.001 | 202% | 0.005 |
| CTGF | 5 | | | 154% | 0.00005 |
| THBS1 | 5 | | | 157% | 0.004 |
| THBS2 | 0.33 | | | 161% | 0.006 |
| IGFBP3 | 0.33 | 131% | 0.03 | | |
| IGFBP5 | 2 | 69% | 0.04 | | |
| IGFBP7 | 0.33 | 78% | 0.03 | | |
| STC2 | 2.5 | 133% | 0.02 | −31% | 0.003 |
| SPON1 | 0.33 | 139% | 0.04 | | |
| MST1 | 0.33 | 144% | 0.02 | | |
| POSTN | 0.33 | 139% | 0.01 | | |
| RARRES2 | 0.33 | 141% | 0.03 | | |
| AGRN | 2.5 | 148% | 0.008 | | |
| CHRDL1 | 1.25 | 150% | 0.00009 | | |
| VTN | 5 | 125% | 0.004 | | |
| FGF17 | 0.5 | 244% | 0.0004 | | |
| ANOS1 | 1 | 165% | 0.03 | | |
| FGF4 | 0.3 | 239% | 4.6E−13 | | |
| HGF | 0.56 | 114% | 1.6E−10 | | |
| IGF2 | 1 | 97% | 1.3E−5 | | |
| FGF1 | 1 | 96% | 2.3E−7 | | |
| FGF6 | 0.002 | 115% | 1.8E−5 | | |
| MST1 | 0.33 | 79% | 0.002 | | |
| PDGFD | 0.1 | 89% | 4E−5 | | |

The effect of the combination of candidate factors on myogenic activity was assayed in biological triplicate across a range of concentrations centered around expected physiological levels by adding each factor to mouse myoblasts for 48 hours or human myoblasts for 72 hours with daily media changes (DMEM+2% horse serum) and a second pulse of factors. After 24 hours, cells were pulsed for 2-5 hours with EdU (30 uM), ethanol fixed, stained with Hoescht 3342, immunostained for proliferation—as measured by the percent of cells staining positive for EdU (% EdU)—, and immunostained for differentiation—as measured by the increase in cellular area staining positive for embryonic myosin heavy chain (% eMyHC) relative to the negative controls, which received media, individual factors, or vehicle only. Wells were imaged on a Keyence BZ-100 at 4×, the images quantified in Cell Profiler, and the statistics were computed in R. FIGS. 7C-7M show examples of the proliferation dose response for two or more of the factors tested individually and as a combination to test for synergy. Statistical metrics for increased myogenetic activity from the pair of factors relative to the controls are summarized below in Tables 10-14. Each polypeptide was produced using the method listed in Table 1. Each polypeptide was produced using the method listed in Table 1. The magnitude of the combinations' effects relative to control (FM—negative control, hESC factors—positive control) is shown. The Combination Index (CI) for synergy was calculated using the Highest Single Agent (HAS) model due to the linear dose responses for the individual factor, e.g. FIG. 7B.

TABLE 10
Synergistic combinations of heparin-associated polypeptides.

| Factor 1 | | Factor 2 | | Factor 3 | | | |
|---|---|---|---|---|---|---|---|
| Name | ug/mL | Name | ug/mL | Name | ug/mL | p-value | CI (HSA) |
| THBS2 | 0.125 | THBS4 | 0.25 | VTN | 1 | <0.05 | 0.886 |
| THBS2 | 0.125 | THBS4 | 0.25 | ANOS1 | 1 | <0.005 | 0.532 |
| THBS2 | 0.125 | THBS4 | 0.25 | | | <0.005 | 0.633 |
| THBS1 | 0.0625 | FGF17 | 0.025 | | | <0.005 | 0.667 |
| THBS1 | 0.125 | FGF17 | 0.1 | | | <0.005 | 0.679 |
| THBS2 | 0.125 | THBS4 | 0.25 | IL-15 | 1 | <0.005 | 0.680 |
| THBS2 | 0.125 | THBS4 | 0.25 | IGF2 | 0.05 | <0.05 | 0.733 |
| THBS2 | 0.0625 | VTN | 1.25 | | | <0.005 | 0.763 |
| THBS2 | 0.125 | VTN | 1.25 | | | <0.005 | 0.763 |
| THBS2 | 0.125 | VTN | 0.3125 | | | <0.005 | 0.771 |
| THBS2 | 0.0625 | THBS4 | 0.125 | | | <0.005 | 0.772 |
| THBS2 | 0.125 | THBS4 | 0.0625 | | | <0.005 | 0.779 |
| THBS1 | 0.0625 | FGF17 | 0.1 | | | <0.005 | 0.796 |
| THBS1 | 0.0625 | VTN | 0.315 | | | <0.005 | 0.840 |
| THBS2 | 0.0625 | THBS4 | 0.25 | | | <0.005 | 0.850 |
| THBS1 | 0.25 | THBS2 | 0.25 | | | <0.05 | 0.859 |
| THBS2 | 0.0625 | FGF17 | 0.1 | | | <0.005 | 0.871 |
| THBS2 | 0.0625 | VTN | 0.625 | | | <0.005 | 0.876 |
| THBS2 | 0.125 | THBS4 | 0.125 | | | <0.005 | 0.878 |
| THBS1 | 0.0625 | VTN | 1.25 | | | <0.05 | 0.880 |
| THBS1 | 0.25 | THBS4 | 0.0625 | | | <0.005 | 0.888 |
| THBS2 | 0.0625 | FGF17 | 0.025 | | | <0.005 | 0.890 |
| THBS1 | 0.0625 | THBS2 | 0.0625 | | | <0.05 | 0.900 |
| THBS1 | 0.25 | VTN | 1.25 | | | <0.005 | 0.907 |
| THBS2 | 0.125 | FGF17 | 0.025 | | | <0.005 | 0.913 |
| VTN | 0.3125 | FGF17 | 0.025 | | | <0.05 | 0.915 |
| THBS4 | 0.25 | VTN | 0.3125 | | | <0.05 | 0.922 |
| THBS2 | 0.125 | FGF17 | 0.1 | | | <0.005 | 0.923 |
| THBS1 | 0.25 | VTN | 0.625 | | | <0.005 | 0.930 |
| THBS2 | 0.0625 | THBS4 | 0.0625 | | | <0.05 | 0.942 |
| THBS4 | 0.25 | FGF17 | 0.1 | | | <0.005 | 0.945 |
| THBS4 | 0.0625 | VTN | 0.625 | | | <0.05 | 0.950 |
| VTN | 0.625 | FGF17 | 0.1 | | | <0.005 | 0.952 |
| THBS4 | 0.25 | VTN | 1.25 | | | <0.05 | 0.954 |

TABLE 11
Additional data regarding synergistic combinations of heparin-associated polypeptides (mouse myoblasts)

| | Single | | | | | | | | |
|---|---|---|---|---|---|---|---|---|---|
| | Dose @ | Single | Single Dose | Single | Combo | Combo | Combo Dose | Combo | |
| Factor | saturation | Dose | % EdU Fold | Dose | Dose | Dose | % EdU Fold | Dose | |
| Name | (ug/mL) | % of EB | Change to FM | % EdU | (ug/mL) | % of EB | Change to FM | % EdU | HSA CI |
| THBS1 | 2 | 50% | 1.7 | 15% | 0.125 | 64% | 1.16 | 14% | |
| FGF17 | 0.5 | 239% | 4.5 | 69% | 0.1 | 148% | 2.89 | 33% | |
| Combo | | | | | | 163% | 3.63 | 29% | 0.679091 |
| THBS2 | 2 | 50% | 1.25 | 13% | 0.125 | 46% | 0.88 | 10% | |
| THBS4 | 2 | 40% | 1.87 | 10% | 0.25 | 53% | 1.11 | 12% | |
| Combo | | | | | | 71% | 1.39 | 17% | 0.632548 |
| THBS1 | 2 | 50% | 1.7 | 15% | 0.0625 | 44.08% | 0.75 | 10.63% | |
| VTN | 10 | 50% | 1.5 | 16% | 10 | 59.18% | 1.01 | 14.27% | |
| Combo | | | | | | 69.47% | 1.19 | 16.75% | 0.851842 |
| THBS2 | 2 | 50% | 1.25 | 13% | 0.125 | 31.08% | 0.85 | 7.86% | |
| THBS4 | 2 | 40% | 1.87 | 10% | 0.25 | 29.33% | 0.8 | 7.41% | |
| IL15 | 0.5 | 52% | 1.4 | 12.50% | 1 | 47.25% | 1.28 | 11.90% | |
| Combo | | | | | | 57.91% | 1.66 | 16.21% | 0.679578 |
| THBS2 | 2 | 50% | 1.25 | 13% | 0.125 | 45.08% | 0.79 | 9.64% | |
| THBS4 | 2 | 40% | 1.87 | 10% | 0.25 | 61.15% | 1.08 | 13.08% | |
| VTN | 10 | 50% | 1.5 | 16% | 10 | 54.46% | 0.94 | 12.67% | |

TABLE 12
Additional data regarding synergistic combinations of
heparin-associated polypeptides (mouse myoblasts)

| | Single | Single | Single | Combo | Combo | Combo Dose | Combo |
|---|---|---|---|---|---|---|---|
| | Dose | Dose | Dose | Dose | Dose | % EdU Fold | Dose |
| Factor | (ug/mL) | % of Eb | % EdU | (ug/mL) | % of EB | Change to FM | % EdU |
| THBS1 | 2 | 50% | 15% | 0.125 | 64% | 1.16 | 14% |
| FGF17 | 0.5 | 239% | 69% | 0.1 | 148% | 2.89 | 33% |
| Combo | | | | | 163% | 3.63 | 29% |
| THBS2 | 2 | 50% | 13% | 0.125 | 46% | 0.88 | 10% |
| THBS4 | 2 | 40% | 10% | 0.25 | 53% | 1.11 | 12% |
| Combo | | | | | 71% | 1.39 | 17% |
| THBS1 | 2 | 50% | 15% | 0.0625 | 44.08% | 0.75 | 10.63% |
| VTN | 10 | 50% | 16% | 10 | 59.18% | 1.01 | 14.27% |
| Combo | | | | | 69.47% | 1.19 | 16.75% |
| THBS2 | 2 | 50% | 13% | 0.125 | 31.08% | 0.85 | 7.86% |
| THBS4 | 2 | 40% | 10% | 0.25 | 29.33% | 0.8 | 7.41% |
| IL15 | 0.5 | 52% | 12.50% | 1 | 47.25% | 1.28 | 11.90% |
| Combo | | | | | 57.91% | 1.66 | 16.21% |
| THBS2 | 2 | 50% | 13% | 0.125 | 45.08% | 0.79 | 9.64% |
| THBS4 | 2 | 40% | 10% | 0.25 | 61.15% | 1.08 | 13.08% |
| VTN | 10 | 50% | 16% | 1 | 54.46% | 0.94 | 12.67% |
| Combo | | | | | 69.02% | 1.22 | 14.76% |
| THBS2 | 2 | 50% | 13% | 0.125 | 58.62% | 0.86 | 15.23% |
| THBS4 | 2 | 40% | 10% | 0.25 | 62.99% | 0.93 | 16.37% |
| IGF2 | 2 | 89% | 23% | 0.5 | 85.78% | 1.27 | 22.29% |
| Combo | | | | | 116.95% | 1.72 | 30.39% |

TABLE 13
Additional data regarding synergistic combinations of heparin-associated polypeptides (human myoblasts)

| | Single | | | | | | | | |
|---|---|---|---|---|---|---|---|---|---|
| | Dose @ | Single | Single Dose | Single | Combo | Combo | Combo Dose | Combo | |
| Factor | saturation | Dose | % EdU Fold | Dose | Dose | Dose | % EdU Fold | Dose | |
| Name | (ug/mL) | % of EB | Change to FM | % EdU | (ug/mL) | % of EB | Change to FM | % EdU | HSA CI |
| THBS1 | 7.5 | 98.93 | 1.61 | 10.99 | 0.5 | 78.22 | 0.85 | 8.95 | |
| FGF17 | 0.25 | 222.09 | 3.62 | 24.67 | 0.05 | 121.74 | 1.33 | 13.93 | |
| Combo | | | | | | 157.29 | 1.72 | 18 | 0.77 |
| THBS2 | 7.5 | 112.8469 | 1.2 | 12.53448 | 0.125 | 57.09 | 0.95 | 7.82 | |
| THBS4 | 0.5 | 126.7002 | 2.07 | 14.07323 | 0.25 | 62.67 | 1.04 | 8.59 | |
| IL15 | 0.1 | 67.81 | 1.13 | 9.29 | 0.1 | 67.81 | 1.13 | 9.29 | |
| Combo | | | | | | 69 | 1.15 | 9.46 | 0.98 |

TABLE 14
Additional data regarding synergistic combinations of
heparin-associated polypeptides (human myoblasts)

| | Single | | | | | | | |
|---|---|---|---|---|---|---|---|---|
| | Dose @ | Single | Single Dose | Single | Combo | Combo | Combo Dose | Combo |
| Factor | saturation | Dose | % EdU Fold | Dose | Dose | Dose | % EdU Fold | Dose |
| Name | (ug/mL) | % of EB | Change to FM | % EdU | (ug/mL) | % of EB | Change to FM | % EdU |
| THBS1 | 7.5 | 98.93 | 1.61 | 10.99 | 0.5 | 78.22 | 0.85 | 8.95 |
| FGF17 | 0.25 | 222.09 | 3.62 | 24.67 | 0.05 | 121.74 | 1.33 | 13.93 |
| Combo | | | | | | 157.29 | 1.72 | 18 |

### Example 8—In Vivo Testing of hPSC Factors Increases Regenerative Index and Reduces Fibrotic Index in an Acute Injury Model in Aged Mice

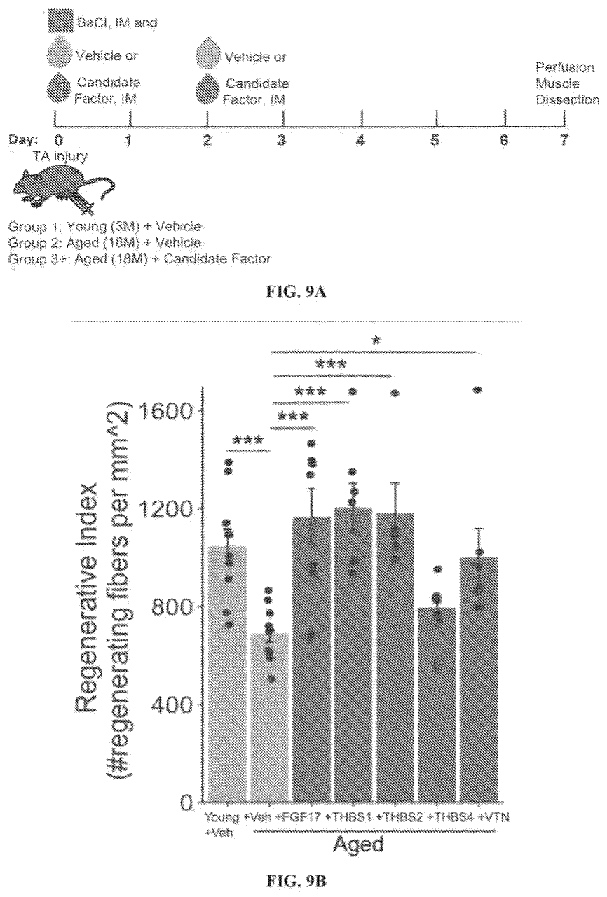

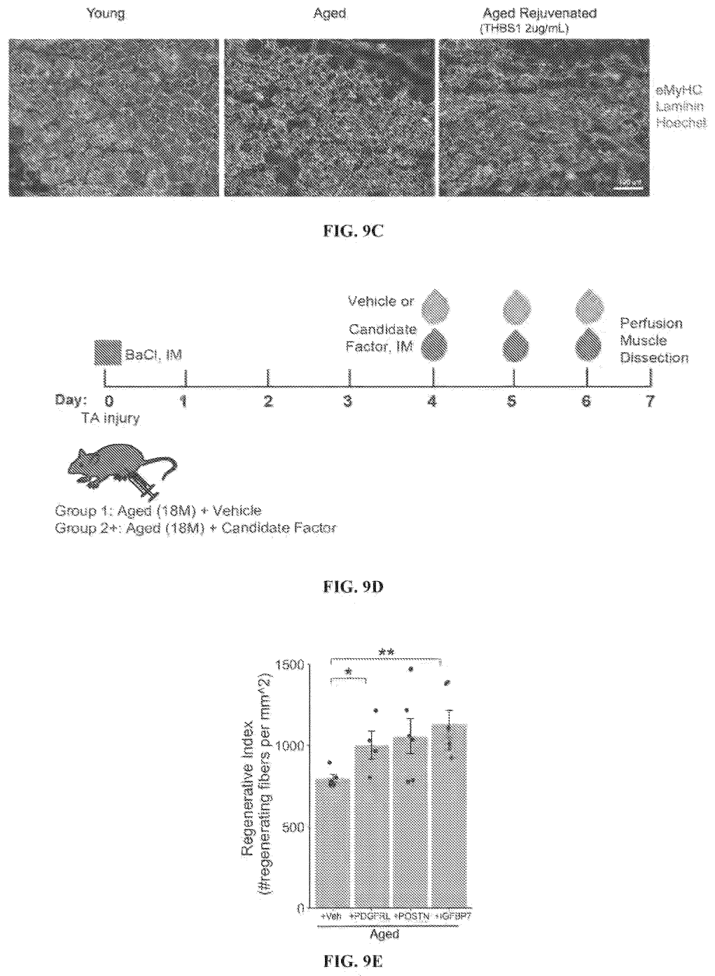

FIGS. 9A-9F show that aged mice (18 months) administered isolated, heparin-agarose bead purified hPSC show improved regenerative index and reduced fibrotic index. FIG. 9A shows a schematic of the experiment in this example. This experiment shows that hPSC derived factors can improve histological metrics of muscle health and function. As shown in FIG. 9B heparin-associated polypeptides isolated from human pluripotent stem cells increase regenerative potential and reduce muscle fibrosis in aged mice subjected to a model of acute muscle injury. As shown in FIG. 9C, there was increased muscle regeneration for injured, aged mouse muscle treated with THBS1 (2 ug/mL) compared to young and vehicle-treated, aged mouse muscle.

FIG. 9D shows the experimental schematic of time-points for dosing and analysis using an acute injury model in aged mice of the effects of individual heparin-associated polypeptides with fusion enhancing effects in vitro. Square denote injury inducing intramuscular injection (IM) with Barium Chloride while circles denote administration of treatment or vehicle. FIG. 9E shows the results of the experiment outlined in FIG. 9D shows that administration of 20 ul of heparin-associated polypeptides PPDGFRL (5 ug/mL, p<3.85E-2) and IGFBP7 (1 ug/mL, p<6.63E-3) resulted in improved new fiber formation (regenerative index compared to vehicle treated aged mice. Stars indicate degree of significance from one-way ANOVA tests. FIG. 9F provides representative images of immunofluorescence staining of sectioned mouse muscle (tibialis anterior) demonstrating increased muscle regeneration for injured, aged mouse muscle treated with POSTN (1 ug/mL) or IGFBP7 (1 ug/mL) compared to vehicle-treated, aged mouse muscle.

Other models for in vivo testing of hSPC factors include.

### Disuse-Reload Injury Model

This mouse model is a way to observe muscle atrophy in a non-invasive way by contracting the hind limbs of a mouse and preventing extension and flexion, thus reducing the size and strength. The model will serve as an important measurement of muscle regeneration with biologic candidates.

The hind limb will be immobilized with Cast Tape extended position using sports tape to prevent flexion of the limb. Once the sports tape is in place, a strip of casting tape will be wrapped over the sports tape from the ankle upward, and air dried. The extension of the hind limb should stay rigid in its position of the duration of the study parameters.

The study begins after mice are acclimated and on Day −3, in which mice from all groups will be weighed. Assigned animals will be given daily i.p. injections of Vehicle control or Candidate Biologic for 3 days before undergoing hind limb immobilization on Day 0 for 7 days with continuous daily i.p. injections. Hind limbs will be observed for any adverse effects due to immobilization. On Day 7 of the study, all animals will be sacrificed and muscle tissue weighed and harvested for further analysis.

### Force Measurement

This study will be used to measure the force of pull in the hind limbs that the animal exerts upon skeletal muscle injury of the tibialis anterior (TA) and gastrocnemius (GA) muscles after injury induction with Barium chloride (BaCl₂). This model will serve to determine which of our biological candidates are efficacious in muscle regeneration.

Skeletal Muscle Injury Induction Under anesthesia, BaCl₂ will be administered in two sites on the TA and four sites on the GA (as previously described). Hair will be shaved on the left and right hind limbs prior to injection with small animal hair clippers. On Day 0 of the study, the TA muscle will undergo BaCl₂ induced injury on two sites (previously described). On Day 4 of the study, the GA muscle will follow with BaCl₂ induced injury on four sites. Candidate biologic will be administered on Days 0 and 2 in the injury sites of the TA and GA muscles. BrdU will be injected via IP (QD) on days 4-7 to label proliferating muscle precursor and fibrotic cells in order to measure their regenerative potential.

On terminal day 7, animals will be deeply anesthetized and a force transducer will be used to measure twitch reactions in the hind limbs of each mouse being tested in the study, via a small incision in the TA to a small metal hook. This will be a terminal procedure. Grip strength measurements: the mice will rest on an angled mesh, facing away from the force meter and with its hind limbs at least one-half of the way down the length of the mesh. The mouse's tail is pulled directly toward the meter and parallel to the mesh. During this procedure, the mouse resists by grasping the mesh with all four limbs. Pulling is continued toward the meter until the hind limbs release.

### Ex Vivo Regenerative Measurement

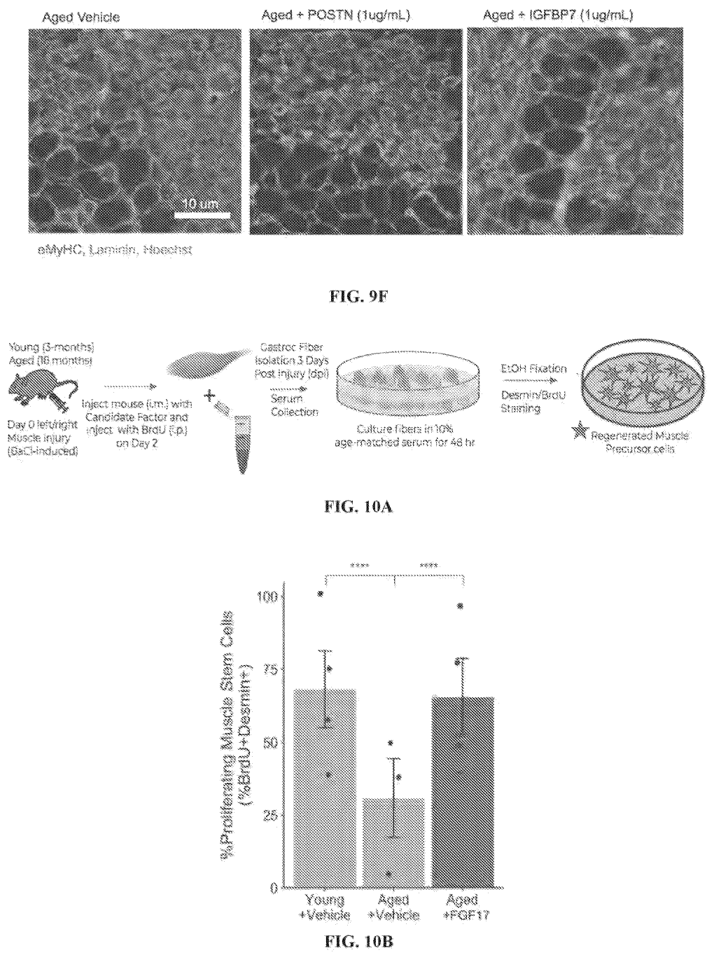

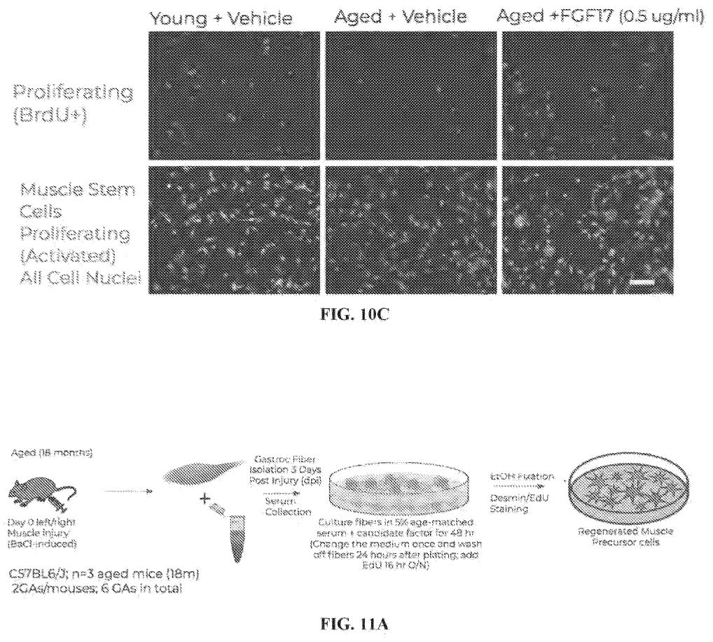

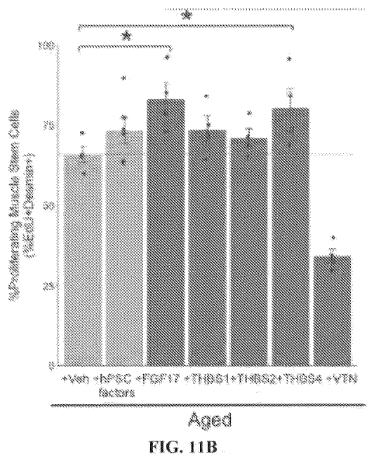

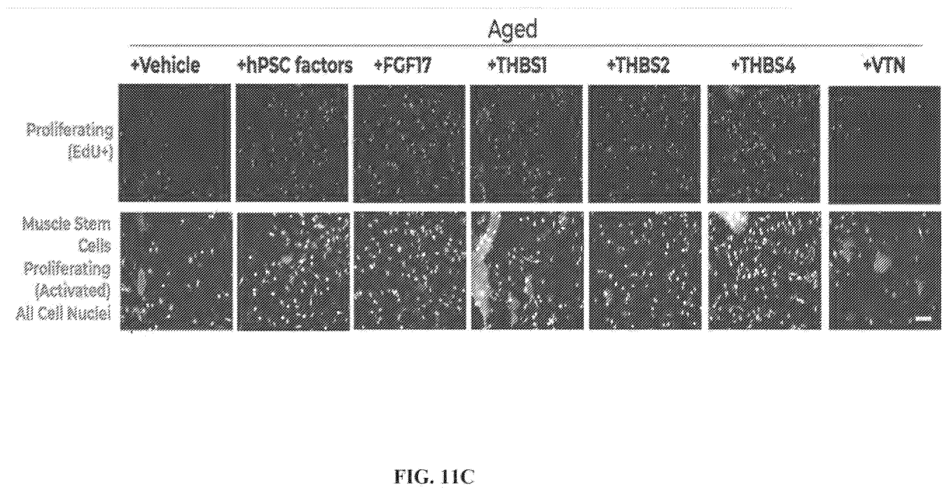

To confirm these data with age matched, primary muscle stem cells, injury-activated satellite cells associated with myofibers will be isolated from young and old muscle by dissecting the muscle groups of interest and dissociating the tissue to single cell suspensions by incubating in digestion medium (250 U/mL Collagenase type II in DMEM medium, buffered with 30 mM HEPES, pH 7.4) at 37C for 1 hr., triturating the cell suspension, the myofibers were collected by centrifugation and myofibers further digested with 1 U/mL Dispase and 40 U/mL Collagenase type II in 30 mM HEPES at 37C for 1 hr to free muscle stem cells. Muscle stem cells can then be plated and cultured growth media containing serum (2-5%) from the same mouse. The regenerative and fusion potential of the cells will then be assayed as described above in in Example 7 and as demonstrated in FIGS. 10B, 10C, 11B, and 11C. This has the advantage of testing the effect of treatment while maintaining the exogenous, often inhibitory extracellular environmental ques contributed by the age appropriate serum.

### Example 9—Modelling Treatment of a Muscular Dystrophy with Pro-Regenerative Factors In Vitro Using High-Throughput Imaging

Muscular dystrophies (MD) encompass a variety of muscular degeneration diseases typically due to genetic mutations in genes encoding proteins responsible for forming and stabilizing skeletal muscle. The phenotypic consequence of these genetic mutations is the progressive loss of muscle mass and strength over time, similar to sarcopenia but with different underlying causes. As heparin-associated polypeptides provided phenotypic improvements on sarcopenic muscle, we tested for similar improvements in a model for MD.

Many of the factors detailed in Table 2 were tested individually for their ability to promote proliferation and/or fusion of human muscle progenitor cells from a patient with myotonic dystrophy type 1 (hMD)—a muscular dystrophy caused by mutations in the DMPK1 gene.

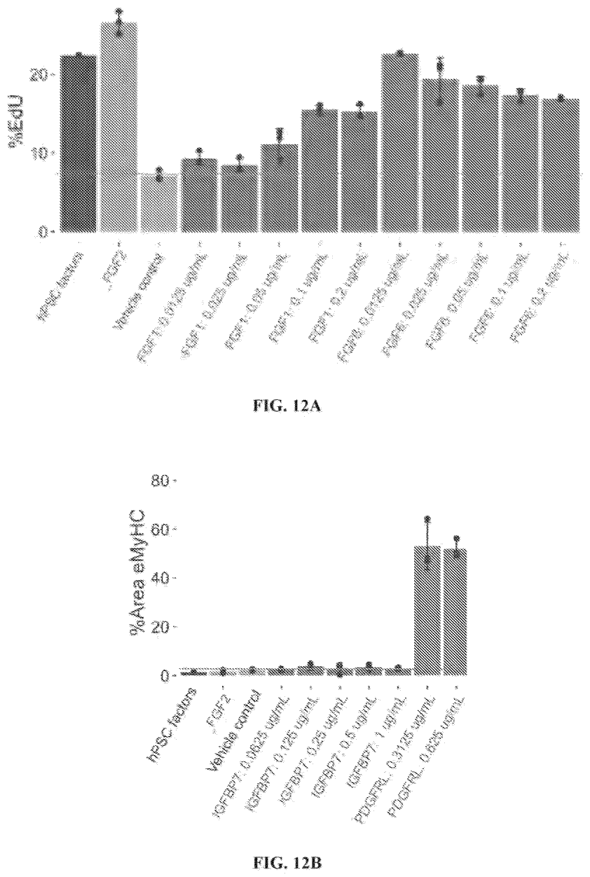

The effect of the candidate factors on myogenic activity was assayed in biological triplicate across a range of concentrations centered around expected physiological levels by adding each factor to hMD myoblasts for 72 hours with daily media changes (DMEM +2% horse serum) and a second pulse of factors at the first media change. After 72 hours, cells were pulsed for 2-5 hours with EdU (30 uM), ethanol fixed, stained with Hoescht 3342, immunostained for proliferation—as measured by the percent of cells staining positive for EdU (% EdU)—, and immunostained for differentiation—as measured by the increase in cellular area staining positive for embryonic myosin heavy chain (% eMyHC) relative to the negative controls, which received media and vehicle only. Wells were imaged on a Keyence BZ-100 at 4×, the images quantified in Cell Profiler. and the statistics were computed in R FIGS. 12A and 12B show examples of the quantitation of the proliferation response and fusion response for several of the factors tested, respectively. Results those and additional factors are summarized below in Table 15

TABLE 15
Effect of individual factors on distrophic
human myoblast growth and fusion

| | | Proliferation (% EdU) | | Fusion (% eMyHC) | |
|---|---|---|---|---|---|
| | | | Statistical | | Statistical |
| | Concen- | Effect Size | Signif- | Effect Size | Signifi- |
| Factor | tration | (% relative | icance | (% relative | cance |
| Name | (ug/mL) | to -control) | (p-value) | to -control) | (p-value) |
| FGF17 | 0.2 | 208% | <9E−14 | | |
| FGF4 | 0.0125 | 269% | 9.52E−13 | | |
| FGF4 | 0.025 | 244% | 6.83E−12 | | |
| FGF4 | 0.05 | 235% | 7.22E−10 | | |
| FGF4 | 0.1 | 213% | 8.11E−07 | | |
| FGF4 | 0.2 | 184% | 1.46E−02 | | |
| IGF2 | 0.2 | 53% | 2.57E−02 | | |
| FGF1 | 0.05 | 157% | 1.65E−06 | | |
| FGF1 | 0.1 | 217% | 2.87E−06 | | |
| FGF1 | 0.2 | 213% | 1.10E−10 | | |
| FGF6 | 0.0125 | 277% | 1.34E−09 | | |
| FGF6 | 0.025 | 272% | 4.54E−09 | | |
| FGF6 | 0.05 | 261% | 4.75E−08 | | |
| FGF6 | 0.1 | 243% | 1.03E−07 | | |
| FGF6 | 0.2 | 237% | 8.49E−06 | | |
| PDGFRL | 0.03125 | | | 2580% | 1.47E−06 |
| PDGFRL | 0.0625 | | | 2240% | 6.34E−07 |
| PDGFRL | 0.125 | | | 1410% | 4.53E−03 |
| PDGFRL | 0.25 | | | 2570% | 1.86E−08 |
| PDGFRL | 0.5 | | | 3440% | 5.75E−11 |

### Example 10 Assess hPSC Factors In Vivo Using Rodent Models of Becker Muscular Dystrophy for their Ability to Improve Muscle Regeneration and Muscle Mass

Mdx−/− mice will receive intramuscular injections of the top 3 select hPSC secreted factors selected from the in vitro screen. Indications of enhanced functional regeneration will be assessed through quantification of twitch force measurements and walking track analysis. The Mdx−/− mouse model for BMD is chosen as it has a spontaneous point mutation in the dystrophin gene leading to near complete absence of dystrophin protein. Despite having phenotypic markers of myopathy there are only mild skeletal muscle defects, suggesting that the model best reflects Becker muscular dystrophy. This animal model also mimics the complex paracrine signaling environment, where immune cells and other systemic factors would be involved. Thus, a screen in injured dystrophic muscle for regeneration will efficiently narrow the candidate list for those proteins needed for the ultimate goal of restoring muscle mass and strength to the BMD patients. Myogenesis in a muscle regeneration model will be compared between controls (vehicle-treated) and with the top 3-5 efficacious, dose-optimized factors. Hind limb muscles, the Tibialis anterior (TA) and gastrocnemius (GA) muscle will receive i.m. injection of vehicle control or candidate factor every day for 7 days (t=5 days). BrdU will be also be injected 3 days prior to tissue collection for ex vivo analysis. Muscle regeneration will be assessed in 10 micrometer muscle cryosections. The histological analysis of hematoxylin and eosin staining and immunodetection of eMyHC and BrdU will be used to quantify the numbers of de-novo eMyHC+ myofibers with centrally-located BrdU+ nuclei throughout the injury site. Additionally, myofiber size and muscle weights will also be assessed. For twitch for measurements, mice hindlimbs will be immobilize to a frame. A small incision will be made in the skin directly above the tibialis anterior (TA) muscle to hook to a thin metal hook and then attached to a 300C-LR force transducer (Aurora Scientific). A bipolar electrical stimulation cuff will wrap around the TA and stimulate with a single 0.1-ms pulse for 3 seconds.

It is expected that administration of select pro-myogenic factors will increase Delta/Notch and MapK signaling, dependent on BMP signaling in Mdx−/− myogenic cells, resulting in increased proliferation, allowing the injury site to heal similar to wildtype muscle—with new muscle fibers—instead of filling with fibrotic scar tissue or adipose tissue as is common in dystrophic muscle. Administration of candidate factors to Mdx−/− injured TA muscle is expected to improve regeneration indicated by quantification of muscle fiber repair and twitch force measurement.

### Example 11—Clinical Testing of Pro-Regenerative Factors

The purpose of this study is to determine the efficacy of repeat dosing with multiple dose levels of heparin-associated proteins on patient physical function, skeletal muscle mass and strength in older adults with sarcopenia. In addition, this study will generate data on the safety, tolerability, and pharmacokinetics of heparin-associated proteins in older adults with sarcopenia. Individuals will administered placebo or heparin-associated binding proteins and monitored for 25 weeks of study. The following primary and secondary outcome measures will be assessed:

### Primary Outcome Measures:

Short Physical Performance Battery (SPPB) [Time Frame Baseline, week 25]. Change from baseline to week 25.

### Secondary Outcome Measures:

Safety and tolerability as assessed by various measures such as adverse events. Assessed by various measures such as adverse events over 24 weeks.

Change in 6-minute walk test [Time Frame-baseline, week 25]. Change from baseline to week 25.

Gait speed [Time Frame: baseline, week 25]. Change from baseline to week 25.

Change in total lean body mass and appendicular skeletal muscle index measured by DXA from baseline to week 25.

### Inclusion Criteria:

Otherwise healthy adults aged at least 70 years; Low muscle mass as confirmed by DXA: Low gait speed, SPPB score less than or equal to 9, Weigh at least 35 kg, with adequate dietary intake as determined by patient interview.

### Protocol

Patients will be i.v.-administered placebo (5% dextrose solution) or heparin-associated binding proteins (in 5% dextrose). Starting on day 1, week 1 and repeated every other week (day one of weeks 3, 5, 7, 9, 11, 13, 15, 17, 19, 21, and 23. At the end of week 12 and 24 patients will be assessed by the above methods for improvement Doses will be selected from a traditional 3+3 design, and selected as the top two-doses that lack dose-limiting toxicity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method for treating sarcopenia in a subject comprising administering to the subject a therapeutically effective amount of a polypeptide comprising an Insulin-like growth factor 2 (IGF-2) amino acid sequence, wherein the subject has a degenerative muscle loss disorder comprising a sarcopenia.

22. The method of claim 21, further comprising increasing proliferation of myoblasts in the subject.

23. The method of claim 21, further comprising increasing proliferation of myoblasts in the subject by at least 20%.

24. The method of claim 21, wherein the subject is at least 70 years old.

25. The method of claim 21, wherein the polypeptide comprising the IGF-2 amino acid sequence comprises at least 90% identity to amino acids 25-91 of SEQ ID NO: 11.

26. The method of claim 21, wherein the polypeptide comprising the IGF-2 amino acid sequence comprises at least 95% identity to amino acids 25-91 of SEQ ID NO: 11.

27. The method of claim 21, wherein the polypeptide comprising the IGF-2 amino acid sequence comprises at least 98% identity to amino acids 25-91 of SEQ ID NO: 11.

28. The method of claim 21, wherein the subject has low muscle mass as measured by a DXA, a low gait speed, a SPPB score less than or equal to nine, or weighs less than 35 kg.

29. The method of claim 28, wherein the subject has low muscle mass as measured by at least two of the following: a DXA, a low gait speed, a SPPB score less than or equal to nine, or weighs less than 35 kg.

30. The method of claim 28, wherein the subject has low muscle mass as measured by a DXA, a low gait speed, a SPPB score less than or equal to nine, and weighs less than 35 kg.

31. The method of claim 21, wherein the polypeptide is administered according to a dosage schedule to the subject weekly, twice weekly, monthly, twice monthly, once every three weeks, or once every four weeks.

32. A method for treating cachexia in a subject comprising administering to the subject a therapeutically effective amount of a polypeptide comprising an Insulin-like growth factor 2 (IGF-2) amino acid sequence, wherein the subject has a muscle wasting disorder comprising a cachexia.

33. The method of claim 32, wherein the cachexia is caused by malnutrition, cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), Crohn's disease, untreated/severe type 1 diabetes mellitus, anorexia nervosa, chemotherapy, muscular dystrophy or other genetic diseases which cause immobility, or hormonal deficiencies.

34. The method of claim 32, wherein the cachexia is a result of a cancer.

35. The method of claim 32, wherein the cachexia is a result of AIDS.

36. The method of claim 32, further comprising increasing proliferation of myoblasts in the subject.

37. The method of claim 32, further comprising increasing proliferation of myoblasts in the subject by at least 20%.

38. A method for treating muscular dystrophy in a subject comprising administering to the subject a therapeutically effective amount of a polypeptide comprising an Insulin-like growth factor 2 (IGF-2) amino acid sequence, wherein the subject has a muscle wasting disorder comprising a muscular dystrophy.

39. The method of claim 38, further comprising increasing proliferation of injury activated myoblasts in the subject.

40. The method of claim 38, further comprising increasing proliferation of injury activated myoblasts in the subject by at least 20%.
